(19) United States
(12) Patent Application Publication
(10) Pub. No.: US 2022/0220215 A1
(43) Pub. Date: Jul. 14, 2022

(54) BINDING MOLECULES

(71) Applicant: CRESCENDO BIOLOGICS LIMITED, Cambridge (GB)

(72) Inventors: Carrie ENEVER, Cambridge (GB); Colette JOHNSTON, Cambridge (GB); James LEGG, Cambridge (GB); Pavel PISA, Cambridge (GB); Martyna LEWANDOWSKA, Cambridge (GB); Iain SCOTT, Cambridge (GB)

(21) Appl. No.: 17/610,330

(22) PCT Filed: May 15, 2020
(86) PCT No.: PCT/GB2020/051201

§ 371 Date: Nov. 10, 2021

(30) Foreign Application Priority Data
May 15, 2019 (GB) 1906870.9
May 15, 2019 (GB) 1906872.5

(51) Int. Cl. C07K 16/28; C07K 16/30; A61P 35/00

(52) U.S. Cl. CPC C07K 16/2878; C07K 16/2818; C07K 16/3069; A61P 35/00; C07K 2317/21; C07K 2317/31; C07K 2319/31; C07K 2317/565; C07K 2317/569; C07K 2317/622; C07K 2317/76; C07K 2317/92; C07K 2317/94; C07K 2317/33

(57) ABSTRACT

The disclosure relates to multispecific agents that simultaneously bind CD137 and PD-1 and which comprise a single domain antibody specific to CD137 and a moiety that binds PD-1, for example a single domain antibody. The disclosure also relates to therapeutic and diagnostic applications of such agents, for example in the treatment of a cancer.

### INTRODUCTION

Cancer remains one of the leading causes of death in the world. Recent studies have shown an estimated 12.7 million cancer cases worldwide. This number is expected to increase to 21 million by 2030 (Vinay and Kwon 2014).

CD137 (4-1BB, TNFRS9) is a type 1 transmembrane glycoprotein belonging to the TNF receptor superfamily. It was originally cloned by Kwon et al (1989) from the cDNA of activated murine T cells. It has subsequently been shown to have a broad immune cell expression pattern found on T cells, B cells, NK and NK T cells, dendritic cells (DC), macrophages, neutrophils and eosinophils. Expression has also been reported on non-haematopoetic cells, for example epithelial, endothelial and smooth muscle cells and on tumour cell lines. CD137 expression is mainly activation induced, although low level constitutive expression has been demonstrated on some cell types including Tregs and DC's.

The 255 amino acid human CD137 protein (Genbank accession NP_001552) consists of a 17 amino acid signal peptide sequence, an extracellular region containing four cysteine rich domains, a 27 amino acid transmembrane region and a short 42 amino acid intracellular domain. It exists as both a monomer and dimer on the cell surface. The main ligand for CD137 is CD137 ligand (CD137L, 4-1BB-L, TNFS9), although interactions with galectin-9 which facilitates receptor aggregation (Madireddi et al 2014) and matrix proteins such as fibronectin (Chalupny et al, 1992) have also been reported. CD137 ligand is predominantly expressed on activated antigen presenting cells such as dendritic cells, B-cells and macrophages.

Interaction of the trimeric CD137 ligand with CD137 results in clustering of the receptor and recruitment of signalling molecules such as the TRAF family of proteins leading to kinase modulation and activation of the NfKB pathway. Thus, clustering of CD137 is crucial for initiation and regulation of downstream signalling.

Studies using agonist anti CD137 monoclonal antibodies in vitro and in vivo have shown that upon activation CD137 is rapidly internalised into an endosomal compartment termed the ‘signalosome’ from which it keeps signalling (reviewed in Sanchez-Paulete et al 2016).

Co-stimulatory TNFR family members such as CD137, CD27, OX40 (CD134), HVEM, CD30, and GITR are involved in sustaining the T cell responses after initial T-cell activation. In CD4+ and CD8+ T cells, CD137 acts as a costimulatory receptor that modulates T-cell receptor (TCR) mediated signalling. Ligation of CD137 together with TCR activation promotes proliferation, cytokine production, and inhibits apoptosis through induction of anti-apoptotic B-cell lymphoma-extra large (Bcl-xl) and B-cell lymphoma 2 (Bcl-2) pathways. Cross-linking of CD137 on NK cells has been shown to stimulate IFN-gamma secretion and proliferation. Dendritic cell responses to CD137 stimulation include enhanced maturation and antigen presentation and secretion of cytokines IL-6, IL12- and IL-27 and enzymes such as indoleamine-2,3-dioxygenase (IDO) which can modulate T-cell function. CD137 can also upregulate intercellular adhesion molecule 1 (ICAM1) and vascular cell adhesion molecule 1 (VCAM1) on tumor vascular endothelium, thus inducing effector cell migration and retention of the activated T-cells in the tumor microenvironment.

Cross linking of CD137 by anti CD137 antibodies has been shown to have potent anti-tumour effects in vivo in a number of models including sarcoma, mastocytoma, glioma, lymphoma, myeloma, and hepatocellular carcinoma. CD8+ cell depletion studies have demonstrated that this effect primarily involves cytolytic T cell expansion and infiltration resulting in tumour cell lysis. However, contributions of other types of cells such as DCs, NK-cells or CD4+ T-cells have been reported in some tumour models. Furthermore, anti CD137 therapy has been shown to trigger an immunologic memory response and to inhibit autoimmune reactions (reviewed in Vinay et al 2012).

It has been shown that existing agonistic therapies result in systemic CD137 effects leading to unwanted side effects. Activation of CD137 signalling has been associated with severe toxicity in murine models. Clinical trials of a fully human IgG4 anti CD137 agonistic antibody (Urelumab®, BMS-663513) reported neutropenia, elevated liver enzymes and at high doses severe hepatic toxicity resulting in trial termination. This severe toxicity has not been observed for a fully human IgG2 (PF-05082566) that is also in clinical trials both as a monotherapy and in combination therapy approaches.

Agonistic antibodies targeting co-stimulatory TNFRs have been shown to require engagement of FcγRs (Bulliard et al). Thus, non-targeted clustering via FcγRs may influence the mechanism by which agonistic antibodies act on these targets.

The Programmed Death 1 (PD-1) protein is encoded by the PDCD1 gene and expressed as a 55 kDa type I transmembrane protein (Agata 1996 Int Immunol 8(5):765-72). PD-1 is an immunoglobulin superfamily member (Ishida 1992 EMBO 11(11):3887-95) and it is an inhibitory member of the extended CD28/CTLA-4 family of T cell regulators. Other members of this family include CD28, CTLA-4, ICOS and BTLA. PD-1 exists as a monomer, lacking the unpaired cysteine residue characteristic of other CD28 family members (Zhang 2004 Immunity 20:337-47). Its cytoplasmic domain contains an immunoreceptor tyrosine-based inhibitory motif (ITIM) and an immunoreceptor tyrosine-based switch motif (ITSM) that are phosphorylated during signal transduction (Riley 2009 Immunol Rev 229(1):114-25).

PD-1 is expressed on B cells, T cells, and monocytes (Agata 1996). The role of PD-1 in maintaining immunologic self-tolerance was demonstrated in PDCD1−/− mice, which develop autoimmune disorders (Nishimura 1999 Immunity 11:141-51, Nishimura 2001 Science 291(5502):319-22). The PD-1 pathway therefore regulates antigen responses, balancing autoimmunity and tolerance.

There are two ligands for PD-1 that mediate its regulatory function. PD-L1 (B7-H1) is normally expressed on dendritic cells, macrophages, resting B cells, bone marrow-derived mast cells and T cells as well as non-hematopoietic cell lineages (reviewed in Francisco 2010 Immunol Rev 236:219-42). PD-L2 (B7-DC) is largely expressed on dendritic cells and macrophages (Tseng 2001 J Exp Med 193(7):839-45). Ligand expression is influenced by local mediators and can be upregulated by inflammatory cytokines.

PD-1 is known as an immunoinhibitory protein that negatively regulates TCR signals. The interaction between PD-1 and PD-L1 can act as an immune checkpoint, which can lead to, e.g., a decrease in tumour infiltrating lymphocytes, a decrease in T-cell receptor mediated proliferation, and/or immune evasion by cancerous cells. Immune suppression can be reversed by inhibiting the local interaction of PD-1 with PD-L1 or PD-L2; the effect is additive when the interaction of PD-1 with both PD-L1 and PD-L2 is blocked.

As T cells become activated and co-stimulated by antigen-presenting cells (APCs), T cell expression of PD-1 is induced. PD-1 engagement with ligand on the APC cross-links PD-1 and clusters it into the T cell receptor (TCR) complex within the immunological synapse (Yokosuka 2012 J Exp Med 209(9):1201-17). Within the T cell cytoplasm, PD-1 signalling domains ITIM and ITSM are phosphorylated. This induces Src-homology-2 domain-containing tyrosine phosphatase (SHP1/2) that attenuates various components of the T cell receptor (TCR) signalling. T cell activation is dampened, which leads to a reduction in cytokine response, proliferation and cytolytic activity. This downregulation of T cell function serves to prevent overstimulation, tolerising cells against weakly immunogenic self-antigen.

The PD-1 pathway can be exploited in cancer or infection, whereby tumours or viruses can evade effective immune recognition and T cells demonstrate an ‘exhausted’ phenotype. PD-L1 has also been shown to be expressed in many tumour types including urothelial, ovarian, breast, cervical, colon, pancreatic, gastric, melanoma, glioblastoma and non-small cell lung carcinoma (reviewed in Callahan 2014 J Leukoc Biol 94(1):41-53). The cytokines produced by cancer stromal cells can further upregulate PD-L1 in the tumour microenvironment (He, 2015 Nature Scientific Reports 5:13110). As a result, tumour-specific T cells become unresponsive through PD-1 signalling and therefore fail to eliminate their target. T regulatory cells (T regs) have also been shown to express high levels of PD-1 and they suppress the anti-tumour response further (Lowther 2016 JCI Insight 1(5):85935).

Disruption of the PD-1:PD-L1 interaction enhances T cell activity. An anti-PD-1 monoclonal antibody demonstrates blockade of the interaction between PD-1 and its ligands (Wang 2014 Cancer Immunol Res 2(9):846-56). T cell function in-vitro can be enhanced by PD-1 blockade, as demonstrated by improved proliferation and cytokine responses in mixed lymphocyte reactions of T cells and dendritic cells. cytotoxic lymphocytes (CTLs) derived from melanoma patients has also been shown to be enhanced by PD-1 blockade in vitro using the antibody OPDIVO (nivolumab), and can become resistant to Treg suppression (Wang 2009 Int Immunol 21(9):1065-1077). This antibody has been tested in clinical dose escalation studies in melanoma, non-small cell lung carcinoma (NSCLC), renal cell cancer (RCC) and others. It shows improved overall survival rates compared to chemotherapy in NSCLC patients. Another PD-1 blocking antibody, KEYTRUDA® (pembrolizumab), demonstrates responses in NSCLC patients refractory to CTLA-4 blockade. OPDIVO® and KEYTRUDA® both functionally block the interaction of human PD-1 with its ligands.

It is possible to induce PD-1 signalling by cross-linking it on the membrane with a combination of anti-PD-1 plus anti-CD3 antibodies (Bennett 2003 J Immunol 170:711-18, Keir 2005 J Immunol 175:7372-7379). This function could be detrimental during an anti-tumour response because T cell activity would be suppressed. If suppression of T cell responses were desired, agonistic anti-PD-1 antibodies or those with effector functions could be used to treat immune-related diseases such as rheumatoid arthritis.

Single domain antibodies that bind PD-1 are described in WO2018/127709 and WO2018/127710 incorporated herein by reference.

In light of the toxicity profile observed with existing therapies, there is a need for alternative cancer therapies based on the use of alternative CD137 binding molecules that have reduced toxicity. In particular, there is a clinical need for targeted CD137 agonists that effectively engage CD137 on the surface of cells and have reduced toxicity, including liver toxicity.

The invention addresses the need for alternative antibody-based treatments for use in the treatment of a cancer.

### SUMMARY

The invention relates to novel antibody binding molecules with specificity for both CD137 and PD-1. The inventors have identified single variable heavy chain domain antibodies that bind to CD137 and inhibit binding of CD137L to CD137. They do not cause CD137 signalling when bound to CD137 in monospecific format, that is without being linked to another moiety that binds a second target. However, when linked to a moiety that binds a tumor specific antigen, the single variable heavy chain domain antibodies elicit CD137 signalling. Thus, whilst the single variable heavy chain domain antibodies that bind to CD137 do not induce clustering of the receptor and do not have agonistic activity when bound to CD137 without a binding partner that targets a second antigen, the dual engagement of CD137 and PD-1 in a bispecific molecule leads to CD137 agonism. The single variable heavy chain domain antibodies that bind to CD137 can therefore be used as a subunit in a multispecific binding molecule that simultaneously engages CD137 and PD-1.

Bi- and multispecific molecules described herein bind to CD137 and PD-1 and simultaneously engage both targets. This dual engagement results in CD137 activation, thus restricting the site of action to a target site and potentially minimising undesirable effects of existing therapies.

In one aspect, there is provided an isolated binding molecule comprising or consisting of

a) an antibody or fragment thereof that binds to CD137 and
b) an antibody or fragment thereof that binds to PD-1.

In one aspect, there is provided an isolated binding molecule comprising or consisting of

a) a single variable heavy chain domain antibody that binds to CD137 and
b) a moiety that binds to PD-1.

In one embodiment, an antibody fragment, i.e. a single variable heavy (V_{H}) domain antibody is used i.e. a V_{H} domain antibody that binds to CD137 is combined with a V_{H} domain antibody that binds PD-1. As explained elsewhere in some embodiments, the molecule such comprises or consists of two V_{H} domain antibodies, it does not comprise any other parts of an antibody; e.g. of and antibody that binds PD-1 and CD137 respectively. As explained elsewhere, an additional moiety to extend half life may be included.

Thus, also provided is an isolated binding molecule comprising

a) a single variable heavy chain domain antibody that binds to CD137 and
b) a single variable heavy chain domain antibody that binds PD-1.

In the aspects of the invention, the single variable heavy chain domain antibody that binds to CD137 does not cause CD137 signalling when bound to CD137 as a monospecific entity.

In the aspects above, the moiety, e.g. an antibody, antibody fragment, single variable heavy chain domain antibody that binds to PD-1 is a moiety, antibody, antibody fragment, such as a single variable heavy chain domain antibody. In one embodiment, this binding moiety binds to PD-1 and does not block PD-1 function when used in on its own (i.e. when not formatted as a multispecific molecule with CD137).

Surprisingly, the inventors have found that although non-blocking PD-1 single variable heavy chain domain antibody as described herein have no effect on PD-1 function when they are used as monospecific entities, when formatted together with a moiety that binds to CD137, the resulting bispecific molecule does have an effect on PD-1 signalling. It was observed that less PD-1 is detected on the cell surface. Without wishing to be bound by theory, this reduction of PD-1 on the cell surface may be due to downregulation of PD-1 surface expression, or internalisation and/or degradation of PD-1, or cleavage of PD-1, or downmodulation of PD-1 activity conferred by the bispecific binding molecule.

In another embodiment, the moiety, e.g. antibody, antibody fragment, single variable heavy chain domain antibody of the multispecific binding molecule that binds to PD-1, binds to PD-1 and blocks PD-1 function, e.g. by blocking the interaction of PD-1 with one of its ligands.

The invention also relates to novel V_{H} single domain antibodies that bind CD137 and do not cause CD137 signalling when bound to CD137 as a monospecific entity. These can be used in multispecific molecules for example with a moiety that binds PSMA or a moiety that binds PD-1.

Thus, in one aspect, the invention relates to an isolated binding molecule comprising

a) a single variable heavy chain domain antibody that binds to CD137 and
b) a single variable heavy chain domain antibody that binds PD-1
wherein the single variable heavy chain domain antibody that binds to CD137 does not cause CD137 signalling when bound to CD137 as a monospecific entity.

In another aspect, the invention relates to a pharmaceutical composition comprising a binding molecule described herein and a pharmaceutical carrier.

In another aspect, the invention relates to a binding molecule as described herein or a pharmaceutical composition as described herein for use in the treatment of disease.

In another aspect, the invention relates to a method for treating cancer comprising administering a therapeutically effective amount of a binding molecule as described herein or a pharmaceutical composition as described herein.

In another aspect, the invention relates to a nucleic acid molecule comprising a nucleic acid sequence encoding the binding molecule as described herein.

In another aspect, the invention relates to a vector comprising nucleic acid molecule as described herein.

In another aspect, the invention relates to a host cell comprising a nucleic acid molecule a vector as described herein.

In another aspect, the invention relates to a method for producing a binding molecule as described herein comprising expressing a nucleic acid encoding said binding molecule in a host cell and isolating the binding molecule from the host cell.

In another aspect, the invention relates to a kit comprising a binding molecule or a pharmaceutical composition as described herein.

In another aspect, the invention relates to a use of a binding molecule or a pharmaceutical composition as described herein for simultaneously activating downstream signalling pathways of CD137 and PD-1.

In another aspect, the invention relates to a method for co-stimulating downstream signalling pathways of CD137 and PD-1 comprising administering a binding molecule or a pharmaceutical composition as described herein.

In another aspect, the invention relates to a use of a binding molecule or a pharmaceutical composition as described herein for inducing a local T cell response in or in the vicinity of a PD-1 positive cell or tissue.

In another aspect, the invention relates to V_{H} single domain antibody that binds to CD137 having a set of CDRs 1, 2 and 3 selected from table 7 and/or which comprises a full length sequence as listed in table 7.

In another aspect, the invention relates to multispecific protein comprising a V_{H} single domain antibody that binds to CD137 having a set of CDRs 1, 2 and 3 selected from table 7 and/or which comprises a full length sequence as listed in table 7.

In another aspect, the invention relates to the multispecific protein above that binds to CD137 having a set of CDRs 1, 2 and 3 selected from table 7 and/or which comprises a full length sequence as listed in table wherein said protein binds to PSMA or PD-1.

In another aspect, the invention relates to binding molecule selected from Table 12.

### FIGURES

The invention is further described in the following non-limiting figures.

FIG. 1 shows that monovalent V_{H}, did not increase reporter gene activity and thus do not have agonistic CD137 activity in this assay. A—CD137 Reporter assay activity in the presence of PD1 expressing cells and CD137-PD-1 bispecific V_{H}-V_{H} molecule. Bispecific molecules activated the Jurkat reporter cells in the presence of PD-1 expressing cells. B—However, when co-cultured parental (non-PD-1) expressing cells, there was no activation of the Jurkat reporter cells.

FIG. 2 CD137 Reporter assay activity in the presence of PSMA expressing cells and CD137-PSMA bispecific V_{H}-V_{H} molecule. Bispecific molecules activated the Jurkat reporter cells in the presence of PSMA expressing cells.

FIG. 3 shows that antagonistic activity of a PD1 mAb in a PD1/PDL1 reporter assay was not impacted by the presence of the bispecific CD137-PD1 V_{H}-V_{H} molecule.

FIG. 4 Induction of IL-2 production by PBMCs following stimulation by SEB in the presence of TPP-972 and TPP-983. A—shows the response in donor cells CB149; B—shows the response in donor cells CB122.

FIG. 5 shows the binding of a panel of bispecific CD137-PD1 Humabody® V_{H} to (FIG. 5A) a CHO-PD1 cell line and (FIG. 5)—a CHO-CD137 cell line

FIG. 6 shows that the binding of a panel of CD137-PD1 bispecific Humabody® V_{H} is unaffected by the presence of Pembrolizumab. This demonstrates that they do not compete for the same epitope on PD1.

FIG. 7 NanoBiT® Cell based Protein Interaction Assay demonstrating that a panel of CD137-PD1 bispecific Humabody® V_{H} can bind both PD-1 and CD137 receptors simultaneously on the same cell. Traces are representative of n=2 similar experiments except for TPP-1410 for which n=1.

FIG. 8 Binding of labelled bispecific CD137-PD1 Humabody® V_{H}, TPP-1246 and TPP-1290 to Human CD8+ T cells. TPP-827 is a negative control Humabody® V_{H}.

FIG. 9 A Activity of a panel of CD137-PD1 bispecific Humabody® V_{H} in the CD137 Jurkat Reporter assay in the presence of CHO-PD1 cells. B Comparison of functional activity between CD137-PD1 bispecific Humabody® V_{H} containing either 4V_{H} (TPP-1438 & TPP-1439) comprising 2×PD1 V_{H} or 3V_{H} (TPP-1246 & TPP-1290) comprising 1×PD1 V_{H} in the CD137 Jurkat Reporter assay in the presence of CHO-PD1 cells.

FIG. 10 Enhancement of IL-2 production in Human CD8+ T cells when co-cultured with PD-1 expressing cells in the presence of plate bound anti CD3 antibody. CD137-PD1 bispecific Humabody® V_{H} (TPP-1246, 1290 and 1293) and Urelumab analog were compared to monovalent control Humabody® V_{H} (TPP-447, 1224 and 996). Representative graph showing a single donor.

FIG. 11 Induction of IFN-γ release from anti-CD3 stimulated PBMCs by CD137-PD1 bispecific Humabody® V_{H} and Urelumab analog. TPP-827 and IgG4 are Humabody® V_{H} and IgG negative controls respectively. Error bars represent the standard deviation of two measurements from the same donor.

FIG. 12 CD137-PD-1 bispecific Humabody® V_{H} induce IL-2 production in repetitively stimulated human T cells in a functional co-culture assay with autologous DC in the presence of SEB.

FIGS. 13 A and B. CD137×PD-1 Humabody® V_{H} downregulate PD-1 expression and increase CD137 expression in human repetitively stimulated CD8+(13A) and CD4+(13B) T cells in a functional co-culture assay with autologous DC in the presence of SEB. The decrease in % positive cells detected with Pembrolizumab analog for PD1+ cells and Urelumab analog for CD137+ cells is due to epitope competition with the detection antibodies. Pembrolizumab and Urelumab analogs were therefore used for gating only in the PD1+ and CD137+ populations, respectively.

FIGS. 14 A and B showing the downregulation of PD-1 expression by CD137-PD-1 bispecific Humabody® V_{H} in a dose-dependent manner on CD8+(FIG. 14A) and CD8− cells (FIG. 14B).

FIG. 15 CD137-PD1 bispecific Humabody V_{H}, TPP-1246 and TPP-1290 stimulate a dose dependent increase in IFNγ secretion compared to negative control Humabody® V_{H} TPP-827 in Exhausted T-cells.

FIG. 16 Serum levels after a single i.v. administration of TPP-1246 at 3 mg/kg in cynomolgus macaque.

### DETAILED DESCRIPTION OF THE EMBODIMENTS

The embodiments of the invention will now be further described. In the following passages, different embodiments are described. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols 1 and 2, Ontermann and Dubel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients. Suitable assays to measure the properties as set out above are also described in the examples.

The term “antibody” as used herein broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule. Also encompassed are scaffolds.

In a full-length antibody, each heavy chain is comprised of a heavy chain variable region or domain (abbreviated herein as HCVR) and a heavy chain constant region. The heavy chain constant region is comprised of three domains, C_{H}1, C_{H}2 and C_{H}3. Each light chain has a light chain variable region or domain (abbreviated herein as LCVR) and a light chain constant region. The light chain constant region is comprised of one domain, CL.

The heavy chain and light chain variable regions can be further subdivided into regions of hypervariability, termed complementarity determining regions (CDR), interspersed with regions that are more conserved, termed framework regions (FR). Each heavy chain and light chain variable region is composed of three CDRs and four FRs, arranged from amino-terminus to carboxy-terminus in the following order: FR1, CDR1, FR2, CDR2, FR3, CDR3, FR4.

Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

The Kabat Complementarity Determining Regions (CDRs) are based on sequence variability and are the most commonly used (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk J. Mol. Biol. 196:901-917 (1987)). The Kabat numbering system is generally used when referring to a residue in the variable domain (approximately residues 1-107 of the light chain and residues 1-113 of the heavy chain). Another system is the ImMunoGeneTics (IMGT) numbering scheme. The IMGT numbering scheme is described in Lefranc et al., Dev. Comp. Immunol., 29, 185-203 (2005).

The system described by Kabat is used herein. The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion.

A chimeric antibody is a recombinant protein that contains the variable domains including the complementarity determining regions (CDRs) of an antibody derived from one species, preferably a rodent antibody, while the constant domains of the antibody molecule are derived from those of a human antibody.

A humanized antibody is a recombinant protein in which the CDRs from an antibody from one species; e.g., a rodent antibody, are transferred from the heavy and light variable chains of the rodent antibody into human heavy and light variable domains (e.g., framework region sequences). The constant domains of the antibody molecule are derived from those of a human antibody. In certain embodiments, a limited number of framework region amino acid residues from the parent (rodent) antibody may be substituted into the human antibody framework region sequences.

The term “antigen binding site” refers to the part of the antibody or antibody fragment that comprises the area that specifically binds to an antigen. An antigen binding site may be provided by one or more antibody variable domains. An antigen binding site is typically comprised within the associated V_{H} and V_{L} of an antibody or antibody fragment.

An antibody fragment is a portion of an antibody, for example as F(ab′)2, Fab, Fv, scFv, heavy chain, light chain, heavy (V_{H}), variable light (V_{L}) chain domain and the like. Functional fragments of a full length antibody retain the target specificity of a full antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs. scFv fragments (˜25 kDa) consist of the two variable domains, V_{H} and V_{L}. Naturally, V_{H} and V_{L} domain are non-covalently associated via hydrophobic interaction and tend to dissociate. However, stable fragments can be engineered by linking the domains with a hydrophilic flexible linker to create a single chain Fv (scFv).

The smallest antigen binding fragment is the single variable fragment, namely the single variable heavy (V_{H}) or single variable light (V_{L}) chain domain. V_{H} and V_{L} domains respectively are capable of binding to an antigen. Binding to a light chain/heavy chain partner respectively or indeed the presence of other parts of the full antibody is not required for target binding. The antigen-binding entity of an antibody, reduced in size to one single domain (corresponding to the V_{H} or V_{L} domain), is generally referred to as a “single domain antibody” or “single immunoglobulin variable domain”. A single domain antibody (˜12 to 15 kDa) thus consists of either the V_{H} or V_{L} domain, but it does not comprise other parts of a full length antibody. Single domain antibodies derived from camelid heavy chain only antibodies that are naturally devoid of light chains as well as single domain antibodies that have a human heavy chain domain have been described (Muyldermans 2001, Holliger 2005). Antigen binding single V_{H} domains have also been identified from, for example, a library of murine V_{H} genes amplified from genomic DNA from the spleens of immunized mice and expressed in E. coli (Ward et al., 1989, Nature 341: 544-546). Ward et al. named the isolated single V_{H} domains “dAbs” for “domain antibodies.” The term “dAb” or “sdAb” generally refers to a single immunoglobulin variable domain (V_{H}, V_{HH} or V_{L}) polypeptide that specifically binds antigen. Such a molecule only has the V_{H} or V_{L} binding domain respectively, but does not comprise other parts of a full length antibody. Unless otherwise specified, as used herein, the term refers to a single domain antibody that has a V_{H} domain. For use in therapy, human single domain antibodies are preferred, primarily because they are not as likely to provoke an immune response when administered to a patient.

The terms “single domain antibody”, “V_{H} domain antibody”, “single V_{H} domain antibody”, “V_{H} single domain antibody”, “single variable domain”, “single variable domain antibody”, “single variable heavy chain domain antibody” or immunoglobulin single variable domain (ISV)” are thus all well known in the art and describe the single variable fragment of an antibody that binds to a target antigen. These terms are used interchangeably herein. These terms above and specifically “single heavy chain domain antibody”, “single variable heavy chain domain antibody” “single V_{H} domain antibody”, “ISV”, and “V_{H} single domain” as used herein describe a part of an antibody, i.e. the single heavy chain variable fragment of an antibody, e.g. the V_{H} domain, which retains binding specificity to the antigen in the absence of light chain or other antibody fragments. Such a molecule, e.g. a single variable heavy chain domain antibody is capable of binding to an antigen in the absence of light chain. A single variable heavy chain domain antibody does not comprise other parts of a full length antibody; it only includes the V_{H} domain. Thus, as used herein, these terms and specifically a single domain antibody, specify a binding moiety that is solely made up of the V_{H} domain and does not have other parts of an antibody. As explained herein, the CD137 binding entity illustrated below is a V_{H} single domain antibody and in preferred embodiments, the PD-1 binding entity is also a V_{H} single domain antibody.

As explained below, the embodiments relate to isolated binding molecules which comprise or consist of a single variable heavy chain domain antibody/immunoglobulin single variable heavy chain domain which bind a CD137 antigen and also comprise a moiety that binds to PD-1. Thus, the single variable heavy chain domain antibody (i.e. a V_{H} domain) is capable of binding to CD137 in the absence of light chain. Human single variable heavy chain domain antibodies (“V_{H} domain antibody”) are particularly preferred. Such binding molecules are also termed Humabody® herein. Humabody® is a registered trademark of Crescendo Biologics Ltd.

The term “isolated” refers to a moiety that is isolated from its natural environment. For example, the term “isolated” refers to a single domain antibody or binding molecule that is substantially free of other single domain antibodies or binding molecule, antibodies or antibody fragments. Moreover, an isolated single domain antibody may be substantially free of other cellular material and/or chemicals.

Each V_{H} domain antibody comprises three CDRs and four FRs, arranged from amino-terminus to carboxy-terminus in the following order: FR1-CDR1-FR2-CDR2-FR3-CDR3-FR4. Thus, in one embodiment of the invention, the domain is a human variable heavy chain (V_{H}) domain with the following formula FR1-CDR1-FR2-CDR2-FR3-CDR3-FR4.

Modifications to the C or N-terminal V_{H} framework sequence may be made to the single domain antibodies of the invention to improve their properties. For example, the V_{H} domain may comprise C or N-terminal extensions. C-terminal extensions can be added to the C-terminal end of a V_{H} domain which terminates with the residues VTVSS (SEQ ID NO: 1881).

In one embodiment, the single domain antibodies used in the invention/the binding molecules of the invention comprise C-terminal extensions of from 1 to 50 residues, for example 1 to 10, e.g. 1, 2, 3, 4, 5, 6, 7, 8, 9 or 10, 1-20, 1-30 or 1-40 additional amino acids. In one embodiment, the single domain antibodies of the invention comprise additional amino acids of the human C_{H}1 domain thus that the C terminal end extends into the C_{H}1 domain. For example, C-terminal extensions may comprise neutral, nonpolar amino acids, such as A, L, V, P, M, G, I, F or W or neutral polar amino acids, such as S or T. C-terminal extensions may also be selected from peptide linkers or tags. Additional C or N-terminal residues can be peptide linkers that are for example used to conjugate the single domain antibodies of the invention to another moiety, or tags that aid the detection of the molecule. Such tags are well known in the art and include for, example, linker His tags, e.g., hexa-His ((SEQ ID NO: 1866) or myc tags.

As used herein, the term “homology” or “identity” as used herein generally refers to the percentage of amino acid residues in a sequence that are identical with the residues of the reference polypeptide with which it is compared, after aligning the sequences and in some embodiments after introducing gaps, if necessary, to achieve the maximum percentage homology, and not considering any conservative substitutions as part of the sequence identity. “Homology” or “identity” are used interchangeably herein. Thus, the percentage homology between two amino acid sequences is equivalent to the percentage identity between the two sequences and where the term sequence percentage homology is used, this can be replaced with sequence percentage identity. Neither N- or C-terminal extensions, tags or insertions shall be construed as reducing identity or homology. Methods and computer programs for the alignment are well known. The percentage identity between two amino acid sequences can be determined using well known mathematical algorithms.

According to some embodiments of the various aspects of the invention, the variable domain of the single domain antibodies as described herein is a human variable domain (as used herein V_{H} refers to a human domain), a camelid variable domain (V_{HH}), a humanised V_{HH} domain, a camelized V_{H} domain, a sequence modified V_{H} or V_{HH} domain. In one embodiment, the variable domain of the single domain antibodies as described herein is a V_{H} domain.

As used herein, a human V_{H} domain includes a fully human or substantially fully human V_{H} domain. As used herein, the term human V_{H} domain also includes V_{H} domains that are isolated from heavy chain only antibodies made by transgenic mice expressing fully human immunoglobulin heavy chain loci, in particular in response to an immunisation with an antigen of interest, for example as described in WO2016/062990 incorporated herein by reference and in the examples below. In one embodiment, a human V_{H} domain can also include a V_{H} domain that is derived from or based on a human V_{H} domain amino acid or produced from a human V_{H} nucleic acid sequence. Thus, the term human V_{H} domain includes variable heavy chain regions derived from or encoded by human germline immunoglobulin sequences and for example obtained from heavy chain only antibodies produced in transgenic mice expressing fully human V_{H} genes. In some embodiments, a substantially human V_{H} domain or V_{H} domain that is derived from or based on a human V_{H} domain may include amino acid residues not encoded by human germline immunoglobulin sequences (e.g., mutations introduced in vitro, e.g. by random or site-specific mutagenesis, or introduced by somatic mutation in vivo). The term “human V_{H} domain” therefore also includes a substantially human V_{H} domain, i.e. human V_{H} domain wherein one or more amino acid residue has been modified, for example to remove sequence liabilities. For example, a substantially human V_{H} domain the V_{H} domain may include up to 10, for example 1, 2, 3, 4, 5, 6, 7, 8, 9 or 10 or up to 20 amino acid modifications compared to a germline human sequence.

However, the term “human V_{H} domain” or “substantially human V_{H} domain”, as used herein, is not intended to include antibodies in which CDR sequences derived from the germline of another mammalian species, such as a mouse, have been grafted onto human framework sequences. In one embodiment, the term “human V_{H} domain”, as used herein, is also not intended to include camelized V_{H} domains, that is human V_{H} domains that have been specifically modified, for example in vitro by conventional mutagenesis methods to select predetermined positions in the V_{H} domains sequence and introduce one or more point mutation at the predetermined position to change one or more predetermined residue to a specific residue that can be found in a camelid V_{HH} domain.

The term “KD” refers to the “equilibrium dissociation constant” and refers to the value obtained in a titration measurement at equilibrium, or by dividing the dissociation rate constant (Koff) by the association rate constant (Kon). “KA” refers to the affinity constant. The association rate constant, the dissociation rate constant and the equilibrium dissociation constant are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® assay can be used.

The term “specific binding” or “specifically binds to” or is “specific for” a particular polypeptide or an epitope on a particular polypeptide target as used in this disclosure can be exhibited, for example, by a molecule having a KD for the target of at least about 10-6 M, alternatively at least about 10-7 M, alternatively at least about 10-8 M, alternatively at least about 10-9 M, alternatively at least about 10-10 M, alternatively at least about 10-11 M, alternatively at least about 10-12 M, or greater affinity. In one embodiment, the term “specific binding” refers to binding where a molecule binds to a particular polypeptide or epitope on a particular polypeptide without substantially binding to any other polypeptide or polypeptide epitope.

In some embodiments, there is provided binding molecule that comprises a V_{H} single domain antibody that is a variant of any of the single V_{H} domain antibodies described herein having one or more amino acid substitutions, deletions, insertions or other modifications, and which retains a biological function of the single domain antibody. Thus, variant V_{H} single domain antibody can be sequence engineered. Modifications may include one or more substitution, deletion or insertion of one or more codons encoding the single domain antibody or polypeptide that results in a change in the amino acid sequence as compared with the native sequence V_{H} single domain antibody or polypeptide. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 25, for example 1 to 5, 1 to 10, 1 to 15 or 1 to 20 amino acids, for example 1, 2, 3, 4, 5, 6, 7, 8, 9 or 10 amino acids. The variation allowed may be determined by systematically making insertions, deletions or substitutions of amino acids in the sequence and testing the resulting variants for activity exhibited by the full-length or mature native sequence. A variant of a V_{H} single domain antibody described herein has at least 50%, for example at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence homology to the non-variant molecule, preferably at least 90%, 91%, 92%, 93%, 94%, 95%, 96%, 97%, 98% or 99% sequence homology.

In one embodiment, the modification is a conservative sequence modification. As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an sdAb of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of a single domain antibody of the invention can be replaced with other amino acid residues from the same side chain family and the altered antibody can be tested for retained function (i.e., CD137 binding) using the functional assays described herein.

Thus, these amino acid changes can typically be made without altering the biological activity, function, or other desired property of the polypeptide, such as its affinity or its specificity for antigen. In some instances these changes are made to improve the affinity of the antibody, e.g., single V_{H} domain antibody, for its target antigen. In general, single amino acid substitutions in nonessential regions of a polypeptide do not substantially alter biological activity. Furthermore, substitutions of amino acids that are similar in structure or function are less likely to disrupt the polypeptides' biological activity. Abbreviations for the amino acid residues that comprise polypeptides and peptides described herein, and conservative substitutions for these amino acid residues are shown in Table 1 below.

TABLE 1
Amino Acid Residues and Examples of
Conservative Amino Acid Substitutions

| Original residue | |
|---|---|
| Three letter code, | Conservative |
| single letter code | substitution |
| Alanine, Ala, A | Gly, Ser |
| Arginine, Arg, R | Lys, His |
| Asparagine, Asn, N | Gln, His |
| Aspartic acid Asp, D | Glu, Asn |
| Cysteine, Cys, C | Ser, Ala |
| Glutamine, Gln, Q | Asn |
| Glutamic acid, Glu, E | Asp, Gln |
| Glycine, Gly, G | Ala |
| Histidein, His, H | Asn, Gln |
| Isoleucine, Ile, I | Leu, Val |
| Leucine, Leu, L | Ile, Val |
| Lysine, lys, K | Ar, His |
| Methionine, Met, M | Leu, Ile, Tyr |
| Phenylalanine, Phe, F | Tyr, Met, Leu |
| Proline, Pro, P | Ala |
| Serine, Ser, S | Thr |
| Threonine, Thr, T | Ser |
| Tryptophan, Trp, W | Tyr, Phe |
| Tyrosine, Tyr, Y | Try, Phe |
| Valine, Val, V | Ile, Leu |

In some embodiments, the binding molecule includes a V_{H} single domain antibody that binds to CD137 and is a variant of a single domain antibody selected from those shown in Tables 2, 3 and 4 and 7 or that binds to PD-1 as shown in tables 8, 9 or 10 or 11 that comprises one or more sequence modification and has improvements in one or more of a property such as binding affinity, specificity, thermostability, expression level, effector function, glycosylation, reduced immunogenicity, or solubility as compared to the unmodified single domain antibody.

A skilled person will know that there are different ways to identify, obtain and optimise the antigen binding molecules as described herein, including in vitro and in vivo expression libraries. This is further described in the examples. Optimisation techniques known in the art, such as display (e.g., ribosome and/or phage display) and/or mutagenesis (e.g., error-prone mutagenesis) can be used. The invention therefore also comprises sequence optimised variants of the single domain antibodies described herein.

In one embodiment, modifications can be made to decrease the immunogenicity of the single domain antibody. For example, one approach is to revert one or more framework residues to the corresponding human germline sequence. More specifically, a single domain antibody that has undergone somatic mutation may contain framework residues that differ from the germline sequence from which the single domain antibody is derived. Such residues can be identified by comparing the single domain antibody framework sequences to the germline sequences from which the single domain antibody is derived. In one embodiment, all framework residues are germline sequence.

To return one or more of the amino acid residues in the framework region sequences to their germline configuration, the somatic mutations can be “backmutated” to the germline sequence by, for example, site-directed mutagenesis or PCR-mediated mutagenesis.

Another type of framework modification involves mutating one or more residues within the framework region, or even within one or more CDR regions, to remove T cell epitopes to thereby reduce the potential immunogenicity of the antibody.

In still another embodiment, the glycosylation is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for the antigen.

In one embodiment, the one or more substitution is in the CDR1, 2 or 3 region. For example, there may be 1, 2, 3, 4 or 5 amino acid substitutions in the CDR1, 2 or 3. In another example, there may be 1 or 2 amino acid deletions. In one embodiment, the one or more substitution is in the framework region. For example, there may be 1 to 10 or more amino acid substitutions in the CDR1, 2 or 3. In another example, there may be 1 to 10 or more amino acid deletions.

The term “epitope” or “antigenic determinant” refers to a site on the surface of an antigen (e.g., PD-1 or CD137) to which an immunoglobulin, antibody or antibody fragment, including a V_{H} single domain antibody specifically binds. Generally, an antigen has several or many different epitopes and reacts with many different antibodies. The term specifically includes linear epitopes and conformational epitopes. Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antibody fragment (i.e., epitope mapping) are well known in the art and include, for example, immunoblotting and immunoprecipitation assays, wherein overlapping or contiguous peptides from are tested for reactivity with a given antibody or antibody fragment. An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in different formats, using either labelled antigen or labelled antibody.

The inventors have surprisingly identified single variable heavy chain domain antibodies that, when targeted to CD137 in a monospecific format, that is without being linked to another moiety specific to a second antigen, bind specifically to CD137, but do not induce clustering of the CD137 receptor. Binding of the single variable heavy chain domain antibodies described herein in a monovalent or monospecific format does therefore not activate CD137 signalling and does not lead to CD137 signalling. Binding of the single variable heavy chain domain antibodies described herein does not agonise CD137 signalling unless they are provided together with another moiety specific to a second antigen, for example as a bispecific fusion protein wherein a single variable heavy chain domain antibody described herein is linked to another moiety that binds PD-1.

When a single variable heavy chain domain antibody as described herein is provided as part of a binding molecule, for example as a fusion protein together with a moiety that binds to PD-1, such as a single variable heavy chain domain antibody that binds to PD-1, binding to CD137 and PD-1 results in clustering of the CD137 receptor and CD137 signalling. Induction of CD137 signalling thus requires dual engagement of both targets, i.e. CD137 and PD-1. This enables CD137 activation on cells that express CD137 and PD-1 and/or on CD137 expressing cells in proximity to cells expressing PD-1. The binding molecules effectively engage CD137 on the surface of cells through mechanisms other than binding to Fc-receptors thus also avoiding unwanted toxicities, including liver toxicity. Simultaneously, they engage cells that express PD-1. The bispecific molecule targets CD137 to PD-1 positive cell and CD137 is thus activated at a localised site, not globally. The bispecific molecule can engage both targets in ‘trans’ on different cells but also in ‘cis; on the same cell.

In one embodiment, the PD-1 moiety is a molecule that binds to PD-1 and does not provide blocking of the PD-1 pathway. Such molecules can be used in combination with PD-1 Abs to achieve dual pathway modulation (PD-1 antagonism and CD137 agonism) and can be used together with another PD-1 therapy as further described herein.

When the single variable heavy chain domain antibody that binds to CD137 is combined, in a single molecule, with a moiety that binds PD-1 and blocks PD-1 signalling/function, this results in blockade of the PD-1 pathway. This enables PD-1 blockade preferentially on PD-1 CD137 double positive cells. This can result in an additive or synergistic effect on T-cell activation as a result of PD-1 blockade and CD137 agonism.

When the single variable heavy chain domain antibody that binds to CD137 is combined, in a single molecule, with a moiety that binds PD-1, but does not block PD-1 signalling/function when used in on its own, this results in blockade of the PD-1 pathway. This can result in an additive or synergistic effect on T-cell activation as a result of PD-1 blockade and CD137 agonism.

Advantages of targeting of both CD137 and PD-1 as described herein also include that targeting is to the activated tumour-infiltrating lymphocytes in the tumour microenvironment agnostic of tumour type. This would provide pan-tumour targeting compared to agonism through a tumour associated antigen. For these particular CD137 and PD-1 combinations which do not antagonise the PD-1/PDL1 interaction, there is also an advantage that it could be used in combination with existing therapeutic options (e.g. anti-PD1 mAbs) or directly following treatment if a patient is or becomes non-responsive without interference from a circulating PD-1 long half life Ab (PD-1 binding site will not be blocked by a PD-1 mAb with long-lived receptor occupancy).

We describe a binding molecule that binds to both CD137 and PD-1. The terms “binding molecule” and “binding agent” are used interchangeably herein. A binding molecule as used herein refers to a binding molecule that specifically binds at least two targets, i.e. CD137 and PD-1, wherein one subunit/entity/moiety that binds to CD137 is conjugated/linked a second subunit/entity/moiety that binds PD-1. As described herein, in some embodiments, the binding molecule is a fusion protein wherein one polypeptide that binds to CD137 is conjugated/linked to a second polypeptide that binds to PD-1.

In one aspect, the invention relates to an isolated multispecific binding molecule that binds to both CD137 and PD-1 and comprises a single variable heavy chain domain antibody that binds to CD137. In some embodiments, the single variable heavy chain domain antibody that binds to CD137 is as described herein.

The properties of the multispecific binding molecules of the invention can be exploited in therapeutic methods and uses as well as in pharmaceutical formulations as described herein.

In one aspect, the invention relates to an isolated binding molecule comprising or consisting of

a) a single variable heavy chain domain antibody that binds to CD137 and
b) a moiety that binds to PD-1 that binds to PD-1 but does not block PD-1 binding to its ligand.

In one aspect, the invention relates to an isolated binding molecule comprising or consisting of

a) a single variable heavy chain domain antibody that binds to CD137 and
b) a moiety that binds to PD-1 and blocks PD-1 binding to its ligand.

In one embodiment, as explained elsewhere, the single variable heavy chain domain antibody that binds to CD137 does not cause CD137 signalling when bound to CD137 as a monospecific entity. In one embodiment, the single variable heavy chain domain antibody binds to PD-1 binds PD-1, but does not block PD-1 function, for example binding to its ligand(s) when used in on its own. In one embodiment, the single variable heavy chain domain antibody binds to PD-1 and blocks PD-1 binding to its ligand(s).

Thus, the entity that binds to CD137 is not a full antibody that comprises light and heavy chains, but a single variable heavy chain domain, i.e. a single V_{H} domain antibody only. In some embodiments, the single variable heavy chain domain antibody that binds to CD137 is selected from one of the single variable heavy chain domain antibodies that bind to CD137 having a sequences as described herein as listed in the tables below (tables 2, 3 and 4) wherein the CDRs are defined according to Kabat.

The single variable heavy chain domain antibody that binds to CD137 and the moiety that binds to PD-1 are linked, for example by a peptide linker. For example, the single variable heavy chain domain antibody that binds to CD137 can be linked at its N or C terminus to the moiety that binds to PD-1.

The moiety that binds PD-1 can be selected from an antibody, antibody mimetic, antibody scaffold, antibody fragment or other polypeptide. An antibody fragment can be selected from a portion of an antibody, for example a F(ab′)2, Fab, Fv, scFv, heavy chain, light chain, heavy or variable light chain domain heavy or variable light chain domain, or part thereof, such as a CDR.

In one embodiment, the entity that binds PD-1 is a single variable heavy chain domain antibody that binds to PD-1, such as a human single V_{H} domain antibody. In some embodiments, the single variable heavy chain domain antibody that binds to PD-1 is selected from one of the single variable heavy chain domain antibodies that bind to PD-1 having a sequence as described herein and in the tables below.

In one embodiment, there is provided a binding molecule comprising or consisting of

a) a single variable heavy chain domain antibody that binds to CD137 and
b) a single variable heavy chain domain antibody that binds to PD-1, e.g. one that blocks or does not block PD-1 function.

Thus, preferably, the binding molecule comprises two single variable heavy chain domains, one that binds to CD137 and one that binds to PD-1 and optionally another moiety as described herein, such as a half life extending moiety. No other domains/chains of a full antibody; e.g. domains/chains of a full antibody that bind to CD137 and PD-1 respectively are present. In some embodiments, the single variable heavy chain domain antibody that binds CD137 and the single variable heavy chain domain antibody that binds PD-1 is a human V_{H} domain antibody.

In one aspect, we provide a binding molecule comprising a single human variable heavy chain domain antibody that binds to CD137, for example having a sequence as described herein (e.g. as set out in table 2, 3, 4 and 7), linked to another moiety that binds to PD-1 (e.g. as set out in table 8, 9 and 10 or in table 11), for example having a sequence as described herein wherein the binding molecule exhibits one or more of the following properties/is capable of:

(a) binds to human CD137 with a KD as measured in the examples;
(b) does not inhibit the interaction between PD-1 and its ligand, e.g. PDL-1 and/or PDL-2 or inhibits the interaction between PD-1 and its ligand;
(c) does not bind to mouse CD137;
(d) binds to cells expressing CD137 and simultaneously binds to cells expressing PD-1;
(e) stimulates IL-2 production from SEB activated PBMCs, for example as measured in the examples;
(f) binds simultaneously to CD137 and PD-1;
(g) binds to cyno CD137;
(h) has agonistic CD137 activity due to dual targeting, i.e. agonism is dependent on the presence of the second target;
i) has in vivo efficacy in a MC38 tumour model in C57BL/6-Pdcd1tm1(PDCD1)Tnfrsf9tm1(TNFRSF9) mice (Crown Bioscience Inc.);
j) downmodulates PD-1 on the cell surface; e.g. when a moiety that binds to PD-1 as e.g. as set out in table 8, 9 or and 10 is used;
k) induces IL-2 release from CD8+ T cells in a co-culture assay with PD-1 cells;
l) induces IFN-γ release from anti-CD3 stimulated PBMCs;
m) induces IL-2 release from PBMCs repetitively stimulated with anti-CD3 and anti-CD28 when co-cultured with autologous DCs in the presence of SEB;
n) induces IFN-γ release in a T cell exhaustion assay;
o) has extended half life in a PK study in genOway human HSA/FcRn Tg mice;
p) has extended half life in PK study in cynomolgus macaque;
q) binds to cells co-expressing CD137 and PD-1 to elicit T cell activation
r) enhances T cell activation and/or
s) blocks PD-1 function.

Surprisingly, the inventors have found that bispecific molecules described herein that comprise a V_{H} single domain antibody that binds to CD137 and a V_{H} single domain antibody that binds to PD-1, where the latter, which when used on its own does not block the PD-1 pathway (i.e. PD-1 function), are capable of downmodulating PD-1 on the cell surface. Without wishing to be bound by theory, this downmodulation may be due to downregulation of PD-1 surface expression, or internalisation and/or degradation of PD1, or cleavage of PD1, or downmodulation of PD-1 activity.

In one embodiment, the binding molecule exhibits more than 1 of the properties above, for example 1, 2, 3, 4, 5, 6, 7, 8, 9, 10, 11, 12, 13, 14, 15, 16, 17, 18, 19 or all of the properties selected from the above list, including any combination of properties. In one embodiment, the binding agent inhibits the interaction between human CD137 ligand and human CD137 expressed on the surface of cells.

In one embodiment, the binding molecule is a fusion protein comprising at least two subunits, i.e. a CD137-binding subunit fused to a PD-1-binding subunit wherein the CD137-binding subunit is a single variable heavy chain domain antibody that binds to CD137. In one embodiment, the binding molecule is a fusion protein comprising a single variable heavy chain domain antibody that binds to CD137 linked to a single variable heavy chain domain antibody that binds to PD-1. The linker is, for example, a peptide linker with GS residues such as (Gly4Ser)n, where n=from 1 to 50, e.g., 1 to 10, 10 to 20, 20 to 30, 30 to 40 or 40 to 50 as further described below.

The binding agent is multispecific, for example bispecific or trispecific. A bispecific molecule binds to 2 different targets. A trispecific molecule binds to 2 different targets. A monovalent molecule has one binding entity. A bivalent molecule has two binding entities which bind to the same or different target.

In one embodiment, the binding molecule comprises a first V_{H} single domain antibody that binds to CD137 (V_{H} (A)) and a second V_{H} single domain antibody (V_{H} (B)) that binds to PD-1 and thus has the following formula: V_{H} (A)-L-V_{H} (B). V_{H} (A) is conjugated to V_{H} (B), that is linked, for example with a peptide linker. L denotes a linker. Alternatively, the order may be V_{H} (B)-L-V_{H} (A).

Each V_{H} comprises CDR and FR regions. Thus, the binding molecule may have the following formula: FR1 (A)-CDR1 (A)-FR2(A)-CDR2(A)-FR3(A)-CDR3(A)-FR4(A)-L-FR1 (B)-CDR1 (B)-FR2(B)-CDR2(B)-FR3(B)-CDR3(B)-FR4(B). The order of the single V_{H} domains A and B is not particularly limited, so that, within a polypeptide of the invention, single variable domain A may be located N-terminally and single variable domain B may be located C-terminally, or vice versa.

The term “peptide linker” as used in the various embodiments described herein refers to a peptide comprising one or more amino acids. A peptide linker comprises 1 to 44 amino acids, more particularly 2 to 20 amino acids. Peptide linkers are known in the art or are described herein. Suitable, non-immunogenic linker peptides are, for example, linkers that include G and/or S residues, (G4S)n, (SG4)n or G4(SG4)n peptide linkers, wherein “n” is generally a number between 1 and 50, e.g., 1, 2, 3, 4, 5, 6, 7, 8, 9 or 10. In one embodiment, the peptide is for example selected from the group consisting of GGGGS (SEQ ID NO: 1867), GGGGSGGGGS (SEQ ID NO: 1868), SGGGGSGGGG (SEQ ID NO: 1869), GGGGSGGGGSGGGG (SEQ ID NO: 1870), GSGSGSGS (SEQ ID NO: 1871), GGSGSGSG (SEQ ID NO: 1872), GGSGSG (SEQ ID NO: 1873), or GGSG (SEQ ID NO: 1874).

In one embodiment, the fusion protein described above is capable of dual, e.g. simultaneous, engagement/binding to CD137 on the surface of effector cells and to PD-1. The dual, e.g. simultaneous binding leads to clustering of the CD137 receptor resulting in CD137 signalling.

In some embodiments, the fusion protein is capable of binding CD137 with an EC50 value that is similar to the EC50 value by which the monovalent single heavy chain domain antibody binds to CD137. In some embodiments, the fusion protein binds CD137 with an EC50 value as shown in the examples.

In some embodiments, the fusion protein may be capable of producing a synergistic effect through dual targeting of the CD137 expressing cell and the PD-1 antigen expressing cell.

In one embodiment, a binding molecule as described herein binds to CD137 with a KD of at least about 10-6 M, alternatively at least about 10-7 M, alternatively at least about 10-8 M, alternatively at least about 10-9 M, alternatively at least about 10-10 M, alternatively at least about 10-11 M, alternatively at least about 10-12 M, or greater affinity as measured according to the methods shown in the examples. In one embodiment, a binding molecule as described herein binds to PD-1 with a KD of at least about 10-6 M, alternatively at least about 10-7 M, alternatively at least about 10-8 M, alternatively at least about 10-9 M, alternatively at least about 10-10 M, alternatively at least about 10-11 M, alternatively at least about 10-12 M, or greater affinity as measured according to the methods shown in the examples. Binding can be measured as in the examples. In some embodiments, the binding molecules of the invention have IC50 and/or EC50 values as further described herein and as shown in the examples. Binding molecules described herein have shown excellent stability.

In another aspect, a nucleic acid molecule encoding a fusion protein described herein is provided. For example, the nucleic acid molecule comprises a nucleic acid encoding a single variable heavy chain domain antibody that binds to CD137 as specified herein and a nucleic acid encoding a single variable heavy chain domain antibody that binds to PD-1 as specified herein. A nucleic acid may comprise DNA or RNA and may be wholly or partially synthetic or recombinantly produced. Reference to a nucleotide sequence as set out herein encompasses a DNA molecule with the specified sequence, and encompasses a RNA molecule with the specified sequence in which U is substituted for T, unless context requires otherwise.

Furthermore, the invention relates to a nucleic acid construct comprising at least one nucleic acid as defined above. The construct may be in the form of a plasmid, vector, transcription or expression cassette.

The invention also relates to an isolated recombinant host cell comprising one or more nucleic acid construct as described above. The host cell may be a bacterial, viral, plant, mammalian or other suitable host cell. In one embodiment, the cell is an E. coli cell. In another embodiment, the cell is a yeast cell. In another embodiment, the cell is a Chinese Hamster Ovary (CHO) cell.

In one embodiment, a method of making the fusion protein as described herein is provided, wherein the method comprises culturing the host cell under conditions suitable for expression of the polynucleotide encoding the fusion protein, and isolating the single domain antibody.

Examples of multispecific molecules are provided herein, for example with reference to Table 12. Also provided below are examples of the CD137-binding subunit and the PD-1 binding subunit of the binding molecule which can each form part of the fusion protein.

### Exemplary Immunoglobulins Included in the Binding Molecule

As described above, the binding molecules comprise a single variable heavy chain domain antibody that binds CD137 (CD137-binding subunit) and a moiety that binds PD-1 (PD-1 binding subunit). For example, the binding molecule can be a fusion protein wherein a single variable heavy chain domain antibody that binds CD137 is linked to another polypeptide that binds to PD-1. The below provides examples of the CD137-binding subunit and the PD-1 binding subunit.

Exemplary Immunoglobulins Included in the Binding Molecule and which Bind CD137

Examples of single variable heavy chain domain antibodies that bind to CD137 and that may form one of the subunits of the binding molecule that bind to both, CD137 and PD-1, are described and can be used in the various embodiments of the invention.

CD137 is an important regulator of immune responses and therefore an important target in cancer therapy. The T cell costimulatory receptor CD137 is induced on activated T cells and plays a variety of crucial roles: preventing activation-induced cell death (AICD), promoting cell cycle progression, enhancing cytotoxicity and the production of type 1 cytokines such as IL-2, IFN-γ, and TNF-α, and increasing the memory CD8+ T cells. In vivo CD137 triggering with agonistic antibodies enhances CD8+ T cell responses against tumors. CD137 mediated anti-cancer effects are based on its ability to induce activation of cytotoxic T lymphocytes (CTL), and among others, high amounts of IFN-γ. CD137/CD137L interactions are also considered positive regulators of CD8+ T cell responses against viruses such as influenza virus, lymphocytic choriomeningitis virus (LCMV), and herpes simplex virus (HSV). CD137 is involved in sustaining the T cell responses after initial T-cell activation.

Importantly, CD137 signalling requires clustering of the CD137 receptor. Such clustering is mediated by the interaction of the trimeric CD137 ligand with the CD137 receptor resulting in recruitment of signalling molecules such as the TRAF family of proteins. This in turn leads to kinase modulation and activation of the Nf-KB signalling pathway. The NF-κB family of transcription factors has an essential role in inflammation and innate immunity. Furthermore, NF-κB is increasingly recognized as a crucial player in many steps of cancer initiation and progression.

Single domain antibodies described herein bind specifically to wild type human CD137 (UniProt Accession No. Q07011, GenBank Accession No. NM_001561). The amino acid sequence and nucleotide sequences for wild type human CD137 are shown in SEQ ID NO: 1875 and SEQ ID NO: 1876.

Unless otherwise specified, the term CD137 as used herein refers to human CD137. CD137 is also known as “4-1BB”, “TNF receptor superfamily member 9”, “TNFRS9”, “induced by lymphocyte activation” and “ILA” these terms are used interchangeably, and include variants, isoforms of human CD137.

The terms “CD137 binding molecule/protein/polypeptide/agent/moiety”, “CD137 antigen binding molecule molecule/protein/polypeptide/agent/moiety”, “anti-CD137 single domain antibody”, “anti-CD137 single immunoglobulin variable domain”, “anti-CD137 heavy chain only antibody” or “anti-CD137 antibody” all refer to a molecule capable of specifically binding to the human CD137 antigen. The binding reaction may be shown by standard methods, for example with reference to a negative control test using an antibody of unrelated specificity.

A multispecific binding agent described herein, “which binds” or is “capable of binding” an antigen of interest, e.g. human CD137, is one that binds the antigen with sufficient affinity such that the antibody is useful as a therapeutic agent in targeting a cell or tissue expressing the antigen CD137. Binding is to the extracellular domain of CD137.

Binding molecules of the invention bind specifically to human CD137. In other words, binding to the CD137 antigen is measurably different from a non-specific interaction. They do not cross react with mouse CD137.

In one embodiment, the sdAb binds to human CD137 and also binds to monkey (e.g., cynomolgous) CD137.

In one aspect, the monovalent single domain antibody used in the multispecific molecule exhibits one or more of the following properties as a monovalent entity (i.e. when it is not provided in a multispecific format together with an entity that binds to PD-1):

(a) binds to human CD137 with a KD as measured in the examples;
(b) binds to cells expressing CD137, but does not bind to cells that do not express CD137. This can be measured in a FMAT assay;
(c) shows minimal cell internalisation;
(d) inhibits the interaction between CD137 ligand and CD137 expressed on the surface of cells. This can be measured in a FMAT assay;
(e) does not activate CD137 signalling in T cells;
(f) does not stimulate IL-2 production from CD8+ cells;
(g) does not bind to mouse CD137;
(h) provides good stability and/or
(i) does not increase reporter gene activity and thus does not elicit CD137 signalling.

### Exemplary Sequence Features

In one aspect, the single variable heavy chain domain antibody comprises a CDR1, CDR2 or CDR3 as shown for one of the single domain antibodies as shown in Table 2, 3, 4 and 7 or having a CDR1, 2 or 3 with at least 75% homology thereto; or a set of CDRs (i.e. CDR1, CDR2, CDR3) wherein said set is as shown for one of the single domain antibodies as shown in Table 2, 3, 4 or 7 or where one or more of the CDRs in the set has at least 75% homology thereto.

Sequence homology with reference to CDR1, 2 or 3 sequences as above and as used generally herein can be at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% for example at least 90%, 91%, 92%, 93%, 94%, 95%, 96%, 97%, 98% or 99% sequence homology. In one example, sequence homology is at least 90%, or 95%. As mentioned elsewhere, homology and identity are used interchangeably herein and the above values also refer to sequence identity.

In another aspect, the single variable heavy chain domain antibody according to the invention comprises or consists of a full length sequence as shown in Table 2, 3, 4 and 7 or a sequence with at least 50% homology thereto. For example, the single variable heavy chain domain antibody has a full length sequence selected from the sequences listed in Table 2.

Sequence homology a full length sequence as above and as used generally herein can be at least 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% for example at least 90%, 91%, 92%, 93%, 94%, 95%, 96%, 97%, 98% or 99% sequence homology. In one example, sequence homology is at least 90%, or 95%.

In one embodiment, the single variable heavy chain domain antibody comprises a CDR1, 2, and 3 sequence as shown for V_{H} single domain antibodies CD137A, B, C or D or comprises or consists of a full length sequence as shown for V_{H} single domain antibodies CD137A, B, C or D (see table 2). In one embodiment, the single variable heavy chain domain antibody comprises a set of CDR1, 2, and 3 sequences as shown for V_{H} single domain antibodies CD137A, B, C or D as shown in table 2 or a sequence with at least 75%, 80%, 90% or 95% sequence homology thereto. Thus, in one embodiment, the V_{H} single domain antibody has a CDR1 with SEQ ID NO. 449 or a sequence with at least 75%, 80%, 90% or 95% sequence homology thereto, a CDR2 with SEQ ID NO. 450 or a sequence with at least 75%, 80%, 90% or 95% sequence homology thereto and a CDR3 with SEQ ID NO. 451 or a sequence with at least 75%, 80%, 90% or 95% sequence homology thereto. In another embodiment, the V_{H} single domain antibody has a CDR1 with SEQ ID NO. 850 or a sequence with at least 75%, 80%, 90% or 95% sequence homology thereto, a CDR2 with SEQ ID NO. 851 or a sequence with at least 75%, 80%, 90% or 95% sequence homology thereto and a CDR3 with SEQ ID NO. 852 or a sequence with at least 75%, 80%, 90% or 95% sequence homology thereto. In another embodiment, the V_{H} single domain antibody has a CDR1 with SEQ ID NO. 854 or a sequence with at least 75%, 80%, 90% or 95% sequence homology thereto, a CDR2 with SEQ ID NO. 855 or a sequence with at least 75%, 80%, 90% or 95% sequence homology thereto and a CDR3 with SEQ ID NO. 856 or a sequence with at least 75%, 80%, 90% or 95% sequence homology thereto. In another embodiment, the V_{H} single domain antibody has a CDR1 with SEQ ID NO. 878 or a sequence with at least 75%, 80%, 90% or 95% sequence homology thereto, a CDR2 with SEQ ID NO. 879 or a sequence with at least 75%, 80%, 90% or 95% sequence homology thereto and a CDR3 with SEQ ID NO. 880 or a sequence with at least 75%, 80%, 90% or 95% sequence homology thereto.

In one embodiment, the single variable heavy chain domain antibody is selected from one of the full length single variable heavy chain domain antibody CD137A, B, C or D as shown in table 2, or from a sequence with at least 75%, 80% or 90% or 95% homology thereto. In one embodiment, the single variable heavy chain domain antibody has a full length sequence as shown for A, B, C or D. Thus, in one embodiment, the V_{H} single domain antibody comprises a sequence selected from SEQ ID Nos: 452, 853, 857 or 881 or a sequence with at least 75%, 80% or 90% or 95% homology thereto.

In one embodiment, the single variable heavy chain domain antibody comprises a CDR1, 2, and 3 as shown for V_{H} single domain antibodies 1.1 to 1.114 or comprises or consists of a full length sequence as shown for V_{H} single domain antibodies 1.1 to 1.114 or a sequence with at least 75%, 80%, 90% or 95% homology thereto.

In one embodiment, the single variable heavy chain domain antibody comprises a CDR1, 2, and 3 as shown for V_{H} single domain antibodies V_{H} 1.107 to 1.114 as shown in table 3 or a sequence with at least 75%, 80%, 90% or 95% homology thereto. In one embodiment, the single variable heavy chain domain antibody comprises a set of CDR1, 2, and 3 sequences as shown for any one of V_{H} single domain antibodies 1.1 to 1.114 or a sequence with at least 75%, 80%, 90% or 95% sequence homology thereto. Thus, the V_{H} single domain antibody has a CDR1 with SEQ ID NO. 1 or a sequence with at least 75%, 80%, 90% or 95% sequence homology thereto, a CDR2 with SEQ ID NO. 2 or a sequence with at least 75%, 80%, 90% or 95% sequence homology thereto and a CDR3 with SEQ ID NO. 3 or a sequence with at least 75%, 80%, 90% or 95% sequence homology thereto; a CDR1 with SEQ ID NO. 5 or a sequence with at least 75%, 80%, 90% or 95% sequence homology thereto, a CDR2 with SEQ ID NO. 6 or a sequence with at least 75%, 80%, 90% or 95% sequence homology thereto and a CDR3 with SEQ ID NO. 7 or a sequence with at least 75%, 80%, 90% or 95% sequence homology thereto, a CDR1 with SEQ ID NO. 9 or a sequence with at least 75%, 80%, 90% or 95% sequence homology thereto, a CDR2 with SEQ ID NO. 10 or a sequence with at least 75%, 80%, 90% or 95% sequence homology thereto and a CDR3 with SEQ ID NO. 11 or a sequence with at least 75%, 80%, 90% or 95% sequence homology thereto and so forth.

In one embodiment, the single variable heavy chain domain antibody is selected from V_{H} 1.107 to 1.114 as shown in table 3, i.e. V_{H} 1.107, 1.108, 1.109, 1.110, 1.111, 1.112, 1.113 or 1.114, or a sequence with at least 75%, 80% 90% or 95% homology thereto. In one embodiment, the single variable heavy chain domain antibody is V_{H} 1.113 or a sequence with at least 75%, 80% 90% or 95% homology thereto. In one embodiment, the single variable heavy chain domain antibody is V_{H} 1.113 or a sequence with at least 75%, 80% 90% or 95% homology thereto.

In one embodiment, the single variable heavy chain domain antibody comprises CDR1, 2, and 3 sequence as shown for V_{H} single domain antibodies 2.1 to 2.51, e.g. 2.41 to 2.51 as shown in table 4 or comprises or consists of a full length sequence as shown for V_{H} single domain antibodies 2.1 to 2.51, e.g. 2.41 to 2.51. In one embodiment, the single variable heavy chain domain antibody comprises a CDR1, 2, and 3 as shown for V_{H} single domain antibodies as shown in table 4 or a sequence with at least 75%, 80% 90% or 95% homology thereto. In one embodiment, the single variable heavy chain domain antibody comprises a set of CDR1, 2, and 3 sequences as shown for any one of V_{H} single domain antibodies 2.1 to 2.51 or a sequence with at least 75%, 80%, 90% or 95% sequence homology thereto. Thus, the V_{H} single domain antibody has a CDR1 with SEQ ID NO. 457 or a sequence with at least 75%, 80%, 90% or 95% sequence homology thereto, a CDR2 with SEQ ID NO. 458 or a sequence with at least 75%, 80%, 90% or 95% sequence homology thereto and a CDR3 with SEQ ID NO. 459 or a sequence with at least 75%, 80%, 90% or 95% sequence homology thereto; a CDR1 with SEQ ID NO. 461, a CDR2 with SEQ ID NO. 462 or a sequence with at least 75%, 80%, 90% or 95% sequence homology thereto or a sequence with at least 75%, 80%, 90% or 95% sequence homology thereto and a CDR3 with SEQ ID NO. 463 or a sequence with at least 75%, 80%, 90% or 95% sequence homology thereto and so forth.

In one embodiment, the single variable heavy chain domain antibody is selected from those shown in table 4 or a sequence with at least 75%, 80% 90% or 95% homology thereto.

TABLE 2
Exemplary sequences for binding molecules that include two single variable heavy chain
domain antibodies, one that binds to CD137 and another that binds to PD-1: exemplary full
length sequences and CDR sequences of V_{H} single domain antibodies that bind to CD137.

| Name- | CDR1 | CDR2 | CDR3 | SEQ ID full VH |
|---|---|---|---|---|
| CD137 | NYWM | NINQDGSER | GGEGYGVDHYGL | EVQLVESGGGLVQPGGSLRLSCAASGFTLSNYWMNWVRQAP |
| A | N SEQ | YYVDSVKG | DV SEQ ID NO. | GKGLEWVANINQDGSERYYVDSVKGRFTISRDNAKNSLYLQMN |
| | ID NO. | SEQ ID NO. | 451 | SLRAEDTAVYYCARGGEGYGVDHYGLDVSGQGTTVTVSS |
| | 449 | 450 | | SEQ ID NO. 452 |
| CD137 | NYGM | VISNDGNTK | DETTTVTASYYFDY | EVQLVESGGGLVQPGGSLRLSCAASGFTFSNYGMWVRQAPG |
| B | H SEQ | TYADSVKG | SEQ ID NO. 852 | KGLEWVAVISNDGNTKTYADSVKGRFTISRDNSKNMLYLQMNS |
| | ID | SEQ ID NO. | | LRTEDTAVYYCAQDETTTVTASYYFDYWGQGTLVTVSS |
| | NO. 850 | 851 | | SEQ ID NO. 853 |
| CD137 | NYGM | VISNDGNTK | DETTTVTASYYFDY | EVQLVESGGGLVQPGGSLRLSCAASGFTFSNYGMHWVRQAPG |
| C | H SEQ | TYADSVKG | SEQ ID NO. 856 | KGLEWVAVISNDGNTKTYADSVKGRFTISRDNSKNTLYLQMNS |
| | ID NO. | SEQ ID NO. | | LRTEDTAVYYCAQDETTTVTASYYFDYWGQGTLVTVSS SEQ ID |
| | 854 | 855 | | NO. 857 |
| CD137 | NYGM | VISNDGNTK | DETTTVTASYYFDY | EVQLVESGGGVVQPGGSLRLSCAASGFTFSNYGMHWVRQAP |
| D | H SEQ | TYADSVKG | SEQ ID NO. 880 | GKGLEWVAVISNDGNTKTYADSVKGRFTISRDNSKNTLYLQMN |
| | ID NO. | SEQ ID NO. | | SLRAEDTAVYYCAQDETTTVTASYYFDYWGQGTLVTVSS SEQ |
| | 878 | 879 | | ID NO. 881 |

TABLE 3
Further exemplary sequences for binding molecules that include two single variable heavy chain
domain antibodies, one that binds to CD137 and another that binds to PD-1 - full length sequences and
CDR sequences of V_{H} single domain antibodies that bind to CD137 (Family 1) shown below

| Name | CDR1 | CDR2 | CDR3 | Full Length |
|---|---|---|---|---|
| 1.1 | SEQ ID NO: | SEQ ID NO: 2 | SEQ ID NO: 3 | SEQ ID NO: 4 |
| | 1 | HIKEDGSEKY | GGDGYSDSHF | EVQLVESGGGLVQPGGSLRLSCAASGFTFSSHWMTWFRQAPGKGLEW |
| | SHWMT | YEDSVEG | GVDV | VAHIKEDGSEKYYEDSVEGRFTVSRDNAKNSVYLQMNSLRAEDTAVYYC |
| | | | | ARGGDGYSDSHFGVDVWGQGTTVTVSS |
| 1.2 | SEQ ID NO: | SEQ ID NO: 6 | SEQ ID NO: 7 | SEQ ID NO: 8 |
| | 5 | HIKEDGSEKY | GGDGYSDSHY | EVQLVESGGGLVQPGGSLRLSCAASGFTFSSYWMTWFRQAPGKGLEW |
| | SYWMT | YVDSVEG | GVDV | VAHIKEDGSEKYYVDSVEGRFTISRDNANNSLYLQMNSLRAEDTAVYYC |
| | | | | ARGGDGYSDSHYGVDVWGQGTTVTVSS |
| 1.3 | SEQ ID NO: | SEQ ID NO: | SEQ ID NO: 11 | SEQ ID NO: 12 |
| | 9 | 10 | GGLGYGDSHY | EVQLVESGGGLVQPGGSLRLSCAASGFTFSSYWMTWFRQAPGRGLEW |
| | SYWMT | NINQDGSEK | GMDV | VANINQDGSEKYYVDSVEGRFTVSRDNAKNSLYLQMNSLRAEDTAVYYC |
| | | YYVDSVEG | | ARGGLGYGDSHYGMDVWGQGTTVTVSS |
| 1.4 | SEQ ID NO: | SEQ ID NO: | SEQ ID NO: 15 | SEQ ID NO: 16 |
| | 13 | 14 | GGLGYGDSHY | EVQLVESGGGLVQPGGSLRLSCAASGFTFSNYWMTWFRQAPGGGLE |
| | NYWMT | NINQDGSEK | GMDV | WVANINQDGSEKYYVDSVEGRFTVSRDNAKNSLYLQMNSLRAEDTAVY |
| | | YYVDSVEG | | YCARGGLGYGDSHYGMDVWGQGTTVTVSS |
| 1.5 | SEQ ID NO: | SEQ ID NO: | SEQ ID NO: 19 | SEQ ID NO: 20 |
| | 17 | 18 | GGDGYSGSHH | EVQLVESGGGLVQPGGSLRLSCAASGFTFSNYWMIWFRQAPGKGLEW |
| | NYWMI | NINQDGSEK | GTDV | VANINQDGSEKYYVDSVEGRFTISRDNAKNSLYLQMNSLRAEDTAVYYC |
| | | YYVDSVEG | | ARGGDGYSGSHHGTDVWGQGTTVTVSS |
| 1.6 | SEQ ID NO: | SEQ ID NO: | SEQ ID NO: 23 | SEQ ID NO: 24 |
| | 21 | 22 | GGEGYSTSHYG | EVQLVESGGGLVQPGGSLRLSCAASGFTFSSYWMSWVRQAPGKGLEW |
| | SYWMS | NIKQDGSEKY | MDV | VANIKQDGSEKYYVDSVKGRFTISRDNAKNSLYLQMNSLRAEDTAVYYC |
| | | YVDSVKG | | ARGGEGYSTSHYGMDVWGQGTTVTVSS |
| 1.7 | SEQ ID NO: | SEQ ID NO: | SEQ ID NO: 27 | SEQ ID NO: 28 |
| | 25 | 26 | GGDGYSDSHF | EVQLVESGGGLVQPGGSLRLSCGASGFTFSSYWMLWFRQAPGKGLEW |
| | SYWML | NINQDGSEK | GTDV | VANINQDGSEKYYVDSVKGRFTISRDNAKNSLYLQMNTLRAEDTAIYYC |
| | | YYVDSVKG | | ARGGDGYSDSHFGTDVWGQGTTVTVSS |
| 1.8 | SEQ ID NO: | SEQ ID NO: | SEQ ID NO: 31 | SEQ ID NO: 32 |
| | 29 | 30 | GGDGYSDSHY | EVQLVESGGGLVQPGGSLRLSCGASGFTFSSYWMFWFRQAPGEGLEW |
| | SYWMF | NINQDGSEK | GTDV | VANINQDGSEKYYVDSVEGRFTISRDNAKNSLYLQMNSLRAEDTAIYYCA |
| | | YYVDSVEG | | RGGDGYSDSHYGTDVWGQGTTVTVSS |
| 1.9 | SEQ ID NO: | SEQ ID NO: | SEQ ID NO: 35 | SEQ ID NO: 36 |
| | 33 | 34 | GGLGYGDSHY | EVQLVESGGGLVQPGGSLRLSCAASGFTFSNYWMTWFRQAPGGGLE |
| | NYWMT | NINQDGSEK | GMDV | WVANINQDGSEKYYVDSVEGRFTVSRDNAKNSLDLQMNSLRAEDTAVY |
| | | YYVDSVEG | | YCARGGLGYGDSHYGMDVWGQGTTVTVSS |
| 1.10 | SEQ ID NO: | SEQ ID NO: | SEQ ID NO: 39 | SEQ ID NO: 40 |
| | 37 | 38 | GGAGYSMSHY | EVQLVESGGGLVQPGGSLRLSCAASGFTFSDYWMNWARQAPGKGLE |
| | DYWMN | NIKEDGSEKY | GMDV | WVANIKEDGSEKYYVDSVEGRFTISRDNAKNSTYLQMNSLRVEDTAVYY |
| | | YVDSVEG | | CARGGAGYSMSHYGMDVWGQGTTVTVSS |
| 1.11 | SEQ ID NO: | SEQ ID NO: | SEQ ID NO: 43 | SEQ ID NO: 44 |
| | 41 | 42 | GGAGYSMSHY | EVQLVESGGGLVQPGGSLRLSCEASGFTFSDYWMNWARQAPGKGLE |
| | DYWMN | NIKEDGSEKY | GMDV | WVANIKEDGSEKYYVDSVEGRFTISRDNAKNSTYLQMNSLRAEDTAVYY |
| | | YVDSVEG | | CARGGAGYSMSHYGMDVWGQGTTVTVSS |
| 1.12 | SEQ ID NO: | SEQ ID NO: | SEQ ID NO: 47 | SEQ ID NO: 48 |
| | 45 | 46 | GGDGYSNSHF | EVQLVESGGGLVQPGGSLRLSCGASGFTFSSYWMLWFRQAPGKGLEW |
| | SYWML | NINQDGSEK | GTDV | VANINQDGSEKYYVDSVKGRFTISRDNAKNSLYLQMNTLRAEDTAIYYC |
| | | YYVDSVKG | | ARGGDGYSNSHFGTDVWGQGTTVTVSS |
| 1.13 | SEQ ID NO: | SEQ ID NO: | SEQ ID NO: 51 | SEQ ID NO: 52 |
| | 49 | 50 | GGDGYSDSHY | EVQLVESGGGLVKPGGSLRLSCGASGFTFSSYWMFWFRQAPGEGLEW |
| | SYWMF | NINQDGSEK | GTDV | VANINQDGSEKYYVDSVEGRFTISRDNAKNSLYLQMNSLRAEDTAIYYCA |
| | | YYVDSVEG | | RGGDGYSDSHYGTDVWGQGTTVTVSS |
| 1.14 | SEQ ID NO: | SEQ ID NO: | SEQ ID NO: 55 | SEQ ID NO: 56 |
| | 53 | 54 | GGEGYSTSHYG | EVQLVESGGGLVQPGGSLRLSCAASGFTFSNYWMNWVRQAPGKGLE |
| | NYWMN | NIKEDGSENY | MDV | WVANIKEDGSENYYVDSVKGRFTISRDNAKNSLYLQMNSLRAEDTAVYY |
| | | YVDSVKG | | CARGGEGYSTSHYGMDVWGQGTTVTVSS |
| 1.15 | SEQ ID NO: | SEQ ID NO: | SEQ ID NO: 59 | SEQ ID NO: 60 |
| | 57 | 58 | GGDGYSDSHF | EVQLVESGGGLVQPGGSLRLSCGASGFTFSTYWMLWFRQAPGKGLEW |
| | TYWML | NINQDGSEK | GTDV | VANINQDGSEKYYVDSVKGRFTISRDNAKNSLSLQMNSLRAEDTATYYC |
| | | YYVDSVKG | | ARGGDGYSDSHFGTDVWGQGTTVTVSS |
| 1.16 | SEQ ID NO: | SEQ ID NO: | SEQ ID NO: 63 | SEQ ID NO: 64 |
| | 61 | 62 | GGDGYSSSHY | EVQLVESGGGLVQPGGSLRLSCVASGFTFSNYWMMWFRQAPGKGLE |
| | NYWMM | NINQDGSEK | GTDV | WVANINQDGSEKYFVDSVEGRFTISRDNAKNSLYLQMNSLRAEDTAVY |
| | | YFVDSVEG | | YCARGGDGYSSSHYGTDVWGQGTTVTVSS |
| 1.17 | SEQ ID NO: | SEQ ID NO: | SEQ ID NO: 67 | SEQ ID NO: 68 |
| | 65 | 66 | GGDSYGYRDY | EVQLVESGGGLVQPGGSLRLSCAASGFTFSSYWMNWVRQAPGKGLEW |
| | SYWMN | NIKEDGSEKY | GMDV | VANIKEDGSEKYYVDSVKGRFTISRDNAKNSLYLQMNSLRAEDTAVYYCA |
| | | YVDSVKG | | RGGDSYGYRDYGMDVWGQGTTVTVSS |
| 1.18 | SEQ ID NO: | SEQ ID NO: | SEQ ID NO: 71 | SEQ ID NO: 72 |
| | 69 | 70 | GGVGYGDSHF | EVQLVESGGGLVQPGGSLRLSCAASGFTFSTHWMNWARQAPGKELE |
| | THWMN | NINQDGSEK | GMDV | WVANINQDGSEKYYVDSVEGRFTISRDNANNSLYLQMNSLRAEDTAVY |
| | | YYVDSVEG | | YCARGGVGYGDSHFGMDVWGLGTTVTVSS |
| 1.19 | SEQ ID NO: | SEQ ID NO: | SEQ ID NO: 75 | SEQ ID NO: 76 |
| | 73 | 74 | GGDDYSNSHY | EVQLVESGGGLVQPGGSLRLSCAASGFTFSSYWMIWVRQAPGKGLEW |
| | SYWMI | NINQDGSEK | GMDV | VANINQDGSEKYYVDSVKGRFTISRDNAKNSLYLQMNSLRAEDTAVYYC |
| | | YYVDSVKG | | ARGGDDYSNSHYGMDVSGQGTTVTVSS |
| 1.20 | SEQ ID NO: | SEQ ID NO: | SEQ ID NO: 79 | SEQ ID NO: 80 |
| | 77 | 78 | GGFGYGDSHY | EVQLVESGGGLVQPGGSLRLSCAASGFTFSSYWMNWVRQAPGKGLEW |
| | SYWMN | NINQDGSEK | GMDV | VANINQDGSEKYYVDSVQGRFTISRDNANNSLYLQMNSLRAEDTAVYYC |
| | | YYVDSVQG | | ARGGFGYGDSHYGMDVWGQGTTVTVSS |
| 1.21 | SEQ ID NO: | SEQ ID NO: | SEQ ID NO: 83 | SEQ ID NO: 84 |
| | 81 | 82 | GGEGYSDSHY | EVQLVESGGGLVQPGGSLRLSCGASGFTFSSYWMFWFRQAPGKELEW |
| | SYWMF | NVNQDGSEK | GTDV | VANVNQDGSEKYYVDSVEGRFTISRDNAKNSLYLQMNSLRAEDTAIYYC |
| | | YYVDSVEG | | ARGGEGYSDSHYGTDVWGQGTTVTVSS |
| 1.22 | SEQ ID NO: | SEQ ID NO: | SEQ ID NO: 87 | SEQ ID NO: 88 |
| | 85 | 86 | GGEGYGDSHY | QVQLVESGGGLVQPGGSLRLSCAASGFTFSNYWMNWVRQAPGKGLE |
| | NYWMN | NIKEDGSEKY | GMDV | WVANIKEDGSEKYYVDSVEGRFTISRDNARNSLYLQMNSLRAEDTAVYY |
| | | YVDSVEG | | CARGGEGYGDSHYGMDVSGQGTTVTVSS |
| 1.23 | SEQ ID NO: | SEQ ID NO: | SEQ ID NO: 91 | SEQ ID NO: 92 |
| | 89 | 90 | GGEGYGDDHY | QVQLVESGGGLVQPGGSLRLSCAASGFTFSSYWMNWVRQAPGKGLE |
| | SYWMN | NIKQDGSEKY | GMDV | WVANIKQDGSEKYYVDSVKGRFTISRDNAKNSLYLQMNSLTAEDTAVYY |
| | | YVDSVKG | | CARGGEGYGDDHYGMDVWGQGTTVTVSS |
| 1.24 | SEQ ID NO: | SEQ ID NO: | SEQ ID NO: 95 | SEQ ID NO: 96 |
| | 93 | 94 | GGEGYGDYHY | QVQLVESGGGLVQPGGSLRLSCAASGFTFSSYWMSWVRQAPGKGLEW |
| | SYWMS | NIKQDGSEKY | GLDV | VANIKQDGSEKYYVDSVKGRFTISRDNAKNSLYLQMNSLRAEDTAVYYC |
| | | YVDSVKG | | ARGGEGYGDYHYGLDVSGQGTTVTVSS |
| 1.25 | SEQ ID NO: | SEQ ID NO: | SEQ ID NO: 99 | SEQ ID NO: 100 |
| | 97 | 98 | GGDSYGYRDY | EVQLVESGGGLVQPGGSLRLSCAASGFTFSSYWMNWVRQAPGKGLEW |
| | SYWMN | NIKQDGSEKY | GMDV | VANIKQDGSEKYYVDSVKGRFTISRDNAKNSLYLQMNSLRAEDTAVYYC |
| | | YVDSVKG | | ARGGDSYGYRDYGMDVWGQGTTVTVSS |
| 1.26 | SEQ ID NO: | SEQ ID NO: | SEQ ID NO: | SEQ ID NO: 104 |
| | 101 | 102 | 103 | EVQLVESGGGLVQPGGSLRLSCVASGFTFSTHWMNWARQAPGKELEW |
| | THWMN | NINQDGSEK | GGVGYGDSHF | VANINQDGSEKYYVDSVEGRFTISRDNANNSLYLQMNSLRAEDTAVYYC |
| | | YYVDSVEG | GMDV | ARGGVGYGDSHFGMDVWGLGTTVTVSS |
| 1.27 | SEQ ID NO: | SEQ ID NO: | SEQ ID NO: | SEQ ID NO: 108 |
| | 105 | 106 | 107 | EVQLVESGGGLVQPGGSLRLSCGASGFTFSSYWMLWFRQAPGEGLEW |
| | SYWML | NINQDGSEK | GGEGYSDSHH | VANINQDGSEKYYVDSVEGRLTISRDNAKNALYLQMNSLRAEDTAIYYC |
| | | YYVDSVEG | GTDV | ARGGEGYSDSHHGTDVWGQGTTVTVSS |
| 1.28 | SEQ ID NO: | SEQ ID NO: | SEQ ID NO: | SEQ ID NO: 112 |
| | 109 | 110 | 111 | EVQLVESGGGLVQPGGSLRLSCAASGFTFSNYWMNWVRQAPGKGLE |
| | NYWMN | NIKQDGSEKY | GGDNYAYRDF | WVANIKQDGSEKYYVDSVKGRFTISRDNAKNSLYLQMNSLRAEDTAVYY |
| | | YVDSVKG | GMDV | CARGGDNYAYRDFGMDVWGQGTTVTVSS |
| 1.29 | SEQ ID NO: | SEQ ID NO: | SEQ ID NO: | SEQ ID NO: 116 |
| | 113 | 114 | 115 | EVQLVESGGGLVQPGGSLRLSCGASGFTFSNYWMFWFRQAPGKELEW |
| | NYWMF | NVNQDGSEK | GGEGYSDSHY | VANVNQDGSEKYYVDSVEGRFTISRDNAKNSLYLQMNSLRAEDTAIYYC |
| | | YYVDSVEG | GTDV | ARGGEGYSDSHYGTDVWGQGTTVTVSS |
| 1.30 | SEQ ID NO: | SEQ ID NO: | SEQ ID NO: | SEQ ID NO: 120 |
| | 117 | 118 | 119 | EVQLVESGGGLVQPGGSLRLSCAASGFTFSSYWMNWVRQAPGKGLEW |
| | SYWMN | NINQDGSEK | GGEEYGSSHYG | VANINQDGSEKYYVDSVKGRFTISRDNAKNSLYLQMNSLRAEDTAVYYC |
| | | YYVDSVKG | MDV | ARGGEEYGSSHYGMDVWGLGTTVTVSS |
| 1.31 | SEQ ID NO: | SEQ ID NO: | SEQ ID NO: | SEQ ID NO: 124 |
| | 121 | 122 | 123 | EVQLVESGGGLVQPGGSLRLSCAASGFTFSSYWMNWVRQAPGKGLEW |
| | SYWMN | NINQDGSEK | GGDSYGYRDY | VANINQDGSEKYYVDSVKGRFTISRDNAKNSLYLQMNSLRAEDTAVYYC |
| | | YYVDSVKG | GMDV | ARGGDSYGYRDYGMDVWGQGTTVTVSS |
| 1.32 | SEQ ID NO: | SEQ ID NO: | SEQ ID NO: | SEQ ID NO: 128 |
| | 125 | 126 | 127 | EVQLVESGGGLVQPGGSLRLSCAASGFTFSSYWMNWVRQTPGKGLEW |
| | SYWMN | NINQNGSEK | GGFGYGDSHY | VANINQNGSEKYYVDSVEGRFNISRDNAKNSLYLQMSSLRAEDTAVYYC |
| | | YYVDSVEG | GMDV | ARGGFGYGDSHYGMDVWGQGTTVTVSS |
| 1.33 | SEQ ID NO: | SEQ ID NO: | SEQ ID NO: | SEQ ID NO: 132 |
| | 129 | 130 | 131 | EVQLVESGGGLVQAGGSLRLSCVASGFTFSNYWMTWFRQAPGKGLEW |
| | NYWMT | NINQDESEEY | GGDGYSDSHY | VANINQDESEEYYVDSVKGRFTISRDNAKNSLFLQMNSLRAEDTAIYYCA |
| | | YVDSVKG | GTDV | RGGDGYSDSHYGTDVWGQGTTVTVSS |
| 1.34 | SEQ ID NO: | SEQ ID NO: | SEQ ID NO: | SEQ ID NO: 136 |
| | 133 | 134 | 135 | QVQLQESGGGLVQPGGSLRLSCTASGFTFSNYWMNWVRQAPGKGLE |
| | NYWMN | NIKEDGSENY | GGEGYSTSHYG | WVANIKEDGSENYYVDSVKGRFTISRDNAKNSLYLQMNSLRAEDTAVYY |
| | | YVDSVKG | MDV | CARGGEGYSTSHYGMDVWGQGTTVTVSS |
| 1.35 | SEQ ID NO: | SEQ ID NO: | SEQ ID NO: | SEQ ID NO: 140 |
| | 137 | 138 | 139 | QVQLVESGGGLVQPGGSLRLSCAASGFTFSNYWMNWVRQAPGKGLE |
| | NYWMN | NIKQDGSEKY | GGEGYGESHY | WVANIKQDGSEKYYVDSVEGRFTISRDNAKNSLYLQMDSLRAEDTAVYY |
| | | YVDSVEG | GMDV | CARGGEGYGESHYGMDVSGQGTTVTVSS |
| 1.36 | SEQ ID NO: | SEQ ID NO: | SEQ ID NO: | SEQ ID NO: 144 |
| | 141 | 142 | 143 | EVQLVESGGGLVQPGGSLRLSCAASGFTFSTYWMNWVRQAPGKGLE |
| | TYWMN | NIKQDGSEKY | GGDSYGYRDY | WVANIKQDGSEKYYVDSVKGRFTISRDNAKNSLYLQMNSLRAEDTAVYY |
| | | YVDSVKG | GMDV | CARGGDSYGYRDYGMDVWGQGTTVTVSS |
| 1.37 | SEQ ID NO: | SEQ ID NO: | SEQ ID NO: | SEQ ID NO: 148 |
| | 145 | 146 | 147 | EVQLVESGGGLVQPGGSLRLSCAASGFTFSYYWMIWFRQAPGEELEWV |
| | YYWMI | NINQDGSEK | GGDGYSNSHF | ANINQDGSEKYYVDSVKGRFIISRDNATNSLFLQMNSLRAEDTAVYYCAR |
| | | YYVDSVKG | GMDV | GGDGYSNSHFGMDVWGQGTTVTVSS |
| 1.38 | SEQ ID NO: | SEQ ID NO: | SEQ ID NO: | SEQ ID NO: 152 |
| | 149 | 150 | 151 | EVQLVESGGGLVQPGGSLRLSCAASGFTFSNYWMIWYRQAPGEELEW |
| | NYWMI | NINQDGSEK | GGEGYSDSHY | VANINQDGSEKYYVDSVKGRFTISRDNAKNSLYLQMNSLRAEDTAIYYCA |
| | | YYVDSVKG | GTDV | RGGEGYSDSHYGTDVWGQGTTVTVSS |
| 1.39 | SEQ ID NO: | SEQ ID NO: | SEQ ID NO: | SEQ ID NO: 156 |
| | 153 | 154 | 155 | EVQLVESGGGLVQPGGSLRLSCAASGFTFSNYWMNWVRQAPGKELE |
| | NYWMN | NINQDESEKY | GGFGYGDSHF | WVANINQDESEKYYVDSVKGRFTVSRDNAKNSLFLQMNSLRADDTAVY |
| | | YVDSVKG | GMDV | YCARGGFGYGDSHFGMDVWGQGTTVTVSS |
| 1.40 | SEQ ID NO: | SEQ ID NO: | SEQ ID NO: | SEQ ID NO: 160 |
| | 157 | 158 | 159 | EVQLVESGGGLVQPGGSLRLSCGASGFTFSNYWMFWFRQAPGKELEW |
| | NYWMF | NVNQDGSEK | GGEGYSDSHY | VANVNQDGSEKYYVDSVEGRFTISRDDAKNSLYLQMNSLRAEDTAIYYC |
| | | YYVDSVEG | GTDV | ARGGEGYSDSHYGTDVWGQGTTVTVSS |
| 1.41 | SEQ ID NO: | SEQ ID NO: | SEQ ID NO: | SEQ ID NO: 164 |
| | 161 | 162 | 163 | EVQLVESGGGLVQPGGSLRLSCGASGFTFSNYWMFWFRQAPGKELEW |
| | NYWMF | NVNQNGSEK | GGEGYSDSHY | VANVNQNGSEKYYVDSVEGRFTISRDNAKNSLYLQMNSLRAEDTAIYYC |
| | | YYVDSVEG | GTDV | ARGGEGYSDSHYGTDVWGQGTTVTVSS |
| 1.42 | SEQ ID NO: | SEQ ID NO: | SEQ ID NO: | SEQ ID NO: 168 |
| | 165 | 166 | 167 | QVQLVESGGGLVQPGGSLRLSCAASGFTFSNYWMNWVRQAPGKGLE |
| | NYWMN | NIKQDGSEKY | GGEGYGDSHY | WVANIKQDGSEKYYVDSVKGRFTISRDNAKDSLYLQMNSLRAEDTAIYY |
| | | YVDSVKG | GMDV | CARGGEGYGDSHYGMDVSGQGTTVTVSS |
| 1.43 | SEQ ID NO: | SEQ ID NO: | SEQ ID NO: | SEQ ID NO: 172 |
| | 169 | 170 | 171 | EVQLVESGGGLVQPGGSLRLSCAASGFTFSNYWMIWYRQAPGEELEW |
| | NYWMI | NINQDGSEK | GGDGYSNSHY | VANINQDGSEKYYVDSVKGRFTISRDNATNSLFLQMNSLRAEDTAVYYC |
| | | YYVDSVKG | GMDV | ARGGDGYSNSHYGMDVWGQGTTVTVSS |
| 1.44 | SEQ ID NO: | SEQ ID NO: | SEQ ID NO: | SEQ ID NO: 176 |
| | 173 | 174 | 175 | EVQLVESGGGLVQPGGSLRLSCAASGFTFSDYWMIWYRQAPGEELEW |
| | DYWMI | NINQDGSEK | GGDGYSNSHY | VANINQDGSEKYYVDSVKGRFTISRDNATNSLFLQMNSLRAEDTAVYYC |
| | | YYVDSVKG | GMDV | ARGGDGYSNSHYGMDVWGQGTTVTVSS |
| 1.45 | SEQ ID NO: | SEQ ID NO: | SEQ ID NO: | SEQ ID NO: 180 |
| | 177 | 178 | 179 | EVQLVESGGGLVQPGGSLRLSCAASGFTFSKYWMIWVRQAPEKGLEW |
| | KYWMI | NINQDGSEK | GGDDYSNSHY | VANINQDGSEKYYVDSVEGRFTISRDNVNNSLYLQMNSLRAEDTAVYYC |
| | | YYVDSVEG | GMDV | ARGGDDYSNSHYGMDVSGQGTTVTVSS |
| 1.46 | SEQ ID NO: | SEQ ID NO: | SEQ ID NO: | SEQ ID NO: 184 |
| | 181 | 182 | 183 | EVQLVESGGGLVQPGGSLRLSCAASGFTFSNYWMSWVRQAPGRGLEW |
| | NYWMS | NINQDGSEK | GGEEYSSSHYG | VANINQDGSEKYYVDSVKGRFTISRDNAKSSLYLQMNSLRAEDTAVYYC |
| | | YYVDSVKG | MDV | ARGGEEYSSSHYGMDVWGQGTTVTVSS |
| 1.47 | SEQ ID NO: | SEQ ID NO: | SEQ ID NO: | SEQ ID NO: 188 |
| | 185 | 186 | 187 | EVQLVESGGGLVQPGGSLRLSCIASGFSFSNYWMNWVRQAPGKGLEW |
| | NYWMN | NIKQDGSEN | GGEGYSTSHYG | VANIKQDGSENYYVDSVKGRFTISRDNAKNSLYLQMNSLRAEDTAVYYC |
| | | YYVDSVKG | MDV | ARGGEGYSTSHYGMDVWGQGTAVTVSS |
| 1.48 | SEQ ID NO: | SEQ ID NO: | SEQ ID NO: | SEQ ID NO: 192 |
| | 189 | 190 | 191 | QVQLVESGGGLVQPGGSLRLSCAASGFTFSSYWMSWVRQAPGKGLEW |
| | SYWMS | NIKQDGSEKY | GGEGYGVDHY | VANIKQDGSEKYYVDSVKGRFTISRDNAKNSLYLQMSSLRAEDTAVYFCA |
| | | YVDSVKG | GLDV | RGGEGYGVDHYGLDVSGQGTTVTVSS |
| 1.49 | SEQ ID NO: | SEQ ID NO: | SEQ ID NO: | SEQ ID NO: 196 |
| | 193 | 194 | 195 | EVQLVESGGGLVQPGGSLRLSCGASGFTFSSYWMLWFRQAPGKELEW |
| | SYWML | NVNQDGSEN | GGEDYGNSHF | VANVNQDGSENYYVDSVEGRFTISRDNAKNSLYLQMHSLRAEDTAVYY |
| | | YYVDSVEG | GMDV | CARGGEDYGNSHFGMDVWGQGTMVIVSS |
| 1.50 | SEQ ID NO: | SEQ ID NO: | SEQ ID NO: | SEQ ID NO: 200 |
| | 197 | 198 | 199 | EVQLVESGGGLVQPGRSLRLSCAASGFTFSNYWMIWYRQAPGEELEWV |
| | NYWMI | NINQDGSEK | GGDGYSNSHY | ANINQDGSEKYYVDSVKGRFTISRDNATNSLFLQMNSLRAEDTAVYYCA |
| | | YYVDSVKG | GMDV | RGGDGYSNSHYGMDVWGQGTTVTVSS |
| 1.51 | SEQ ID NO: | SEQ ID NO: | SEQ ID NO: | SEQ ID NO: 204 |
| | 201 | 202 | 203 | EVQLVESGGGLVKPGGSLRLSCAASGFTFSNYWMIWYRQAPGEELEWV |
| | NYWMI | NINQDGSEK | GGDGYSNSHY | ANINQDGSEKYYVDSVKGRFTISRDNATNSLFLQMNSLRAEDTAVYYCA |
| | | YYVDSVKG | GMDV | RGGDGYSNSHYGMDVWGQGTTVTVSS |
| 1.52 | SEQ ID NO: | SEQ ID NO: | SEQ ID NO: | SEQ ID NO: 208 |
| | 205 | 206 | 207 | EVQLVESGGGLVQIGGSLRLSCAASGFTFSKYWMIWVRQAPEKGLEWV |
| | KYWMI | NINQDGSEK | GGDDYSISHFG | ANINQDGSEKYYVDSVEGRFTISRDNANNSLFLQMNSLRAEDTAVYYCA |
| | | YYVDSVEG | MDV | RGGDDYSISHFGMDVSGQGTRVTVSS |
| 1.53 | SEQ ID NO: | SEQ ID NO: | SEQ ID NO: | SEQ ID NO: 212 |
| | 209 | 210 | 211 | EVQLVESGGGLVQIGGSLRLSCVASGFTFSKYWMIWVRQAPEKGLEWV |
| | KYWMI | NINQDGSEK | GGDDYSHSHY | ANINQDGSEKYYVDSVEGRFTISRDNANNSLYLQMNSLRAEDTAVYYCA |
| | | YYVDSVEG | GMDV | RGGDDYSHSHYGMDVSGQGTTVTVSS |
| 1.54 | SEQ ID NO: | SEQ ID NO: | SEQ ID NO: | SEQ ID NO: 216 |
| | 213 | 214 | 215 | EVQLVESGGGLVQPGGSLRLSCAASGFNFSNYWMNWVRQAPGKELE |
| | NYWMN | NINQDGSEK | GGFGYGDSHY | WVANINQDGSEKYYVDSVKGRFTISRDNAKNSLFLQMNSLRADDTAVY |
| | | YYVDSVKG | GMDV | YCARGGFGYGDSHYGMDVWGQGTTVTVSS |
| 1.55 | SEQ ID NO: | SEQ ID NO: | SEQ ID NO: | SEQ ID NO: 220 |
| | 217 | 218 | 219 | EVQLVESGGGLVQPGGSLRLSCAASGFTFGSYWLNWVRQAPGKGLEW |
| | SYWLN | NINQDGSEN | GGEDYGNSHF | VANINQDGSENYYVDSVEGRFTISRDNAKNSLYLQMHSLRAEDTAVYYC |
| | | YYVDSVEG | GMDV | ARGGEDYGNSHFGMDVWGQGTMVTVSS |
| 1.56 | SEQ ID NO: | SEQ ID NO: | SEQ ID NO: | SEQ ID NO: 224 |
| | 221 | 222 | 223 | QVQLVESGGGLVKPGGSLRLSCAASGFTFSSYWMSWVRQAPGKGLEW |
| | SYWMS | NIKQDGSEKY | GGEGYGVDHY | VANIKQDGSEKYYVDSVKGRFTISRDNAKNSLYLQMSSLRAEDTAVYFCA |
| | | YVDSVKG | GLDV | RGGEGYGVDHYGLDVSGQGTTVTVSS |
| 1.57 | SEQ ID NO: | SEQ ID NO: | SEQ ID NO: | SEQ ID NO: 228 |
| | 225 | 226 | 227 | QVQLVESGGGLVQPGGSLRLSCTASGFTFSDYWMNWVRQAPGKGLE |
| | DYWMN | NIKEDGSEKY | GGEGYGDNHY | WVANIKEDGSEKYYVDSVEGRFTISRDNARNSLYLQMTSLREEDTAMYY |
| | | YVDSVEG | GMDV | CARGGEGYGDNHYGMDVSGQGTTVTVSS |
| 1.58 | SEQ ID NO: | SEQ ID NO: | SEQ ID NO: | SEQ ID NO: 232 |
| | 229 | 230 | 231 | EVQLVESGGGLVQPGGSLRLSCAASGFTFRSYWMNWVRQAPGKEAE |
| | SYWMN | NINQDGSEK | GGPDYGDLHY | WVANINQDGSEKYYVDSVEGRFTISRDNAKNSLFLQMNSLRDEDTAVY |
| | | YYVDSVEG | GMDV | YCARGGPDYGDLHYGMDVWGQGTTVTVSS |
| 1.59 | SEQ ID NO: | SEQ ID NO: | SEQ ID NO: | SEQ ID NO: 236 |
| | 233 | 234 | 235 | QVQLVESGGGLVQPGGSLRLSCAASGFTFSRYWMSWVRQAPGKGLER |
| | RYWMS | NINQDGREK | GGEGYGDYHY | VANINQDGREKYYVDSVKGRFTISRDNAKNSLYLQMNSLRAEDTAVYYC |
| | | YYVDSVKG | GMDV | ARGGEGYGDYHYGMDVSGQGTTVTVSS |
| 1.60 | SEQ ID NO: | SEQ ID NO: | SEQ ID NO: | SEQ ID NO: 240 |
| | 237 | 238 | 239 | EVQLVESGGGLVQPGGSPRLSCAASGFTLSNYWMIWYRQAPGEKLEW |
| | NYWMI | NINQDGSEK | GGDGYSNSHY | VANINQDGSEKYYVDSVKGRFTISRDNATNSLFLQMNSLRAEDTAVYYC |
| | | YYVDSVKG | GMDV | ARGGDGYSNSHYGMDVWGQGTTVTVSS |
| 1.61 | SEQ ID NO: | SEQ ID NO: | SEQ ID NO: | SEQ ID NO: 244 |
| | 241 | 242 | 243 | EVQLVESGGGLVQPGGSLRLSCVASGFNFSNYWMNWVRQAPGKELE |
| | NYWMN | NINQDESEKY | GGFGYGDSHF | WVANINQDESEKYYVDSVKGRFTISRDNAKNSLFLQMNSLRADDTAVY |
| | | YVDSVKG | GMDV | YCARGGFGYGDSHFGMDVWGQGTTVTVSS |
| 1.62 | SEQ ID NO: | SEQ ID NO: | SEQ ID NO: | SEQ ID NO: 248 |
| | 245 | 246 | 247 | EVQLVESGGGLVQPGGSLRLSCAASGFNFSNYWMNWVRQAPGKELE |
| | NYWMN | NINQDESEKY | GGFGYGDSHF | WVANINQDESEKYYVDSVKGRFTIFRDNAKNSLFLQMNSLRADDTAVY |
| | | YVDSVKG | GMDV | YCARGGFGYGDSHFGMDVWGQGTTVTVSS |
| 1.63 | SEQ ID NO: | SEQ ID NO: | SEQ ID NO: | SEQ ID NO: 252 |
| | 249 | 250 | 251 | EVQLVESGGGLVQPGGSLSLSCAASGFTFRSFWMNWVRQAPGKEAEW |
| | SFWMN | NINQDGSEK | GGPDYGDLHY | VANINQDGSEKYYVDSVKGRFTISRDNAKNSLFLQMNSLRAEDTAVYYC |
| | | YYVDSVKG | GMDV | ARGGPDYGDLHYGMDVWGQGTTVTVSS |
| 1.64 | SEQ ID NO: | SEQ ID NO: | SEQ ID NO: | SEQ ID NO: 256 |
| | 253 | 254 | 255 | EVQLVESGGGLVQPGGSLRLSCAASGFTFRSYWMNWVRQAPGKEAE |
| | SYWMN | NINQDGSEK | GGPDYGDLHY | WVANINQDGSEKYYVDSVKGRFTISRDNAKNSLFLQMNSLRDEDTAVY |
| | | YYVDSVKG | GMDV | YCARGGPDYGDLHYGMDVWGQGTTVTVSS |
| 1.65 | SEQ ID NO: | SEQ ID NO: | SEQ ID NO: | SEQ ID NO: 260 |
| | 257 | 258 | 259 | EVQLVESGGGLVQPGGSLRLSCAASGFTFSNYWMIWYRQAPGEELEW |
| | NYWMI | NINQDGSEK | GGEDYGNSHY | VANINQDGSEKYYVDSVKGRFTISRDNAKNSLYLQMHSLRAEDTAVYYC |
| | | YYVDSVKG | GMDV | ARGGEDYGNSHYGMDVWGQGTMVIVSS |
| 1.66 | SEQ ID NO: | SEQ ID NO: | SEQ ID NO: | SEQ ID NO: 264 |
| | 261 | 262 | 263 | EVQLVESGGGLVQPGGSLRLSCAASGFTFSNYWMNWVRQAPGKGLE |
| | NYWMN | NINQDGSER | GGEGYGIDHY | WVANINQDGSERYYVDSVKGRFTISRDNAKSSLYLQMNSLRAEDTAVYF |
| | | YYVDSVKG | GLDV | CARGGEGYGIDHYGLDVSGQGTTVTVSS |
| 1.67 | SEQ ID NO: | SEQ ID NO: | SEQ ID NO: | SEQ ID NO: 268 |
| | 265 | 266 | 267 | QVQLVESGGGLVQPGGSLRLSCAASGFTFSSYWMSWVRQAPGKGLEW |
| | SYWMS | NINQDGSER | GGEGYGVDHY | VANINQDGSERYYVDSVKGRFTISRDNAKSSLYLQMSSLRAEDTAVYFCA |
| | | YYVDSVKG | GLDV | RGGEGYGVDHYGLDVSGQGTTVTVSS |
| 1.68 | SEQ ID NO: | SEQ ID NO: | SEQ ID NO: | SEQ ID NO: 272 |
| | 269 | 270 | 271 | QVQLVESGGGLVQPGGSLRLSCAASGFTFSNYWMIWVRQAPGKGLEW |
| | NYWMI | NINQDGSEK | GGEGYGVDHY | VANINQDGSEKYYVDSVEGRFTISRDNAKSSLYLQMSNLRAEDTAVYFC |
| | | YYVDSVEG | GLDV | ARGGEGYGVDHYGLDVSGQGTTVTVSS |
| 1.69 | SEQ ID NO: | SEQ ID NO: | SEQ ID NO: | SEQ ID NO: 276 |
| | 273 | 274 | 275 | QVQLVESGGGLVQPGGSLRLSCAATGFTLSNYWMNWVRQAPGKGLE |
| | NYWMN | NINQDGSEK | GGTGYGSDHY | WVANINQDGSEKYYVDSVKGRFTISRDNAKNSLFLQMNSLRAEDTAVY |
| | | YYVDSVKG | GMDV | YCARGGTGYGSDHYGMDVSGQGTTVTVSS |
| 1.70 | SEQ ID NO: | SEQ ID NO: | SEQ ID NO: | SEQ ID NO: 280 |
| | 277 | 278 | 279 | EVQLVESGGGLVQPGGSLRLSCAASGFNFSNYWMNWVRQAPGKELE |
| | NYWMN | NINQDGSEN | GGFGYGDSHY | WVANINQDGSENYYVDSVKGRFTISRDNVKNSLFLQMNRLRADDTAVY |
| | | YYVDSVKG | GMDV | YCARGGFGYGDSHYGMDVWGQGTTVTVSS |
| 1.71 | SEQ ID NO: | SEQ ID NO: | SEQ ID NO: | SEQ ID NO: 284 |
| | 281 | 282 | 283 | EVQLVESGGGLVQPGGSLRLSCAASGFTFGNYWMIWVRQAPGKELEW |
| | NYWMI | NINQNGSER | GGADYSNSHY | LANINQNGSERYYVDSVQGRFTISRDNAKNSLYLQMNSLRAEDTAVYYC |
| | | YYVDSVQG | GMDV | ARGGADYSNSHYGMDVSGQGTTVTVSS |
| 1.72 | SEQ ID NO: | SEQ ID NO: | SEQ ID NO: | SEQ ID NO: 288 |
| | 285 | 286 | 287 | QVQLVESGGGLVQPGGSLRLSCAASGFTFSSYWMSWVRQAPGKGLEW |
| | SYWMS | NINQDGSER | GGEGYGVDHY | VANINQDGSERYYVDSVKGRFTISRDNANNSLHLQMSSLRAEDTAVYFC |
| | | YYVDSVKG | GLDV | ARGGEGYGVDHYGLDVSGQGTTVTVSS |
| 1.73 | SEQ ID NO: | SEQ ID NO: | SEQ ID NO: | SEQ ID NO: 292 |
| | 289 | 290 | 291 | EVQLVESGGGLVQPGGSLRLSCAASGFTFRSYWMNWVRQAPGKEAE |
| | SYWMN | NINPDGSEKY | GGPGYGDLHY | WVANINPDGSEKYYVDSVQGRHTISRDNAKNSLFLEMNSLRVEDTALYY |
| | | YVDSVQG | GMDV | CARGGPGYGDLHYGMDVWGQGTTVTVSS |
| 1.74 | SEQ ID NO: | SEQ ID NO: | SEQ ID NO: | SEQ ID NO: 296 |
| | 293 | 294 | 295 | QVQLVESGGGLVQPGGSLRLSCAATGFTLSNYWMNWVRQAPGKGLE |
| | NYWMN | NINQDGSEK | GGEGYGVDHY | WVANINQDGSEKYYVDSVEGRFTISRDNAKSSLYLQMSSLRAEDTAVYF |
| | | YYVDSVEG | GLDV | CARGGEGYGVDHYGLDVSGQGTTVTVSS |
| 1.75 | SEQ ID NO: | SEQ ID NO: | SEQ ID NO: | SEQ ID NO: 300 |
| | 297 | 298 | 299 | QVQLVESGGGLVQPGGSLRLSCAASGFTFSDYYMSWIRQAPGKGLEWV |
| | DYYMS | NINQDGSER | GGEGYGVDHY | ANINQDGSERYYVDSVKGRFTISRDNAKNSLYLQMSSLRAEDTAVYFCA |
| | | YYVDSVKG | GLDV | RGGEGYGVDHYGLDVSGQGTTVTVSS |
| 1.76 | SEQ ID NO: | SEQ ID NO: | SEQ ID NO: | SEQ ID NO: 304 |
| | 301 | 302 | 303 | EVQLVESGGGLVQPGGSLRLSCAATGFTLSNYWMNWVRQAPGKGLE |
| | NYWMN | NINQDGSER | GGEGYGVDHY | WVANINQDGSERYYVDSVKGRFTISRDNAKNSLYLQMSSLRAEDTAVYF |
| | | YYVDSVKG | GLDV | CARGGEGYGVDHYGLDVSGQGTTVTVSS |
| 1.77 | SEQ ID NO: | SEQ ID NO: | SEQ ID NO: | SEQ ID NO: 308 |
| | 305 | 306 | 307 | QVQLVESGGGLVQPGGSLRLSCAASGFTLSNYWMNWVRQAPGKGLE |
| | NYWMN | NINQDGSER | GGEGYGVDHY | WVANINQDGSERYYVDSVKGRFTISRDNAKNSLYLQMSSLRAEDTAVYF |
| | | YYVDSVKG | GLDV | CARGGEGYGVDHYGLDVSGQGTTVTVSS |
| 1.78 | SEQ ID NO: | SEQ ID NO: | SEQ ID NO: | SEQ ID NO: 312 |
| | 309 | 310 | 311 | QVQLVESGGGLVQPGGSLRLSCAATGFTLSNYWMNWVRQAPGKGLE |
| | NYWMN | NINQDGSER | GGEGYGVDHY | WVANINQDGSERYYVDSVKGRFTISRDNAKNSLYLQMSSLRAEDTAVYF |
| | | YYVDSVKG | GLDV | CARGGEGYGVDHYGLDVSGQGTTVTVSS |
| 1.79 | SEQ ID NO: | SEQ ID NO: | SEQ ID NO: | SEQ ID NO: 316 |
| | 313 | 314 | 315 | QVQLVESGGGLVQPGGSLRLSCAATGFTLSNYWMNWVRQAPGKGLE |
| | NYWMN | NINQDGSER | GGEGYGVNHY | WVANINQDGSERYYVDSVKGRFTISRDNAKNSLYLQMSSLRAEDTAVYF |
| | | YYVDSVKG | GLDV | CARGGEGYGVNHYGLDVSGQGTTVTVSS |
| 1.80 | SEQ ID NO: | SEQ ID NO: | SEQ ID NO: | SEQ ID NO: 320 |
| | 317 | 318 | 319 | QVQLVESGGGLVKPGGSLRLSCVASGFTFSDYYMSWIRQAPGKGLEWV |
| | DYYMS | NIKQDGSERY | GGEGYGVDHY | ANIKQDGSERYYVDSVKGRFTISRDNAKSSLYLQMSSLRAEDTAVYFCAR |
| | | YVDSVKG | GLDV | GGEGYGVDHYGLDVSGQGTTVTVSS |
| 1.81 | SEQ ID NO: | SEQ ID NO: | SEQ ID NO: | SEQ ID NO: 324 |
| | 321 | 322 | 323 | QVQLVESGGGLVQPGGSLRLSCAATGFTLSNYWMNWVRQAPGKGLE |
| | NYWMN | NINQDGSER | GGEGYGVDHY | WVANINQDGSERYYVDSVEGRFTISRDNAKSSLYLQMSNLRAEDTAVYF |
| | | YYVDSVEG | GLDV | CARGGEGYGVDHYGLDVSGQGTTVTVSS |
| 1.82 | SEQ ID NO: | SEQ ID NO: | SEQ ID NO: | SEQ ID NO: 328 |
| | 325 | 326 | 327 | QVQLVESGGGLVQPGGSLRLSCAATGFTLSNYWMNWVRQAPGKGLE |
| | NYWMN | NINQDGSER | GGEGYGVDHY | WVANINQDGSERYYVDSVKGRFTISRDNAKSSLYLQMSSLRAEDTAVYF |
| | | YYVDSVKG | GLDV | CARGGEGYGVDHYGLDVSGQGTTVTVSS |
| 1.83 | SEQ ID NO: | SEQ ID NO: | SEQ ID NO: | SEQ ID NO: 332 |
| | 329 | 330 | 331 | QVQLQESGGGLVQPGGSLRLSCAATGFTLSNYWMNWVRQAPGKGLE |
| | NYWMN | NINQDGSER | GGEGYGVDHY | WVANINQDGSERYYVDSVKGRFTISRDNAKNSLYLQMSSLRAEDTAVYF |
| | | YYVDSVKG | GLDV | CARGGEGYGVDHYGLDVSGQGTTVTVSS |
| 1.84 | SEQ ID NO: | SEQ ID NO: | SEQ ID NO: | SEQ ID NO: 336 |
| | 333 | 334 | 335 | QVQLVESGGGLVKPGGSLRLSCAATGFTLSNYWMNWVRQAPGKGLE |
| | NYWMN | NINQDGSER | GGEGYGVDHY | WVANINQDGSERYYVDSVKGRFTISRDNAKNSLYLQMSSLRAEDTAVYF |
| | | YYVDSVKG | GLDV | CARGGEGYGVDHYGLDVSGQGTTVTVSS |
| 1.85 | SEQ ID NO: | SEQ ID NO: | SEQ ID NO: | SEQ ID NO: 340 |
| | 337 | 338 | 339 | QVQLVESGGGLVQPGGSLRLSCAATGFTLSNYWMNWVRQAPGKGLE |
| | NYWMN | NINQDGSER | GGEGYGVDHY | WVANINQDGSERYYVDSVKGRFTISRDNANNSLHLQMSSLRAEDTAVY |
| | | YYVDSVKG | GLDV | FCARGGEGYGVDHYGLDVSGQGTTVTVSS |
| 1.86 | SEQ ID NO: | SEQ ID NO: | SEQ ID NO: | SEQ ID NO: 344 |
| | 341 | 342 | 343 | QVQLGESGGGLVQPGGSLRLSCAATGFTLSNYWMNWVRQAPGKGLE |
| | NYWMN | NINQDGSER | GGEGYGVDHY | WVANINQDGSERYYVDSVEGRFTISRDNAKSSLYLQMSNLRAEDTAVYF |
| | | YYVDSVEG | GLDV | CARGGEGYGVDHYGLDVSGQGTTVTVSS |
| 1.87 | SEQ ID NO: | SEQ ID NO: | SEQ ID NO: | SEQ ID NO: 348 |
| | 345 | 346 | 347 | QVQLVESGGGLVQPGGSLKLSCAATGFTLSNYWMNWVRQAPGKGLE |
| | NYWMN | NINQDGSER | GGEGYGVDHY | WVANINQDGSERYYVDSVKGRFTISRDNAKSSLYLQMSSLRAEDTAVYF |
| | | YYVDSVKG | GLDV | CARGGEGYGVDHYGLDVSGQGTTVTVSS |
| 1.88 | SEQ ID NO: | SEQ ID NO: | SEQ ID NO: | SEQ ID NO: 352 |
| | 349 | 350 | 351 | QVQLQESGGGLVQPGGSLRLSCAATGFTLSNYWMNWVRQAPGKGLE |
| | NYWMN | NINQDGSER | GGEGYGVDHY | WVANINQDGSERYYVDSVKGRFTISRGNAKNSLYLQMSSLRAEDTAVYF |
| | | YYVDSVKG | GLDV | CARGGEGYGVDHYGLDVSGQGTTVTVSS |
| 1.89 | SEQ ID NO: | SEQ ID NO: | SEQ ID NO: | SEQ ID NO: 356 |
| | 353 | 354 | 355 | QVQLVESGGGVVQPGRSLRLSCAASGFTFDDYGMSWVRQAPGKGLE |
| | DYGMS | NINQDGSER | GGEGYGVDHY | WVANINQDGSERYYVDSVKGRFTISRDNAKNSLYLQMSSLRAEDTAVYF |
| | | YYVDSVKG | GLDV | CARGGEGYGVDHYGLDVSGQGTTVTVSS |
| 1.90 | SEQ ID NO: | SEQ ID NO: | SEQ ID NO: | SEQ ID NO: 360 |
| | 357 | 358 | 359 | EVQLVESGGGLVQPGGSLRLSCAASGFTFSSHWMTWVRQAPGKGLEW |
| | SHWMT | HIKEDGSEKY | GGDGYSDSHF | VAHIKEDGSEKYYEDSVEGRFTVSRDNAKNSVYLQMNSLRAEDTAVYYC |
| | | YEDSVEG | GVDV | ARGGDGYSDSHFGVDVWGQGTTVTVSS |
| 1.91 | SEQ ID NO: | SEQ ID NO: | SEQ ID NO: | SEQ ID NO: 364 |
| | 361 | 362 | 363 | EVQLVESGGGLVQPGGSLRLSCAASGFTFSSHWMTWFRQAPGKGLEW |
| | SHWMT | HIKEDGSEKY | GGDGYSDSHF | VAHIKEDGSEKYYVDSVKGRFTVSRDNAKNSVYLQMNSLRAEDTAVYYC |
| | | YVDSVKG | GVDV | ARGGDGYSDSHFGVDVWGQGTTVTVSS |
| 1.92 | SEQ ID NO: | SEQ ID NO: | SEQ ID NO: | SEQ ID NO: 368 |
| | 365 | 366 | 367 | EVQLVESGGGLVQPGGSLRLSCAASGFTFSSHWMTWFRQAPGKGLEW |
| | SHWMT | HIKEDGSEKY | GGDGYSDSHF | VAHIKEDGSEKYYEDSVKGRFTVSRDNAKNSVYLQMNSLRAEDTAVYYC |
| | | YEDSVKG | GVDV | ARGGDGYSDSHFGVDVWGQGTTVTVSS |
| 1.93 | SEQ ID NO: | SEQ ID NO: | SEQ ID NO: | SEQ ID NO: 372 |
| | 369 | 370 | 371 | EVQLVESGGGLVQPGGSLRLSCAASGFTFSSHWMTWFRQAPGKGLEW |
| | SHWMT | HIKEDGSEKY | GGDGYSDSHF | VAHIKEDGSEKYYEDSVEGRFTISRDNAKNSVYLQMNSLRAEDTAVYYCA |
| | | YEDSVEG | GVDV | RGGDGYSDSHFGVDVWGQGTTVTVSS |
| 1.94 | SEQ ID NO: | SEQ ID NO: | SEQ ID NO: | SEQ ID NO: 376 |
| | 373 | 374 | 375 | EVQLVESGGGLVQPGGSLRLSCAASGFTFSSHWMTWFRQAPGKGLEW |
| | SHWMT | HIKEDGSEKY | GGDGYSDSHF | VAHIKEDGSEKYYEDSVEGRFTVSRDNAKNSLYLQMNSLRAEDTAVYYC |
| | | YEDSVEG | GVDV | ARGGDGYSDSHFGVDVWGQGTTVTVSS |
| 1.95 | SEQ ID NO: | SEQ ID NO: | SEQ ID NO: | SEQ ID NO: 380 |
| | 377 | 378 | 379 | EVQLVESGGGLVQPGGSLRLSCAASGFTFSSHWMTWVRQAPGKGLEW |
| | SHWMT | HIKEDGSEKY | GGDGYSDSHF | VAHIKEDGSEKYYVDSVKGRFTISRDNAKNSLYLQMNSLRAEDTAVYYCA |
| | | YVDSVKG | GVDV | RGGDGYSDSHFGVDVWGQGTTVTVSS |
| 1.96 | SEQ ID NO: | SEQ ID NO: | SEQ ID NO: | SEQ ID NO: 384 |
| | 381 | 382 | 383 | EVQLVESGGGLVQPGGSLRLSCAASGFTFSSHWMTWFRQAPGKGLEW |
| | SHWMT | HIKEDGSEKY | GGDGYSDSHF | VAHIKEDGSEKYYVDSVKGRFTISRDNAKNSLYLQMNSLRAEDTAVYYCA |
| | | YVDSVKG | GVDV | RGGDGYSDSHFGVDVWGQGTTVTVSS |
| 1.97 | SEQ ID NO: | SEQ ID NO: | SEQ ID NO: | SEQ ID NO: 388 |
| | 385 | 386 | 387 | EVQLVESGGGLVQPGGSLRLSCAASGFTFSSHWMTWVRQAPGKGLEW |
| | SHWMT | HIKEDESEKY | GGVGYSISHFG | VAHIKEDESEKYYVDSVKGRFTISRDNAKNSLYLQMNSLRAEDTAVYYCA |
| | | YVDSVKG | VDV | RGGVGYSISHFGVDVWGQGTTVTVSS |
| 1.98 | SEQ ID NO: | SEQ ID NO: | SEQ ID NO: | SEQ ID NO: 392 |
| | 389 | 390 | 391 | EVQLVESGGGLVQPGGSLRLSCAASGFTFSSHWMTWVRQAPGKGLEW |
| | SHWMT | HIKEDESEKY | GGEGYSISHFG | VAHIKEDESEKYYVDSVKGRFTISRDNAKNSLYLQMNSLRAEDTAVYYCA |
| | | YVDSVKG | VDV | RGGEGYSISHFGVDVWGQGTTVTVSS |
| 1.99 | SEQ ID NO: | SEQ ID NO: | SEQ ID NO: | SEQ ID NO: 396 |
| | 393 | 394 | 395 | EVQLVESGGGLVQPGGSLRLSCAASGFTFSSHWMTWFRQAPGKGLEW |
| | SHWMT | HIKEDESEKY | GGDGYSDSHF | VAHIKEDESEKYYVDSVKGRFTISRDNAKNSLYLQMNSLRAEDTAVYYCA |
| | | YVDSVKG | GVDV | RGGDGYSDSHFGVDVWGQGTTVTVSS |
| 1.100 | SEQ ID NO: | SEQ ID NO: | SEQ ID NO: | SEQ ID NO: 400 |
| | 397 | 398 | 399 | EVQLVESGGGLVQPGGSLRLSCAASGFTFSSHWMTWFRQAPGKGLEW |
| | SHWMT | HIKEDESEKY | GGDGYSISHFG | VAHIKEDESEKYYVDSVKGRFTISRDNAKNSLYLQMNSLRAEDTAVYYCA |
| | | YVDSVKG | VDV | RGGDGYSISHFGVDVWGQGTTVTVSS |
| 1.101 | SEQ ID NO: | SEQ ID NO: | SEQ ID NO: | SEQ ID NO: 404 |
| | 401 | 402 | 403 | EVQLVESGGGLVQPGGSLRLSCAASGFTFSSHWMTWFRQAPGKGLEW |
| | SHWMT | HIKEGGSEKY | GGDGYSDSHF | VAHIKEGGSEKYYVDSVKGRFTISRDNAKNSLYLQMNSLRAEDTAVYYCA |
| | | YVDSVKG | GVDV | RGGDGYSDSHFGVDVWGQGTTVTVSS |
| 1.102 | SEQ ID NO: | SEQ ID NO: | SEQ ID NO: | SEQ ID NO: 408 |
| | 405 | 406 | 407 | EVQLVESGGGLVQPGGSLRLSCAASGFTFSSHWMTWFRQAPGKGLEW |
| | SHWMT | HIKEEGSEKY | GGDGYSDSHF | VAHIKEEGSEKYYVDSVKGRFTISRDNAKNSLYLQMNSLRAEDTAVYYCA |
| | | YVDSVKG | GVDV | RGGDGYSDSHFGVDVWGQGTTVTVSS |
| 1.103 | SEQ ID NO: | SEQ ID NO: | SEQ ID NO: | SEQ ID NO: 412 |
| | 409 | 410 | 411 | EVQLVESGGGLVQPGGSLRLSCAASGFTFSSHWMTWFRQAPGKGLEW |
| | SHWMT | HIKEDGSEKY | GGEGYSDSHF | VAHIKEDGSEKYYVDSVKGRFTISRDNAKNSLYLQMNSLRAEDTAVYYCA |
| | | YVDSVKG | GVDV | RGGEGYSDSHFGVDVWGQGTTVTVSS |
| 1.104 | SEQ ID NO: | SEQ ID NO: | SEQ ID NO: | SEQ ID NO: 416 |
| | 413 | 414 | 415 | EVQLVESGGGLVQPGGSLRLSCAASGFTFSSHWMTWFRQAPGKGLEW |
| | SHWMT | HIKEDGSEKY | GGVGYSDSHF | VAHIKEDGSEKYYVDSVKGRFTISRDNAKNSLYLQMNSLRAEDTAVYYCA |
| | | YVDSVKG | GVDV | RGGVGYSDSHFGVDVWGQGTTVTVSS |
| 1.105 | SEQ ID NO: | SEQ ID NO: | SEQ ID NO: | SEQ ID NO: 420 |
| | 417 | 418 | 419 | EVQLVESGGGLVQPGGSLRLSCAASGFTFSSHWMTWFRQAPGKGLEW |
| | SHWMT | HIKEDESEKY | GGVGYSISHFG | VAHIKEDESEKYYVDSVKGRFTISRDNAKNSLYLQMNSLRAEDTAVYYCA |
| | | YVDSVKG | VDV | RGGVGYSISHFGVDVWGQGTTVTVSS |
| 1.106 | SEQ ID NO: | SEQ ID NO: | SEQ ID NO: | SEQ ID NO: 424 |
| | 421 | 422 | 423 | EVQLVESGGGLVQPGGSLRLSCAASGFTFSSHWMTWFRQAPGKGLEW |
| | SHWMT | HIKEDESEKY | GGEGYSISHFG | VAHIKEDESEKYYVDSVKGRFTISRDNAKNSLYLQMNSLRAEDTAVYYCA |
| | | YVDSVKG | VDV | RGGEGYSISHFGVDVWGQGTTVTVSS |
| 1.107 | SEQ ID NO: | SEQ ID NO: | SEQ ID NO: | SEQ ID NO: 428 |
| | 425 | 426 | 427 | EVQLVESGGGLVQPGGSLRLSCAASGFTLSNYWMNWVRQAPGKGLE |
| | NYWMN | NINQDGSER | GGEGYGVDHY | WVANINQDGSERYYVDSVKGRFTISRDNAKNSLYLQMSSLRAEDTAVYF |
| | | YYVDSVKG | GLDV | CARGGEGYGVDHYGLDVSGQGTTVTVSS |
| 1.108 | SEQ ID NO: | SEQ ID NO: | SEQ ID NO: | SEQ ID NO: 432 |
| | 429 | 430 | 431 | EVQLVESGGGLVQPGGSLRLSCAATGFTLSNYWMNWVRQAPGKGLE |
| | NYWMN | NINQDGSER | GGEGYGVDHY | WVANINQDGSERYYVDSVKGRFTISRDNAKNSLYLQMNSLRAEDTAVY |
| | | YYVDSVKG | GLDV | FCARGGEGYGVDHYGLDVSGQGTTVTVSS |
| 1.109 | SEQ ID NO: | SEQ ID NO: | SEQ ID NO: | SEQ ID NO: 436 |
| | 433 | 434 | 435 | EVQLVESGGGLVQPGGSLRLSCAATGFTLSNYWMNWVRQAPGKGLE |
| | NYWMN | NINQDGSER | GGEGYGVDHY | WVANINQDGSERYYVDSVKGRFTISRDNAKNSLYLQMSSLRAEDTAVYY |
| | | YYVDSVKG | GLDV | CARGGEGYGVDHYGLDVSGQGTTVTVSS |
| 1.110 | SEQ ID NO: | SEQ ID | SEQ ID NO: | SEQ ID NO: 440 |
| | 437 | NO:438 | 439 | EVQLVESGGGLVQPGGSLRLSCAASGFTLSNYWMNWVRQAPGKGLE |
| | NYWMN | NINQDGSER | GGEGYGVDHY | WVANINQDGSERYYVDSVKGRFTISRDNAKNSLYLQMNSLRAEDTAVY |
| | | YYVDSVKG | GLDV | FCARGGEGYGVDHYGLDVSGQGTTVTVSS |
| 1.111 | SEQ ID NO: | SEQ ID NO: | SEQ ID NO: | SEQ ID NO: 444 |
| | 441 | 442 | 443 | EVQLVESGGGLVQPGGSLRLSCAASGFTLSNYWMNWVRQAPGKGLE |
| | NYWMN | NINQDGSER | GGEGYGVDHY | WVANINQDGSERYYVDSVKGRFTISRDNAKNSLYLQMSSLRAEDTAVYY |
| | | YYVDSVKG | GLDV | CARGGEGYGVDHYGLDVSGQGTTVTVSS |
| 1.112 | SEQ ID NO: | SEQ ID NO: | SEQ ID NO: | SEQ ID NO: 448 |
| | 445 | 446 | 447 | EVQLVESGGGLVQPGGSLRLSCAATGFTLSNYWMNWVRQAPGKGLE |
| | NYWMN | NINQDGSER | GGEGYGVDHY | WVANINQDGSERYYVDSVKGRFTISRDNAKNSLYLQMNSLRAEDTAVY |
| | | YYVDSVKG | GLDV | YCARGGEGYGVDHYGLDVSGQGTTVTVSS |
| 1.113 | SEQ ID NO: | SEQ ID NO: | SEQ ID NO: | SEQ ID NO: 452 |
| | 449 | 450 | 451 | EVQLVESGGGLVQPGGSLRLSCAASGFTLSNYWMNWVRQAPGKGLE |
| | NYWMN | NINQDGSER | GGEGYGVDHY | WVANINQDGSERYYVDSVKGRFTISRDNAKNSLYLQMNSLRAEDTAVY |
| | | YYVDSVKG | GLDV | YCARGGEGYGVDHYGLDVSGQGTTVTVSS |
| 1.114 | SEQ ID NO: | SEQ ID NO: | SEQ ID NO: | SEQ ID NO: 456 |
| | 453 | 454 | 455 | EVQLVESGGGLVQPGGSLRLSCAASGFTLSNYWMNWVRQAPGKGLE |
| | NYWMN | NINQDESERY | GGEGYGVDHY | WVANINQDESERYYVDSVKGRFTISRDNAKNSLYLQMNSLRAEDTAVYY |
| | | YVDSVKG | GLDV | CARGGEGYGVDHYGLDVSGQGTTVTVSS |

TABLE 4
Further exemplary sequences for binding molecules that include two single variable heavy chain
domain antibodies, one that binds to CD137 and another that binds to PD-1 - Full length sequences and
CDR sequences of V_{H} single domain antibodies (Family 2) shown below

| Name | CDR1 | CDR2 | CDR3 | Full Length |
|---|---|---|---|---|
| 2.1 | SEQ ID NO: | SEQ ID NO: 458 | SEQ ID NO: 459 | SEQ ID NO: 460 |
| | 457 | YISGSGDIIDYA | EDSRLTGTTDFD | EVQLVESGGGLVKPGGSLRVSCAASGFTFSDYYMSWFRQAPGK |
| | DYYMS | DSVKG | N | GLEWVSYISGSGDIIDYADSVKGRFTISRDNAKNSLYLQMNNLR |
| | | | | AEDTAVYHCAREDSRLTGTTDFDNWGQGTLVTVSS |
| 2.2 | SEQ ID NO: | SEQ ID NO: 462 | SEQ ID NO: 463 | SEQ ID NO: 464 |
| | 461 DYYMS | YISGSGDIIDYA | EDSRIPGTTDFD | EVQLVESGGGLVKPGGSLRVSCAASGFTFSDYYMSWFRQAPGK |
| | | DSVKG | N | GLEWVSYISGSGDIIDYADSVKGRFTISRDNAKNSLYLQMNSLRA |
| | | | | EDTAVYHCAKEDSRIPGTTDFDNWGQGTLVTVSS |
| 2.3 | SEQ ID NO: | SEQ ID NO: 466 | SEQ ID NO: 467 | SEQ ID NO: 468 |
| | 465 DYYMS | YISGSGDIIDYA | EDSRIPGTTDFD | EVQLVESGGGLVKPGGSLRVSCAASGFTFSDYYMSWFRQAPGK |
| | | DSVKG | N | GLEWISYISGSGDIIDYADSVKGRFTISRDNAKNSLYLQMNSLRA |
| | | | | EDTAVYHCAKEDSRIPGTTDFDNWGQGTLVTVSS |
| 2.4 | SEQ ID NO: | SEQ ID NO: 470 | SEQ ID NO: 471 | SEQ ID NO: 472 |
| | 469 DYYMS | YISGSGDVIDYA | EDSRIPGTTDFD | EVQLVESGGGLVKPGGSLRVSCAASGFTFSDYYMSWFRQAPGK |
| | | DSVKG | N | GLEWVSYISGSGDVIDYADSVKGRFTISRDNAKNSLYLQMNSLR |
| | | | | AEDTAVYHCAKEDSRIPGTTDFDNWGQGTLVTVSS |
| 2.5 | SEQ ID NO: | SEQ ID NO: 474 | SEQ ID NO: 475 | SEQ ID NO: 476 |
| | 473 DYYMS | YISGSGDIIDYA | EDSRIPGTTDFD | EVQLVESGGGLVKPGGSLRLSCAVSGFTFSDYYMSWFRQAPGK |
| | | DSVKG | N | GLEWVSYISGSGDIIDYADSVKGRFTISRDNAKNSLYLQMNSLRA |
| | | | | EDTAVYHCAKEDSRIPGTTDFDNWGQGTLVTVSS |
| 2.6 | SEQ ID NO: | SEQ ID NO: 478 | SEQ ID NO: 479 | SEQ ID NO: 480 |
| | 477 DYYMS | YISGSGDIIDYA | EDSRIPGTTDFDS | EVQLVESGGGLVKPGGSLRVSCAASGFTFSDYYMSWFRQAPGK |
| | | DSVKG | | GLEWVSYISGSGDIIDYADSVKGRFTISRDNAKNSLYLQMNSLRA |
| | | | | EDTAVYHCAKEDSRIPGTTDFDSWGQGTMVTVSS |
| 2.7 | SEQ ID NO: | SEQ ID NO: 482 | SEQ ID NO: 483 | SEQ ID NO: 484 |
| | 481 DYYMS | YISGSGTTIDYA | EDIRMTGTTDFD | EVQLVESGGGLVKPGGSLRLSCAASGFTFSDYYMSWFRQAPGK |
| | | DSVKG | N | GLEWVSYISGSGTTIDYADSVKGRFTISRDNARNSLYLQMNSLRA |
| | | | | EDTAVYYCAREDIRMTGTTDFDNWGQGTLVTVSS |
| 2.8 | SEQ ID NO: | SEQ ID NO: | SEQ ID NO: 487 | SEQ ID NO: 488 |
| | 485 DYYMS | 486 | EDSRMPGTTDFD | EVQLVESGGGLVKPGGSLRLSCAASGFAFSDYYMSWFRQAPGK |
| | | HISGSGTTIDYA | N | GLEWVSHISGSGTTIDYADSVKGRFTISRDNAKNSLYLQMNSLR |
| | | DSVKG | | AEDTAVYHCAREDSRMPGTTDFDNWGQGTLVTVSS |
| 2.9 | SEQ ID NO: | SEQ ID NO: | SEQ ID NO: 491 | SEQ ID NO: 492 |
| | 489 DYYMT | 490 | EDSRIAGTTDFD | EVQLVESGGGLVKPGGSLRLSCAASGFTFSDYYMTWFRQAPGK |
| | | YISGSGDTIDYA | N | GLEWISYISGSGDTIDYAESVKGRFTISRDNAKNSLYLQMNSLRA |
| | | ESVKG | | EDTAVYHCAREDSRIAGTTDFDNWGPGTLVTVSS |
| 2.10 | SEQ ID NO: | SEQ ID NO: | SEQ ID NO: 495 | SEQ ID NO: 496 |
| | 493 DYYMT | 494 | EDSRLSGTTDFDY | EVQLVESGGGLVKPGGSLRLSCAASGFTFSDYYMTWFRQAPGK |
| | | YISSSGSNIDYA | | GLEWVSYISSSGSNIDYADSVKGRFTISRDNAKNSLYLQMNSLRA |
| | | DSVKG | | EDTAVYYCAREDSRLSGTTDFDYWGQGTLVTVSS |
| 2.11 | SEQ ID NO: | SEQ ID NO: | SEQ ID NO: 499 | SEQ ID NO: 500 |
| | 497 DYYMT | 498 | EDSRIAGTTDFD | EVQLVESGGGLVQPGGSLRLSCAASGFTFSDYYMTWFRQAPGK |
| | | YISGSGDTIDYA | N | GLEWISYISGSGDTIDYAESVKGRFTISRDNAKNSLYLQMNSLRA |
| | | ESVKG | | EDTAVYHCAREDSRIAGTTDFDNWGPGTLVTVSS |
| 2.12 | SEQ ID NO: | SEQ ID NO: | SEQ ID NO: 503 | SEQ ID NO: 504 |
| | 501 DYYMS | 502 | EDIRMTGTTDFD | EVQLVESGGGLVKPGGSLRLSCAASGFTFSDYYMSWFRQAPGK |
| | | HISGSGTTIDYA | H | GLEWVSHISGSGTTIDYADSVKGRFTISRDNARKSLYLQMNSLR |
| | | DSVKG | | AEDTAVYYCAREDIRMTGTTDFDHWGQGTLVTVSS |
| 2.13 | SEQ ID NO: | SEQ ID NO: | SEQ ID NO: 507 | SEQ ID NO: 508 |
| | 505 DYYMS | 506 | EDPRLPGTTDFD | QVQLVESGGGLVKPGGSLRLSCAASGFTFSDYYMSWFRQAPGK |
| | | HISSSGNTIDYA | Y | GLEWVSHISSSGNTIDYADSVKGRFTISRDNAKNSLYLQMNSLR |
| | | DSVKG | | AEDTAVYYCAREDPRLPGTTDFDYWGQGTLVTVSS |
| 2.14 | SEQ ID NO: | SEQ ID NO: | SEQ ID NO: 511 | SEQ ID NO: 512 |
| | 509 DYYMT | 510 | EDIRMPGTTDFD | EVQLVESGGGLVKPGGSLRLSCAASGFTFSDYYMTWFRQAPGK |
| | | YISGSGDTIDYA | H | GLEWISYISGSGDTIDYAESVKGRFTISRDNAKNSLYLQMNSLRA |
| | | ESVKG | | EDTAVYYCAREDIRMPGTTDFDHWGQGTLVTVSS |
| 2.15 | SEQ ID NO: | SEQ ID NO: | SEQ ID NO: 515 | SEQ ID NO: 516 |
| | 513 DYYMS | 514 | EDIRMPGTTDFD | EVQLVESGGGLVKPGGSLRLSCAVSGFTFSDYYMSWFRQAPGK |
| | | HISGSGTTIDYA | H | GLEWVSHISGSGTTIDYADSVKGRFTISRDNARNSLYLQMNSLR |
| | | DSVKG | | AEDTAVYYCAREDIRMPGTTDFDHWGQGTLVTVSS |
| 2.16 | SEQ ID NO: | SEQ ID NO: | SEQ ID NO: 519 | SEQ ID NO: 520 |
| | 517 DYYMS | 518 | EDPRLTGTTDFD | QVQLVESGGGLVKPGGSLRLSCAASGFTFSDYYMSWIRQAPGK |
| | | HISSSGSTIDYA | Y | GLEWVSHISSSGSTIDYADSVKGRFTISRDNAKNSLYLQMNSLRA |
| | | DSVKG | | EDTAVYYCAREDPRLTGTTDFDYWGQGALVTVSS |
| 2.17 | SEQ ID NO: | SEQ ID NO: | SEQ ID NO: 523 | SEQ ID NO: 524 |
| | 521 DYYMS | 522 | EDPRISGTTDFD | QVQLVESGGGLVKPGGSLRLSCAASGFTFSDYYMSWIRQAPGK |
| | | YISSSGSTISYAD | N | GLEWVSYISSSGSTISYADSVKGRFTISRDNAKNSLYLQMNSLRA |
| | | SVKG | | EDTAVYYCAREDPRISGTTDFDNWGQGTLVTVSS |
| 2.18 | SEQ ID NO: | SEQ ID NO: 526 | SEQ ID NO: 527 | SEQ ID NO: 528 |
| | 525 DYYMS | HISSSGNTIDYA | EDPRLPGTTDFD | QVQLQESGGGLVKPGGSLRLSCAASGFTFSDYYMSWFRQAPGK |
| | | DSVKG | Y | GLEWVSHISSSGNTIDYADSVKGRFTISRDNAKNSLYLQMNSLR |
| | | | | AEDTAVYYCAREDPRLPGTTDFDYWGQGTLVTVSS |
| 2.19 | SEQ ID NO: | SEQ ID NO: | SEQ ID NO: 531 | SEQ ID NO: 532 |
| | 529 DYYMS | 530 | EDPRLPGTSEFD | QVQLVESGGGLVKPGGSLRLSCAASGFTFSDYYMSWFRQAPGK |
| | | YISGTGITTDYA | N | GLEWVSYISGTGITTDYADSVKGRFTISRDNAKNSLYLQMNSLRA |
| | | DSVKG | | EDTAVYYCAREDPRLPGTSEFDNWGQGTLVTVSS |
| 2.20 | SEQ ID NO: | SEQ ID NO: | SEQ ID NO: 535 | SEQ ID NO: 536 |
| | 533 DYYMS | 534 | EDPRMPGTFDFD | EVQLVESGGGLVKPGGSLRLSCAASGFTFSDYYMSWIRQAPGK |
| | | HISSSGSTIDYA | N | GLEWVSHISSSGSTIDYADSVKGRFTISRDNAKNSLYLQMNSLRA |
| | | DSVKG | | EDTAVYYCAREDPRMPGTFDFDNWGQGTLVTVSS |
| 2.21 | SEQ ID NO: | SEQ ID NO: | SEQ ID NO: 539 | SEQ ID NO: 540 |
| | 537 DYYMS | 538 | EDIRMPGTTDFD | EVQLVESGGGLVKPGGSLRLSCAASGFAFSDYYMSWFRQAPGK |
| | | HISGSGTTIDYA | H | GLEWVSHISGSGTTIDYADSVKGRFTISRDNARNSLYLQMNSLR |
| | | DSVKG | | AEDTAVYYCAREDIRMPGTTDFDHWGQGTLVTVSS |
| 2.22 | SEQ ID NO: | SEQ ID NO: | SEQ ID NO: 543 | SEQ ID NO: 544 |
| | 541 DYYMS | 542 | EDIRMPGTTDFD | EVQLVESGGGLVKPGGSLRLSCAVSGFTFSDYYMSWFRQAPGK |
| | | HISGSGTTIDYA | H | GLEWVSHISGSGTTIDYADSVKGRFTISRDNARDSLYLQMNSLR |
| | | DSVKG | | AEDTAVYYCAREDIRMPGTTDFDHWGQGTLVTVSS |
| 2.23 | SEQ ID NO: | SEQ ID NO: | SEQ ID NO: 547 | SEQ ID NO: 548 |
| | 545 DYYMS | 546 | EDIRMPGTTDFD | EVQLVESGGGLVTPGGSLRLSCAVSGFTFSDYYMSWFRQAPGK |
| | | HISGSGTTIDYA | H | GLEWVSHISGSGTTIDYADSVKGRFTISRDNARNSLYLQMNSLR |
| | | DSVKG | | AEDTAVYYCAREDIRMPGTTDFDHWGQGTLVTVSS |
| 2.24 | SEQ ID NO: | SEQ ID NO: | SEQ ID NO: 551 | SEQ ID NO: 552 |
| | 549 DYYMS | 550 | EDIRMPGTTDFD | EVQLVESGGGLVKPGGSLRLSCAVSGFTFSDYYMSWFRQAPGK |
| | | HISGSGTTIDYA | H | GLEWVSHISGSGTTIDYADSVKGRFTISRDNARNSLYLQMNSLR |
| | | DSVKG | | AEDTAMYYCAREDIRMPGTTDFDHWGQGTLVTVSS |
| 2.25 | SEQ ID NO: | SEQ ID NO: | SEQ ID NO: 555 | SEQ ID NO: 556 |
| | 553 DYYMS | 554 | EDIRMPGTTDFD | EVQLVESGGGLVKPGGSLRLSCAASGFAFSDYYMSWFRQAPGK |
| | | HISGSGTTIDYA | H | GLEWVSHISGSGTTIDYADSVKDRFTISRDNARNSLYLQMNSLR |
| | | DSVKD | | AEDTAVYYCAREDIRMPGTTDFDHWGQGTLVTVSS |
| 2.26 | SEQ ID NO: | SEQ ID NO: | SEQ ID NO: 559 | SEQ ID NO: 560 |
| | 557 DYYMS | 558 | EDIRMPGTTDFD | EVQLVESGGGLVKPGGSLRLSCTASGFTFTDYYMSWFRQAPGK |
| | | HISSSGTTIDYA | N | GLEWVSHISSSGTTIDYADSVKGRFTISRDNAKNSLYLQMNSLRA |
| | | DSVKG | | DDTAVYYCAREDIRMPGTTDFDNWGQGTLVTVSS |
| 2.27 | SEQ ID NO: | SEQ ID NO: | SEQ ID NO: 563 | SEQ ID NO: 564 |
| | 561 DYYMT | 562 | EDIRMSGTTDFD | QVQLVESGGGLVKPGGSLRLSCAASGFTFSDYYMTWFRQAPGK |
| | | YISSSGSTISYAD | Y | GLEWVSYISSSGSTISYADSVKGRFTISRDNANNSLYLQMNSLRA |
| | | SVKG | | EDTAVYHCAREDIRMSGTTDFDYWGQGTLVTVSS |
| 2.28 | SEQ ID NO: | SEQ ID NO: | SEQ ID NO: 567 | SEQ ID NO: 568 |
| | 565 DYYMS | 566 | EDPRLSGTIDFDS | QVQLVESGGGLVKPGGSLRLSCAASGFIFSDYYMSWFRQAPGK |
| | | HISSSGSSIDYA | | GLEWVSHISSSGSSIDYADSVKGRFTISRDNAKNSLYLQMNSLRA |
| | | DSVKG | | EDTAVYYCAREDPRLSGTIDFDSWGQGTLVTVSS |
| 2.29 | SEQ ID NO: | SEQ ID NO: | SEQ ID NO: 571 | SEQ ID NO: 572 |
| | 569 DYYMS | 570 | EDIRMPGTTDFD | EVQLVESGGGLVKPGGSLRLSCAASGFAFSDYYMSWFRQAPGK |
| | | HIGGSGTTIDYA | H | GLEWVSHIGGSGTTIDYADSVKGRFTISRDNARNSLYLQMNSLR |
| | | DSVKG | | AEDTAVYYCAREDIRMPGTTDFDHWGQGTLVTVSS |
| 2.30 | SEQ ID NO: | SEQ ID NO: | SEQ ID NO: 575 | SEQ ID NO: 576 |
| | 573 DYYMS | 574 | EDPRVPGTTNFD | QVQLVESGGGLVKPGGSLRLSCAASGFTFSDYYMSWIRQAPGK |
| | | YISSSGSTIYYAD | Y | GLEWVSYISSSGSTIYYADSVKGRFTISRDNAKNSLYLQMNSLRA |
| | | SVKG | | EDTAVYYCAREDPRVPGTTNFDYWGQGTLVTVSS |
| 2.31 | SEQ ID NO: | SEQ ID NO: | SEQ ID NO: 579 | SEQ ID NO: 580 |
| | 577 DYYMT | 578 | EDGRIPGTTDFD | QVQLVESGGGLVKPGGSLRLSCAASGFTFSDYYMTWMRQAPG |
| | | YISGSGSTIDYA | H | KGLEWVSYISGSGSTIDYADSVKGRFTISRDNAKNSLYLQMNSLR |
| | | DSVKG | | PEDTAVYYCAKEDGRIPGTTDFDHWGQGTLVTVSS |
| 2.32 | SEQ ID NO: | SEQ ID NO: | SEQ ID NO: 583 | SEQ ID NO: 584 |
| | 581 DYYMS | 582 | EDIRMPGTTDFD | EVQLVESGGGLVQPGGSLRLSCAASGFAFSDYYMSWFRQAPGK |
| | | HISGSGTTIDYA | H | GLEWVSHISGSGTTIDYADSVKDRFTISRDNARNSLYLQMNSLR |
| | | DSVKD | | AEDTAVYYCAREDIRMPGTTDFDHWGQGTLVTVSS |
| 2.33 | SEQ ID NO: | SEQ ID NO: | SEQ ID NO: 587 | SEQ ID NO: 588 |
| | 585 DYFMS | 586 | EDPRLPGTTDFD | QVQLVESGGGLVKPGGSLRLSCAASGFPFSDYFMSWFRQAPGK |
| | | HISSSGNSIDYA | Y | GLEWVSHISSSGNSIDYADSVKGRFTISRDNAKNSLYLQMNSLR |
| | | DSVKG | | AEDTAVYYCAKEDPRLPGTTDFDYWGQGTLVTVSS |
| 2.34 | SEQ ID NO: | SEQ ID NO: | SEQ ID NO: 591 | SEQ ID NO: 592 |
| | 589 DSYMS | 590 | EDPRLPGTSDFD | QVQLVESGGGLVKPGGSLRLSCAASGFTFSDSYMSWIRQAPGK |
| | | HISNSGSTISYA | Y | GLEWVSHISNSGSTISYADSVKGRFTISRDNAKNSLYLQMNSLRA |
| | | DSVKG | | EDTAVYYCAREDPRLPGTSDFDYWGQGTLVTVSS |
| 2.35 | SEQ ID NO: | SEQ ID NO: | SEQ ID NO: 595 | SEQ ID NO: 596 |
| | 593 DYYMS | 594 | EDPRLSGTTDFD | QVQLVESGGGVVQPGRSLRLSCAASGFTFSDYYMSWIRQAPGK |
| | | HISSSGSSIDYA | Q | GLEWVSHISSSGSSIDYADSVKGRFTISRDNAKNSLYLQMNSLRD |
| | | DSVKG | | EDTAVYYCAREDPRLSGTTDFDQWGQGTLVTVSS |
| 2.36 | SEQ ID NO: | SEQ ID NO: | SEQ ID NO: 599 | SEQ ID NO: 600 |
| | 597 SYWMS | 598 | EDPRMTGTTDFD | QVQLVESGGGLVQPGGSLRLSCAASGFTFSSYWMSWVRQAPG |
| | | HISSSGSTIDYAE | Y | KGLEWVSHISSSGSTIDYAESVKGRFTISRDNAKNSLYLQMNSLR |
| | | SVKG | | AEDTAVYYCAREDPRMTGTTDFDYWGQGTLVTVSS |
| 2.37 | SEQ ID NO: | SEQ ID NO: | SEQ ID NO: 603 | SEQ ID NO: 604 |
| | 601 NYFMS | 602 | EDPRLPGTTDFD | QVQLQESGGGLVKPGGSLRLSCAASGFTFSNYFMSWIRQAPGK |
| | | HISSSGNTIDYA | Y | GLEWVSHISSSGNTIDYADSVKGRFTISRDNAKNSLYLQMDSLR |
| | | DSVKG | | AEDTAVYYCSREDPRLPGTTDFDYWGQGTLVTVSS |
| 2.38 | SEQ ID NO: | SEQ ID NO: | SEQ ID NO: 607 | SEQ ID NO: 608 |
| | 605 DYYMT | 606 | ENPRLPGTMDFD | EVQLVESGGGVVKPGGSLRLSCAASGFTFSDYYMTWIRQGPGK |
| | | YISSGGSTIHYA | Y | GQEWISYISSGGSTIHYADSVKGRFTISRDNAKNSLYLQMNSLRA |
| | | DSVKG | | EDTAVYYCARENPRLPGTMDFDYWGQGTLVTVSS |
| 2.39 | SEQ ID NO: | SEQ ID NO: | SEQ ID NO: 611 | SEQ ID NO: 612 |
| | 609 DHFMS | 610 | EDPRLTGTTDFD | QVQLVESGGGLVQPGGSLRLSCAASGFTFSDHFMSWFRQAPG |
| | | NIKQDGSEKYY | N | KGLEWVANIKQDGSEKYYVDSVKGRFTISRDNAKNSLFLQMNSL |
| | | VDSVKG | | RAEDTAMYYCAREDPRLTGTTDFDNWGQGTLVTVSS |
| 2.40 | SEQ ID NO: | SEQ ID NO: | SEQ ID NO: 615 | SEQ ID NO: 616 |
| | 613 NYWMT | 614 | EDPRLPGTMDFD | EVQLVESGGGLVQAGGSLRLSCVASGFTFSNYWMTWFRQAPG |
| | | HISSTGSTIDYA | Y | RGLEWVSHISSTGSTIDYADSVKGRFTISRDNAENSLYLQMNSLR |
| | | DSVKG | | AEDTAVYYCAREDPRLPGTMDFDYWGQGTLVTVSS |
| 2.41 | SEQ ID NO: | SEQ ID NO: | SEQ ID NO: 619 | SEQ ID NO: 620 |
| | 617 DYYMS | 618 | EDSRLTGTTDFD | EVQLVESGGGLVKPGGSLRLSCAASGFTFSDYYMSWFRQAPGK |
| | | YISGSGDIIDYA | N | GLEWVSYISGSGDIIDYADSVKGRFTISRDNAKNSLYLQMNNLR |
| | | DSVKG | | AEDTAVYHCAREDSRLTGTTDFDNWGQGTLVTVSS |
| 2.42 | SEQ ID NO: | SEQ ID NO: 622 | SEQ ID NO: 623 | SEQ ID NO:624 |
| | 621 DYYMS | YISGSGDIIDYA | EDSRLTGTTDFD | EVQLVESGGGLVKPGGSLRVSCAASGFTFSDYYMSWIRQAPGK |
| | | DSVKG | N | GLEWVSYISGSGDIIDYADSVKGRFTISRDNAKNSLYLQMNNLR |
| | | | | AEDTAVYHCAREDSRLTGTTDFDNWGQGTLVTVSS |
| 2.43 | SEQ ID NO: | SEQ ID NO: 626 | SEQ ID NO: 627 | SEQ ID NO: 628 |
| | 625 | YISGSGDIIDYA | EDSRLTGTTDFD | EVQLVESGGGLVKPGGSLRVSCAASGFTFSDYYMSWFRQAPGK |
| | DYYMS | DSVKG | N | GLEWVSYISGSGDIIDYADSVKGRFTISRDNAKNSLYLQMNNLR |
| | | | | AEDTAVYYCAREDSRLTGTTDFDNWGQGTLVTVSS |
| 2.44 | SEQ ID NO: | SEQ ID NO: 630 | SEQ ID NO: 631 | SEQ ID NO: 632 |
| | 629 | YISGSGDIIDYA | EDSRLTGTTDFD | EVQLVESGGGLVKPGGSLRVSCAASGFTFSDYYMSWFRQAPGK |
| | DYYMS | DSVKG | N | GLEWVSYISGSGDIIDYADSVKGRFTISRDNAKNSLYLQMNSLRA |
| | | | | EDTAVYHCAREDSRLTGTTDFDNWGQGTLVTVSS |
| 2.45 | SEQ ID NO: | SEQ ID NO: 634 | SEQ ID NO: 635 | SEQ ID NO: 636 |
| | 633 | YISGSGDIIDYA | EDSRLTGTTDFD | EVQLVESGGGLVKPGGSLRLSCAASGFTFSDYYMSWFRQAPGK |
| | DYYMS | DSVKG | N | GLEWVSYISGSGDIIDYADSVKGRFTISRDNAKNSLYLQMNNLR |
| | | | | AEDTAVYYCAREDSRLTGTTDFDNWGQGTLVTVSS |
| 2.46 | SEQ ID NO: | SEQ ID NO: 638 | SEQ ID NO: 639 | SEQ ID NO: 640 |
| | 637 | YISGSGDIIDYA | EDSRLTGTTDFD | EVQLVESGGGLVKPGGSLRLSCAASGFTFSDYYMSWIRQAPGK |
| | DYYMS | DSVKG | N | GLEWVSYISGSGDIIDYADSVKGRFTISRDNAKNSLYLQMNSLRA |
| | | | | EDTAVYYCAREDSRLTGTTDFDNWGQGTLVTVSS |
| 2.47 | SEQ ID NO: | SEQ ID NO: 642 | SEQ ID NO: 643 | SEQ ID NO: 644 |
| | 641 | YISGSGDIIDYA | EDSRLTGTTDFD | EVQLVESGGGLVKPGGSLRLSCAASGFTFSDYYMSWFRQAPGK |
| | DYYMS | DSVKG | N | GLEWVSYISGSGDIIDYADSVKGRFTISRDNAKNSLYLQMNSLRA |
| | | | | EDTAVYYCAREDSRLTGTTDFDNWGQGTLVTVSS |
| 2.48 | SEQ ID NO: | SEQ ID NO: 646 | SEQ ID NO: 647 | SEQ ID NO: 648 |
| | 645 | YISGSGDIIDYA | EDARLTGTTDFD | EVQLVESGGGLVKPGGSLRLSCAASGFTFSDYYMSWIRQAPGK |
| | DYYMS | DSVKG | N | GLEWVSYISGSGDIIDYADSVKGRFTISRDNAKNSLYLQMNSLRA |
| | | | | EDTAVYYCAREDARLTGTTDFDNWGQGTLVTVSS |
| 2.49 | SEQ ID NO: | SEQ ID NO: 650 | SEQ ID NO: 651 | SEQ ID NO: 652 |
| | 649 | YISGSGDIIDYA | EDPRLTGTTDFD | EVQLVESGGGLVKPGGSLRLSCAASGFTFSDYYMSWIRQAPGK |
| | DYYMS | DSVKG | N | GLEWVSYISGSGDIIDYADSVKGRFTISRDNAKNSLYLQMNSLRA |
| | | | | EDTAVYYCAREDPRLTGTTDFDNWGQGTLVTVSS |
| 2.50 | SEQ ID NO: | SEQ ID NO: 654 | SEQ ID NO: 655 | SEQ ID NO: 656 |
| | 653 | YISGSGDIIDYA | EDARLTGTTDFD | EVQLVESGGGLVKPGGSLRLSCAASGFTFSDYYMSWFRQAPGK |
| | DYYMS | DSVKG | N | GLEWVSYISGSGDIIDYADSVKGRFTISRDNAKNSLYLQMNSLRA |
| | | | | EDTAVYYCAREDARLTGTTDFDNWGQGTLVTVSS |
| 2.51 | SEQ ID NO: | SEQ ID NO: 658 | SEQ ID NO: 659 | SEQ ID NO: 660 |
| | 657 | YISGSGDIIDYA | EDPRLTGTTDFD | EVQLVESGGGLVKPGGSLRLSCAASGFTFSDYYMSWFRQAPGK |
| | DYYMS | DSVKG | N | GLEWVSYISGSGDIIDYADSVKGRFTISRDNAKNSLYLQMNSLRA |
| | | | | EDTAVYYCAREDPRLTGTTDFDNWGQGTLVTVSS |

In one embodiment, the binding molecule that binds to CD137 and PD-1 comprises one or more single domain antibodies that bind to CD137, for example a single domain antibody selected from shown in any of in Tables 2, 3 and 4 or a sequence with at least 70%, 80&, 85%, 90%, 95% sequence homology thereto. In some embodiments, single domain antibody is a variant of any of the above single V_{H} domain antibodies shown in Table 2, 3 and 4 having one or more amino acid substitutions, deletions, insertions or other modifications, and which retains a biological function of the single domain antibody. Thus, variant V_{H} single domain antibodies can be sequence engineered. Modifications are described elsewhere herein. In one embedment, there is provided a variant of V_{Hs} 1.07 to 1.113, for example V_{H}1.113 which has 1, 2, 3, 4, 5, 6, 7, 8, 9 or 10 substitutions.

### Examples of Substitutions

In one embodiment, the variant comprises one or more the following substitutions with reference to V_{H}1.1 or combinations thereof:
F37V
E61V+E65K
E65K
V70I
V79L
F37V+E61V+E65K+V70I+V79L
E61V+E65K+V70I+V79L
F37V+E61V+E65K+V70I+V79L+G55E+D101V+D105I
F37V+E61V+E65K+V70I+V79LFGL+G55E+D101E+D105I
E61V+E65K+V70I+V79L+G55E
E61V+E65K+V70I+V79L+G55E+D105I
E61V+E65K+V70I+V79L+D54G, E61V+E65K+V70I+V79L+D54E,
E61V+E65K+V70I+V79L+D101E
E61V+E65K+V70I+V79L+D101V or
E61V+E65K+V70I+V79L+G55E+D101V+D105I, E61V+E65K+V70I+V79L+G55E+D101E+D105I

In one embodiment, the variant comprises one or more the following substitutions with reference to V_{H}1.1 or combinations thereof:

a) E61V+E65K+V70I+V79L+G55E+D101→any amino acid selected from the following F, L, I, M, V, S, P, T, A, Y, H, Q, K, D, W, R, G;
b) E61V+E65K+V70I+V79L+G55E+D105→any amino acid selected from the following F, L, M, S, P, T, A, Y, H, Q, N, K, D, E, W, R, G or
c) E61V+E65K+V70I+V79L+G55E, D101→any amino acid selected from the following F, L, I, M, V, S, P, T, A, Y, H, Q, K, D, W, R, G+D105→any amino acid selected from the following F, L, M, S, P, T, A, Y, H, Q, N, K, D, E, W, R, G.

In one embodiment, the variant comprises one or more the following substitutions with reference to V_{H}1.78 or combinations thereof:
Q1E+T25S
Q1E+S84N
Q1E+F95Y
Q1E+T25S+S84N
Q1E+T25S+F95Y
Q1E+S84N+F95Y
Q1E+T25S+S84N+F95Y or
Q1E+T25S+G55E+S84N+F95Y

In one embodiment, the variant comprises one or more the following substitutions with reference to V_{H}2.1 or combinations thereof:
V20L,
F37I,
N85S,
N95Y,
V20L+H95Y,
V20L+F37I+N85S+H95Y, V20L+N85S+H95Y,
V20L+F37I+N85S+H95Y+S101A,
V20L+F37I+N85S+H95Y+S101P or
V20L+N85S+H95Y+S101A, V20L+N85S+H95Y+S101A.

In one embodiment, the variant comprises one or more the following substitutions with reference to V_{H}2.50 or combinations thereof:
S35T+V48I+I57T+L103M+T104R+T107I
S35T+A101T+L103V+T104R+T107V
S35T+V48I+I57T+A101E+L103L+T104W+T107V
S35T+V48I+L103T+T104R+T107V
Y32H+S35T+V48I+S52G+S54D+D56A+I58L+A101L+T104P+T107I+N111H
S35T+A101T+L103V+T104R+T107V+N111S
A28T+S30T+Y33W+S35T+V48I+G53S+S54D+D56K+I57T+L103M+T104P+T107I+N111Y
S35T+V48I+L103T+T104R+T107V+N111S
S35T+V48I+I57T+S101E+T104W+T107V+N111S or
S35T+V48I+I57T+L103M+T104R+T107I+N111S

### Exemplary Nucleic Acids Encoding CD137 Polypeptide

An isolated nucleic acid encoding a single domain antibody as shown above is set out below. Nucleic acid may include DNA and/or RNA. In one aspect, the present invention provides a nucleic acid that codes for a CDR, for example CDR3, a set of two or three CDRs or a V_{H} single domain antibody of the invention as shown above. Exemplary sequences are shown in SEQ ID Nos 661 to 774 and 775 to 825.

TABLE 5
Nucleic acids encoding V_{H} 1.1 to 1.114 as shown in Table 4

| Name | Nucleotide Sequence |
|---|---|
| 1.1 | SEQ ID NO: 661 |
| 1.2 | SEQ ID NO: 662 |
| 1.3 | SEQ ID NO: 663 |
| 1.4 | SEQ ID NO: 664 |
| 1.5 | SEQ ID NO: 665 |
| 1.6 | SEQ ID NO: 666 |
| 1.7 | SEQ ID NO: 667 |
| 1.8 | SEQ ID NO: 668 |
| 1.9 | SEQ ID NO: 669 |
| 1.10 | SEQ ID NO: 670 |
| 1.11 | SEQ ID NO: 671 |
| 1.12 | SEQ ID NO: 672 |
| 1.13 | SEQ ID NO: 673 |
| 1.14 | SEQ ID NO: 674 |
| 1.15 | SEQ ID NO: 675 |
| 1.16 | SEQ ID NO: 676 |
| 1.17 | SEQ ID NO: 677 |
| 1.18 | SEQ ID NO: 678 |
| 1.19 | SEQ ID NO: 679 |
| 1.20 | SEQ ID NO: 680 |
| 1.21 | SEQ ID NO: 681 |
| 1.22 | SEQ ID NO: 682 |
| 1.23 | SEQ ID NO: 683 |
| 1.24 | SEQ ID NO: 684 |
| 1.25 | SEQ ID NO: 685 |
| 1.26 | SEQ ID NO: 686 |
| 1.27 | SEQ ID NO: 687 |
| 1.28 | SEQ ID NO: 688 |
| 1.29 | SEQ ID NO: 689 |
| 1.30 | SEQ ID NO: 690 |
| 1.31 | SEQ ID NO: 691 |
| 1.32 | SEQ ID NO: 692 |
| 1.33 | SEQ ID NO: 693 |
| 1.34 | SEQ ID NO: 694 |
| 1.35 | SEQ ID NO: 695 |
| 1.36 | SEQ ID NO: 696 |
| 1.37 | SEQ ID NO: 697 |
| 1.38 | SEQ ID NO: 698 |
| 1.39 | SEQ ID NO: 699 |
| 1.40 | SEQ ID NO: 700 |
| 1.41 | SEQ ID NO: 701 |
| 1.42 | SEQ ID NO: 702 |
| 1.43 | SEQ ID NO: 703 |
| 1.44 | SEQ ID NO: 704 |
| 1.45 | SEQ ID NO: 705 |
| 1.46 | SEQ ID NO: 706 |
| 1.47 | SEQ ID NO: 707 |
| 1.48 | SEQ ID NO: 708 |
| 1.49 | SEQ ID NO: 709 |
| 1.50 | SEQ ID NO: 710 |
| 1.51 | SEQ ID NO: 711 |
| 1.52 | SEQ ID NO: 712 |
| 1.53 | SEQ ID NO: 713 |
| 1.54 | SEQ ID NO: 714 |
| 1.55 | SEQ ID NO: 715 |
| 1.56 | SEQ ID NO: 716 |
| 1.57 | SEQ ID NO: 717 |
| 1.58 | SEQ ID NO: 718 |
| 1.59 | SEQ ID NO: 719 |
| 1.60 | SEQ ID NO: 720 |
| 1.61 | SEQ ID NO: 721 |
| 1.62 | SEQ ID NO: 722 |
| 1.63 | SEQ ID NO: 723 |
| 1.64 | SEQ ID NO: 724 |
| 1.65 | SEQ ID NO: 725 |
| 1.66 | SEQ ID NO: 726 |
| 1.67 | SEQ ID NO: 727 |
| 1.68 | SEQ ID NO: 728 |
| 1.69 | SEQ ID NO: 729 |
| 1.70 | SEQ ID NO: 730 |
| 1.71 | SEQ ID NO: 731 |
| 1.72 | SEQ ID NO: 732 |
| 1.73 | SEQ ID NO: 733 |
| 1.74 | SEQ ID NO: 734 |
| 1.75 | SEQ ID NO: 735 |
| 1.76 | SEQ ID NO: 736 |
| 1.77 | SEQ ID NO: 737 |
| 1.78 | SEQ ID NO: 738 |
| 1.79 | SEQ ID NO: 739 |
| 1.80 | SEQ ID NO: 740 |
| 1.81 | SEQ ID NO: 741 |
| 1.82 | SEQ ID NO: 742 |
| 1.83 | SEQ ID NO: 743 |
| 1.84 | SEQ ID NO: 744 |
| 1.85 | SEQ ID NO: 745 |
| 1.86 | SEQ ID NO: 746 |
| 1.87 | SEQ ID NO: 747 |
| 1.88 | SEQ ID NO: 748 |
| 1.89 | SEQ ID NO: 749 |
| 1.90 | SEQ ID NO: 750 |
| 1.91 | SEQ ID NO: 751 |
| 1.92 | SEQ ID NO: 752 |
| 1.93 | SEQ ID NO: 753 |
| 1.94 | SEQ ID NO: 754 |
| 1.95 | SEQ ID NO: 755 |
| 1.96 | SEQ ID NO: 756 |
| 1.97 | SEQ ID NO: 757 |
| 1.98 | SEQ ID NO: 758 |
| 1.99 | SEQ ID NO: 759 |
| 1.100 | SEQ ID NO: 760 |
| 1.101 | SEQ ID NO: 761 |
| 1.102 | SEQ ID NO: 762 |
| 1.103 | SEQ ID NO: 763 |
| 1.104 | SEQ ID NO: 764 |
| 1.105 | SEQ ID NO: 765 |
| 1.106 | SEQ ID NO: 766 |
| 1.107 | SEQ ID NO: 767 |
| 1.108 | SEQ ID NO: 768 |
| 1.109 | SEQ ID NO: 769 |
| 1.110 | SEQ ID NO: 770 |
| 1.111 | SEQ ID NO: 771 |
| 1.112 | SEQ ID NO: 772 |
| 1.113 | SEQ ID NO: 773 |
| 1.114 | SEQ ID NO: 774 |

TABLE 6
Nucleic acids encoding V_{H} 2.1 to 2.51 as shown in table 4

| Name | Nucleotide Sequence |
|---|---|
| 2.1 | SEQ ID NO: 775 |
| 2.2 | SEQ ID NO: 776 |
| 2.3 | SEQ ID NO: 777 |
| 2.4 | SEQ ID NO: 778 |
| 2.5 | SEQ ID NO: 779 |
| 2.6 | SEQ ID NO: 780 |
| 2.7 | SEQ ID NO: 781 |
| 2.8 | SEQ ID NO: 782 |
| 2.9 | SEQ ID NO: 783 |
| 2.10 | SEQ ID NO: 784 |
| 2.11 | SEQ ID NO: 785 |
| 2.12 | SEQ ID NO: 786 |
| 2.13 | SEQ ID NO: 787 |
| 2.14 | SEQ ID NO: 788 |
| 2.15 | SEQ ID NO: 789 |
| 2.16 | SEQ ID NO: 790 |
| 2.17 | SEQ ID NO: 791 |
| 2.18 | SEQ ID NO: 792 |
| 2.19 | SEQ ID NO: 793 |
| 2.20 | SEQ ID NO: 794 |
| 2.21 | SEQ ID NO: 795 |
| 2.22 | SEQ ID NO: 796 |
| 2.23 | SEQ ID NO: 797 |
| 2.24 | SEQ ID NO: 798 |
| 2.25 | SEQ ID NO: 799 |
| 2.26 | SEQ ID NO: 800 |
| 2.27 | SEQ ID NO: 801 |
| 2.28 | SEQ ID NO: 802 |
| 2.29 | SEQ ID NO: 803 |
| 2.30 | SEQ ID NO: 804 |
| 2.31 | SEQ ID NO: 805 |
| 2.32 | SEQ ID NO: 806 |
| 2.33 | SEQ ID NO: 807 |
| 2.34 | SEQ ID NO: 808 |
| 2.35 | SEQ ID NO: 809 |
| 2.36 | SEQ ID NO: 810 |
| 2.37 | SEQ ID NO: 811 |
| 2.38 | SEQ ID NO: 812 |
| 2.39 | SEQ ID NO: 813 |
| 2.40 | SEQ ID NO: 814 |
| 2.41 | SEQ ID NO: 815 |
| 2.42 | SEQ ID NO: 816 |
| 2.43 | SEQ ID NO: 817 |
| 2.44 | SEQ ID NO: 818 |
| 2.45 | SEQ ID NO: 819 |
| 2.46 | SEQ ID NO: 820 |
| 2.47 | SEQ ID NO: 821 |
| 2.48 | SEQ ID NO: 822 |
| 2.49 | SEQ ID NO: 823 |
| 2.50 | SEQ ID NO: 824 |
| 2.51 | SEQ ID NO: 825 |

In one embodiment, the nucleic acid sequence has at least 50% sequence homology to one of the sequences selected above. In one embodiment, said sequence homology is at least 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Novel Single Variable Heavy Chain Domain Antibodies that Bind to CD137

Whilst the CD137 binding single domain antibodies described above are used as building blocks for multispecific molecules that bind to both, CD137 and PD-1, in accordance with this invention we also provide novel single variable heavy chain domain antibodies that bind to CD137. These can be used as building blocks for multispecific molecules that bind to CD137 and a second target, such as both, CD137 and PD-1 or CD137 and PSMA as shown herein. These molecules bind to CD137, but do not cause CD137 signalling when bound to CD137 in monospecific format, that is without being linked to another moiety that binds a second target. The dual engagement of CD137 and, for example, a tumor specific antigen in a bispecific molecule leads to CD137 agonism. The isolated single variable heavy chain domain antibody binds to human CD137 but does not elicit CD137 signalling when bound to CD137 as a monospecific entity. In one embodiment, said single variable heavy chain domain antibody inhibits the binding of CD137L to CD137.

In one embodiment, the single variable heavy chain domain antibody comprises a CDR1 sequence selected from table 7 or a sequence with at least 75%, v homology/identity thereto, a CDR2 sequence selected from table 7 or a sequence with at least 75%, 80%, 90% or 95% homology/identity thereto and a CDR3 sequence selected from table 7 or a sequence with at least 75% homology/identity thereto. In one embodiment, the single variable heavy chain domain antibody comprises a full length sequence as shown in table 7 or a sequence with at least 75%, 80%, 90% or 95% homology/identity thereto. In one embodiment, the single variable heavy chain domain antibody comprises a set of CDR1, 2, and 3 sequences as shown for V_{H} single domain antibodies as shown in table 7 or a sequence with at least 75%, 80%, 90% or 95% sequence homology thereto. Thus, in one embodiment, the V_{H} single domain antibody has a CDR1 with SEQ ID NO. 850 or a sequence with at least 75%, 80%, 90% or 95% sequence homology thereto, a CDR2 with SEQ ID NO. 851 or a sequence with at least 75%, 80%, 90% or 95% sequence homology thereto and a CDR3 with SEQ ID NO. 852 or a sequence with at least 75%, 80%, 90% or 95% sequence homology thereto. In another embodiment, the V_{H} single domain antibody has a CDR1 with SEQ ID NO. 854 or a sequence with at least 75%, 80%, 90% or 95% sequence homology thereto, a CDR2 with SEQ ID NO. 855 and a CDR3 with SEQ ID NO. 856 or a sequence with at least 75%, 80%, 90% or 95% sequence homology thereto and so forth.

In one embodiment, the V_{H} comprises a CDR1, 2 and/or 3 sequence as shown for 003BB172-E01-13 in Table 7. In one embodiment, the V_{H} comprises sequence as shown for the full V_{H} sequence for 003BB172-E01-13 in Table 7. In one embodiment, the V_{H} is a variant of 003BB172-E01-13 that has a substitution at one or more of the positions L11V, G16R, R44G, M78T, T84N, T88A. In one embodiment, the variant is as shown in Table 7.

TABLE 7

| NAME | CDR1 | CDR2 | CDR3 | Full VH Sequence |
|---|---|---|---|---|
| 003BB1 | SEQ ID NO: | SEQ ID NO: | SEQ ID NO: | SEQ ID NO: 829 |
| 72-E01 | 826 | 827 | 828 | EVQLVESGGGLVQPGGSLRLSCAASGFTFSNYGMHWVRQA |
| | NYGMH | VISNDGNTKTY | DETTTVTAS | PGKRLEWVAVISNDGNTKTYADSVKGRFTISRDNSKNMLYLQ |
| | | ADSVKG | YYFDY | MTSLRTEDTAVYYCAQDETTTVTASYYFDYWGQGTLVTVSS |
| 003BB1 | SEQ ID NO: | SEQ ID NO: | SEQ ID NO: | SEQ ID NO: 833 |
| 72-E01- | 830 | 831 | 832 | EVQLVESGGGLVQPGGSLRLSCAASGFTFSNYGMHWVRQA |
| 1 | NYGMH | VISNDGNTKTY | DETTTVTAS | PGKGLEWVAVISNDGNTKTYADSVKGRFTISRDNSKNMLYL |
| | | ADSVKG | YYFDY | QMTSLRTEDTAVYYCAQDETTTVTASYYFDYWGQGTLVTVS |
| | | | | S |
| 003BB1 | SEQ ID NO: | SEQ ID NO: | SEQ ID NO: | SEQ ID NO: 837 |
| 72-E01- | 834 | 835 | 836 | EVQLVESGGGLVQPGGSLRLSCAASGFTFSNYGMHWVRQA |
| 2 | NYGMH | VISNDGNTKTY | DETTTVTAS | PGKRLEWVAVISNDGNTKTYADSVKGRFTISRDNSKNTLYLQ |
| | | ADSVKG | YYFDY | MTSLRTEDTAVYYCAQDETTTVTASYYFDYWGQGTLVTVSS |
| 003BB1 | SEQ ID NO: | SEQ ID NO: | SEQ ID NO: | SEQ ID NO: 841 |
| 72-E01- | 838 | 839 | 840 | EVQLVESGGGLVQPGGSLRLSCAASGFTFSNYGMHWVRQA |
| 3 | NYGMH | VISNDGNTKTY | DETTTVTAS | PGKRLEWVAVISNDGNTKTYADSVKGRFTISRDNSKNMLYLQ |
| | | ADSVKG | YYFDY | MNSLRTEDTAVYYCAQDETTTVTASYYFDYWGQGTLVTVSS |
| 003BB1 | SEQ ID NO: | SEQ ID NO: | SEQ ID NO: | SEQ ID NO: 845 |
| 72-E01- | 842 | 843 | 844 | EVQLVESGGGLVQPGGSLRLSCAASGFTFSNYGMHWVRQA |
| 4 | NYGMH | VISNDGNTKTY | DETTTVTAS | PGKGLEWVAVISNDGNTKTYADSVKGRFTISRDNSKNTLYLQ |
| | | ADSVKG | YYFDY | MTSLRTEDTAVYYCAQDETTTVTASYYFDYWGQGTLVTVSS |
| 003BB1 | SEQ ID NO: | SEQ ID NO: | SEQ ID NO: | SEQ ID NO: 849 |
| 72-E01- | 846 | 847 | 848 | EVQLVESGGGLVQPGGSLRLSCAASGFTFSNYGMHWVRQA |
| 5 | NYGMH | VISNDGNTKTY | DETTTVTAS | PGKRLEWVAVISNDGNTKTYADSVKGRFTISRDNSKNTLYLQ |
| | | ADSVKG | YYFDY | MNSLRTEDTAVYYCAQDETTTVTASYYFDYWGQGTLVTVSS |
| 003BB1 | SEQ ID NO: | SEQ ID NO: | SEQ ID NO: | SEQ ID NO: 853 |
| 72-E01- | 850 | 851 | 852 | EVQLVESGGGLVQPGGSLRLSCAASGFTFSNYGMHWVRQA |
| 6 | NYGMH | VISNDGNTKTY | DETTTVTAS | PGKGLEWVAVISNDGNTKTYADSVKGRFTISRDNSKNMLYL |
| | | ADSVKG | YYFDY | QMNSLRTEDTAVYYCAQDETTTVTASYYFDYWGQGTLVTVS |
| | | | | S |
| 003BB1 | SEQ ID NO: | SEQ ID NO: | SEQ ID NO: | SEQ ID NO: 857 |
| 72-E01- | 854 | 855 | 856 | EVQLVESGGGLVQPGGSLRLSCAASGFTFSNYGMHWVRQA |
| 7 | NYGMH | VISNDGNTKTY | DETTTVTAS | PGKGLEWVAVISNDGNTKTYADSVKGRFTISRDNSKNTLYLQ |
| | | ADSVKG | YYFDY | MNSLRTEDTAVYYCAQDETTTVTASYYFDYWGQGTLVTVSS |
| 003BB1 | SEQ ID NO: | SEQ ID NO: | SEQ ID NO: | SEQ ID NO: 861 |
| 72-E01- | 858 | 859 | 860 | EVQLVESGGGVVQPGGSLRLSCAASGFTFSNYGMHWVRQA |
| 8 | NYGMH | VISNDGNTKTY | DETTTVTAS | PGKGLEWVAVISNDGNTKTYADSVKGRFTISRDNSKNTLYLQ |
| | | ADSVKG | YYFDY | MNSLRTEDTAVYYCAQDETTTVTASYYFDYWGQGTLVTVSS |
| 003BB1 | SEQ ID NO: | SEQ ID NO: | SEQ ID NO: | SEQ ID NO: 865 |
| 72-E01- | 862 | 863 | 864 | EVQLVESGGGLVQPGGSLRLSCAASGFTFSNYGMHWVRQA |
| 9 | NYGMH | VISNDGNTKTY | DETTTVTAS | PGKGLEWVAVISNDGNTKTYADSVKGRFTISRDNSKNTLYLQ |
| | | ADSVKG | YYFDY | MNSLRAEDTAVYYCAQDETTTVTASYYFDYWGQGTLVTVSS |
| 003BB1 | SEQ ID NO: | SEQ ID NO: | SEQ ID NO: | SEQ ID NO: 869 |
| 72-E01- | 866 | 867 | 868 | EVQLVESGGGLVQPGRSLRLSCAASGFTFSNYGMHWVRQAP |
| 10 | NYGMH | VISNDGNTKTY | DETTTVTAS | GKGLEWVAVISNDGNTKTYADSVKGRFTISRDNSKNTLYLQM |
| | | ADSVKG | YYFDY | NSLRTEDTAVYYCAQDETTTVTASYYFDYWGQGTLVTVSS |
| 003BB1 | SEQ ID NO: | SEQ ID NO: | SEQ ID NO: | SEQ ID NO: 873 |
| 72-E01- | 870 | 871 | 872 | EVQLVESGGGVVQPGRSLRLSCAASGFTFSNYGMHWVRQA |
| 11 | NYGMH | VISNDGNTKTY | DETTTVTAS | PGKGLEWVAVISNDGNTKTYADSVKGRFTISRDNSKNTLYLQ |
| | | ADSVKG | YYFDY | MNSLRTEDTAVYYCAQDETTTVTASYYFDYWGQGTLVTVSS |
| 003BB1 | SEQ ID NO: | SEQ ID NO: | SEQ ID NO: | SEQ ID NO: 877 |
| 72-E01- | 874 | 875 | 876 | EVQLVESGGGLVQPGRSLRLSCAASGFTFSNYGMHWVRQAP |
| 12 | NYGMH | VISNDGNTKTY | DETTTVTAS | GKGLEWVAVISNDGNTKTYADSVKGRFTISRDNSKNTLYLQM |
| | | ADSVKG | YYFDY | NSLRAEDTAVYYCAQDETTTVTASYYFDYWGQGTLVTVSS |
| 003BB1 | SEQ ID NO: | SEQ ID NO: | SEQ ID NO: | SEQ ID NO: 881 |
| 72-E01- | 878 | 879 | 880 | EVQLVESGGGVVQPGGSLRLSCAASGFTFSNYGMHWVRQA |
| 13 | NYGMH | VISNDGNTKTY | DETTTVTAS | PGKGLEWVAVISNDGNTKTYADSVKGRFTISRDNSKNTLYLQ |
| | | ADSVKG | YYFDY | MNSLRAEDTAVYYCAQDETTTVTASYYFDYWGQGTLVTVSS |
| 003BB1 | SEQ ID NO: | SEQ ID NO: | SEQ ID NO: | SEQ ID NO: 885 |
| 72-E01- | 882 | 883 | 884 | EVQLVESGGGVVQPGRSLRLSCAASGFTFSNYGMHWVRQA |
| 14 | NYGMH | VISNDGNTKTY | DETTTVTAS | PGKGLEWVAVISNDGNTKTYADSVKGRFTISRDNSKNTLYLQ |
| | | ADSVKG | YYFDY | MNSLRAEDTAVYYCAQDETTTVTASYYFDYWGQGTLVTVSS |

Thus, we also provide multispecific molecules comprising a single variable heavy chain domain antibody described above and comprising another moiety. The other moiety can be selected from an antibody, an antibody fragment, an antibody mimetic or other polypeptide. The antibody fragment can be selected from a Fab, F(ab′)2, Fv, a single chain Fv fragment (scFv), a single domain antibody or fragment thereof. For example, it is a single V_{H} domain antibody. Non-limiting examples of other moieties include PD-1 as shown herein and PSMA. Exemplary V_{H} single domain antibodies that bind to PSMA and can be combined in a single molecule with the CD137 binding are shown herein. We also provide a V_{H} single domain antibody that binds to CD137 as described above and shown in table 7 for use in a multispecific molecule, for example with a single domain antibody as shown in table 8, 9, 10 or 11.

Nucleic acids encoding the single variable heavy chain domain antibody, vectors and host cells are also envisaged. Examples of host cells are described herein. Pharmaceutical compositions and kits comprising such a single variable heavy chain domain antibody are also provided. These can have the features provided below. Also provided are methods for treating a disease, e.g. a cancer using V_{H} single domain antibody that binds to CD137 as described above and shown in table 8, for example when used in a multispecific molecule. Also provided is V_{H} single domain antibody that binds to CD137 as described above and shown in table 8 for use in the treatment of disease, e.g. a cancer. Exemplary cancers are discussed herein, e.g. when the V_{H} single domain antibody that binds to CD137 is used in a multispecific molecule that binds PD-1. When used with PSMA as shown herein, the use is for treating prostate cancer.

Exemplary Immunoglobulins Included in the Binding Molecule and which Bind PD-1

Examples of moieties for example, single variable heavy chain domain antibodies, that bind to PD-1 and that may form one of the subunits of the binding molecules disclosed herein, such as a molecule that comprises or consists of two single domain antibodies that bind to both CD137 and PD-1, are described below and can be used in the various embodiments of the invention.

Molecules of the invention bind specifically to wild type human PD-1 (UniProt Accession No. Q15116, GenBank Accession No. U64863). Residues 1-20 correspond to the pre-sequence, residues 171 and beyond make up the transmembrane helix and the intracellular domain of PD-1. The sequence is shown in SEQ ID NO: 1877.

Unless otherwise specified, the term PD-1 as used herein refers to human PD-1. The terms “Programmed Death 1,” “Programmed Cell Death 1”, “Protein PD-1,” “PD-1,” “PD1,” “PDCD1,” “hPD-1” and “hPD-1” are used interchangeably, and include variants, isoforms, species homologs of human PD-1.

In one embodiment, the PD-1 binding subunit binds to wild type human PD-1 and/or cyno PD-1. The terms “PD-1 binding molecule”, “PD-1 binding protein” “anti-PD-1 single domain antibody”, “PD-1 binding subunit”, or “anti-PD-1 antibody” as used herein all refer to a molecule capable of binding to the human PD-1 antigen. The term “PD-1 binding molecule” includes a PD-1 binding protein. The binding reaction may be shown by standard methods (qualitative assays) including, for example, a binding assay, competition assay or a bioassay for determining the inhibition of PD-1 binding to its receptor or any kind of binding assays, with reference to a negative control test in which an antibody of unrelated specificity. Suitable assays are shown in the examples.

A binding molecule of the invention, including a single domain antibody and multivalent or multispecific binding agent described herein, “which binds” or is “capable of binding” an antigen of interest, e.g. “PD-1 binding molecule”, is one that binds, i.e. targets, the PD-1 antigen with sufficient affinity such that it is useful in therapy in targeting a cell or tissue expressing the antigen.

A single V_{H} domain antibody that binds to PD-1 may be as described in WO2018/127709 or WO2018/127710 both incorporated herein in their entirety by reference.

### PD-1 Non-Blocking Molecules

According to one aspect of the invention, the subunit that binds to PD-1, such as a single domain antibody, for example generated in vivo in transgenic mice, binds to human PD-1, but does not block the functional interaction between human PD-1 and its ligands, i.e. the interaction of human PD-1 with human PD-L1 and/or PD-L2. Thus, the anti-PD-1 V_{H} single domain antibodies bind an epitope that is distant from the part of the PD-1 protein that interacts with its ligands PD-L1 and PD-L2 and that is therefore outside the region of binding of known therapeutics targeting PD-1. This renders a human variable single domain antibody as described herein particularly useful in anchoring the multispecific binding molecules to human PD-1. Surprisingly, the inventors have also found that this binding to PD-1 also enables PD-1 to be down-modulated from the surface when used in a bispecific molecule with a CD137 binding V_{H}. Thus, the inventors have found that bispecific molecules described herein that comprise a V_{H} single domain antibody that binds to CD137 and a V_{H} single domain antibody that binds to PD-1, but which when used on its own does not block PD-1 signalling/activity when used in on its own, are capable of modulating the activity of PD-1; e.g. down-regulating the presence of PD-1 on the cell surface.

The V_{H} single domain antibody can exhibit one or more of the following properties when use don its own:

(a) binds to human PD-1 with a KD as shown in the examples;
(b) does not block the functional interaction of PD-1 with its ligands;
(c) binds to human PD-1 and cynomolgus monkey PD-1;
(d) does not bind to mouse PD-1;
(e) is capable of enhancing antagonistic action of an antagonistic human V_{H} single domain antibody when linked to such antibody;
(f) does not enhance T cell activation;
(g) binds to a human PD-1 with a binding affinity of KD 10⁻⁸ to 10⁻¹⁰;
(h) has an EC50 value in the subnanomolar range as determined in binding to CHO-PD-1 cell line
(i) binds to an epitope comprising one or more residue selected from one or more of R¹⁰⁴, D¹⁰⁵, F¹⁰⁶, H¹⁰⁷, M¹⁰⁸, S¹⁰⁹ and V¹¹⁰ of human PD-1 optionally where the epitope further comprises one or more or all of residues S⁶⁰, E⁶¹, S⁶², F⁶³, V⁶⁴, L⁶⁵, N⁶⁶, W⁶⁷, Y⁶⁸, R⁶⁹, M⁷⁰, S⁷¹, G⁹⁰, Q⁹¹, D⁹², C⁹³, R⁹⁴, F⁹⁵, R⁹⁶, V⁹⁷, T⁹⁸, V¹¹¹, R¹¹², A¹¹³ and R¹¹⁴, N¹⁰², D¹⁰⁵, F¹⁰⁶, H¹⁰⁷, M¹⁰⁸, R¹¹⁵, N³³, P³⁴, P³⁵, T³⁶, F³⁷, S³⁸, C⁵⁶, F⁵⁶, S⁵⁷, N⁵⁵, T⁵⁹, P¹⁰¹ and G¹⁰³ and/or
(j) is produced in a transgenic rodent as described herein and/or
(k) is capable to down-regulating the PD-1 on the cell surface when combined with a moiety targeting another cell surface receptor such as CD-137.

### Exemplary Sequences

In one embodiment, the single variable heavy chain domain antibody comprises human framework regions. In one embodiment, the single variable heavy chain domain antibody is selected from single variable heavy chain domain antibody as shown in table 8, 9 or 10 or from a sequence with at least 75%, 80%, 90% or 95% homology thereto. The PD-1 binding entity can also be selected from a part thereof, such as a CDR3.

In one embodiment, the single variable heavy chain domain antibody that binds to PD-1 and is used in the multispecific molecules that bind both, CD137 and PD-1, as described herein, comprises a CDR1 sequence selected from one of the CDR1 sequences shown in table 8 or a sequence with at least 75% homology thereto, a CDR2 sequence selected from one of the CDR2 sequences shown in table 8 or a sequence with at least 75%, 80%, 90% or 95% homology thereto and a CDR3 sequence selected from one of the CDR3 sequences shown in table 8 or a sequence with at least 75%, 80%, 90% or 95% homology thereto. In one embodiment, the single variable heavy chain domain antibody comprises a set of CDR1, 2, and 3 sequences as shown for V_{H} single domain antibodies PDA, PDA2 or PDA138G7(G109D) as shown in table 8 or a sequence with at least 75%, 80%, 90% or 95% sequence homology thereto. Thus, in one embodiment, the V_{H} single domain antibody has a CDR1 with SEQ ID NO. 1038 or a sequence with at least 75%, 80%, 90% or 95% sequence homology thereto, a CDR2 with SEQ ID NO. 1039 or a sequence with at least 75%, 80%, 90% or 95% sequence homology thereto and a CDR3 with SEQ ID NO. 1040 or a sequence with at least 75%, 80%, 90% or 95% sequence homology thereto. In another embodiment, the VH single domain antibody has a CDR1 with SEQ ID NO. 1130 or a sequence with at least 75%, 80%, 90% or 95% sequence homology thereto, a CDR2 with SEQ ID NO. 1131 or a sequence with at least 75%, 80%, 90% or 95% sequence homology thereto and a CDR3 with SEQ ID NO. 1132 or a sequence with at least 75%, 80%, 90% or 95% sequence homology thereto. In another embodiment, the V_{H} single domain antibody has a CDR1 with SEQ ID NO. 1294 or a sequence with at least 75%, 80%, 90% or 95% sequence homology thereto, a CDR2 with SEQ ID NO. 1295 and a CDR3 with SEQ ID NO. 1296 or a sequence with at least 75%, 80%, 90% or 95% sequence homology thereto.

In one embodiment, the single variable heavy chain domain antibody that binds to PD-1 and is used in the multispecific molecules that bind both, CD137 and PD-1, as described herein comprises a CDR1 sequence selected from one of the CDR1 sequences shown in table 9 or a sequence with at least 75%, 80%, 90% or 95% homology thereto, a CDR2 sequence selected from one of the CDR2 sequences shown in table 9 or a sequence with at least 75% homology thereto and a CDR3 sequence selected from one of the CDR3 sequences shown in table 9 or a sequence with at least 75%, 80%, 90% or 95% homology thereto. In one embodiment, the single variable heavy chain domain antibody comprises a set of CDR1, 2, and 3 sequences as shown for V_{H} single domain antibodies as shown in table 9 or a sequence with at least 75%, 80%, 90% or 95% sequence homology thereto. Thus, in one embodiment, the V_{H} single domain antibody has a CDR1 with SEQ ID NO. 886 or a sequence with at least 75%, 80%, 90% or 95% sequence homology thereto, a CDR2 with SEQ ID NO. 887 or a sequence with at least 75%, 80%, 90% or 95% sequence homology thereto and a CDR3 with SEQ ID NO: 889 or a sequence with at least 75%, 80%, 90% or 95% sequence homology thereto a CDR1 with SEQ ID NO., 891 or a sequence with at least 75%, 80%, 90% or 95% sequence homology thereto a CDR2 with SEQ ID NO. 892 and a CDR3 with SEQ ID NO: 893 or a sequence with at least 75%, 80%, 90% or 95% sequence homology thereto a CDR1 with SEQ ID NO. 895 or a sequence with at least 75%, 80%, 90% or 95% sequence homology thereto, a CDR2 with SEQ ID NO. 896 or a sequence with at least 75%, 80%, 90% or 95% sequence homology thereto and a CDR3 with SEQ ID NO. 897 or a sequence with at least 75%, 80%, 90% or 95% sequence homology thereto and so forth.

In one embodiment, the single variable heavy chain domain antibody that binds to PD-1 and is used in the multispecific molecules that bind both, CD137 and PD-1, as described herein comprises a CDR1 sequence selected from one of the CDR1 sequences shown in table 10 or a sequence with at least 75%, 80%, 90% or 95% homology thereto, a CDR2 sequence selected from one of the CDR2 sequences shown in table 10 or a sequence with at least 75%, 80%, 90% or 95% homology thereto and a CDR3 sequence selected from one of the CDR3 sequences shown in table 10 or a sequence with at least 75%, 80%, 90% or 95% homology thereto. In one embodiment, the single variable heavy chain domain antibody comprises a set of CDR1, 2, and 3 sequences as shown for V_{H} single domain antibodies as shown in table 9 or a sequence with at least 75%, 80%, 90% or 95% sequence homology thereto. Thus, in one embodiment, the V_{H} single domain antibody has a CDR1 with SEQ ID NO. 1086 or a sequence with at least 75%, 80%, 90% or 95% sequence homology thereto, a CDR2 with SEQ ID NO. 1087 or a sequence with at least 75%, 80%, 90% or 95% sequence homology thereto and a CDR3 with SEQ ID NO: 1088 or a sequence with at least 75%, 80%, 90% or 95% sequence homology thereto a CDR1 with SEQ ID NO. 1090 or a sequence with at least 75%, 80%, 90% or 95% sequence homology thereto a CDR2 with SEQ ID NO. 1091 and a CDR3 with SEQ ID NO: 1092 or a sequence with at least 75%, 80%, 90% or 95% sequence homology thereto a CDR1 with SEQ ID NO. 1094 or a sequence with at least 75%, 80%, 90% or 95% sequence homology thereto, a CDR2 with SEQ ID NO. 1095 or a sequence with at least 75%, 80%, 90% or 95% sequence homology thereto and a CDR3 with SEQ ID NO. 1096 or a sequence with at least 75%, 80%, 90% or 95% sequence homology thereto and so forth.

In one embodiment, the single variable heavy chain domain antibody that binds to PD-1 and is used in the multispecific molecules that bind both, CD137 and PD-1, as described herein comprises a full length sequence selected from one of the full length sequences shown in table 8, 9 or 10. Sequence homology can be at least 75%, that is at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% for example at least 90%, 91%, 92%, 93%, 94%, 95%, 96%, 97%, 98% or at least 99% or 100% sequence homology or identity.

TABLE 8
Exemplary PD-1 binding single V_{H} domain
antibodies that do not block the PD-1
function and its in on their own. These
single V_{H} domain antibodies can be used
in the multispecific binding molecules
described herein, e.g. by fusion to a
CD137 binding single V_{H} domain antibody
as described herein, e.g. in Table 2.

| | | | | SEQ ID |
|---|---|---|---|---|
| | | | | V_{H} |
| | | | | Full |
| Name | CDR1 | CDR2 | CDR3 | length |
| PDA | SEQ ID | SEQ ID | SEQ ID | SEQ ID |
| | NO: | NO: | NO: | NO: |
| | 1038 | 1039 | 1040 | 1041 |
| | DYALH | GISWN | EKGPGL | EVQLVE |
| | | GGSYG | TGSTAD | SGGGLV |
| | | YADSV | YYGLDA | QPGRSL |
| | | KG | | RLSCAA |
| | | | | SGFTFD |
| | | | | DYALHW |
| | | | | VRQAPG |
| | | | | KDLEWV |
| | | | | SGISWN |
| | | | | GGSYGY |
| | | | | ADSVKG |
| | | | | RFTISR |
| | | | | DNAKNS |
| | | | | LYLQMN |
| | | | | SLRAED |
| | | | | TALYYC |
| | | | | VREKGP |
| | | | | GLTGST |
| | | | | ADYYGL |
| | | | | DAWGQG |
| | | | | TMVTVS |
| | | | | S |
| PDA2 | SEQ ID | SEQ ID | SEQ ID | SEQ ID |
| | NO: | NO: | NO: | NO: |
| | 1130 | 1131 | 1132 | 1133 |
| | DYAMS | GITW | DKYSYA | EVQLVE |
| | | NAGS | WSYDDF | SGGGVV |
| | | TGYA | Dl | RPGGSL |
| | | DSVKG | | RLSCAA |
| | | | | SGFTFD |
| | | | | DYAMSW |
| | | | | VRQAPG |
| | | | | KGLEWV |
| | | | | SGITWN |
| | | | | AGSTGY |
| | | | | ADSVKG |
| | | | | RFTISR |
| | | | | DNAKNS |
| | | | | LYLQMN |
| | | | | SLRAED |
| | | | | TALYYC |
| | | | | VRDKYS |
| | | | | YAWSYD |
| | | | | DFDIWG |
| | | | | QGTMVT |
| | | | | VSS |
| PD138G7 | SEQ ID | SEQ ID | SEQ ID | SEQ ID |
| (G109D) | NO: | NO: | NO: | NO: |
| | 1294 | 1295 | 1296 | 1297 |
| | DYAMS | GITWN | DKYSYA | EVQLVE |
| | | GGSTG | WSYDDF | SGGGVV |
| | | YADSV | Dl | RPGGSL |
| | | KD | | RLSCAA |
| | | | | SGFTFD |
| | | | | DYAMSW |
| | | | | VRQAPG |
| | | | | KGLEWV |
| | | | | SGITWN |
| | | | | GGSTGY |
| | | | | ADSVKD |
| | | | | RFTISR |
| | | | | DNAKNS |
| | | | | LYLQMN |
| | | | | SLRAED |
| | | | | TALYYC |
| | | | | VRDKYS |
| | | | | YAWSYD |
| | | | | DFDIWG |
| | | | | QGTMVT |
| | | | | VSS |

TABLE 9
Further exemplary PD-1 binding single V_{H} domain antibodies
that do not block the function of human PD-1 when use don
their own. These V_{H} domain antibodies bind to an epitope
comprising one or more or all residues selected from N¹⁰²,
D¹⁰⁵, F¹⁰⁶, H¹⁰⁷, M¹⁰⁸, R¹¹⁴ and R¹¹⁵ of human PD-1. These
single V_{H} domain antibodies can be used in the
multispecific binding molecules described herein,
e.g. by fusion to a CD137 binding single V_{H} domain
antibody as described herein, e.g. in Table 2.

| | CDR1 | CDR2 | CDR3 | Full length V_{H} sequence with |
|---|---|---|---|---|
| | Sequence | Sequence | Sequence | formula 1: FR1-CDR1-FR2- |
| Name | of V_{H} | Of V_{H} | Of V_{H} | CDR2-FR3-CDR3-FR4 |
| PD1.1 | SEQ ID NO: | SEQ ID NO: | SEQ ID NO: | SEQ ID NO: 889 |
| | 886 DHAMH | 887 | 888 | EVQLLESGGGSVQPGRSLRLSCAASGFTFDDHAMHWVRQ |
| | | GISWNSGSM | EKGPGLTGSTA | APGKGLEWVSGISWNSGSMGYADSVKDRFTISRDNAKSSL |
| | | GYADSVKD | DYYGLDV | YLQMNSLRAEDTALYYCVREKGPGLTGSTADYYGLDVWGQ |
| | | | | GTMVTVSS |
| PD 1.2 | SEQ ID NO: | SEQ ID NO: | SEQ ID NO: | SEQ ID NO: 893 |
| | 890 DYAMH | 891 | 892 | EVQLVESGGGLVQPGRSLRLSCAASGFTFDDYAMHWVRQ |
| | | GISWNGGS | DKGPGLIGSTA | APGKDLEWVSGISWNGGSMGYAASVKGRFTISRDNAKNSL |
| | | MGYAASVK | DYYGLDV | YLQMNSLRAEDTALYYCVKDKGPGLIGSTADYYGLDVWGQ |
| | | G | | GTTVTVSS |
| PD 1.3 | SEQ ID NO: | SEQ ID NO: | SEQ ID NO: | SEQ ID NO: 897 |
| | 894 DYAMH | 895 | 896 | EVQLLESGGGLVQPGRSLRLSCAASGFTFDDYAMHWVRQA |
| | | GISWNSGSM | DKGPGLIGSTA | PGKGREWVSGISWNSGSMGYADSVKDRFTISRDNAKNSLY |
| | | GYADSVKD | DYHGLDV | LQMNSLRAEDTALYYCVKDKGPGLIGSTADYHGLDVWGQG |
| | | | | TTVTVSS |
| PD 1.4 | SEQ ID NO: | SEQ ID NO: | SEQ ID NO: | SEQ ID NO: 901 |
| | 898 DYAMH | 899 | 900 | EVQLLESGGGLVQPGRSLRLSCAASGFTFADYAMHWVRQA |
| | | GISWNGGS | DKGPGLTGTTA | PGKGREWVSGISWNGGSMGYAESVKGRFTISRDNAKNSLY |
| | | MGYAESVKG | DYYGMDV | LQMNSLRAEDSALYYCVKDKGPGLTGTTADYYGMDVWGQ |
| | | | | GTTVTVSS |
| PD 1.5 | SEQ ID NO: | SEQ ID NO: | SEQ ID NO: | SEQ ID NO: 905 |
| | 902 DYAMH | 903 | 904 | EVQLVESGGGLVQPGRSLRLSCAASGFTFDDYAMHWVRQ |
| | | GISWNGGS | DKGPGLIGSTA | APGKGLEWVSGISWNGGSMGYADSVKDRFTISRDNAKNSL |
| | | MGYADSVK | DYHGLDV | YLQMNRLRAEDTALYYCVKDKGPGLIGSTADYHGLDVWGQ |
| | | D | | GTTVTVSS |
| PD 1.6 | SEQ ID NO: | SEQ ID NO: | SEQ ID NO: | SEQ ID NO: 909 |
| | 906 SYAMH | 907 | 908 | EVQLVESGGGVVQPGRSLRLSCAASGFTFSSYAMHWVRQA |
| | | GISWNSGSM | DKGPGLTGTTA | PGKGREWVSGISWNSGSMGYAESVKGRFTISRDNAKNSLY |
| | | GYAESVKG | DYYGMDV | LQMNSLRAEDSALYYCVKDKGPGLTGTTADYYGMDVWGQ |
| | | | | GTTVTVSS |
| PD 1.7 | SEQ ID NO: | SEQ ID NO: | SEQ ID NO: | SEQ ID NO: 913 |
| | 910 DYAMH | 911 | 912 | EVQLVESGGGVIQPGRSLRLSCAASGFTFDDYAMHWVRQA |
| | | GISWNGGS | DKGPGLTGTTA | PGKGREWVSGISWNGGSMGYAESVKGRFTISRDNAQNSL |
| | | MGYAESVKG | DYYGMDV | YLQMNSLRAEDSALYYCVKDKGPGLTGTTADYYGMDVWG |
| | | | | QGTTVTVSS |
| PD 1.8 | SEQ ID NO: | SEQ ID NO: | SEQ ID NO: | SEQ ID NO: 917 |
| | 914 DYAMH | 915 | 916 | EVQLVESGGGLVQPGRSLRLSCAASGFTFDDYAMHWVRQ |
| | | GISWNSGSM | DKGPGLIGSTA | APGKGLEWVSGISWNSGSMGYADSVKDRFTISRDNAKNSL |
| | | GYADSVKD | DYHGLDV | YLQMNSLRAEDTALYYCVKDKGPGLIGSTADYHGLDVWGQ |
| | | | | GTTVTVSS |
| PD 1.9 | SEQ ID NO: | SEQ ID NO: | SEQ ID NO: | SEQ ID NO: 921 |
| | 918 DYAMH | 919 | 920 | EVQLVESGGGCVQPGRSLRISCAASGFTFDDYAMHWVRQ |
| | | GISWNGGS | DKGPGLIGSTA | APGKGREWVSGISWNGGSMGYAESVKGRFTISRDNAKNSL |
| | | MGYAESVKG | DYYGMDV | YLQMNSLRAEDSALYYCVKDKGPGLIGSTADYYGMDVWG |
| | | | | QGTTVTVSS |
| PD 1.10 | SEQ ID NO: | SEQ ID NO: | SEQ ID NO: | SEQ ID NO: 925 |
| | 922 DYAMH | 923 | 924 | EVQLVESGGGLVQPGRSLRLSCAASGFTFADYAMHWVRQ |
| | | GISWNGGS | DKGPGLTGTTA | APGKGREWVSGISWNGGSMGYAESVKGRFTISRDNAKNSL |
| | | MGYAESVKG | DYYGMDV | YLQMNSLRAEDSALYYCVKDKGPGLTGTTADYYGMDVWG |
| | | | | QGTTVTVSS |
| PD 1.11 | SEQ ID NO: | SEQ ID NO: | SEQ ID NO: | SEQ ID NO: 929 |
| | 926 DYAMH | 927 | 928 | EVQLLESGGGFVQPGRSLRISCAASGFTFDDYAMHWVRQA |
| | | GISWNGGS | DKGPGLIGSTA | PGKDLEWVSGISWNGGSMGYAESVKGRFTISRDNAKNSLY |
| | | MGYAESVKG | DYYGLDV | LKMNSLRVEDTALYYCVKDKGPGLIGSTADYYGLDVWGQG |
| | | | | TTVTVSS |
| PD 1.12 | SEQ ID NO: | SEQ ID NO: | SEQ ID NO: | SEQ ID NO: 933 |
| | 930 DYAMH | 931 | 932 | QVQLVESGGGLVQPGRSLRLSCAASGFTFDDYAMHWVRQ |
| | | GISWNGGS | DKGPGLTGSTA | APGKGREWVSGISWNGGSMGYADSVKDRFTISRDNAKNS |
| | | MGYADSVK | DYHGMDV | LYLQMNSLRAEDTALYYCVKDKGPGLTGSTADYHGMDVW |
| | | D | | G QGTTVTVSS |
| PD 1.13 | SEQ ID NO: | SEQ ID NO: | SEQ ID NO: | SEQ ID NO: 937 |
| | 934 DYAMH | 935 | 936 | QVQLVESGGGLVQPGRSLRLSCAASGFTFDDYAMHWVRQ |
| | | GISWNGGS | DKGPGLIGSTA | APGKDLEWVSGISWNGGSMGYAASVKGRFTISRDNAQNS |
| | | MGYAASVK | DYYGLDV | LYLQMNSLRAEDTALYYCVKDKGPGLIGSTADYYGLDVWG |
| | | G | | QGTTVTVSS |
| PD 1.14 | SEQ ID NO: | SEQ ID NO: | SEQ ID NO: | SEQ ID NO: 941 |
| | 938 GYAMH | 939 | 940 | EVQLVESGGGLVQPGRSLRLSCAASGFTFDGYAMHWVRQ |
| | | GISWNSGSM | DKGPGLTGSTA | APGKGREWVSGISWNSGSMGYAESVKGRFTISRDNAKNSL |
| | | GYAESVKG | DYYGMDV | YLQMNSLRAEDSALYYCVKDKGPGLTGSTADYYGMDVWG |
| | | | | QGTTVTVSS |
| PD 1.15 | SEQ ID NO: | SEQ ID NO: | SEQ ID NO: | SEQ ID NO: 945 |
| | 942 DYAMH | 943 | 944 | EVQLLESGGGLVQPGRSLRLSCTASGFTFDDYAMHWVRQA |
| | | GISWNSGSM | DKGPGLIGSTA | PGKGREWVSGISWNSGSMGYAESVKGRFTISRDNAKNSLY |
| | | GYAESVKG | DYYGMDV | LQMNSLRAEDSALYYCVKDKGPGLIGSTADYYGMDVWGQ |
| | | | | GTTVTVSS |
| PD 1.16 | SEQ ID NO: | SEQ ID NO: | SEQ ID NO: | SEQ ID NO: 949 |
| | 946 DYAMH | 947 | 948 | QVQLVESGGGLVQPGRSLRLSCAASGFTFDDYAMHWVRQ |
| | | GISWNGGS | DKGPGLIGSTA | APGKGREWVSGISWNGGSMGYAESVKGRFTISRDNAKNSL |
| | | MGYAESVKG | DYYGMDV | YLQMNSLRAEDSALYYCVKDKGPGLIGSTADYYGMDVWG |
| | | | | QGTTVTVSS |
| PD 1.17 | SEQ ID NO: | SEQ ID NO: | SEQ ID NO: | SEQ ID NO: 953 |
| | 950 DYAMH | 951 | 952 | EVQLLESGGGLVQPGGSLRLSCAASGFTFDDYAMHWVRQ |
| | | GISWNSGSM | DKGPGLIGSTA | APGKGLEWVSGISWNSGSMGYAASVKDRFTISRDNAKNSL |
| | | GYAASVKD | DYHGLDV | YLQMNSLTTEDTALYYCVKDKGPGLIGSTADYHGLDVWGQ |
| | | | | GTTVTVSS |
| PD 1.18 | SEQ ID NO: | SEQ ID NO: | SEQ ID NO: | SEQ ID NO: 957 |
| | 954 DYAMH | 955 | 956 | QVQLVESGGGLVQPGRSLRLSCAASGFTFDDYAMHWVRQ |
| | | GISWNGGS | DKGPGLIGSTA | APGKDLEWVSGISWNGGSMGYAASVKGRFTISRDNAKNSL |
| | | MGYAASVK | DYYGLDV | YLQMNSLRAEDTALYYCVKDKGPGLIGSTADYYGLDVWGQ |
| | | G | | GTTVTVSS |
| PD 1.19 | SEQ ID NO: | SEQ ID NO: | SEQ ID NO: | SEQ ID NO: 961 |
| | 958 DYAMH | 959 | 960 | EVQLVESGGGLVQPGRSLRLSCAASGFTFDDYAMHWVRQ |
| | | GISWNGGS | EKGPGLTGSTA | APGKDLEWVSGISWNGGSMGYADSVKGRFTISRDNAKNSL |
| | | MGYADSVK | DYYGLDV | YLQMNSLRAEDTALYYCVREKGPGLTGSTADYYGLDVWGQ |
| | | G | | GTMVTVSS |
| PD 1.20 | SEQ ID NO: | SEQ ID NO: | SEQ ID NO: | SEQ ID NO: 965 |
| | 962 DYAMH | 963 | 964 | EVQLVESGGGLVQPGRSLRLSCAASGFTFDDYAMHWVRQ |
| | | GISWNGGS | EKGPGLTGSTA | APGKDLEWVSGISWNGGSMGYADSVKGRFTISRDNAKNSL |
| | | MGYADSVK | DYYGLDV | YLQMNSLRAEDTALYYCVREKGPGLTGSTADYYGLDVWGQ |
| | | G | | GTMVTVSS |
| PD 1.21 | SEQ ID NO: | SEQ ID NO: | SEQ ID NO: | SEQ ID NO: 969 |
| | 966 | 967 | 968 | EVQLVESGGGLVQPGRSLRLSCAASGFTFDDYAMHWVRQ |
| | DYAMH | GISWNGGS | EKGPGLTGSTA | APGKDLEWVSGISWNGGSMGYAASVKGRFTISRDNAKNSL |
| | | MGYAASVK | DYYGLDV | YLQMNSLRAEDTALYYCVREKGPGLTGSTADYYGLDVWGQ |
| | | G | | GTMVTVSS |
| PD 1.22 | SEQ ID NO: | SEQ ID NO: | SEQ ID NO: | SEQ ID NO: 973 |
| | 970 | 971 | 972 | EVQLVESGGGLVQPGRSLRLSCAASGFTFDDYAMHWVRQ |
| | DYAMH | GISWNGGS | EKGPGLTGSTA | APGKDLEWVSGISWNGGSMGYAASVKGRFTISRDNAKNSL |
| | | MGYAASVK | DYYGLDA | YLQMNSLRAEDTALYYCVREKGPGLTGSTADYYGLDAWGQ |
| | | G | | GTMVTVSS |
| PD 1.23 | SEQ ID NO: | SEQ ID NO: | SEQ ID NO: | SEQ ID NO: 977 |
| | 974 | 975 | 976 | EVQLVESGGGLVQPGRSLRLSCAASGFTFDDYAMHWVRQ |
| | DYAMH | GISWNSGSM | EKGPGLTGSTA | APGKDLEWVSGISWNSGSMGYAASVKGRFTISRDNAKNSL |
| | | GYAASVKG | DYYGLDV | YLQMNSLRAEDTALYYCVREKGPGLTGSTADYYGLDVWGQ |
| | | | | GTMVTVSS |
| PD 1.24 | SEQ ID NO: | SEQ ID NO: | SEQ ID NO: | SEQ ID NO: 981 |
| | 978 | 979 | 980 | EVQLVESGGGLVQPGRSLRLSCAASGFTFDDYAMHWVRQ |
| | DYAMH | GISWNSGSM | EKGPGLTGSTA | APGKDLEWVSGISWNSGSMGYAASVKGRFTISRDNAKNSL |
| | | GYAASVKG | DYYGLDA | YLQMNSLRAEDTALYYCVREKGPGLTGSTADYYGLDAWGQ |
| | | | | GTMVTVSS |
| PD 1.25 | SEQ ID NO: | SEQ ID NO: | SEQ ID NO: | SEQ ID NO: 985 |
| | 982 | 983 | 984 | EVQLVESGGGLVQPGRSLRLSCAASGFTFDDYAMHWVRQ |
| | DYAMH | GISWNGGSQ | EKGPGLTGSTA | APGKDLEWVSGISWNGGSQGYAASVKGRFTISRDNAKNSL |
| | | GYAASVKG | DYYGLDA | YLQMNSLRAEDTALYYCVREKGPGLTGSTADYYGLDAWGQ |
| | | | | GTMVTVSS |
| PD 1.26 | SEQ ID NO: | SEQ ID NO: | SEQ ID NO: | SEQ ID NO: 989 |
| | 986 | 987 | 988 | EVQLVESGGGLVQPGRSLRLSCAASGFTFDDYAMHWVRQ |
| | DYAMH | GISWNGGS | EKGPGLTGSTA | APGKDLEWVSGISWNGGSMGYADSVKGRFTISRDNAKNSL |
| | | MGYADSVK | DYYGLDA | YLQMNSLRAEDTALYYCVREKGPGLTGSTADYYGLDAWGQ |
| | | G | | GTMVTVSS |
| PD 1.27 | SEQ ID NO: | SEQ ID NO: | SEQ ID NO: | SEQ ID NO: 993 |
| | 990 | 991 | 992 | EVQLVESGGGLVQPGRSLRLSCAASGFTFDDYAMHWVRQ |
| | DYAMH | GISWNGGSR | EKGPGLTGSTA | APGKDLEWVSGISWNGGSRGYAASVKGRFTISRDNAKNSL |
| | | GYAASVKG | DYYGLDA | YLQMNSLRAEDTALYYCVREKGPGLTGSTADYYGLDAWGQ |
| | | | | GTMVTVSS |
| PD 1.28 | SEQ ID NO: | SEQ ID NO: | SEQ ID NO: | SEQ ID NO: |
| | 994 | 995 | 996 | 7EVQLVESGGGLVQPGRSLRLSCAASGFTFDDYAMHWVR |
| | DYAMH | GISWNAGS | EKGPGLTGSTA | QAPGKDLEWVSGISWNAGSMGYAASVKGRFTISRDNAKN |
| | | MGYAASVK | DYYGLDA | SLYLQMNSLRAEDTALYYCVREKGPGLTGSTADYYGLDAW |
| | | G | | GQGTMVTVSS |
| PD 1.29 | SEQ ID NO: | SEQ ID NO: | SEQ ID NO: | SEQ ID NO: 1001 |
| | 998 | 999 | 1000 | EVQLVESGGGLVQPGRSLRLSCAASGFTFDDYAMHWVRQ |
| | DYAMH | GISWNSGSM | EKGPGLTGSTA | APGKDLEWVSGISWNSGSMGYADSVKGRFTISRDNAKNSL |
| | | GYADSVKG | DYYGLDV | YLQMNSLRAEDTALYYCVREKGPGLTGSTADYYGLDVWGQ |
| | | | | GTMVTVSS |
| PD 1.30 | SEQ ID NO: | SEQ ID NO: | SEQ ID NO: | SEQ ID NO: 1005 |
| | 1002 | 1003 | 1004 | EVQLVESGGGLVQPGRSLRLSCAASGFTFDDYAGHWVRQA |
| | DYAGH | GISWNGGS | EKGPGLTGSTA | PGKDLEWVSGISWNGGSMGYAASVKGRFTISRDNAKNSLY |
| | | MGYAASVK | DYYGLDA | LQMNSLRAEDTALYYCVREKGPGLTGSTADYYGLDAWGQ |
| | | G | | GTMVTVSS |
| PD 1.31 | SEQ ID NO: | SEQ ID NO: | SEQ ID NO: | SEQ ID NO: 1009 |
| | 1006 | 1007 | 1008 | EVQLVESGGGLVQPGRSLRLSCAASGFTFDDYALHWVRQA |
| | DYALH | GISWNGGS | EKGPGLTGSTA | PGKDLEWVSGISWNGGSMGYAASVKGRFTISRDNAKNSLY |
| | | MGYAASVK | DYYGLDA | LQMNSLRAEDTALYYCVREKGPGLTGSTADYYGLDAWGQ |
| | | G | | GTMVTVSS |
| PD 1.32 | SEQ ID NO: | SEQ ID NO: | SEQ ID NO: | SEQ ID NO: 1013 |
| | 1010 | 1011 | 1012 | EVQLVESGGGLVQPGRSLRLSCAASGFTFDDYAMHWVRQ |
| | DYAMH | GISWNSGSM | EKGPGLTGSTA | APGKDLEWVSGISWNSGSMGYADSVKGRFTISRDNAKNSL |
| | | GYADSVKG | DYYGLDA | YLQMNSLRAEDTALYYCVREKGPGLTGSTADYYGLDAWGQ |
| | | | | GTMVTVSS |
| PD 1.33 | SEQ ID NO: | SEQ ID NO: | SEQ ID NO: | SEQ ID NO: 1017 |
| | 1014 | 1015 | 1016 | EVQLVESGGGLVQPGRSLRLSCAASGFTFDDYAMHWVRQ |
| | DYAMH | GISWNGGSY | EKGPGLTGSTA | APGKDLEWVSGISWNGGSYGYADSVKGRFTISRDNAKNSL |
| | | GYADSVKG | DYYGLDA | YLQMNSLRAEDTALYYCVREKGPGLTGSTADYYGLDAWGQ |
| | | | | GTMVTVSS |
| PD 1.34 | SEQ ID NO: | SEQ ID NO: | SEQ ID NO: | SEQ ID NO: 1021 |
| | 1018 | 1019 | 1020 | EVQLVESGGGLVQPGRSLRLSCAASGFTFDDYAMHWVRQ |
| | DYAMH | GISWNGGSQ | EKGPGLTGSTA | APGKDLEWVSGISWNGGSQGYADSVKGRFTISRDNAKNSL |
| | | GYADSVKG | DYYGLDA | YLQMNSLRAEDTALYYCVREKGPGLTGSTADYYGLDAWGQ |
| | | | | GTMVTVSS |
| PD 1.35 | SEQ ID NO: | SEQ ID NO: | SEQ ID NO: | SEQ ID NO: 1025 |
| | 1022 | 1023 | 1024 | EVQLVESGGGLVQPGRSLRLSCAASGFTFDDYAMHWVRQ |
| | DYAMH | GISWNGGSK | EKGPGLTGSTA | APGKDLEWVSGISWNGGSKGYADSVKGRFTISRDNAKNSL |
| | | GYADSVKG | DYYGLDA | YLQMNSLRAEDTALYYCVREKGPGLTGSTADYYGLDAWGQ |
| | | | | GTMVTVSS |
| PD 1.36 | SEQ ID NO: | SEQ ID NO: | SEQ ID NO: | SEQ ID NO: 1029 |
| | 1026 | 1027 | 1028 | EVQLVESGGGLVQPGRSLRLSCAASGFTFDDYAMHWVRQ |
| | DYAMH | GISWNAGS | EKGPGLTGSTA | APGKDLEWVSGISWNAGSMGYADSVKGRFTISRDNAKNSL |
| | | MGYADSVK | DYYGLDA | YLQMNSLRAEDTALYYCVREKGPGLTGSTADYYGLDAWGQ |
| | | G | | GTMVTVSS |
| PD 1.37 | SEQ ID NO: | SEQ ID NO: | SEQ ID NO: | SEQ ID NO: 1033 |
| | 1030 | 1031 | 1032 | EVQLVESGGGLVQPGRSLRLSCAASGFTFDDYAFHWVRQA |
| | DYAFH | GISWNGGS | EKGPGLTGSTA | PGKDLEWVSGISWNGGSMGYADSVKGRFTISRDNAKNSLY |
| | | MGYADSVK | DYYGLDA | LQMNSLRAEDTALYYCVREKGPGLTGSTADYYGLDAWGQ |
| | | G | | GTMVTVSS |
| PD 1.38 | SEQ ID NO: | SEQ ID NO: | SEQ ID NO: | SEQ ID NO: 1037 |
| | 1034 | 1035 | 1036 | EVQLVESGGGLVQPGRSLRLSCAASGFTFDDYALHWVRQA |
| | DYALH | GISWNGGS | EKGPGLTGSTA | PGKDLEWVSGISWNGGSMGYADSVKGRFTISRDNAKNSLY |
| | | MGYADSVK | DYYGLDA | LQMNSLRAEDTALYYCVREKGPGLTGSTADYYGLDAWGQ |
| | | G | | GTMVTVSS |
| PD 1.39 | SEQ ID NO: | SEQ ID NO: | SEQ ID NO: | SEQ ID NO: 1041 |
| | 1038 | 1039 | 1040 | EVQLVESGGGLVQPGRSLRLSCAASGFTFDDYALHWVRQA |
| | DYALH | GISWNGGSY | EKGPGLTGSTA | PGKDLEWVSGISWNGGSYGYADSVKGRFTISRDNAKNSLYL |
| | | GYADSVKG | DYYGLDA | QMNSLRAEDTALYYCVREKGPGLTGSTADYYGLDAWGQG |
| | | | | TMVTVSS |
| PD 1.40 | SEQ ID NO: | SEQ ID NO: | SEQ ID NO: | SEQ ID NO: 1045 |
| | 1042 | 1043 | 1044 | EVQLVESGGGLVQPGRSLRLSCAASGFTFDDYALHWVRQA |
| | DYALH | GISWNGGSQ | EKGPGLTGSTA | PGKDLEWVSGISWNGGSQGYADSVKGRFTISRDNAKNSLY |
| | | GYADSVKG | DYYGLDA | LQMNSLRAEDTALYYCVREKGPGLTGSTADYYGLDAWGQ |
| | | | | GTMVTVSS |
| PD 1.41 | SEQ ID NO: | SEQ ID NO: | SEQ ID NO: | SEQ ID NO: 1049 |
| | 1046 | 1047 | 1048 | EVQLVESGGGLVQPGRSLRLSCAASGFTFDDYAFHWVRQA |
| | DYAFH | GISWNGGSY | EKGPGLTGSTA | PGKDLEWVSGISWNGGSYGYADSVKGRFTISRDNAKNSLYL |
| | | GYADSVKG | DYYGLDA | QMNSLRAEDTALYYCVREKGPGLTGSTADYYGLDAWGQG |
| | | | | TMVTVSS |
| PD 1.42 | SEQ ID NO: | SEQ ID NO: | SEQ ID NO: | SEQ ID NO: 1053 |
| | 1050 | 1051 | 1052 | EVQLVESGGGLVQPGRSLRLSCAASGFTFDDYAFHWVRQA |
| | DYAFH | GISWNGGSQ | EKGPGLTGSTA | PGKDLEWVSGISWNGGSQGYADSVKGRFTISRDNAKNSLY |
| | | GYADSVKG | DYYGLDA | LQMNSLRAEDTALYYCVREKGPGLTGSTADYYGLDAWGQ |
| | | | | GTMVTVSS |
| PD 1.43 | SEQ ID NO: | SEQ ID NO: | SEQ ID NO: | SEQ ID NO: 1057 |
| | 1054 | 1055 | 1056 | EVQLVESGGGLVQPGRSLRLSCAASGFTFDDYAFHWVRQA |
| | DYAFH | GISWNAGSY | EKGPGLTGSTA | PGKDLEWVSGISWNAGSYGYADSVKGRFTISRDNAKNSLYL |
| | | GYADSVKG | DYYGLDA | QMNSLRAEDTALYYCVREKGPGLTGSTADYYGLDAWGQG |
| | | | | TMVTVSS |
| PD 1.44 | SEQ ID NO: | SEQ ID NO: | SEQ ID NO: | SEQ ID NO: 1061 |
| | 1058 | 1059 | 1060 | EVQLVESGGGLVQPGRSLRLSCAASGFTFDDYALHWVRQA |
| | DYALH | GISWNSGSY | EKGPGLTGSTA | PGKDLEWVSGISWNSGSYGYADSVKGRFTISRDNAKNSLYL |
| | | GYADSVKG | DYYGLDA | QMNSLRAEDTALYYCVREKGPGLTGSTADYYGLDAWGQG |
| | | | | TMVTVSS |
| PD 1.45 | SEQ ID NO: | SEQ ID NO: | SEQ ID NO: | SEQ ID NO: 1065 |
| | 1062 | 1063 | 1064 | EVQLVESGGGLVQPGRSLRLSCAASGFTFDDYALHWVRQA |
| | DYALH | GISWNAGSQ | EKGPGLTGSTA | PGKDLEWVSGISWNAGSQGYADSVKGRFTISRDNAKNSLY |
| | | GYADSVKG | DYYGLDA | LQMNSLRAEDTALYYCVREKGPGLTGSTADYYGLDAWGQ |
| | | | | GTMVTVSS |
| PD 1.46 | SEQ ID NO: | SEQ ID NO: | SEQ ID NO: | SEQ ID NO: 1069 |
| | 1066 | 1067 | 1068 | EVQLVESGGGLVQPGRSLRLSCAASGFTFDDYALHWVRQA |
| | DYALH | GISWNAGSY | EKGPGLTGSTA | PGKDLEWVSGISWNAGSYGYADSVKGRFTISRDNAKNSLYL |
| | | GYADSVKG | DYYGLDA | QMNSLRAEDTALYYCVREKGPGLTGSTADYYGLDAWGQG |
| | | | | TMVTVSS |
| PD 1.47 | SEQ ID NO: | SEQ ID NO: | SEQ ID NO: | SEQ ID NO: 1073 |
| | 1070 | 1071 | 1072 | EVQLVESGGGLVQPGRSLRLSCAASGFTFDDYAFHWVRQA |
| | DYAFH | GISWNSGSY | EKGPGLTGSTA | PGKDLEWVSGISWNSGSYGYADSVKGRFTISRDNAKNSLYL |
| | | GYADSVKG | DYYGLDA | QMNSLRAEDTALYYCVREKGPGLTGSTADYYGLDAWGQG |
| | | | | TMVTVSS |
| PD 1.48 | SEQ ID NO: | SEQ ID NO: | SEQ ID NO: | SEQ ID NO: 1077 |
| | 1074 | 1075 | 6EKGPGLTGST | EVQLVESGGGLVQPGRSLRLSCAASGFTFDDYALHWVRQA |
| | DYALH | GISWNSGSQ | ADYYGLDA | PGKDLEWVSGISWNSGSQGYADSVKGRFTISRDNAKNSLYL |
| | | GYADSVKG | | QMNSLRAEDTALYYCVREKGPGLTGSTADYYGLDAWGQG |
| | | | | TMVTVSS |
| PD 1.49 | SEQ ID NO: | SEQ ID NO: | SEQ ID NO: | SEQ ID NO: 1081 |
| | 1078 | 1079 | 1080 | EVQLVESGGGLVQPGRSLRLSCAASGFTFDDYAFHWVRQA |
| | DYAFH | GISWNSGSQ | EKGPGLTGSTA | PGKDLEWVSGISWNSGSQGYADSVKGRFTISRDNAKNSLYL |
| | | GYADSVKG | DYYGLDA | QMNSLRAEDTALYYCVREKGPGLTGSTADYYGLDAWGQG |
| | | | | TMVTVSS |
| PD 1.50 | SEQ ID NO: | SEQ ID NO: | SEQ ID NO: | SEQ ID NO: 1085 |
| | 1082 | 1083 | 1084 | EVQLVESGGGLVQPGRSLRLSCAASGFTFDDYAFHWVRQA |
| | DYAFH | GISWNAGSQ | EKGPGLTGSTA | PGKDLEWVSGISWNAGSQGYADSVKGRFTISRDNAKNSLY |
| | | GYADSVKG | DYYGLDA | LQMNSLRAEDTALYYCVREKGPGLTGSTADYYGLDAWGQ |
| | | | | GTMVTVSS |

TABLE 10
Further exemplary PD-1 binding single V_{H} domain antibodies
that do not block PD-1 function, e.g. the functional
interaction between human PD-1 and its ligands PD-L1
and/or PD-L2 when used in on their own. These bind to
an epitope comprising one or more or all residues
selected from R¹⁰⁴, D¹⁰⁵, F¹⁰⁶, H¹⁰⁷, M¹⁰⁸, S¹⁰⁹ and V¹¹⁰
of human PD-1. These single VH domain antibodies can
be used in the multispecific binding molecules described
herein, e.g. by fusion to a CD137 binding single
VH domain antibody as described herein, e.g. in Table 2.

| | CDR1 | CDR2 | CDR3 | |
|---|---|---|---|---|
| | sequence | sequence | sequence | |
| Name | of V_{H} | of V_{H} | of V_{H} | Full length V_{H} sequence |
| PD2. | SEQ ID NO: | SEQ ID NO: | SEQ ID NO: | SEQ ID NO: 1089 |
| 1 | 1086 | 1087 | 1088 | EVQLVESGGGVVRPGGSLRLSCAASGFTFDDYAMSWVRQAPGK |
| | DYAMS | GITWNGGST | DKYSYAWSY | GLEWVSGITWNGGSTGYADSVKDRFTISRDNAKNSLYLQMNSLR |
| | | GYADSVKD | DGFDI | AEDTALYYCVRDKYSYAWSYDGFDIWGQGTMVTVSS |
| PD2. | SEQ ID NO: | SEQ ID NO: | SEQ ID NO: | SEQ ID NO: 1093 |
| 2 | 1090 | 1091 | 1092 | EVQLLESGGGWRPGGSLRLSCAASGFTFDDYGMSWVRQPPGK |
| | DYGMS | GISRNGGSA | EKYSSGWSY | GLEWVSGISRNGGSAGYSDSAKDRFTISRDNAKNSLYLQMNSLRA |
| | | GYSDSAKD | DDFDI | DDTAMYYCAREKYSSGWSYDDFDIWGQGTMVTVSS |
| PD2. | SEQ ID | SEQ ID NO: | SEQ ID NO: | SEQ ID NO: 1097 |
| 3 | NO: 1094 | 1095 | 1096 | QVQLVESGGGWRPGGSLRLSCAASGFTFDDYGMSWVRQSPGK |
| | DYGMS | GISRNGGSA | EKYSSGWSY | GLEWVSGISRNGGSAGYSDSAKDRFTISRDNAKNSLYLQMNSLRA |
| | | GYSDSAKD | DDFDI | DDTAMYYCAREKYSSGWSYDDFDIWGQGTMVTVSS |
| PD2. | SEQ ID NO: | SEQ ID NO: | SEQ ID NO: | SEQ ID NO: 1101 |
| 4 | 1098 | 1099 | 1100 | QVQLVESGGGWRPGGSLRLSCAASGFTFDDYGMSWVRQPPGK |
| | DYGMS | GISRNGGSA | EKYSSGWSY | GLEWVSGISRNGGSAGYSDSAKDRFTISRDNAKNSLYLQMNSLRA |
| | | GYSDSAKD | DDFDI | DDTAMYYCAREKYSSGWSYDDFDIWGQGTMVTVSS |
| PD2. | SEQ ID NO: | SEQ ID NO: | SEQ ID NO: | SEQ ID NO: 1105 |
| 5 | 1102 | 1103 | 1104 | EVQLVESGGGVVRPGGSLRLSCAASGFTFDDYGMSWVRQPPGK |
| | DYGMS | GISRNGGSA | EKYSSGWSY | GLEWVSGISRNGGSAGYSDSAKDRFTISRDNAKNSLYLQMNSLRA |
| | | GYSDSAKD | DDFDI | DDTAMYYCAREKYSSGWSYDDFDIWGQGTMVTVSS |
| PD | SEQ ID NO: | SEQ ID NO: | SEQ ID NO: | SEQ ID NO: 1109 |
| 2.6 | 1106 | 1107 | 1108 | QVQLVESGGGWRLGGSLRLSCAASGFSFVDYGMSWVRQAPGQ |
| | DYGMS | GISRNGGST | DPYSSGWSY | GLEWVSGISRNGGSTGYADSVKDRFTISRDNAKNTLYLQMNSLRA |
| | | GYADSVKD | DSFDI | EDTALYYCARDPYSSGWSYDSFDIWGQGTMVTVSS |
| PD | SEQ ID NO: | SEQ ID NO: | SEQ ID NO: | SEQ ID NO: 1113 |
| 2.7 | 1110 | 1111 | 1112 | QVQLVESGGGWRPGGSLRLSCAASGFTFDDYGMSWVRQAPGK |
| | DYGMS | GISRNGGST | EKYSSGWSY | GLEWVSGISRNGGSTGYTASVKDRFTISRDNAKNSLYLQMNSLRA |
| | | GYTASVKD | DDFDI | DDTAMYYCAREKYSSGWSYDDFDIWGQGTMVTVSS |
| PD | SEQ ID NO: | SEQ ID NO: | SEQ ID NO: | SEQ ID NO: 1117 |
| 2.8 | 1114 | 1115 | 1116 | QVQLVESGGGLVQPGGSLRLSCAASGFTFDDYAMSWVRQAPGK |
| | DYAMS | GISWNGGSA | DPHSSAWSY | GLEWVSGISWNGGSAGYADSVKDRFTISRDNAKNSLYLQMNSLR |
| | | GYADSVKD | DAFDI | AEDTALYYCARDPHSSAWSYDAFDIWGQGTMVTVSS |
| PD | SEQ ID NO: | SEQ ID NO: | SEQ ID NO: | SEQ ID NO: 1121 |
| 2.9 | 1118 | 1119 | 1120 | QVQLVESGGGWRPGGSLRLSCAASGFTFDDYAMSWVRQAPGK |
| | DYAMS | GISWNGGSK | DPYSGAWSY | GLEWVSGISWNGGSKGYADSVKDRFTISRDNAKNSLYLQMNSLR |
| | | GYADSVKD | DAFDI | AEDTALYYCARDPYSGAWSYDAFDIWGQGTMVTVSS |
| PD | SEQ ID NO: | SEQ ID NO: | SEQ ID NO: | SEQ ID NO: 1125 |
| 2.10 | 1122 | 1123 | 1124 | QVQLVESGGGWRPGGSLRLSCAASGFTFDDYAMSWVRQAPGK |
| | DYAMS | GISWNGGST | DPYSGAWSY | GLEWVSGISWNGGSTGYADSVKDRFTISRDNAKNSLYLQMNSLR |
| | | GYADSVKD | DAFDI | AEDTALYYCARDPYSGAWSYDAFDIWGQGTMVTVSS |
| PD | SEQ ID NO: | SEQ ID NO: | SEQ ID NO: | SEQ ID NO: 1129 |
| 2.11 | 1126 | 1127 | 1128 | QVQLVESGGGWRPGGSLRLSCAASGFTFDNYAMSWVRQAPGK |
| | NYAMS | GITWNGGST | DKYSYAWSY | GLEWVSGITWNGGSTGYADSVKDRFTISRDNAKNSLYLQMNSLR |
| | | GYADSVKD | DTFDI | AEDTALYYCARDKYSYAWSYDTFDIRGQGTMVTVSS |
| PD | SEQ ID NO: | SEQ ID NO: | SEQ ID NO: | SEQ ID NO: 1133 |
| 2.12 | 1130 | 1131 | 1132 | EVQLVESGGGVVRPGGSLRLSCAASGFTFDDYAMSWVRQAPGK |
| | DYAMS | GITWNAGST | DKYSYAWSY | GLEWVSGITWNAGSTGYADSVKGRFTISRDNAKNSLYLQMNSLR |
| | | GYADSVKG | DDFDI | AEDTALYYCVRDKYSYAWSYDDFDIWGQGTMVTVSS |
| PD | SEQ ID NO: | SEQ ID NO: | SEQ ID NO: | SEQ ID NO: 1137 |
| 2.13 | 1134 | 1135 | 1136 | EVQLVESGGGVVRPGGSLRLSCAASGFTFDDYAMSWVRQAPGK |
| | DYAMS | GITWNRGST | DKYSYAWSY | GLEWVSGITWNRGSTGYADSVKGRFTISRDNAKNSLYLQMNSLR |
| | | GYADSVKG | DDFDI | AEDTALYYCVRDKYSYAWSYDDFDIWGQGTMVTVSS |
| PD | SEQ ID NO: | SEQ ID NO: | SEQ ID NO: | SEQ ID NO: 1141 |
| 2.14 | 1138 | 1139 | 1140 | EVQLVESGGGVVRPGGSLRLSCAASGFTFDDYAFSWVRQAPGKG |
| | DYAFS | GITWNGGST | DKYSYAWSY | LEWVSGITWNGGSTGYADSVKGRFTISRDNAKNSLYLQMNSLRA |
| | | GYADSVKG | DGFDI | EDTALYYCVRDKYSYAWSYDGFDIWGQGTMVTVSS |
| PD | SEQ ID NO: | SEQ ID NO: | SEQ ID NO: | SEQ ID NO: 1145 |
| 2.15 | 1142 | 1143 | 1144 | EVQLVESGGGVVRPGGSLRLSCAASGFTFDDYAQSWVRQAPGK |
| | DYAQS | GITWNGGST | DKYSYAWSY | GLEWVSGITWNGGSTGYADSVKGRFTISRDNAKNSLYLQMNSLR |
| | | GYADSVKG | DGFDI | AEDTALYYCVRDKYSYAWSYDGFDIWGQGTMVTVSS |
| PD | SEQ ID NO: | SEQ ID NO: | SEQ ID NO: | SEQ ID NO: 1149 |
| 2.16 | 1146 | 1147 | 1148 | EVQLVESGGGVVRPGGSLRLSCAASGFTFDDYANSWVRQAPGK |
| | DYANS | GITWNGGST | DKYSYAWSY | GLEWVSGITWNGGSTGYADSVKGRFTISRDNAKNSLYLQMNSLR |
| | | GYADSVKG | DGFDI | AEDTALYYCVRDKYSYAWSYDGFDIWGQGTMVTVSS |
| PD | SEQ ID NO: | SEQ ID NO: | SEQ ID NO: | SEQ ID NO: 1153 |
| 2.17 | 1150 | 1151 | 1152 | EVQLVESGGGVVRPGGSLRLSCAASGFTFDDYAGSWVRQAPGK |
| | DYAGS | GITWNGGST | DKYSYAWSY | GLEWVSGITWNGGSTGYADSVKGRFTISRDNAKNSLYLQMNSLR |
| | | GYADSVKG | DGFDI | AEDTALYYCVRDKYSYAWSYDGFDIWGQGTMVTVSS |
| PD | SEQ ID NO: | SEQ ID NO: | SEQ ID NO: | SEQ ID NO: 1157 |
| 2.18 | 1154 | 1155 | 1156 | EVQLVESGGGVVRPGGSLRLSCAASGFTFDDYAESWVRQAPGKG |
| | DYAES | GITWNGGST | DKYSYAWSY | LEWVSGITWNGGSTGYADSVKGRFTISRDNAKNSLYLQMNSLRA |
| | | GYADSVKG | DGFDI | EDTALYYCVRDKYSYAWSYDGFDIWGQGTMVTVSS |
| PD | SEQ ID NO: | SEQ ID NO: | SEQ ID NO: | SEQ ID NO: 1161 |
| 2.19 | 1158 | 1159 | 1160 | EVQLVESGGGVVRPGGSLRLSCAASGFTFDDYAWSWVRQAPGK |
| | DYAWS | GITWNGGST | DKYSYAWSY | GLEWVSGITWNGGSTGYADSVKGRFTISRDNAKNSLYLQMNSLR |
| | | GYADSVKG | DGFDI | AEDTALYYCVRDKYSYAWSYDGFDIWGQGTMVTVSS |
| PD | SEQ ID NO: | SEQ ID NO: | SEQ ID NO: | SEQ ID NO: 1165 |
| 2.20 | 1162 | 1163 | 1164 | EVQLVESGGGVVRPGGSLRLSCAASGFTFDDYAVSWVRQAPGKG |
| | DYAVS | GITWNGGST | DKYSYAWSY | LEWVSGITWNGGSTGYADSVKGRFTISRDNAKNSLYLQMNSLRA |
| | | GYADSVKG | DGFDI | EDTALYYCVRDKYSYAWSYDGFDIWGQGTMVTVSS |
| PD | SEQ ID NO: | SEQ ID NO: | SEQ ID NO: | SEQ ID NO: 1169 |
| 2.21 | 1166 | 1167 | 1168 | EVQLVESGGGVVRPGGSLRLSCAASGFTFDDYALSWVRQAPGKG |
| | DYALS | GITWNGGST | DKYSYAWSY | LEWVSGITWNGGSTGYADSVKGRFTISRDNAKNSLYLQMNSLRA |
| | | GYADSVKG | DGFDI | EDTALYYCVRDKYSYAWSYDGFDIWGQGTMVTVSS |
| PD | SEQ ID NO: | SEQ ID NO: | SEQ ID NO: | SEQ ID NO: 1173 |
| 2.22 | 1170 | 1171 | 1172 | EVQLVESGGGVVRPGGSLRLSCAASGFTFDDYASSWVRQAPGKG |
| | DYASS | GITWNGGST | DKYSYAWSY | LEWVSGITWNGGSTGYADSVKGRFTISRDNAKNSLYLQMNSLRA |
| | | GYADSVKG | DGFDI | EDTALYYCVRDKYSYAWSYDGFDIWGQGTMVTVSS |
| PD | SEQ ID NO: | SEQ ID NO: | SEQ ID NO: | SEQ ID NO: 1177 |
| 2.23 | 1174 | 1175 | 1176 | EVQLVESGGGVVRPGGSLRLSCAASGFTFDDYARSWVRQAPGKG |
| | DYARS | GITWNGGST | DKYSYAWSY | LEWVSGITWNGGSTGYADSVKGRFTISRDNAKNSLYLQMNSLRA |
| | | GYADSVKG | DGFDI | EDTALYYCVRDKYSYAWSYDGFDIWGQGTMVTVSS |
| PD | SEQ ID NO: | SEQ ID NO: | SEQ ID NO: | SEQ ID NO: 1181 |
| 2.24 | 1178 | 1179 | 1180 | EVQLVESGGGVVRPGGSLRLSCAASGFTFDDYAMSWVRQAPGK |
| | DYAMS | GITWNSGST | DKYSYAWSY | GLEWVSGITWNSGSTGYADSVKDRFTISRDNAKNSLYLQMNSLR |
| | | GYADSVKD | DDFDI | AEDTALYYCVRDKYSYAWSYDDFDIWGQGTMVTVSS |
| PD | SEQ ID NO: | SEQ ID NO: | SEQ ID NO: | SEQ ID NO: 1185 |
| 2.25 | 1182 | 1183 | 1184 | EVQLVESGGGVVRPGGSLRLSCAASGFTFDDYAMSWVRQAPGK |
| | DYAMS | GITWNQGST | DKYSYAWSY | GLEWVSGITWNQGSTGYADSVKDRFTISRDNAKNSLYLQMNSLR |
| | | GYADSVKD | DDFDI | AEDTALYYCVRDKYSYAWSYDDFDIWGQGTMVTVSS |
| PD | SEQ ID NO: | SEQ ID NO: | SEQ ID NO: | SEQ ID NO: 1189 |
| 2.26 | 1186 | 1187 | 1188 | EVQLVESGGGVVRPGGSLRLSCAASGFTFDDYAMSWVRQAPGK |
| | DYAMS | GITWNHGST | DKYSYAWSY | GLEWVSGITWNHGSTGYADSVKDRFTISRDNAKNSLYLQMNSLR |
| | | GYADSVKD | DVFDI | AEDTALYYCVRDKYSYAWSYDVFDIWGQGTMVTVSS |
| PD | SEQ ID NO: | SEQ ID NO: | SEQ ID NO: | SEQ ID NO: 1193 |
| 2.27 | 1190 | 1191 | 1192 | EVQLVESGGGVVRPGGSLRLSCAASGFTFDDYAMSWVRQAPGK |
| | DYAMS | GITWNAGST | DKYSYAWSY | GLEWVSGITWNAGSTGYADSVKDRFTISRDNAKNSLYLQMNSLR |
| | | GYADSVKD | DDFDI | AEDTALYYCVRDKYSYAWSYDDFDIWGQGTMVTVSS |
| PD | SEQ ID NO: | SEQ ID NO: | SEQ ID NO: | SEQ ID NO: 1197 |
| 2.28 | 1194 | 1195 | 1196 | EVQLVESGGGVVRPGGSLRLSCAASGFTFDDYAMSWVRQAPGK |
| | DYAMS | GITWNGGST | DKYSYAWSY | GLEWVSGITWNGGSTGYADSVKGRFTISRDNAKNSLYLQMNSLR |
| | | GYADSVKG | DVFDI | AEDTALYYCVRDKYSYAWSYDVFDIWGQGTMVTVSS |
| PD | SEQ ID NO: | SEQ ID NO: | SEQ ID NO: | SEQ ID NO: 1201 |
| 2.29 | 1198 | 1199 | 1200 | EVQLVESGGGVVRPGGSLRLSCAASGFTFDDYAMSWVRQAPGK |
| | DYAMS | GITWNGGST | DKYSYAWSY | GLEWVSGITWNGGSTGYADSVKGRFTISRDNAKNSLYLQMNSLR |
| | | GYADSVKG | DDFDI | AEDTALYYCVRDKYSYAWSYDDFDIWGQGTMVTVSS |
| PD | SEQ ID NO: | SEQ ID | SEQ ID NO: | SEQ ID NO: 1205 |
| 2.30 | 1202 | N0:1203 | 1204 | EVQLVESGGGVVRPGGSLRLSCAASGFTFDDYAMSWVRQAPGK |
| | DYAMS | GITWNKGST | DKYSYAWSY | GLEWVSGITWNKGSTGYADSVKDRFTISRDNAKNSLYLQMNSLR |
| | | GYADSVKD | DDFDI | AEDTALYYCVRDKYSYAWSYDDFDIWGQGTMVTVSS |
| PD | SEQ ID NO: | SEQ ID NO: | SEQ ID NO: | SEQ ID NO: 1209 |
| 2.31 | 1206 | 1207 | 1208 | EVQLVESGGGVVRPGGSLRLSCAASGFTFDDYAMSWVRQAPGK |
| | DYAMS | GITWNRGST | DKYSYAWSY | GLEWVSGITWNRGSTGYADSVKDRFTISRDNAKNSLYLQMNSLR |
| | | GYADSVKD | DDFDI | AEDTALYYCVRDKYSYAWSYDDFDIWGQGTMVTVSS |
| PD | SEQ ID NO: | SEQ ID NO: | SEQ ID NO: | SEQ ID NO: 1213 |
| 2.32 | 1210 | 1211 | 1212 | EVQLVESGGGVVRPGGSLRLSCAASGFTFDDYAISWVRQAPGKG |
| | DYAIS | GITWNGGST | DKYSYAWSY | LEWVSGITWNGGSTGYADSVKDRFTISRDNAKNSLYLQMNSLRA |
| | | GYADSVKD | DGFDI | EDTALYYCVRDKYSYAWSYDGFDIWGQGTMVTVSS |
| PD | SEQ ID NO: | SEQ ID NO: | SEQ ID NO: | SEQ ID NO: 1217 |
| 2.33 | 1214 | 1215 | 1216 | EVQLVESGGGVVRPGGSLRLSCAASGFTFDDYATSWVRQAPGKG |
| | DYATS | GITWNGGST | DKYSYAWSY | LEWVSGITWNGGSTGYADSVKDRFTISRDNAKNSLYLQMNSLRA |
| | | GYADSVKD | DGFDI | EDTALYYCVRDKYSYAWSYDGFDIWGQGTMVTVSS |
| PD | SEQ ID NO: | SEQ ID NO: | SEQ ID NO: | SEQ ID NO: 1221 |
| 2.34 | 1218 | 1219 | 1220 | EVQLVESGGGVVRPGGSLRLSCAASGFTFDDYANSWVRQAPGK |
| | DYANS | GITWNGGST | DKYSYAWSY | GLEWVSGITWNGGSTGYADSVKDRFTISRDNAKNSLYLQMNSLR |
| | | GYADSVKD | DGFDI | AEDTALYYCVRDKYSYAWSYDGFDIWGQGTMVTVSS |
| PD | SEQ ID | SEQ ID NO: | SEQ ID NO: | SEQ ID NO: 1225 |
| 2.35 | NO:1222 | 1223 | 1224 | EVQLVESGGGVVRPGGSLRLSCAASGFTFDDYADSWVRQAPGKG |
| | DYADS | GITWNGGST | DKYSYAWSY | LEWVSGITWNGGSTGYADSVKDRFTISRDNAKNSLYLQMNSLRA |
| | | GYADSVKD | DGFDI | EDTALYYCVRDKYSYAWSYDGFDIWGQGTMVTVSS |
| PD | SEQ ID NO: | SEQ ID NO: | SEQ ID NO: | SEQ ID NO: 1229 |
| 2.36 | 1226 | 1227 | 1228 | EVQLVESGGGVVRPGGSLRLSCAASGFTFDDYASSWVRQAPGKG |
| | DYASS | GITWNGGST | DKYSYAWSY | LEWVSGITWNGGSTGYADSVKDRFTISRDNAKNSLYLQMNSLRA |
| | | GYADSVKD | DGFDI | EDTALYYCVRDKYSYAWSYDGFDIWGQGTMVTVSS |
| PD | SEQ ID NO: | SEQ ID NO: | SEQ ID NO: | SEQ ID NO: 1233 |
| 2.37 | 1230 | 1231 | 1232 | EVQLVESGGGVVRPGGSLRLSCAASGFTFDDYALSWVRQAPGKG |
| | DYALS | GITWNGGST | DKYSYAWSY | LEWVSGITWNGGSTGYADSVKDRFTISRDNAKNSLYLQMNSLRA |
| | | GYADSVKD | DGFDI | EDTALYYCVRDKYSYAWSYDGFDIWGQGTMVTVSS |
| PD | SEQ ID NO: | SEQ ID NO: | SEQ ID NO: | SEQ ID NO: 1237 |
| 2.38 | 1234 | 1235 | 1236 | EVQLVESGGGVVRPGGSLRLSCAASGFTFDDYAGSWVRQAPGK |
| | DYAGS | GITWNGGST | DKYSYAWSY | GLEWVSGITWNGGSTGYADSVKDRFTISRDNAKNSLYLQMNSLR |
| | | GYADSVKD | DGFDI | AEDTALYYCVRDKYSYAWSYDGFDIWGQGTMVTVSS |
| PD | SEQ ID NO: | SEQ ID NO: | SEQ ID NO: | SEQ ID NO: 1241 |
| 2.39 | 1238 | 1239 | 1240 | EVQLVESGGGVVRPGGSLRLSCAASGFTFDDYAESWVRQAPGKG |
| | DYAES | GITWNGGST | DKYSYAWSY | LEWVSGITWNGGSTGYADSVKDRFTISRDNAKNSLYLQMNSLRA |
| | | GYADSVKD | DGFDI | EDTALYYCVRDKYSYAWSYDGFDIWGQGTMVTVSS |
| PD | SEQ ID NO: | SEQ ID NO: | SEQ ID NO: | SEQ ID NO: 1245 |
| 2.40 | 1242 | 1243 | 1244 | EVQLVESGGGVVRPGGSLRLSCAASGFTFDDYAWSWVRQAPGK |
| | DYAWS | GITWNGGST | DKYSYAWSY | GLEWVSGITWNGGSTGYADSVKDRFTISRDNAKNSLYLQMNSLR |
| | | GYADSVKD | DGFDI | AEDTALYYCVRDKYSYAWSYDGFDIWGQGTMVTVSS |
| PD | SEQ ID NO: | SEQ ID NO: | SEQ ID NO: | SEQ ID NO: 1249 |
| 2.41 | 1246 | 1247 | 1248 | EVQLVESGGGVVRPGGSLRLSCAASGFTFDDYARSWVRQAPGKG |
| | DYARS | GITWNGGST | DKYSYAWSY | LEWVSGITWNGGSTGYADSVKDRFTISRDNAKNSLYLQMNSLRA |
| | | GYADSVKD | DGFDI | EDTALYYCVRDKYSYAWSYDGFDIWGQGTMVTVSS |
| PD | SEQ ID NO: | SEQ ID NO: | SEQ ID NO: | SEQ ID NO: 1253 |
| 2.42 | 1250 | 1251 | 1252 | EVQLVESGGGVVRPGGSLRLSCAASGFTFDDYAFSWVRQAPGKG |
| | DYAFS | GITWNGGST | DKYSYAWSY | LEWVSGITWNGGSTGYADSVKDRFTISRDNAKNSLYLQMNSLRA |
| | | GYADSVKD | DGFDI | EDTALYYCVRDKYSYAWSYDGFDIWGQGTMVTVSS |
| PD | SEQ ID NO: | SEQ ID NO: | SEQ ID NO: | SEQ ID NO: 1257 |
| 2.43 | 1254 | 1255 | 1256 | EVQLVESGGGVVRPGGSLRLSCAASGFTFDDYAVSWVRQAPGKG |
| | DYAVS | GITWNGGST | DKYSYAWSY | LEWVSGITWNGGSTGYADSVKDRFTISRDNAKNSLYLQMNSLRA |
| | | GYADSVKD | DGFDI | EDTALYYCVRDKYSYAWSYDGFDIWGQGTMVTVSS |
| PD | SEQ ID NO: | SEQ ID NO: | SEQ ID NO: | SEQ ID NO: 1261 |
| 2.44 | 1258 | 1259 | 1260 | EVQLVESGGGVVRPGGSLRLSCAASGFTFDDYAMSWVRQAPGK |
| | DYAMS | GITWTGGST | DKYSYAWSY | GLEWVSGITWTGGSTGYADSVKDRFTISRDNAKNSLYLQMNSLR |
| | | GYADSVKD | DGFDI | AEDTALYYCVRDKYSYAWSYDGFDIWGQGTMVTVSS |
| PD | SEQ ID NO: | SEQ ID NO: | SEQ ID NO: | SEQ ID NO: 1265 |
| 2.45 | 1262 | 1263 | 1264 | EVQLVESGGGVVRPGGSLRLSCAASGFTFDDYAMSWVRQAPGK |
| | DYAMS | GITWSGGST | DKYSYAWSY | GLEWVSGITWSGGSTGYADSVKDRFTISRDNAKNSLYLQMNSLR |
| | | GYADSVKD | DGFDI | AEDTALYYCVRDKYSYAWSYDGFDIWGQGTMVTVSS |
| PD | SEQ ID NO: | SEQ ID NO: | SEQ ID NO: | SEQ ID NO: 1269 |
| 2.46 | 1266 | 1267 | 1268 | EVQLVESGGGVVRPGGSLRLSCAASGFTFDDYAMSWVRQAPGK |
| | DYAMS | GITWPGGST | DKYSYAWSY | GLEWVSGITWPGGSTGYADSVKDRFTISRDNAKNSLYLQMNSLR |
| | | GYADSVKD | DGFDI | AEDTALYYCVRDKYSYAWSYDGFDIWGQGTMVTVSS |
| PD | SEQ ID NO: | SEQ ID NO: | SEQ ID NO: | SEQ ID NO: 1273 |
| 2.47 | 1270 | 1271 | 1272 | EVQLVESGGGVVRPGGSLRLSCAASGFTFDDYAMSWVRQAPGK |
| | DYAMS | GITWIGGST | DKYSYAWSY | GLEWVSGITWIGGSTGYADSVKDRFTISRDNAKNSLYLQMNSLRA |
| | | GYADSVKD | DGFDI | EDTALYYCVRDKYSYAWSYDGFDIWGQGTMVTVSS |
| PD | SEQ ID NO: | SEQ ID NO: | SEQ ID NO: | SEQ ID NO: 1277 |
| 2.48 | 1274 | 1275 | 1276 | EVQLVESGGGVVRPGGSLRLSCAASGFTFDDYAMSWVRQAPGK |
| | DYAMS | GITWLGGST | DKYSYAWSY | GLEWVSGITWLGGSTGYADSVKDRFTISRDNAKNSLYLQMNSLR |
| | | GYADSVKD | DGFDI | AEDTALYYCVRDKYSYAWSYDGFDIWGQGTMVTVSS |
| PD | SEQ ID NO: | SEQ ID NO: | SEQ ID NO: | SEQ ID NO: 1281 |
| 2.49 | 1278 | 1279 | 1280 | EVQLVESGGGVVRPGGSLRLSCAASGFTFDDYAMSWVRQAPGK |
| | DYAMS | GITWNGGST | DKYSYAWSY | GLEWVSGITWNGGSTGYADSVKGRFTISRDNAKNSLYLQMNSLR |
| | | GYADSVKG | DGFDI | AEDTALYYCVRDKYSYAWSYDGFDIWGQGTMVTVSS |
| PD | SEQ ID | SEQ ID NO: | SEQ ID NO: | SEQ ID NO: 1285 |
| 2.50 | NO:1282 | 1283 | 1284 | EVQLVESGGGVVRPGGSLRLSCAASGFTFDDYAMSWVRQAPGK |
| | DYAMS | GITWKGGST | DKYSYAWSY | GLEWVSGITWKGGSTGYADSVKDRFTISRDNAKNSLYLQMNSLR |
| | | GYADSVKD | DGFDI | AEDTALYYCVRDKYSYAWSYDGFDIWGQGTMVTVSS |
| PD | SEQ ID NO: | SEQ ID NO: | SEQ ID NO: | SEQ ID NO: 1289 |
| 2.51 | 1286 | 1287 | 1288 | EVQLVESGGGVVRPGGSLRLSCAASGFTFDDYAMSWVRQAPGK |
| | DYAMS | GITWRGGST | DKYSYAWSY | GLEWVSGITWRGGSTGYADSVKDRFTISRDNAKNSLYLQMNSLR |
| | | GYADSVKD | DGFDI | AEDTALYYCVRDKYSYAWSYDGFDIWGQGTMVTVSS |
| PD | SEQ ID NO: | SEQ ID NO: | SEQ ID NO: | SEQ ID NO: 1293 |
| 2.52 | 1290 | 1291 | 1292 | EVQLVESGGGVVRPGGSLRLSCAASGFTFDDYAMSWVRQAPGK |
| | DYAMS | GITWNGGST | DKYSYAWSY | GLEWVSGITWNGGSTGYADSVKDRFTISRDNAKNSLYLQMNSLR |
| | | GYADSVKD | DVFDI | AEDTALYYCVRDKYSYAWSYDVFDIWGQGTMVTVSS |
| PD | SEQ ID NO: | SEQ ID NO: | SEQ ID NO: | SEQ ID NO: 1297 |
| 2.53 | 1294 | 1295 | 1296 | EVQLVESGGGVVRPGGSLRLSCAASGFTFDDYAMSWVRQAPGK |
| | DYAMS | GITWNGGST | DKYSYAWSY | GLEWVSGITWNGGSTGYADSVKDRFTISRDNAKNSLYLQMNSLR |
| | | GYADSVKD | DDFDI | AEDTALYYCVRDKYSYAWSYDDFDIWGQGTMVTVSS |

Exemplary nucleic acid sequences are

SEQ ID NO: 1878 (his encodes PD1.1) and SEQ ID NO: 1879) (this encodes PD2.1).

In one embodiment, the PD-1 binding V_{H} single domain antibody is a variant of one of the V_{H} single domain antibodies shown in table 9 or 10 which comprises one or more substitution in the amino acid sequence. Typical modifications are explained elsewhere herein. For example, a variant of PD1.1 can have amino acid substitutions at one or more or all of the following positions: 5L, 32H, 44G, 55S, 66D, 77S and/or 105T. In one embodiment, the V_{H} single domain antibody comprises PD1.1 with amino acid substitutions selected from one of the following:
a) 5L→V, 11S→L, 32H→Y, 44G→D, 55S→G, 62D→A, 66D→G and 77S→N or
b) 5L→V, 11S→L, 32H→Y, 44G→D, 55S→G, 66D→G and 77S→N or
c) 1E→Q, 5L→V, 11S→L, 32H→Y, 44G→D, 55S→G, 66D→G, 77S→N, 98R→K, 99E→D, 105T→I and 102M→T.

In one embodiment, a variant or PD1.29 has amino acid substitutions at one or more or all of the following positions: M34, M58, V102, V116. In one embodiment, the V_{H} single domain antibody comprises SEQ ID No 136 with amino acid substitutions selected from one of the following:

### a) M34→L, M58→Y, V116A (PD 1.39);

### b) M34→F, G54→A, M58→Q, V102A (PD1.50);

### c) V1116A (PD 1.26);

### d) M34→L, M58→Q, V1116A (PD 1.40).

In one embodiment, the V_{H} single domain antibody comprises PD2.1 with amino acid substitutions at one or more or all of the following positions: G109, D66, G55. In one embodiment, the V_{H} single domain antibody comprises SEQ ID No 254 with amino acid substitutions selected from one of the following:
a) G109→D, D66→G, G55→A (PD 2.12);
b) G109→D, G55→A (PD 2.27);
c) G109→D (PD 2.53);
d) G109→D, D66→G (PD 2.29);
e) G109→V, D66→G (PD 2.28).

In one embodiment, when Q is found at position 1, it is changed to E or another residue. The numbering used above is based on the actual position of the residue in the molecule.

In one embodiment, the binding molecule may comprise one or more single domain antibodies that bind to PD-1, for example one or two single domain antibodies as described above.

In one embodiment, the binding molecule may comprise one or more single domain antibodies that bind to PD-1, for example one or two single domain antibodies as described above. In one embodiment the binding molecule comprises two single domain antibodies that bind to PD-1 wherein each binds to a different epitope of PD-1, thus providing a biparatopic PD-1 binder.

### PD-1 Blocking Molecules

In another embodiment, the PD-1 binding moiety is a single V_{H} domain antibody that blocks the functional interaction between PD-1 and at least one of its ligands, e.g. blocks PD-1 signalling. PD-1 “blocking single domain antibody or antibody” or a “neutralizing single domain antibody or antibody”, as used herein refers to an antibody whose binding to PD-1 results in inhibition of at least one biological activity of PD-1. For example, a single domain antibody of the invention may prevent or block PD-1 binding to PD-L1 and/or PD-L2. In one embodiment, the single domain antibody of the invention blocks PD-1 binding to PD-L1. In one embodiment, the single domain antibody of the invention blocks PD-1 binding to PD-L2.

### Exemplary Sequences of PD-1 Blocking Molecules

In one aspect, the single variable heavy chain domain antibody comprises a CDR1, CDR2 or CDR3 as shown for one of the single domain antibodies as shown in Table 11 or having a CDR1, 2 or 3 with at least 75% homology thereto; or a set of CDRs (i.e. CDR1, CDR2, CDR3) wherein said set is as shown for one of the single domain antibodies as shown in Table 11 or with at least 75% 80%, 90% or 95% homology thereto.

In one embodiment, the single variable heavy chain domain antibody comprises a set of CDR1, 2, and 3 sequences as shown for V_{H} single domain antibodies in table 11 or a sequence with at least 75%, 80%, 90% or 95% sequence homology thereto. Thus, in one embodiment, the V_{H} single domain antibody has a CDR1 with SEQ ID NO. 1298, a CDR2 with SEQ ID NO. 1299 and a CDR3 with SEQ ID NO. 1300. In another embodiment, the V_{H} single domain antibody has a CDR1 with SEQ ID NO. 1302, a CDR2 with SEQ ID NO. 1303 and a CDR3 with SEQ ID NO. 1304. In another embodiment, the V_{H} single domain antibody has a CDR1 with SEQ ID NO. 1306, a CDR2 with SEQ ID NO. 1307 and a CDR3 with SEQ ID NO. 1308.

In another aspect, the single variable heavy chain domain antibody according to the invention comprises or consists of a full length sequence as shown in Table 11 or a sequence with at least 50%, 80%, 90% or 95% homology thereto.

Sequence homology can be at least 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% for example at least 90%, 91%, 92%, 93%, 94%, 95%, 96%, 97%, 98% or 99% sequence homology. As mentioned elsewhere, homology and identity are used interchangeably herein and the above values also refer to sequence identity.

TABLE 11
PD-1 blockers

| Name | | | | |
|---|---|---|---|---|
| Blocker | CDR1 | CDR2 | CDR3 | Full length |
| PD1 | SEQ ID | SEQ ID NO: | SEQ ID NO: 1300 | SEQ ID NO: 1301 |
| blocker | NO: | 1299 | EAPLRLGESPHDA | QVQLVESGGGLVKPGGSLRLSCAASGFTFSDYTMTWMRQAP |
| 1.1 | 1298 | YISTGGTIKYY | FDI | GKGLEWVSYISTGGTIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1304 | SEQ ID NO: 1305 |
| blocker | NO: | 1303 | EAPLRLGESPHDA | EVQLLESGGGLVKPGGSLRLSCAASGFTFSDYTMTWMRQAP |
| 1.2 | 1302 | YISTGGSIKYY | FDI | GKGLEWVSYISTGGSIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1308 | SEQ ID NO: 1309 |
| blocker | NO: | 1307 | EAPLRLGESPHDA | QVQLVESGGGLVKPGGSLRLSCAASGFTFSDYTMTWMRQAP |
| 1.3 | 1306 | YISTGGSIKYY | FDI | GKGLEWVSYISTGGSIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1312 | SEQ ID NO: 1313 |
| blocker | NO: | 1311 | EAPLRLGETPHDA | QVQLVESGGGLVKPGGSLRLSCAASGFTFSDYYMIWMRQAP |
| 1.4 | 1310 | YISGGGTTKYY | FDI | GKGLEWVSYISGGGTTKYYTDSVKGRFTISRDNAKNSLYLQMN |
| | DYYMI | TDSVKG | | SLRADDTAVYYCAREAPLRLGETPHDAFDIWGQGTMVTVSS |
| PD1 | SEQ ID | SEQ ID NO: | SEQ ID NO: 1316 | SEQ ID NO: 1317 |
| blocker | NO: | 1315 | EAPLRLGESPHDA | EVQLVESGGGLVKPGGSLRLSCAASGFTFSDYTMTWMRQAP |
| 1.5 | 1314 | YISTGGNTKYY | FDI | GKGLEWVSYISTGGNTKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1320 | SEQ ID NO: 1321 |
| blocker | NO: | 1319 | EAPLRLGESPHDA | EVQLLESGGGLVKPGGSLRLSCAASGFTFSDYTMTWMRQAP |
| 1.6 | 1318 | YISTGGTIKYY | FDI | GKGLEWVSYISTGGTIKYYTDSVKGRFTISRDNAKNSLYLQMSS |
| | DYTMT | TDSVKG | | LRADDTAVYYCAREAPLRLGESPHDAFDIWGQGTMVTVSS |
| PD1 | SEQ ID | SEQ ID NO: | SEQ ID NO: 1324 | SEQ ID NO: 1325 |
| blocker | NO: | 1323 | EAPLRLGESPHDA | EVQLVESGGGLVKPGGSLRLSCAASGFTFSDYTMTWMRQAP |
| 1.7 | 1322 | YISTGGTIKYY | FDI | GKGLEWVSYISTGGTIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1328 | SEQ ID NO: 1329 |
| blocker | NO: | 1327 | EAPLRLGESPHDA | EVQLVESGGGLVKPGGSLRLSCAASGFTFSDYTMTWMRQAP |
| 1.8 | 1326 | YISTGGSIKYY | FDI | GKGLEWVSYISTGGSIKYYTDSVKGRFTISRDNAKNSLYLQMN |
| | DYTMT | TDSVKG | | SLRAGDTAVYYCAREAPLRLGESPHDAFDIWGQGTMVTVSS |
| PD1 | SEQ ID | SEQ ID NO: | SEQ ID NO: 1332 | SEQ ID NO: 1333 |
| blocker | NO: | 1331 | EAPLRLGESPHDA | QVQLVESGGGLVKPGGSLRLSCAASGFTFGDYTMTWMRQAP |
| 1.9 | 1330 | YISTGGTIKYY | FDI | GKGLEWVSYISTGGTIKYYTDSVKGRFTISRDNAKNSLYLQMD |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1336 | SEQ ID NO: 1337 |
| blocker | NO: | 1335 | EAPLRLGESPHDA | QVQLVESGGGLVKPGGSLRLSCAASGFTFSDYTMSWMRQAP |
| 1.10 | 1334 | YISLGGNTKYY | FDI | GKGLEWISYISLGGNTKYYTDSVKGRFTISRDNAKNSLYLQMN |
| | DYTMS | TDSVKG | | SLRADDTAVYYCAREAPLRLGESPHDAFDIWGQGTMVTVSS |
| PD1 | SEQ ID | SEQ ID NO: | SEQ ID NO: 1340 | SEQ ID NO: 1341 |
| blocker | NO: | 1339 | EAPLRLGETPHDA | EVQLLESGGGLVKPGGSLRLSCAASGFTFSDYDMTWIRQAPG |
| 1.11 | 1338 | YISRGGSTKYY | FDI | KGQEWVSYISRGGSTKYYADSVKGRFTISRDNAKNSLYLQMN |
| | DYDMT | ADSVKG | | SLRAEDTAVYYCAREAPLRLGETPHDAFDIWGQGTMVTVSS |
| PD1 | SEQ ID | SEQ ID NO: | SEQ ID NO: 1344 | SEQ ID NO: 1345 |
| blocker | NO: | 1343 | EAPLRLGESPHDA | EVQLLESGGGVVKPGGSLRLSCAASGFTFSDYYMGWIRQAPG |
| 1.12 | 1342 | YISSSGSTIYYA | FDI | KGLEWISYISSSGSTIYYADSVKGRFTISRDNAKNSLYLQMNSLR |
| | DYYMG | DSVKG | | AEDTAVYYCAREAPLRLGESPH DAFDIWGQGTMVTVSS |
| PD1 | SEQ ID | SEQ ID NO: | SEQ ID NO: 1348 | SEQ ID NO: 1349 |
| blocker | NO: | 1347 | EAPLRLGESPHDA | EVQLLESGGGLVKPGGSLRLSCAASGFTFSDYTMTWMRQAP |
| 1.13 | 1346 | YISTGGTIKYY | FDI | GKGLEWVSYISTGGTIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1352 | SEQ ID NO: 1353 |
| blocker | NO: | 1351 | EAPLRLGESPHDA | EVQLVESGGGLVQPGRSLRLSCAASGFTFSDYTMTWMRQAP |
| 1.14 | 1350 | YISTGGTIKYY | FDI | GKGLEWVSYISTGGTIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1356 | SEQ ID NO: 1357 |
| blocker | NO: | 1355 | EAPLRLGESPHDA | QVQLVESGGGLVKPGGSLRLSCAASGFTFSDNSMSWIRQAPG |
| 1.15 | 1354 | YISSSGSTIYYA | FDI | KGLEWVSYISSSGSTIYYADSVKGRFTISRDNAKNSLYLQMNTL |
| | DNSMS | DSVKG | | RAEDTAVYYCAKEAPLRLGESPH DAFDIWGQGTMVTVSS |
| PD1 | SEQ ID | SEQ ID NO: | SEQ ID NO: 1360 | SEQ ID NO: 1361 |
| blocker | NO: | 1359 | EAPLRLGESPHDA | EVQLVESGGGLVKPGGSLRLSCAASGFTFSDYTMSWMRQAP |
| 1.16 | 1358 | YISTGGSIKYY | FDI | GKGLEWVSYISTGGSIKYYTDSVKGRFTISRDNAKNSLYLQMN |
| | DYTMS | TDSVKG | | SLRADDTAVYYCAREAPLRLGESPHDAFDIWGQGTMVTVSS |
| PD1 | SEQ ID | SEQ ID NO: | SEQ ID NO: 1364 | SEQ ID NO: 1365 |
| blocker | NO: | 1363 | EAPLRLGESPHDA | EVQLVESGGGLVKPGGSLRLSCAASGFTFSDYTMTWMRQAP |
| 1.17 | 1362 | YISTGGSIKYY | FDI | GKGLEWVSYISTGGSIKYYTDSVKGRFTISRDNAKNSLYLQMN |
| | DYTMT | TDSVKG | | SLRVDDTAVYYCAREAPLRLGESPHDAFDIWGQGTMVTVSS |
| PD1 | SEQ ID | SEQ ID NO: | SEQ ID NO: 1368 | SEQ ID NO: 1369 |
| blocker | NO: | 1367 | EAPLRLGESPHDA | QVQLLESGGGLVKPGGSLRLSCAASGFTFSDYTMTWMRQAP |
| 1.18 | 1366 | YISTGGTIKYY | FDI | GKGLEWVSYISTGGTIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1372 | SEQ ID NO: 1373 |
| blocker | NO: | 1371 | EAPLRLGETPHDA | QVQLVESGGGLVKPGGSLRLSCAASGFTFSDYTMTWMRQAP |
| 1.19 | 1370 | YISTGGSIKYY | FDI | GKGLEWVSYISTGGSIKYYTDSVKGRFTISRDNAKNSLYLQMN |
| | DYTMT | TDSVKG | | SLRAEDTAVYYCAREAPLRLGETPHDAFDIWGQGTMVTVSS |
| PD1 | SEQ ID | SEQ ID NO: | SEQ ID NO: 1376 | SEQ ID NO: 1377 |
| blocker | NO: | 1375 | EAPLRLGESPHDA | EVQLLESGGGLVKPGGSLRLSCAASGFTFSDYTMTWMRQAP |
| 1.20 | 1374 | YISSGGSIKFY | FDI | GKGLEWVSYISSGGSIKFYADSVKGRFTISRDNAKNSLYLQMN |
| | DYTMT | ADSVKG | | SLRAEDTAVYYCAREAPLRLGESPHDAFDIWGQGTMVTVSS |
| PD1 | SEQ ID | SEQ ID NO: | SEQ ID NO: 1380 | SEQ ID NO: 1381 |
| blocker | NO: | 1379 | EAPLRLGESPHDA | EVQLVESGGGLVKPGGSLRLSCAASGFTFSDYTMTWMRQAP |
| 1.21 | 1378 | YISTGGSIKYY | FDT | GKGLEWVSYISTGGSIKYYTDSVKGRFTISRDNAKNSLYLQMN |
| | DYTMT | TDSVKG | | SLRADDTAVYYCAREAPLRLGESPHDAFDTWGQGTMVTVSS |
| PD1 | SEQ ID | SEQ ID NO: | SEQ ID NO: 1384 | SEQ ID NO: 1385 |
| blocker | NO: | 1383 | EAPLRLGESPHDA | EVQLVESGGGLVKPGGSLRLSCAASGFTFSDYTMTWMRQAP |
| 1.22 | 1382 | YISTGGSIKYY | FDI | GKGLEWVSYISTGGSIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1388 | SEQ ID NO: 1389 |
| blocker | NO: | 1387 | EAPLRLGESPHDA | QITLKESGGGLVKPGGSLRLSCAASGFTFSDYTMSWMRQAPG |
| 1.23 | 1386 | YISTGGTIKYY | FDI | KGLEWVSYISTGGTIKYYTDSVKGRFTISRDNAKNSLYLQMNSL |
| | DYTMS | TDSVKG | | RADDTAVYYCAREAPLRLGESPHDAFDIWGQGTMVTVSS |
| PD1 | SEQ ID | SEQ ID NO: | SEQ ID NO: | SEQ ID NO: 1393 |
| blocker | NO: | 1391 | 1392 | QVTLKESGGGLVKPGGSLRLSCAASGFTFSDYDMYWIRQAPG |
| 1.24 | 1390 | YISRGGSVTYY | EAPLRLGETPHAA | KGLEWVSYISRGGSVTYYADSVKGRFTISRDNAKNALYLQMNS |
| | DYDMY | ADSVKG | FDI | LRAEDMAVYFCATEAPLRLGETPHAAFDIWGQGTMVTVSS |
| PD1 | SEQ ID | SEQ ID NO: | SEQ ID NO: 1396 | SEQ ID NO: 1397 |
| blocker | NO: | 1395 | EAPLRLGESPHDA | QVTLKESGGGLVKPGGSLRLSCAASGFTFSDYYMSWFRQAPG |
| 1.25 | 1394 | FISSSGSTTYY | FDF | KEREWISFISSSGSTTYYADSVKGRFTISRDNAKNSLYLQMNSL |
| | DYYMS | ADSVKG | | RAEDTAVYYCAREAPLRLGESPHDAFDFWGQGTMVTVSS |
| PD1 | SEQ ID | SEQ ID NO: | SEQ ID NO: 1400 | SEQ ID NO: 1401 |
| blocker | NO: | 1399 | EAPLRLGESPHDA | QVTLKESGGGLVKPGGSLRLSCAASGFTFSDNSMSWIRQAPG |
| 1.26 | 1398 | YISSSGSTIYYA | FDI | KGLEWVSYISSSGSTIYYADSVKGRFTISRDNAKNSLYLQMNTL |
| | DNSMS | DSVKG | | RAEDTAVYYCAKEAPLRLGESPH DAFDIWGQGTMVTVSS |
| PD1 | SEQ ID | SEQ ID NO: | SEQ ID NO: 1404 | SEQ ID NO: 1405 |
| blocker | NO: | 1403 | EAPLRLGESPHDA | QVTLKESGGGLVKPGGSLRLSCAASGFTFSDYTMTWMRQAP |
| 1.27 | 1402 | YISTGGSIKYY | FDI | GKGLEWVSYISTGGSIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1408 | SEQ ID NO: 1409 |
| blocker | NO: | 1407 | EAPLRLGETPHAA | QITLKESGGGLVKPGGSLRLSCAASGFTFSDYDMYWIRQAPGK |
| 1.28 | 1406 | YISRGGSVTYY | FDI | GLEWVSYISRGGSVTYYADSVKGRFTISRDNAKNALYLQMNSL |
| | DYDMY | ADSVKG | | RAEDMAVYFCATEAPLRLGETPHAAFDIWGQGTMVTVSS |
| PD1 | SEQ ID | SEQ ID NO: | SEQ ID NO: 1412 | SEQ ID NO: 1413 |
| blocker | NO: | 1411 | EAPLRLGESPHDA | QVTLKESGGGLVKPGGSLRLSCAASGFTFSDYTMSWMRQAP |
| 1.29 | 1410 | YISTGGTIKYY | FDI | GKGLEWVSYISTGGTIKYYTDSVKGRFTISRDNAKNSLYLQMN |
| | DYTMS | TDSVKG | | SLRADDTAVYYCAREAPLRLGESPHDAFDIWGQGTMVTVSS |
| PD1 | SEQ ID | SEQ ID NO: | SEQ ID NO: 1416 | SEQ ID NO: 1417 |
| blocker | NO: | 1415 | EAPLRLGESPHDA | QVTLKESGGGLVKPGGSLRLSCAASGFTFSDYTMTWMRQAP |
| 1.30 | 1414 | YISTGGSIKYY | FDI | GKGLEWVSYISTGGSIKYYTDSVKGRFTISRDNARNSLYLQMN |
| | DYTMT | TDSVKG | | SLRAEDTAVYYCAREAPLRLGESPHDAFDIWGQGTMVTVSS |
| PD1 | SEQ ID | SEQ ID NO: | SEQ ID NO: 1420 | SEQ ID NO: 1421 |
| blocker | NO: | 1419 | EAPLRLGESPHDA | QVTLKESGGGLVKPGGSLRLSCAASGFTFSDYTMTWMRQAP |
| 1.31 | 1418 | YISTGGSTKYY | FDI | GKGLEWVSYISTGGSTKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1424 | SEQ ID NO: 1425 |
| blocker | NO: | 1423 | EAPLRLGESPHDA | QVTLKESGGGLVKPGGSLRLSCAASGFTFSDDYMMWIRQAP |
| 1.32 | 1422 | YISSGGSIIYYA | FDI | GKGLEWVSYISSGGSIIYYADSVKGRFTISRDNAKNSLYLQMNS |
| | DDYMM | DSVKG | | LRAEDTAVYYCAREAPLRLGESPHDAFDIRGQGTMVTVSS |
| PD1 | SEQ ID | SEQ ID NO: | SEQ ID NO: 1428 | SEQ ID NO: 1429 |
| blocker | NO: | 1427 | EAPLRLGETPHAA | QITLKESGGGLVKPGGSLRLSCAASGFTFSDYDMYWVRQAPG |
| 1.33 | 1426 | YISRGGSVTYY | FDI | KGLEWVSYISRGGSVTYYADSVKGRFTISRDNAKNALYLQMNS |
| | DYDMY | ADSVKG | | LRAEDMAVYFCATEAPLRLGETPHAAFDIWGQGTMVTVSS |
| PD1 | SEQ ID | SEQ ID NO: | SEQ ID NO: 1432 | SEQ ID NO: 1433 |
| blocker | NO: | 1431 | EAPLRLGESPHDA | EVQLVESGGGLVKPGGSLRLSCAASGFTFSDYTMTWMRQAP |
| 1.34 | 1430 | YISTGGSVKYY | FDI | GKGLEWVSYISTGGSVKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1436 | SEQ ID NO: 1437 |
| blocker | NO: | 1435 | EAPLRLGESPHDA | EVQLLESGGGLVKPGGSLRLSCAASGFTFSDYTMTWMRQAP |
| 1.35 | 1434 | YISTGGSIKYY | FDI | GKGLEWVSYISTGGSIKYYTDSVKGRFTISRDNAKNSLYLQMN |
| | DYTMT | TDSVKG | | SLRVDDTAVYYCAREAPLRLGESPHDAFDIWGQGTMVTVSS |
| PD1 | SEQ ID | SEQ ID NO: | SEQ ID NO: 1440 | SEQ ID NO: 1441 |
| blocker | NO: | 1439 | EAPLRLGESPHDA | EVQLVESGGGLVKPGGSLRLSCAASGFTFSDYTMTWMRQAP |
| 1.36 | 1438 | YISTGGTIKYY | FDI | GKGLEWVSYISTGGTIKYYTDSVKGRFTISRDNAKNSLFLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1444 | SEQ ID NO: 1445 |
| blocker | NO: | 1443 | EAPLRLGESPHDA | QVQLVESGGGLVKPGGSLRLSCAASGFTFSDYTMTWMRQAP |
| 1.37 | 1442 | YISTGGSIKYY | FDI | GKGLEWVSYISTGGSIKYYTDSVKGRFTISRDNAKNSLFLQMNS |
| | DYTMT | TDSVKG | | LRADDTAVYYCAREAPLRLGESPHDAFDIWGQGTMVTVSS |
| PD1 | SEQ ID | SEQ ID NO: | SEQ ID NO: 1448 | SEQ ID NO: 1449 |
| blocker | NO: | 1447 | EAPLRLGESPHDA | EVQLVESGGGLVQPGGSLRLSCAASGFTFSDYTMTWMRQAP |
| 1.38 | 1446 | YISTGGTIKYY | FDI | GKGLEWVSYISTGGTIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1452 | SEQ ID NO: 1453 |
| blocker | NO: | 1451 | EAPLRLGESPHDA | QVQLQESGGGLVKPGGSLRLSCAASGFTFSDYTMTWMRQAP |
| 1.39 | 1450 | YISTGGSIKYY | FDI | GKGLEWVSYISTGGSIKYYTDSVKGRFTISRDNAKNSLYLQMN |
| | DYTMT | TDSVKG | | SLRADDTAVYYCAREAPLRLGESPHDAFDIWGQGTMVTVSS |
| PD1 | SEQ ID | SEQ ID NO: | SEQ ID NO: 1456 | SEQ ID NO: 1457 |
| blocker | NO: | 1455 | EAPLRLGESPHDA | EVQLVESGGGLVKPGGSLRLSCAASGFTFSDSSMSWIRQAPG |
| 1.40 | 1454 | YISSGGGIIYYT | FDI | RGLEWISYISSGGGIIYYTDSVKGRFTISRDNAKNSLYLQMNSLR |
| | DSSMS | DSVKG | | VEDTAVYYCAKEAPLRLGESPHDAFDIWGHGTMVTVSS |
| PD1 | SEQ ID | SEQ ID NO: | SEQ ID NO: 1460 | SEQ ID NO: 1461 |
| blocker | NO: | 1459 | EAPLRLGESPHDA | QVQLVESGGGLVKPGGSLRLSCAASGFTFSDNSMTWMRQAP |
| 1.41 | 1458 | YISSGGGVIFY | FDI | GKGLEWVSYISSGGGVIFYADSVKGRFTISRDNAKNSLYLQMN |
| | DNSMT | ADSVKG | | SLRADDTAVYYCAREAPLRLGESPHDAFDIWGQGTMVTVSS |
| PD1 | SEQ ID | SEQ ID NO: | SEQ ID NO: 1464 | SEQ ID NO: 1465 |
| blocker | NO: | 1463 | EAPLRLGESPHDA | QVQLVESGGGLVKPGGSLRLSCAASGFTFSDNSMTWMRQAP |
| 1.42 | 1462 | YISSGGGVKFY | FDI | GKGLEWVSYISSGGGVKFYADSVKGRFTISRDNAKNSLYLQM |
| | DNSMT | ADSVKG | | NSLRADDTAVYYCAREAPLRLGESPHDAFDIWGQGTMVTVSS |
| PD1 | SEQ ID | SEQ ID NO: | SEQ ID NO: 1468 | SEQ ID NO: 1469 |
| blocker | NO: | 1467 | EAPLRLGESPHDA | QVQLVESGGGLVKPGGSLRLSCAASGFTFSDSSMTWMRQAP |
| 1.43 | 1466 | YISSGGGVIFY | FDI | GKGLEWVSYISSGGGVIFYADSVKGRFTISRDNAKNSLYLQMN |
| | DSSMT | ADSVKG | | SLRADDTAVYYCAREAPLRLGESPHDAFDIWGQGTMVTVSS |
| PD1 | SEQ ID | SEQ ID NO: | SEQ ID NO: 1472 | SEQ ID NO: 1473 |
| blocker | NO: | 1471 | EAPLRLGESPHDA | QVQLVESGGGLVKPGGSLRLSCAASGFTFSDNSMTWMRQAP |
| 1.44 | 1470 | YISSGGAVKFY | FDI | GKGLEWVSYISSGGAVKFYADSVKGRFTISRDNAKNSLYLQM |
| | DNSMT | ADSVKG | | NSLRADDTAVYYCAREAPLRLGESPHDAFDIWGQGTMVTVSS |
| PD1 | SEQ ID | SEQ ID NO: | SEQ ID NO: 1476 | SEQ ID NO: 1477 |
| blocker | NO: | 1475 | EAPLRLGESPHDA | QVQLVESGGGLVKPGGSLRLSCAASGFTFSDYSMSWMRQAP |
| 1.45 | 1474 | YISSGGGVIFY | FDI | GKGLEWVSYISSGGGVIFYADSVKGRFTISRDNAKNSLYLQMN |
| | DYSMS | ADSVKG | | SLRADDTAVYYCAREAPLRLGESPHDAFDIWGQGTMVTVSS |
| PD1 | SEQ ID | SEQ ID NO: | SEQ ID NO: 1480 | SEQ ID NO: 1481 |
| blocker | NO: | 1479 | EAPLRLGESPHDA | QVQLVESGGGLVKPGGSLRLSCAASGFTFSDSSMSWMRQAP |
| 1.46 | 1478 | YISSGGGVIFY | FDI | GKGLEWVSYISSGGGVIFYADSVKGRFTISRDNAKNSLYLQMN |
| | DSSMS | ADSVKG | | SLRADDTAVYYCAREAPLRLGESPHDAFDIWGQGTMVTVSS |
| PD1 | SEQ ID | SEQ ID NO: | SEQ ID NO: 1484 | SEQ ID NO: 1485 |
| blocker | NO: | 1483 | EAPLRLGESPHDA | QVQLVESGGGLVKPGGSLRLSCAASGFTFSDNSMSWMRQAP |
| 1.47 | 1482 | YISSGGGVIFY | FDI | GKGLEWVSYISSGGGVIFYADSVKGRFTISRDNAKNSLYLQMN |
| | DNSMS | ADSVKG | | SLRADDTAVYYCAREAPLRLGESPHDAFDIWGQGTMVTVSS |
| PD1 | SEQ ID | SEQ ID NO: | SEQ ID NO: 1488 | SEQ ID NO: 1489 |
| blocker | NO: | 1487 | EAPLRLGESPHDA | QVQLVESGGGLVKPGGSLRLSCAASGFTFSDSSMTWMRQAP |
| 1.48 | 1486 | YISSGGGVKFY | FDI | GKGLEWVSYISSGGGVKFYADSVKGRFTISRDNAKNSLYLQM |
| | DSSMT | ADSVKG | | NSLRADDTAVYYCAREAPLRLGESPHDAFDIWGQGTMVTVSS |
| PD1 | SEQ ID | SEQ ID NO: | SEQ ID NO: 1492 | SEQ ID NO: 1493 |
| blocker | NO: | 1491 | EAPLRLGESPHDA | QVQLVESGGGLVKPGGSLRLSCAASGFTFSDSSMSWMRQAP |
| 1.49 | 1490 | YISTGGGVKF | FDI | GKGLEWVSYISTGGGVKFYADSVKGRFTISRDNAKNSLYLQM |
| | DSSMS | YADSVKG | | NSLRADDTAVYYCAREAPLRLGESPHDAFDIWGQGTMVTVSS |
| PD1 | SEQ ID | SEQ ID NO: | SEQ ID NO: 1496 | SEQ ID NO: 1497 |
| blocker | NO: | 1495 | EAPLRLGESPHDA | QVQLVESGGGLVKPGGSLRLSCAASGFTFSDNSMTWMRQAP |
| 1.50 | 1494 | YISSGGTIKFY | FDI | GKGLEWVSYISSGGTIKFYADSVKGRFTISRDNAKNSLYLQMN |
| | DNSMT | ADSVKG | | SLRADDTAVYYCAREAPLRLGESPHDAFDIWGQGTMVTVSS |
| PD1 | SEQ ID | SEQ ID NO: | SEQ ID NO: 1500 | SEQ ID NO: 1501 |
| blocker | NO: | 1499 | EAPLRLGESPHDA | QVQLVESGGGLVKPGGSLRLSCAASGFTFSDSSMTWMRQAP |
| 1.51 | 1498 | YISSGGAVKFY | FDI | GKGLEWVSYISSGGAVKFYTDSVKGRFTISRDNAKNSLYLQMN |
| | DSSMT | TDSVKG | | SLRADDTAVYYCAREAPLRLGESPHDAFDIWGQGTMVTVSS |
| PD1 | SEQ ID | SEQ ID NO: | SEQ ID NO: 1504 | SEQ ID NO: 1505 |
| blocker | NO: | 1503 | EAPLRLGESPHDA | QVQLVESGGGLVKPGGSLRLSCAASGFTFSDNSMTWMRQAP |
| 1.52 | 1502 | YISSGGGVKY | FDI | GKGLEWVSYISSGGGVKYYADSVKGRFTISRDNAKNSLYLQM |
| | DNSMT | YADSVKG | | NSLRADDTAVYYCAREAPLRLGESPHDAFDIWGQGTMVTVSS |
| PD1 | SEQ ID | SEQ ID NO: | SEQ ID NO: 1508 | SEQ ID NO: 1509 |
| blocker | NO: | 1507 | EAPLRLGESPHDA | QVQLVESGGGLVKPGGSLRLSCAASGFTFSDNSMTWMRQAP |
| 1.53 | 1506 | YISSGGSVKFY | FDI | GKGLEWVSYISSGGSVKFYADSVKGRFTISRDNAKNSLYLQMN |
| | DNSMT | ADSVKG | | SLRADDTAVYYCAREAPLRLGESPHDAFDIWGQGTMVTVSS |
| PD1 | SEQ ID | SEQ ID NO: | SEQ ID NO: 1512 | SEQ ID NO: 1513 |
| blocker | NO: | 1511 | EAPLRLGESPHDA | QVQLVESGGGLVKPGGSLRLSCAASGFTFSDDSMTWMRQAP |
| 1.54 | 1510 | YISSGGGVIFY | FDI | GKGLEWVSYISSGGGVIFYADSVKGRFTISRDNAKNSLYLQMN |
| | DDSMT | ADSVKG | | SLRADDTAVYYCAREAPLRLGESPHDAFDIWGQGTMVTVSS |
| PD1 | SEQ ID | SEQ ID NO: | SEQ ID NO: 1516 | SEQ ID NO: 1517 |
| blocker | NO: | 1515 | EAPLRLGESPHDA | QVQLVESGGGLVKPGGSLRLSCAASGFTFSDNSMTWMRQAP |
| 1.55 | 1514 | YISSGGGVKFY | FDI | GKGLEWVSYISSGGGVKFYADSVKGRFTISRDNAKNSLYLQM |
| | DNSMT | ADSVKG | | NSLRADDTAVYYCAREAPLRLGESPHDAFDISGQGTMVTVSS |
| PD1 | SEQ ID | SEQ ID NO: | SEQ ID NO: 1520 | SEQ ID NO: 1521 |
| blocker | NO: | 1519 | EAPLRLGESPHDA | QVQLVESGGGLVKPGGSLRLSCAASGFTFSDNSMTWMRQAP |
| 1.56 | 1518 | YISSGGAVKFY | FDI | GKGLEWVSYISSGGAVKFYADSVKGRFTISRDNAKNSLYLQM |
| | DNSMT | ADSVKG | | NSLRADDTAVYYCAREAPLRLGESPHDAFDILGQGTMVTVSS |
| PD1 | SEQ ID | SEQ ID NO: | SEQ ID NO: 1524 | SEQ ID NO: 1525 |
| blocker | NO: | 1523 | EAPLRLGESPHDA | QVQLVESGGGLVKPGGSLRLSCAASGFTFSDNSMTWMRQAP |
| 1.57 | 1522 | YISSGGGVIFY | FDI | GKGLEWVSYISSGGGVIFYADSVKGRFTISRDNAKNSLYLQMN |
| | DNSMT | ADSVKG | | SLRADDTAVYYCAREAPLRLGESPHDAFDISGQGTMVTVSS |
| PD1 | SEQ ID | SEQ ID NO: | SEQ ID NO: 1528 | SEQ ID NO: 1529 |
| blocker | NO: | 1527 | EAPLRLGESPHDA | QVQLVESGGGLVKPGGSLRLSCAASGFTFSDYTMTWMRQAP |
| 1.58 | 1526 | YISTGGGVKF | FDI | GKGLEWVSYISTGGTIKYYTDSVKGRFTISRDNAKNSLYLQMN |
| | DNTMT | YADSVKG | | SLRADDTAVYYCAREAPLRLGESPHDAFDIWGQGTMVTVSS |
| PD1 | SEQ ID | SEQ ID NO: | SEQ ID NO: 1532 | SEQ ID NO: 1533 |
| blocker | NO: | 1531 | EAPLRLGESPHDA | QVQLVESGGGLVKPGGSLRLSCAASGFTFSDNTMTWMRQAP |
| 1.59 | 1530 | YISSGGSVKFY | FDI | GKGLEWVSYISTGGGVKFYADSVKGRFTISRDNAKNSLYLQM |
| | DNSMS | ADSVKG | | NSLRADDTAVYYCAREAPLRLGESPHDAFDIWGQGTMVTVSS |
| PD1 | SEQ ID | SEQ ID NO: | SEQ ID NO: 1536 | SEQ ID NO: 1537 |
| blocker | NO: | 1535 | EAPLRLGESPHDA | QVQLVESGGGLVKPGGSLRLSCAASGFTFSDNSMSWMRQAP |
| 1.60 | 1534 | YISTGGGVKY | FDI | GKGLEWVSYISSGGSVKFYADSVKGRFTISRDNAKNSLYLQMN |
| | DNSMT | YADSVKG | | SLRADDTAVYYCAREAPLRLGESPHDAFDIWGQGTMVTVSS |
| PD1 | SEQ ID | SEQ ID NO: | SEQ ID NO: 1540 | SEQ ID NO: 1541 |
| blocker | NO: | 1539 | EAPLRLGESPHDA | QVQLVESGGGLVKPGGSLRLSCAASGFTFSDNSMTWMRQAP |
| 1.61 | 1538 | YISTGGGVKF | FDI | GKGLEWVSYISTGGGVKYYADSVKGRFTISRDNAKNSLYLQM |
| | DYTMS | YADSVKG | | NSLRADDTAVYYCAREAPLRLGESPHDAFDIWGQGTMVTVSS |
| PD1 | SEQ ID | SEQ ID NO: | SEQ ID NO: 1544 | SEQ ID NO: 1545 |
| blocker | NO: | 1543 | EAPLRLGESPHDA | QVQLVESGGGLVKPGGSLRLSCAASGFTFSDSSMTWMRQAP |
| 1.62 | 1542 | YISSGGAVKFY | FDI | GKGLEWVSYISSGGAVKFYTDSVKGRFTISRDNAKNSLYLQMN |
| | DSSMT | TDSVKG | | SLRADDTAVYYCAREAPLRLGESPHDAFDISGQGTMVTVSS |
| PD1 | SEQ ID | SEQ ID NO: | SEQ ID NO: 1548 | SEQ ID NO: 1549 |
| blocker | NO: | 1547 | EAPLRLGESPHDA | EVQLVESGGGLVKPGGSLRLSCAASGFTFSDSSMTWMRQAP |
| 1.63 | 1546 | YISSGGGVKFY | FDI | GKGLEWVSYISSGGGVKFYTDSVKGRFTISRDNAKNSLYLQMN |
| | DSSMT | TDSVKG | | SLRADDTAVYYCAREAPLRLGESPHDAFDISGQGTMVTVSS |
| PD1 | SEQ ID | SEQ ID NO: | SEQ ID NO: 1552 | SEQ ID NO: 1553 |
| blocker | NO: | 1551 | EAPLRLGESPHDA | EVQLVESGGGLVKPGGSLRLSCAASGFTFSDSSMTWMRQAP |
| 1.64 | 1550 | YISSGGGVKFY | FDI | GKGLEWVSYISSGGGVKFYADSVKGRFTISRDNAKNSLYLQM |
| | DSSMT | ADSVKG | | NSLRADDTAVYYCAREAPLRLGESPHDAFDISGQGTMVTVSS |
| PD1 | SEQ ID | SEQ ID NO: | SEQ ID NO: 1556 | SEQ ID NO: 1557 |
| blocker | NO: | 1555 | EAPLRLGESPHDA | QVQLVESGGGLVKPGGSLRLSCAASGFTFSDSSMTWMRQAP |
| 1.65 | 1554 | YISSGGGVKFY | FDI | GKGLEWVSYISSGGGVKFYADSVKGRFTISRDNAKNSLYLQM |
| | DSSMT | ADSVKG | | NSLRADDTAVYYCAREAPLRLGESPHDAFDISGQGTMVTVSS |
| PD1 | SEQ ID | SEQ ID NO: | SEQ ID NO: 1560 | SEQ ID NO: 1561 |
| blocker | NO: | 1559 | EAPLRLGESPHDA | QVQLVESGGGLVKPGGSLRLSCAASGFTFSDSSMTWMRQAP |
| 1.66 | 1558 | YISSGGGVKFY | FDT | GKGLEWVSYISSGGGVKFYTDSVKGRFTISRDNAKNSLYLQMD |
| | DSSMT | TDSVKG | | SLRADDTAVYYCAREAPLRLGESPHDAFDTSGQGTMVTVSS |
| PD1 | SEQ ID | SEQ ID NO: | SEQ ID NO: 1564 | SEQ ID NO: 1565 |
| blocker | NO: | 1563 | EAPLRLGESPHDA | QVQLVESGGGLVKPGGSLRLSCAASGFTFSGSSMTWMRQAP |
| 1.67 | 1562 | YISSGGGVIFY | FDI | GKGLEWVSYISSGGGVIFYADSVKGRFTISRDNAKNSLYLQMN |
| | GSSMT | ADSVKG | | SLRADDTAVYYCAREAPLRLGESPHDAFDISGQGTMVTVSS |
| PD1 | SEQ ID | SEQ ID NO: | SEQ ID NO: 1568 | SEQ ID NO: 1569 |
| blocker | NO: | 1567 | EAPLRLGESPHDA | QVQLVESGGGLVKPGGSLRLSCAASGFTFGDNSMTWMRQA |
| 1.68 | 1566 | YISSGGGVIFY | FDI | PGKGLEWVSYISSGGGVIFYADSVKGRFTISRDNAKNSLYLQM |
| | DNSMT | ADSVKG | | NSLRADDTAVYYCAREAPLRLGESPHDAFDISGRGTTVTVSS |
| PD1 | SEQ ID | SEQ ID NO: | SEQ ID NO: 1572 | SEQ ID NO: 1573 |
| blocker | NO: | 1571 | EAPLRLGESPHDA | QVQLVESGGGLVKPGGSLRLSCAASGFTFSDNSMSWMRQAP |
| 1.69 | 1570 | YISSGGGVIFY | FDI | GKGLEWVSYISSGGGVIFYADSVKGRFTISRDNAKNSLYLQMN |
| | DNSMS | ADSVKG | | SLRADDTAVYYCAREAPLRLGESPHDAFDISGQGTMVTVSS |
| PD1 | SEQ ID | SEQ ID NO: | SEQ ID NO: 1576 | SEQ ID NO: 1577 |
| blocker | NO: | 1575 | EAPLRLGESPHDA | QVQLVESGGGLVKPGGSLRLSCAASGFTFGDSSMTWMRQAP |
| 1.70 | 1574 | YISSGGAVKFY | FDI | GKGLEWVSYISSGGAVKFYTDSVKGRFTISRDNAKNSLYLQMN |
| | DSSMT | TDSVKG | | SLRADDTAVYYCAREAPLRLGESPHDAFDISGQGTMVTVSS |
| PD1 | SEQ ID | SEQ ID NO: | SEQ ID NO: 1580 | SEQ ID NO: 1581 |
| blocker | NO: | 1579 | EAPLRLGESPHDA | QVQLVESGGGLVKPGGSLRLSCAASGFTFGGSSMTWMRQAP |
| 1.71 | 1578 | YISSGGGVKFY | FDI | GKGLEWVSYISSGGGVKFYTDSVKGRFTISRDNAKNSLYLQMN |
| | GSSMT | TDSVKG | | SLRADDTAVYYCAREAPLRLGESPHDAFDISGQGTMVTVSS |
| PD1 | SEQ ID | SEQ ID NO: | SEQ ID NO: 1584 | SEQ ID NO: 1585 |
| blocker | NO: | 1583 | EAPLRLGESPHDA | QVQLVESGGGLVKPGGSLRLSCAASGFTFSDSSMSWMRQAP |
| 1.72 | 1582 | YISSGGGVIFY | FDI | GKGLEWVSYISSGGGVIFYADSVKGRFTISRDNAKNSLYLQMN |
| | DSSMS | ADSVKG | | SLRADDTAVYYCAREAPLRLGESPHDAFDISGQGTMVTVSS |
| PD1 | SEQ ID | SEQ ID NO: | SEQ ID NO: 1588 | SEQ ID NO: 1589 |
| blocker | NO: | 1587 | EAPLRLGESPHDA | QVQLVESGGGLVKPGGSLRLSCAATGFTFSDSSMTWMRQAP |
| 1.73 | 1586 | YISAGGGVRF | FDI | GKGLEWVSYISAGGGVRFYTDSVKGRFTISRDNAKNSLYLQM |
| | DSSMT | YTDSVKG | | NSLRADDTAVYYCAREAPLRLGESPHDAFDISGQGTMVTVSS |
| PD1 | SEQ ID | SEQ ID NO: | SEQ ID NO: 1592 | SEQ ID NO: 1593 |
| blocker | NO: | 1591 | EAPLRLGESPHDA | EVQLVESGGGLVKPGGSLRLSCAASGFTFSDSSMTWIRQAPG |
| 1.74 | 1590 | YISSGGGVKFY | FDI | KGLEWVSYISSGGGVKFYTDSVKGRFTISRDNAKNSLYLQMNS |
| | DSSMT | TDSVKG | | LRADDTAVYYCAREAPLRLGESPHDAFDISGQGTMVTVSS |
| PD1 | SEQ ID | SEQ ID NO: | SEQ ID NO: 1596 | SEQ ID NO: 1597 |
| blocker | NO: | 1595 | EAPLRLGESPHDA | EVQLVESGGGLVKPGGSLRLSCAASGFTFSDSSMTWIRQAPG |
| 1.75 | 1594 | YISSGGGVKFY | FDI | KGLEWVSYISSGGGVKFYTDSVKGRFTISRDNAKNSLYLQMNS |
| | DSSMT | TDSVKG | | LRAEDTAVYYCAREAPLRLGESPHDAFDISGQGTMVTVSS |
| PD1 | SEQ ID | SEQ ID NO: | SEQ ID NO: 1600 | SEQ ID NO: 1601 |
| blocker | NO: | 1599 | EAPLRLGESPHDA | EVQLVESGGGLVKPGGSLRLSCAASGFTFSDSSMTWIRQAPG |
| 1.76 | 1598 | YISSGGGVKFY | FDI | KGLEWVSYISSGGGVKFYADSVKGRFTISRDNAKNSLYLQMNS |
| | DSSMT | ADSVKG | | LRADDTAVYYCAREAPLRLGESPHDAFDISGQGTMVTVSS |
| PD1 | SEQ ID | SEQ ID NO: | SEQ ID NO: 1604 | SEQ ID NO: 1605 |
| blocker | NO: | 1603 | EAPLRLGESPHDA | EVQLVESGGGLVKPGGSLRLSCAASGFTFSDSSMTWMRQAP |
| 1.77 | 1602 | YISSGGGVKFY | FDI | GKGLEWVSYISSGGGVKFYTDSVKGRFTISRDNAKNSLYLQMN |
| | DSSMT | TDSVKG | | SLRAEDTAVYYCAREAPLRLGESPHDAFDISGQGTMVTVSS |
| PD1 | SEQ ID | SEQ ID NO: | SEQ ID NO: 1608 | SEQ ID NO: 1609 |
| blocker | NO: | 1607 | EAPLRLGESPHDA | EVQLVESGGGLVKPGGSLRLSCAASGFTFSDSSMTWIRQAPG |
| 1.78 | 1606 | YISSGGGVKFY | FDI | KGLEWVSYISSGGGVKFYADSVKGRFTISRDNAKNSLYLQMNS |
| | DSSMT | ADSVKG | | LRAEDTAVYYCAREAPLRLGESPHDAFDISGQGTMVTVSS |
| PD1 | SEQ ID | SEQ ID NO: | SEQ ID NO: 1612 | SEQ ID NO: 1613 |
| blocker | NO: | 1611 | EAPLRLGESPHDA | EVQLVESGGGLVKPGGSLRLSCAASGFTFSDSSMTWKRQAPG |
| 1.79 | 1610 | YISSGGGVKFY | FDI | KGLEWVSYISSGGGVKFYADSVKGRFTISRDNAKNSLYLQMNS |
| | DSSMT | ADSVKG | | LRAEDTAVYYCAREAPLRLGESPHDAFDISGQGTMVTVSS |
| PD1 | SEQ ID | SEQ ID NO: | SEQ ID NO: 1616 | SEQ ID NO: 1617 |
| blocker | NO: | 1615 | EAPLRLGESPHDA | EVQLVESGGGLVKPGGSLRLSCAASGFTFSDSSMTWMRQAP |
| 1.80 | 1614 | YISSGGGVKFY | FDI | GKGLEWVSYISSGGGVKFYADSVKGRFTISRDNAKNSLYLQM |
| | DSSMT | ADSVKG | | NSLRAEDTAVYYCAREAPLRLGESPHDAFDISGQGTMVTVSS |
| PD1 | SEQ ID | SEQ ID NO: | SEQ ID NO: 1620 | SEQ ID NO: 1621 |
| blocker | NO: | 1619 | EAPLRLGESPHDA | EVQLVESGGGLVKPGGSLRLSCAASGFTFSDSSMTWVRQAPG |
| 1.81 | 1618 | YISSGGGVKFY | FDI | KGLEWVSYISSGGGVKFYADSVKGRFTISRDNAKNSLYLQMNS |
| | DSSMT | ADSVKG | | LRAEDTAVYYCAREAPLRLGESPHDAFDISGQGTMVTVSS |
| PD1 | SEQ ID | SEQ ID NO: | SEQ ID NO: 1624 | SEQ ID NO: 1625 |
| blocker | NO: | 1623 | EAPLRLGESPHDA | EVQLVESGGGLVKPGGSLRLSCAASGFTFSDTSMTWMRQAP |
| 1.82 | 1622 | YISSGGGVKFY | FDI | GKGLEWVSYISSGGGVKFYTDSVKGRFTISRDNAKNSLYLQMN |
| | DTSMT | TDSVKG | | SLRAEDTAVYYCAREAPLRLGESPHDAFDISGQGTMVTVSS |
| PD1 | SEQ ID | SEQ ID NO: | SEQ ID NO: 1628 | SEQ ID NO: 1629 |
| blocker | NO: | 1627 | EAPLRLGESPHDA | EVQLVESGGGLVKPGGSLRLSCAASGFTFSDESMTWFRQAPG |
| 1.83 | 1626 | YISSGGGVKFY | FDI | KGLEWVSYISSGGGVKFYTDSVKGRFTISRDNAKNSLYLQMNS |
| | DESMT | TDSVKG | | LRAEDTAVYYCAREAPLRLGESPHDAFDISGQGTMVTVSS |
| PD1 | SEQ ID | SEQ ID NO: | SEQ ID NO: 1632 | SEQ ID NO: 1633 |
| blocker | NO: | 1631 | EAPLRLGESPHDA | EVQLVESGGGLVKPGGSLRLSCAASGFTFSDESMTWMRQAP |
| 1.84 | 1630 | YISSGGGVKFY | FDI | GKGLEWVSYISSGGGVKFYTDSVKGRFTISRDNAKNSLYLQMN |
| | DESMT | TDSVKG | | SLRAEDTAVYYCAREAPLRLGESPHDAFDISGQGTMVTVSS |
| PD1 | SEQ ID | SEQ ID NO: | SEQ ID NO: 1636 | SEQ ID NO: 1637 |
| blocker | NO: | 1635 | EAPLRLGESPHDA | EVQLVESGGGLVKPGGSLRLSCAASGFTFSDYSMTWMRQAP |
| 1.85 | 1634 | YISSGGGVKFY | FDI | GKGLEWVSYISSGGGVKFYTDSVKGRFTISRDNAKNSLYLQMN |
| | DYSMT | TDSVKG | | SLRAEDTAVYYCAREAPLRLGESPHDAFDISGQGTMVTVSS |
| PD1 | SEQ ID | SEQ ID NO: | SEQ ID NO: 1640 | SEQ ID NO: 1641 |
| blocker | NO: | 1639 | EAPLRLGESPHDA | EVQLVESGGGLVKPGGSLRLSCAASGFTFSDASMTWMRQAP |
| 1.86 | 1638 | YISSGGGVKFY | FDI | GKGLEWVSYISSGGGVKFYTDSVKGRFTISRDNAKNSLYLQMN |
| | DASMT | TDSVKG | | SLRAEDTAVYYCAREAPLRLGESPHDAFDISGQGTMVTVSS |
| PD1 | SEQ ID | SEQ ID NO: | SEQ ID NO: 1644 | SEQ ID NO: 1645 |
| blocker | NO: | 1643 | EAPLRLGESPHDA | EVQLVESGGGLVKPGGSLRLSCAASGFTFSDKSMTWMRQAP |
| 1.87 | 1642 | YISSGGGVKFY | FDI | GKGLEWVSYISSGGGVKFYTDSVKGRFTISRDNAKNSLYLQMN |
| | DKSMT | TDSVKG | | SLRAEDTAVYYCAREAPLRLGESPHDAFDISGQGTMVTVSS |
| PD1 | SEQ ID | SEQ ID NO: | SEQ ID NO: 1648 | SEQ ID NO: 1649 |
| blocker | NO: | 1647 | EAPLRLGESPHDA | EVQLVESGGGLVKPGGSLRLSCAASGFTFSDRSMTWMRQAP |
| 1.88 | 1646 | YISSGGGVKFY | FDI | GKGLEWVSYISSGGGVKFYTDSVKGRFTISRDNAKNSLYLQMN |
| | DRSMT | TDSVKG | | SLRAEDTAVYYCAREAPLRLGESPHDAFDISGQGTMVTVSS |
| PD1 | SEQ ID | SEQ ID NO: | SEQ ID NO: 1652 | SEQ ID NO: 1653 |
| blocker | NO: | 1651 | EAPLRLGESPHDA | EVQLVESGGGLVKPGGSLRLSCAASGFTFSDYSMTWMRQAP |
| 1.89 | 1650 | YISSGGGVKFY | FDI | GKGLEWVSYISSGGGVKFYADSVKGRFTISRDNAKNSLYLQM |
| | DYSMT | ADSVKG | | NSLRAEDTAVYYCAREAPLRLGESPHDAFDISGQGTMVTVSS |
| PD1 | SEQ ID | SEQ ID NO: | SEQ ID NO: 1656 | SEQ ID NO: 1657 |
| blocker | NO: | 1655 | EAPLRLGESPHDA | EVQLVESGGGLVKPGGSLRLSCAASGFTFSDVSMTWMRQAP |
| 1.90 | 1654 | YISSGGGVKFY | FDI | GKGLEWVSYISSGGGVKFYADSVKGRFTISRDNAKNSLYLQM |
| | DVSMT | ADSVKG | | NSLRAEDTAVYYCAREAPLRLGESPHDAFDISGQGTMVTVSS |
| PD1 | SEQ ID | SEQ ID NO: | SEQ ID NO: 1660 | SEQ ID NO: 1661 |
| blocker | NO: | 1659 | EAPLRLGESPHDA | EVQLVESGGGLVKPGGSLRLSCAASGFTFSDQSMTWMRQAP |
| 1.91 | 1658 | YISSGGGVKFY | FDI | GKGLEWVSYISSGGGVKFYADSVKGRFTISRDNAKNSLYLQM |
| | DQSMT | ADSVKG | | NSLRAEDTAVYYCAREAPLRLGESPHDAFDISGQGTMVTVSS |
| PD1 | SEQ ID | SEQ ID NO: | SEQ ID NO: 1664 | SEQ ID NO: 1665 |
| blocker | NO: | 1663 | EAPLRLGESPHDA | EVQLVESGGGLVKPGGSLRLSCAASGFTFSDESMTWMRQAP |
| 1.92 | 1662 | YISSGGGVKFY | FDI | GKGLEWVSYISSGGGVKFYADSVKGRFTISRDNAKNSLYLQM |
| | DESMT | ADSVKG | | NSLRAEDTAVYYCAREAPLRLGESPHDAFDISGQGTMVTVSS |
| PD1 | SEQ ID | SEQ ID NO: | SEQ ID NO: 1668 | SEQ ID NO: 1669 |
| blocker | NO: | 1667 | EAPLRLGESPHDA | EVQLVESGGGLVKPGGSLRLSCAASGFTFSDASMTWMRQAP |
| 1.93 | 1666 | YISSGGGVKFY | FDI | GKGLEWVSYISSGGGVKFYADSVKGRFTISRDNAKNSLYLQM |
| | DASMT | ADSVKG | | NSLRAEDTAVYYCAREAPLRLGESPHDAFDISGQGTMVTVSS |
| PD1 | SEQ ID | SEQ ID NO: | SEQ ID NO: 1672 | SEQ ID NO: 1673 |
| blocker | NO: | 1671 | EAPLRLGESPHDA | EVQLVESGGGLVKPGGSLRLSCAASGFTFSDWSMTWMRQAP |
| 1.94 | 1670 | YISSGGGVKFY | FDI | GKGLEWVSYISSGGGVKFYADSVKGRFTISRDNAKNSLYLQM |
| | DWSMT | ADSVKG | | NSLRAEDTAVYYCAREAPLRLGESPHDAFDISGQGTMVTVSS |
| PD1 | SEQ ID | SEQ ID NO: | SEQ ID NO: 1676 | SEQ ID NO: 1677 |
| blocker | NO: | 1675 | EAPLRLGESPHDA | EVQLVESGGGLVKPGGSLRLSCAASGFTFSDGSMTWMRQAP |
| 1.95 | 1674 | YISSGGGVKFY | FDI | GKGLEWVSYISSGGGVKFYADSVKGRFTISRDNAKNSLYLQM |
| | DGSMT | ADSVKG | | NSLRAEDTAVYYCAREAPLRLGESPHDAFDISGQGTMVTVSS |
| PD1 | SEQ ID | SEQ ID NO: | SEQ ID NO: 1680 | SEQ ID NO: 1681 |
| blocker | NO: | 1679 | EAPLRLGESPHDA | EVQLVESGGGLVKPGGSLRLSCAASGFTFSDTSMTWMRQAP |
| 1.96 | 1678 | YISSGGGVKFY | FDI | GKGLEWVSYISSGGGVKFYADSVKGRFTISRDNAKNSLYLQM |
| | DTSMT | ADSVKG | | NSLRAEDTAVYYCAREAPLRLGESPHDAFDISGQGTMVTVSS |
| PD1 | SEQ ID | SEQ ID NO: | SEQ ID NO: 1684 | SEQ ID NO: 1685 |
| blocker | NO: | 1683 | EAPLRLGESPHDA | EVQLVESGGGLVKPGGSLRLSCAASGFTFSDISMTWMRQAPG |
| 1.97 | 1682 | YISSGGGVKFY | FDI | KGLEWVSYISSGGGVKFYADSVKGRFTISRDNAKNSLYLQMNS |
| | DISMT | ADSVKG | | LRAEDTAVYYCAREAPLRLGESPHDAFDISGQGTMVTVSS |
| PD1 | SEQ ID | SEQ ID NO: | SEQ ID NO: 1688 | SEQ ID NO: 1689 |
| blocker | NO: | 1687 | EAPLRLGESPHDA | EVQLVESGGGLVKPGGSLRLSCAASGFTFSDKSMTWMRQAP |
| | 1686 | YISSGGGVKFY | FDI | GKGLEWVSYISSGGGVKFYADSVKGRFTISRDNAKNSLYLQM |
| | DKSMT | ADSVKG | | NSLRAEDTAVYYCAREAPLRLGESPHDAFDISGQGTMVTVSS |
| PD1 | SEQ ID | SEQ ID NO: | SEQ ID NO: 1692 | SEQ ID NO: 1693 |
| blocker | NO: | 1691 | EAPLRLGESPHDA | EVQLVESGGGLVKPGGSLRLSCAASGFTFSDRSMTWMRQAP |
| 1.99 | 1690 | YISSGGGVKFY | FDI | GKGLEWVSYISSGGGVKFYADSVKGRFTISRDNAKNSLYLQM |
| | DRSMT | ADSVKG | | NSLRAEDTAVYYCAREAPLRLGESPHDAFDISGQGTMVTVSS |
| PD1 | SEQ ID | SEQ ID NO: | SEQ ID NO: 1696 | SEQ ID NO: 1697 |
| blocker | NO: | 1695 | EAPLRLGESPHDA | EVQLVESGGGLVKPGGSLRLSCAASGFTFSDLSMTWMRQAP |
| 1.100 | 1694 | YISSGGGVKFY | FDI | GKGLEWVSYISSGGGVKFYADSVKGRFTISRDNAKNSLYLQM |
| | DLSMT | ADSVKG | | NSLRAEDTAVYYCAREAPLRLGESPHDAFDISGQGTMVTVSS |
| PD1 | SEQ ID | SEQ ID NO: | SEQ ID NO: 1700 | SEQ ID NO: 1701 |
| blocker | NO: | 1699 | EAPLRLGESPHDA | EVQLVESGGGLVKPGGSLRLSCAASGFTFSDFSMTWMRQAP |
| 1.101 | 1698 | YISSGGGVKFY | FDI | GKGLEWVSYISSGGGVKFYADSVKGRFTISRDNAKNSLYLQM |
| | DFSMT | ADSVKG | | NSLRAEDTAVYYCAREAPLRLGESPHDAFDISGQGTMVTVSS |
| PD1 | SEQ ID | SEQ ID NO: | SEQ ID NO: 1704 | SEQ ID NO: 1705 |
| blocker | NO: | 1703 | EAPLRLGESPHDA | EVQLVESGGGLVKPGGSLRLSCAASGFTFSDESVTWMRQAPG |
| 1.102 | 1702 | YISSGGGVKFY | FDI | KGLEWVSYISSGGGVKFYTDSVKGRFTISRDNAKNSLYLQMNS |
| | DESVT | TDSVKG | | LRAEDTAVYYCAREAPLRLGESPHDAFDISGQGTMVTVSS |
| PD1 | SEQ ID | SEQ ID NO: | SEQ ID NO: 1708 | SEQ ID NO: 1709 |
| blocker | NO: | 1707 | EAPLRLGESPHDA | EVQLVESGGGLVKPGGSLRLSCAASGFTFSDESQTWMRQAPG |
| 1.103 | 1706 | YISSGGGVKFY | FDI | KGLEWVSYISSGGGVKFYTDSVKGRFTISRDNAKNSLYLQMNS |
| | DESQT | TDSVKG | | LRAEDTAVYYCAREAPLRLGESPHDAFDISGQGTMVTVSS |
| PD1 | SEQ ID | SEQ ID NO: | SEQ ID NO: 1712 | SEQ ID NO: 1713 |
| blocker | NO: | 1711 | EAPLRLGESPHDA | EVQLVESGGGLVKPGGSLRLSCAASGFTFSDESFTWMRQAPG |
| 1.104 | 1710 | YISSGGGVKFY | FDI | KGLEWVSYISSGGGVKFYTDSVKGRFTISRDNAKNSLYLQMNS |
| | DESFT | TDSVKG | | LRAEDTAVYYCAREAPLRLGESPHDAFDISGQGTMVTVSS |
| PD1 | SEQ ID | SEQ ID NO: | SEQ ID NO: 1716 | SEQ ID NO: 1717 |
| blocker | NO: | 1715 | EAPLRLGESPHDA | EVQLVESGGGLVKPGGSLRLSCAASGFTFSDESLTWMRQAPG |
| 1.105 | 1714 | YISSGGGVKFY | FDI | KGLEWVSYISSGGGVKFYTDSVKGRFTISRDNAKNSLYLQMNS |
| | DESLT | TDSVKG | | LRAEDTAVYYCAREAPLRLGESPHDAFDISGQGTMVTVSS |
| PD1 | SEQ ID | SEQ ID NO: | SEQ ID NO: 1720 | SEQ ID NO: 1721 |
| blocker | NO: | 1719 | EAPLRLGESPHDA | EVQLVESGGGLVKPGGSLRLSCAASGFTFSDESKTWMRQAPG |
| 1.106 | 1718 | YISSGGGVKFY | FDI | KGLEWVSYISSGGGVKFYTDSVKGRFTISRDNAKNSLYLQMNS |
| | DESKT | TDSVKG | | LRAEDTAVYYCAREAPLRLGESPHDAFDISGQGTMVTVSS |
| PD1 | SEQ ID | SEQ ID NO: | SEQ ID NO: 1724 | SEQ ID NO: 1725 |
| blocker | NO: | 1723 | EAPLRLGESPHDA | EVQLVESGGGLVKPGGSLRLSCAASGFTFSDESYTWMRQAPG |
| 1.107 | 1722 | YISSGGGVKFY | FDI | KGLEWVSYISSGGGVKFYTDSVKGRFTISRDNAKNSLYLQMNS |
| | DESYT | TDSVKG | | LRAEDTAVYYCAREAPLRLGESPHDAFDISGQGTMVTVSS |
| PD1 | SEQ ID | SEQ ID NO: | SEQ ID NO: 1728 | SEQ ID NO: 1729 |
| blocker | NO: | 1727 | EAPLRLGESPHDA | EVQLVESGGGLVKPGGSLRLSCAASGFTFSDESATWMRQAPG |
| 1.108 | 1726 | YISSGGGVKFY | FDI | KGLEWVSYISSGGGVKFYADSVKGRFTISRDNAKNSLYLQMNS |
| | DESAT | ADSVKG | | LRAEDTAVYYCAREAPLRLGESPHDAFDISGQGTMVTVSS |
| PD1 | SEQ ID | SEQ ID NO: | SEQ ID NO: 1732 | SEQ ID NO: 1733 |
| blocker | NO: | 1731 | EAPLRLGESPHDA | EVQLVESGGGLVKPGGSLRLSCAASGFTFSDESFTWMRQAPG |
| 1.109 | 1730 | YISSGGGVKFY | FDI | KGLEWVSYISSGGGVKFYADSVKGRFTISRDNAKNSLYLQMNS |
| | DESFT | ADSVKG | | LRAEDTAVYYCAREAPLRLGESPHDAFDISGQGTMVTVSS |
| PD1 | SEQ ID | SEQ ID NO: | SEQ ID NO: 1736 | SEQ ID NO: 1737 |
| blocker | NO: | 1735 | EAPLRLGESPHDA | EVQLVESGGGLVKPGGSLRLSCAASGFTFSDESNTWMRQAPG |
| 1.110 | 1734 | YISSGGGVKFY | FDI | KGLEWVSYISSGGGVKFYADSVKGRFTISRDNAKNSLYLQMNS |
| | DESNT | ADSVKG | | LRAEDTAVYYCAREAPLRLGESPHDAFDISGQGTMVTVSS |
| PD1 | SEQ ID | SEQ ID NO: | SEQ ID NO: 1740 | SEQ ID NO: 1741 |
| blocker | NO: | 1739 | EAPLRLGESPHDA | EVQLVESGGGLVKPGGSLRLSCAASGFTFSDESWTWMRQAP |
| 1.111 | 1738 | YISSGGGVKFY | FDI | GKGLEWVSYISSGGGVKFYADSVKGRFTISRDNAKNSLYLQM |
| | DESWT | ADSVKG | | NSLRAEDTAVYYCAREAPLRLGESPHDAFDISGQGTMVTVSS |
| PD1 | SEQ ID | SEQ ID NO: | SEQ ID NO: 1744 | SEQ ID NO: 1745 |
| blocker | NO: | 1743 | EAPLRLGESPHDA | EVQLVESGGGLVKPGGSLRLSCAASGFTFSDESITWMRQAPG |
| 1.112 | 1742 | YISSGGGVKFY | FDI | KGLEWVSYISSGGGVKFYADSVKGRFTISRDNAKNSLYLQMNS |
| | DESIT | ADSVKG | | LRAEDTAVYYCAREAPLRLGESPHDAFDISGQGTMVTVSS |
| PD1 | SEQ ID | SEQ ID NO: | SEQ ID NO: 1748 | SEQ ID NO: 1749 |
| blocker | NO: | 1747 | EAPLRLGESPHDA | EVQLVESGGGLVKPGGSLRLSCAASGFTFSDESSTWMRQAPG |
| 1.113 | 1746 | YISSGGGVKFY | FDI | KGLEWVSYISSGGGVKFYADSVKGRFTISRDNAKNSLYLQMNS |
| | DESST | ADSVKG | | LRAEDTAVYYCAREAPLRLGESPHDAFDISGQGTMVTVSS |
| PD1 | SEQ ID | SEQ ID NO: | SEQ ID NO: 1752 | SEQ ID NO: 1753 |
| blocker | NO: | 1751 | EAPLRLGESPHDA | EVQLVESGGGLVKPGGSLRLSCAASGFTFSDESHTWMRQAPG |
| 1.114 | 1750 | YISSGGGVKFY | FDI | KGLEWVSYISSGGGVKFYADSVKGRFTISRDNAKNSLYLQMNS |
| | DESHT | ADSVKG | | LRAEDTAVYYCAREAPLRLGESPHDAFDISGQGTMVTVSS |
| PD1 | SEQ ID | SEQ ID NO: | SEQ ID NO: 1756 | SEQ ID NO: 1757 |
| blocker | NO: | 1755 | EAPLRLGESPHDA | EVQLVESGGGLVKPGGSLRLSCAASGFTFSDESGTWMRQAPG |
| 1.115 | 1754 | YISSGGGVKFY | FDI | KGLEWVSYISSGGGVKFYADSVKGRFTISRDNAKNSLYLQMNS |
| | DESGT | ADSVKG | | LRAEDTAVYYCAREAPLRLGESPHDAFDISGQGTMVTVSS |

An exemplary nucleic acid is SEQ ID NO: 1880.

In one embodiment, the variant V_{H} single domain antibody is selected from any one of the full length sequences shown in the table above with or without one or more amino acid substitutions, for example 1 to 20 for example 1, 2, 3, 4, 5, 6, 7, 8, 9 or 10 amino acid substitutions compared to these sequences. In the below, the numbering refers to sequences for clones in table 11. In one embodiment, the variant V_{H} single domain antibody is selected from Blocker 1.1. In one embodiment, the variant V_{H} single domain antibody is selected from Blocker 1.57,1.64, or 1.92. In one embodiment, the variant V_{H} single domain antibody comprises the sequence as shown for Blocker 1.1 but with amino acid substitutions at one or more the following positions: Y32, T33, T53, T56, I57, K58, Y59, T61 and/or W115.

In one embodiment, the V_{H} single domain antibody comprises the sequence as shown for Blocker 1.1, but with the following amino acid substitutions: Y32→N, T33→S, T53→S, T56→G, I57→V, K58→I, Y59→F, T61→A, W115→S (Humabody® 1.57)

In one embodiment, the variant V_{H} single domain antibody comprises the sequence as shown for Blocker 1.57) but with amino acid substitutions at one or more or all of the following positions: D31, N32, I58, A61, T35, S30, G56, S25Q117, M120 and/or Q1.

In one embodiment, the variant V_{H} single domain antibody comprises the sequence as shown for Blocker 1.57) but with amino acid substitutions selected from one of the following
1) D31→G, N32→S (Blocker 1.67);
2) S30→G, D31→G, N32→S, I58→K, A61→T (Blocker 1.71);
3) T35→S (1.69);
4) S30→G, N32→S, G56→A, I58→K, A61→T (Blocker 1.70);
5) S25→T, N32→S, S53→A, I58→R, A61→T (Blocker 1.73);
6) N32→S, I58→K, A61→T, N84→D, I114→T (Blocker 1.66);
7) S30→G, Q117→R, M120→T (Blocker 1.68);
8) N32→S, T35→S (Blocker 1.72);
9) N32→S, G56→A, I58→K, A61→T (Blocker 1.62);
10) N32→S, I58→K (Blocker 1.65);
11) Q1→E, N32→S, I58→K (Blocker 1.64);
12) Q1→E, N32→S, I58→K, A61→T (Blocker 1.63).

In one embodiment, the variant V_{H} single domain antibody comprises the sequence as shown for Blocker 1.64) but with amino acid substitutions at one or more or all of the following positions: S32, D90 and/or A61.

In one embodiment, the variant V_{H} single domain antibody comprises the sequence as shown for Blocker 1.64) but with amino acid substitutions selected from one of the following

### 1. S32→E, D90→E (Blocker 1.92)

### 2. S32→R, D90→E (Blocker 1.99)

### 3. S32→Y, D90→E (Blocker 1.89)

### 4. S32→E, D90→E, A61→T (Blocker 1.84)

### 5. D85→E A60→T (Blocker 1.77)

### 6. S32→T, D90→E, A60→T (Blocker 1.82)

The numbering used above is based on the actual position of the residue.

Exemplary Binding Molecules that Bind Both CD137 and PD-1

Any combination of the aforesaid single variable heavy chain domain antibodies that bind to CD137 and PD-1 respectively can be used in a binding agent for dual engagement of CD137 and PD-1 expressing cells. Thus, in one embodiment, any of the single variable heavy chain domain antibodies disclosed above, for example as listed in any of Tables 2, 3, 4 or 7 can be combined in a fusion protein with any of the single variable heavy chain domain antibodies as listed in any of Tables 8, 9, 10 or 11.

In one embodiment, a single variable heavy chain domain antibody having a set of CDR1, CDR2, CDR3 or a full length sequence as shown Table 2 or a sequence with at least 75%, 80%, 90% or 95% sequence homology thereto can be combined in a fusion protein with a single variable heavy chain domain antibody as shown in Table 8 having a set of CDR1, CDR2, CDR3 or a full length sequence or a sequence with at least 75%, 80%, 90% or 95% sequence homology thereto.

In one embodiment, a single variable heavy chain domain antibody having a set of CDR1, CDR2, CDR3 or a full length sequence as shown Table 2, 3, 4 or 7 or a sequence with at least 75%, 80%, 90% or 95% sequence homology thereto can be combined in a fusion protein with a single variable heavy chain domain antibody as shown in Table 8, 9 or 10 having a set of CDR1, CDR2, CDR3 or a full length sequence or a sequence with at least 75%, 80%, 90% or 95% sequence homology thereto.

In one embodiment, a single variable heavy chain domain antibody having a set of CDR1, CDR2, CDR3 or a full length sequence as shown Table 2, 3, 4 or 7 or a sequence with at least 75%, 80%, 90% or 95% sequence homology thereto can be combined in a fusion protein with a single variable heavy chain domain antibody as shown in Table 11 having a set of CDR1, CDR2, CDR3 or a full length sequence or a sequence with at least 75%, 80%, 90% or 95% sequence homology thereto.

In one embodiment, the multispecific molecule that binds both CD137 and PD-1 may comprise or consist of PDA and CD137A, CD137B, CD137C or CD137D. For example, the molecule comprises or consists of PDA and CD137A or CD137D. PDA may be located C or N terminal of the CD137 binder. PDA and CD137A, CD137B, CD137C or CD137D respectively are linked using a linker, e.g. a peptide linker, e.g. a (G4S)n linker wherein n is 1, 2, 3, 4, 5, or 6. In one embodiment, the linker is (G4S)n=6.

In another embodiment, the multispecific molecule may comprise or consist of PDA2 and CD137A, CD137B, CD137C or CD137D. For example, the construct comprises PDA2 and CD137A or CD137D. PDA2 may be located C or N terminal of the CD137 binder. PDA2 and CD137A, CD137B, CD137C or CD137D respectively are linked using a linker, e.g. a peptide linker, e.g. a (G4S)n linker wherein n is 1, 2, 3, 4, 5, or 6.

In one embodiment, the linker is (G4S)n=6. In another embodiment, the multispecific molecule may comprise or consist of PD138G7(G109D) and CD137A, CD137B, CD137C or CD137D. For example, the construct comprises PD138G7(G109D) and CD137A or CD137D. PD138G7(G109D) may be located C or N terminal of the CD137 binder. PD138G7(G109D) and CD137A, CD137B, CD137C or CD137D respectively are linked using a linker, e.g. a peptide linker, e.g. a (G4S)n linker wherein n is 1, 2, 3, 4, 5, or 6. In one embodiment, the linker is (G4S)n=6. In these constructs, an HSA binder. E.g. HSA1, HSA2 or HSA3 as disclosed herein may be included. This can be located at the N terminus, at the C terminus or in the centre; i.e. between the CD137 and PD-1 binder. In one embodiment, the multispecific molecule has SEQ ID NO: 1862.

In another embodiment, the multispecific molecule may comprise or consist of PDBlocker 1.92 as and CD137A, CD137B, CD137C or CD137D. For example, the construct comprises PDBlocker 1.92 and CD137A or CD137D. PD138G7(G109D) may be located C or N terminal of the CD137 binder. PDBlocker 1.92 and CD137A, CD137B, CD137C or CD137D respectively are linked using a linker, e.g. a peptide linker, e.g. a (G4S)n linker wherein n is 1, 2, 3, 4, 5, or 6. In one embodiment, the linker is (G4S)n=6.

In these constructs above, an HSA binder. e.g. HSA1, HSA2 or HSA3 as described herein or variants thereof may be included. This can be located at the N terminus, at the C terminus or in the centre using a linker, e.g. a peptide linker, e.g. a (G4S)n linker wherein n is 1, 2, 3, 4, 5, or 6. In one embodiment, the linker is (G4S)n=6.

For example, provided are multispecific protein or nucleic acid molecules that bind PD-1 and CD37 having a sequence as specified in table 12 or a sequence with at least 80%, 90%, 95% sequence homology thereto. For example, the sequence may have up to 5, 10, 15 or 20 amino acid substitutions, for example outside the CDR regions. These include single domain antibodies that bind to HSA. Also included in the invention are molecules that comprise the PD-1 and CD137 binders but without the HSA binding moiety as shown below. Some molecules shown below include a His tag linked to the C terminal VTVSS of the molecule. Also provided are the molecules as shown below but without the AAA linked His tag. Further envisaged are such molecules with a C terminal extension as described herein. In one embodiment, the molecule is selected from TPP-969, TPP-972, TPP-973, TPP-1010, TPP-1027, TPP1246 and TPP-1028 as shown below, but without the AAA linked His tag being present at the C terminus.

The two single domain antibodies can be linked with a peptide linker resulting in a fusion protein. The linker can be a (G4S)n linker; (G4S)n=6, as described herein. In one embodiment, the invention provides a fusion protein as shown in table 12 (with or without C terminal tag) or a fusion protein with 80%, 80%, 90% or 95% sequence homology thereto and nucleic acids encoding such fusion protein.

TABLE 12
TPP-942: PDA-6GS-172E1-13-6GS-HSA2-2-His; SEQ ID NO: 1758 Protein
TPP-942; PDA-6GS-172E1-13-6GS-HSA2-His; SEQ ID NO: 1759 Nucleotide
TPP-943: PDA-6GS-172E1-13-6GS-HSA2-His; SEQ ID NO: 1760 Protein
TPP-943: PDA-6GS-172E1-13-6GS-HSA3-His; SEQ ID NO: 1761 Nucleotide
TPP-950: PDA2-6GS-172E1-13-6GS-HSA2-His; SEQ ID NO: 1762 Protein
TPP-950: PDA2-6GS-172E1-13-6GS-HSA2-His; SEQ ID NO: 1763 Nucleotide
TPP-951: PDA2-6GS-172E1-13-6GS-HSA3-His; SEQ ID NO: 1764 Protein
TPP-951: PDA2-6GS-172E1-13-6GS-HSA3-His; SEQ ID NO: 1765 Nucleotide
TPP-957; PD138G7(G109D)-6GS-172E1-13-6GS-HSA2-His; SEQ ID NO: 1766 Protein
TPP-957: PD138G7(G109D)-6GS-172E1-13-6GS-HSA2-His; SEQ ID NO: 1767 Nucleotide
TPP-958: PD138G7(G109D)-6GS-172E1-13-6GS-HSA3-His; SEQ ID NO: 1768 Protein
TPP-958: PD138G7(G109D)-6GS-172E1-13-6GS-HSA3-His; SEQ ID NO: 1769 Nucleotide
TPP-969: PDA-6GS-HSA2-6GS-172E1-13-His; SEQ ID NO: 1770 Protein
TPP-969: PDA-6GS-HSA2-6GS-172E1-13-His; SEQ ID NO: 1771 Nucleotide
TPP-971: PDA2-6GS-HSA2-6GS-172E1-13-His; SEQ ID NO: 1772 Protein
TPP-971: PDA2-6GS-HSA2-6GS-172E1-13-His; SEQ ID NO: 1773 Nucleotide
TPP-972: CD137A-6GS-PDA-6GS-HSA2-His; SEQ ID NO: 1774 Protein
TPP-972: CD137A -6GS-PDA-6GS-HSA22-His; SEQ ID NO: 1775 Nucleotide
TPP-973: CD137A -6GS-PDA2-6GS-HSA2-His; SEQ ID NO: 1776 Protein
TPP-973: CD137A -6GS-PDA2-6GS-HSA2-His; SEQ ID NO: 1777 Nucleotide
TPP-975: CD137D-6GS-PDA2-6GS-HSA2-His; SEQ ID NO: 1778 Protein
TPP-975: CD137D -6GS-PDA2-6GS-HSA2-His; SEQ ID NO: 1779 Nucleotide
TPP-982: CD137A -6GS-HSA2-6GS-PDA-His. SEQ ID NO: 1780 Protein
TPP-982: CD137A -6GS-HSA2-6GS-PDA-His; SEQ ID NO: 1781 Nucleotide
TPP-983: CD137A -6GS-HSA2-6GS-PDA2-His; SEQ ID NO: 1782 Protein
TPP-983: CD137A -6GS-HSA2-6GS-PDA2-His; SEQ ID NO: 1783 Nucleotide
TPP-984: CD137D -6GS-HSA2-6GS-PDA-His; SEQ ID NO: 1784 Protein
TPP-984: CD137D -6GS-HSA2-2-6GS-PDA-His; SEQ ID NO: 1785 Nucleotide
TPP-985: CD137D -6GS-HSA2-2-6GS-PDA2-His; SEQ ID NO: 1786 Protein
TPP-985: CD137D -6GS-HSA2-6GS-PDA2-His; SEQ ID NO: 1787 Nucleotide
TPP-1008: PDA-6GS-HSA3-6GS- CD137D -His; SEQ ID NO: 1788 Protein
TPP-1008: PDA-6GS-HSA3-6GS- CD137D -His; SEQ ID NO: 1789 Nucleotide
TPP-1009: PDA-6GS-hsa1-6GS- CD137D -His; SEQ ID NO: 1790 Protein
TPP-1009: PDA-6GS-hsa1-6GS- CD137D -His; SEQ ID NO: 1791 Nucleotide
TPP-1010: PD138G7(G109D)-6GS-HSA2-6GS- CD137D -His; SEQ ID NO: 1792 Protein
TPP-1010: PD138G7(G109D)-6GS-HSA2-6GS- CD137D -His; SEQ ID NO: 1793 Nucleotide
SEQ ID NO: TPP-1011: PD138G7(G109D)-6GS-HSA3-6GS-CD137D-His; SEQ ID NO: 1794 Protein
TPP-1011: PD138G7(G109D)-6GS-HSA3-6GS- CD137D -His; SEQ ID NO: 1795 Nucleotide
TPP-1012PD138G7(G109D)-6GS-HSA1-6GS- CD137D -His; SEQ ID NO: 1796 Protein
TPP-1012PD138G7(G109D)-6GS-HSA1-6GS- CD137D -His; SEQ ID NO: 1797 Nucleotide
TPP-1014: CD137A -6GS-PDA-6GS-HSA-His; SEQ ID NO: 1798 Protein
TPP-1014: CD137A -6GS-PDA-6GS-HSA1-His; SEQ ID NO: 1799 Nucleotide
TPP-1015: CD137A -6GS-PDA2-6GS-HSA3-His, SEQ ID NO: 1800Protein
TPP-1015: CD137A -6GS-PDA2-6GS-HSA3-His. SEQ ID NO: 1801Nucleotide
TPP-1016: CD137A -6GS-PDA2-6GS-HSA1-His; SEQ ID NO: 1802 Protein
TPP-1016: CD137A -6GS-PDA2-6GS-HSA1-His; SEQ ID NO: 1803 Nucleotide
TPP-1017: CD137A -6GS-PD138G7(G109D)-6GS-HSA2-His; SEQ ID NO: 1804 Protein
TPP-1017: CD137A -6GS-PD138G7(G109D)-6GS-HSA2-His; SEQ ID NO: 1805 Nucleotide
TPP-1018: CD137A -6GS-PD138G7(G109D)-6GS-HSA3-His; SEQ ID NO: 1806 Protein
TPP-1018: CD137A -6GS-PD138G7(G109D)-6GS-HSA3-His; SEQ ID NO: 1807 Nucleotide
TPP-1023: CD137A -6GS-HSA3-6GS-PDA-His; SEQ ID NO: 1808 Protein
TPP-1023: CD137A -6GS-HSA3-6GS-PDA-His; SEQ ID NO: 1809 Nucleotide
TPP-1025: CD137A -6GS-HSA3-2-6GS-PDA2-His; SEQ ID NO: 1810 Protein
TPP-1025: CD137A -6GS-HSA3-6GS-PDA2-His; SEQ ID NO: 1811 Nucleotide
TPP-1027: CD137A -6GS-HSA2-6GS-PD138G7(G109D)-His; SEQ ID NO: 1812 Protein
TPP-1027: CD137A -6GS-HSA2-6GS-PD138G7(G109D)-His; SEQ ID NO: 1813 Nucleotide
TPP-1028: CD137A -6GS-HSA3-6GS-PD138G7(G109D)-His; SEQ ID NO: 1814 Protein
TPP-1028: CD137A -6GS-HSA3-6GS-PD138G7(G109D)-His; SEQ ID NO: 1815 Nucleotide
TPP-1052: PDA2-6GS-HSA3-6GS- CD137D- His; SEQ ID NO: 1816 Protein
TPP-1052: PDA2-6GS-HSA3-6GS- CD137D -His; SEQ ID NO: 1817 Nucleotide
TPP-1294 (PDBlocker1.92-6GS- CD137A -7-6GS-HSA1943) SEQ ID NO: 1818 Protein
TPP-1293 (PDBlocker1.92-6GS- CD137A -6GS-HSA2) SEQ ID NO: 1819 Nucleic acid
TPP-1439 PD138G7(G109D)-6GS-PD138G7(G109D)-6GS-CD137A-7-6GS-HSA2
SEQ ID NO: 1852 Protein
SEQ ID NO: 1882 Nucleic acid
TPP-1438 CD137A-6GS-HSA2-6GS-PD138G7(G109D)-6GS-PD138G7(G109D)
SEQ ID NO: 1853 Protein
SEQ ID NO: 1883 Nucleic acid
TPP-1411 PD138G7(G109D)-1GS-CD137A-1GS-HSA2
SEQ ID NO: 1854 Protein
SEQ ID NO: 1884 Nucleic acid
TPP-1410 PD138G7(G109D)-1GS-CD137A-6GS-HSA2
SEQ ID NO: 1855 Protein
SEQ ID NO: 1885 Nucleic acid
TPP-1409 CD137A-1GS-HSA2-1GS-PD138G7(G109D)
SEQ ID NO: 1856 Protein
SEQ ID NO: 1886 Nucleic acid
TPP-1408 PD138G7(G109D)-1GS-HSA2-1GS-CD137D
SEQ ID NO: 1857 Protein
SEQ ID NO: 1887 Nucleic acid
TPP-1294 PDBlocker1.92-6GS-CD137A-7-6GS-HSA3
SEQ ID NO: 1858 Protein
SEQ ID NO: 1888 Nucleic acid
TPP-1291 PD138G7(G109D)-6GS-CD137A-6GS-HSA1943
SEQ ID NO: 1859 Protein
SEQ ID NO: 1889 Nucleic acid
TPP-1290 PD138G7(G109D)-6GS-CD137A-6GS-HSA2
SEQ ID NO: 1860 Protein
SEQ ID NO: 1890 Nucleic acid
TPP-1247 CD137A-6GS-HSA3-6GS-PD138G7(G109D)
SEQ ID NO: 1861 Protein
SEQ ID NO: 1891 Nucleic acid
TPP-1246 CD137A-6GS-HSA2-6GS-PD138G7(G109D)
SEQ ID NO: 1862 Protein
SEQ ID NO: 1892 Nucleic acid
TPP-1245 CD137A-6GS-PDA2-6GS-HSA2
SEQ ID NO: 1863 Protein
SEQ ID NO: 1893 Nucleic acid
TPP-1242 PD138G7(G109D)-6GS-HSA2-6GS-CD137D
SEQ ID NO: 1864 Protein
SEQ ID NO: 1894 Nucleic acid
TPP-1241 PDA-6GS-HSA2-6GS-CD137D
SEQ ID NO: 1865 Protein
SEQ ID NO: 1895 Nucleic acid

TPP-1293, TPP-1294 both include PD-1 binders that block PD-1 function when used on its own (i.e. not in format with a CD13 binder). The other constructs in the table include PD-1 binders that do not block PD-1 binding to PD-L1.

### Exemplary Modifications of the Binding Molecule

In one embodiment, a binding agent described above comprises further binding molecules. Thus, the binding agent can, for example, be trispecific or tetraspecific.

In one embodiment, the binding molecule comprises a first V_{H} single domain antibody that binds to CD137 (V_{H} (A)) and a second moiety, for example a V_{H} single domain antibody, that binds to PD-1 (V_{H} (B)). It further comprises a third, fourth, fifth etc moiety, for example a V_{H} single domain antibody (i.e. V_{H} (C), V_{H} (D), V_{H} (E)) that binds to another antigen. Thus, in one embodiment, the binding molecule has the following formula (wherein V_{H} stands for a single domain antibody as defined herein, that is the single V_{H} domain that does not comprise other parts of a full antibody and retains binding to the antigen): V_{H} (A)-L-V_{H} (B)-L-V_{H} (X)n wherein X denotes a V_{H} binding to a target other than the target V_{H} (A) and V_{H} (B) bind to and wherein X is 1 to 10, for example, 1, 2, 3, 4, 5, 6, 7, 8, 9 or 10. L denotes a linker, for example a peptide linker. As explained elsewhere, a moiety that binds to PD-1 or another target can be selected from an antibody or fragment thereof or other polypeptide.

In another embodiment, the further moiety may serve to prolong the half-life of the binding molecule. The further moiety may comprise a protein, for example a peptide, antibody, or part thereof, such as a V_{H} or CDR, that binds a serum albumin, e.g., human serum albumin (HSA) or mouse serum albumin (MSA). In one embodiment, the further moiety may comprise a V H domain that binds serum albumin, e.g., human serum albumin (HSA) or mouse serum albumin (MSA). The further moiety may comprise a serum albumin, e.g. a HSA or a variant thereof such as HSA C34S. The HSA binder may a single domain antibody, for example a variable heavy chain single domain antibody. Exemplary antibodies are provided herein as HSA1, HSA2 and HSA3. Further provided is a binding molecule as described herein comprising a V_{H} domain and an Fc domain, e.g., wherein the V_{H} domain is fused to an Fc domain.

The term “half-life” as used herein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms. Half-life may be increased by at least 1.5 times, preferably at least 2 times, such as at least 5 times, for example at least 10 times or more than 20 times, greater than the half-life of the corresponding V_{H} single domain antibodies of the invention. For example, increased half-life may be more than 1 hours, preferably more than 2 hours, more preferably more than 6 hours, such as more than 12 hours, or even more than 24, 48 or 72 hours, compared to the corresponding V_{H} single domain antibodies or fusion protein of the invention. The in vivo half-life of an amino acid sequence, compound or polypeptide of the invention can be determined in any manner known per se, such as by pharmacokinetic analysis. Suitable techniques will be clear to the person skilled in the art. Half life can for example be expressed using parameters such as the t1/2-alpha t1/2-beta and the area under the curve (AUC).

In one embodiment, the binding agents are labelled with a detectable or functional label. A label can be any molecule that produces or can be induced to produce a signal, including but not limited to fluorophores, fluorescers, radiolabels, enzymes, chemiluminescers, a nuclear magnetic resonance active label or photosensitizers. Thus, the binding may be detected and/or measured by detecting fluorescence or luminescence, radioactivity, enzyme activity or light absorbance.

In still other embodiments binding agents are coupled to at least one therapeutic moiety, such as a drug, an enzyme or a toxin. In one embodiment, the therapeutic moiety is a toxin, for example a cytotoxic radionuclide, chemical toxin or protein toxin.

In another aspect, the binding agents of the invention are modified to increase half-life, for example by a chemical modification, especially by PEGylation, or by incorporation in a liposome or using a serum albumin protein. Increased half life can also be conferred by conjugating the molecule to a n antibody fragment, for example a V_{H} domain that increases half life.

To generate multivalent binding agents as described above, two binding molecules can be connected by a linker, for example a polypeptide linker. Suitable linkers include for example a linker with GS residues such as (Gly4Ser)n, where n=from 1 to 10 or more, e.g., 1, 2, 3, 4, 5, 6, 7, 8, 9 or 10 and specific sequences of examples are provided elsewhere.

### Exemplary Methods for Making Single Domain Antibody and Binding Molecules

A single domain antibody described herein for use in the multispecific molecule can be obtained from a transgenic rodent that expresses heavy chain only antibodies upon stimulation with a CD137 or PD-1 antigen respectively. The transgenic rodent, for example a mouse, preferably has a reduced capacity to express endogenous antibody genes. Thus, in one embodiment, the rodent has a reduced capacity to express endogenous light and/or heavy chain antibody genes. The rodent may therefore comprise modifications to disrupt expression of endogenous kappa and lambda light and/or heavy chain antibody genes so that no functional light and/or heavy chains are produced, for example as further explained below.

A method for producing a human heavy chain only antibody capable of binding the target antigen comprises
a) immunising a transgenic rodent, e.g. a mouse, with target antigen (i.e. PD-1 or CD137) wherein said rodent expresses a nucleic acid construct comprising unrearranged human heavy chain V genes and is not capable of making functional endogenous light or heavy chains,
b) isolating human heavy chain only antibodies.

Further steps can include isolating a V_{H} domain from said heavy chain only antibody, for example by generating a library of sequences comprising V_{H} domain sequences from said rodent, e.g. mouse, and isolating sequences comprising V_{H} domain sequences from said libraries.

A method for producing a single V_{H} domain antibody capable of binding to the target antigen comprises
a) immunising a transgenic rodent, e.g. a mouse with a target antigen wherein said rodent, e.g. mouse, expresses a nucleic acid construct comprising unrearranged human heavy chain V genes and is not capable of making functional endogenous light or heavy chains,
b) generating a library of sequences comprising V_{H} domain sequences from said rodent, e.g. mouse, and
c) isolating sequences comprising V_{H} domain sequences from said libraries.

Further steps may include identifying a single V_{H} domain antibody or heavy chain only antibody that binds to the target antigen, for example by using functional assays as shown in the examples.

Methods for preparing or generating the polypeptides, nucleic acids, host cells, products and compositions described herein using in vitro expression libraries can comprise the steps of:

a) providing a set, collection or library of nucleic acid sequences encoding amino acid sequences; and
b) screening said set, collection or library for amino acid sequences that can bind to/have affinity for the target antigen and
c) isolating the amino acid sequence(s) that can bind to/have affinity for target antigen.

In the above method, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art (see for example Phage Display of Peptides and Proteins: A Laboratory Manual, Academic Press; 1st edition (Oct. 28, 1996) Brian K. Kay, Jill Winter, John McCafferty).

Libraries, for example phage libraries, are generated by isolating a cell or tissue expressing an antigen-specific, heavy chain-only antibody, cloning the sequence encoding the V_{H} domain(s) from mRNA derived from the isolated cell or tissue and displaying the encoded protein using a library. The V_{H} domain(s) can be expressed in bacterial, yeast or other expression systems.

In the various aspects and embodiments as out herein, the term rodent may relate to a mouse or a rat. In one embodiment, the rodent is a mouse. The mouse may comprise a non-functional endogenous lambda light chain locus. Thus, the mouse does not make a functional endogenous lambda light chain. In one embodiment, the lambda light chain locus is deleted in part or completely or rendered non-functional through insertion, inversion, a recombination event, gene editing or gene silencing. For example, at least the constant region genes C1, C2 and C3 may be deleted or rendered non-functional through insertion or other modification as described above. In one embodiment, the locus is functionally silenced so that the mouse does not make a functional lambda light chain.

Furthermore, the mouse may comprise a non-functional endogenous kappa light chain locus. Thus, the mouse does not make a functional endogenous kappa light chain. In one embodiment, the kappa light chain locus is deleted in part or completely or rendered non-functional through insertion, inversion, a recombination event, gene editing or gene silencing. In one embodiment, the locus is functionally silenced so that the mouse does not make a functional kappa light chain.

The mouse having functionally-silenced endogenous lambda and kappa L-chain loci may, for example, be made as disclosed in WO 2003/000737, which is hereby incorporated by reference in its entirety.

Furthermore, the mouse may comprise a non-functional endogenous heavy chain locus. Thus, the mouse does not make a functional endogenous heavy chain. In one embodiment, the heavy chain locus is deleted in part or completely or rendered non-functional through insertion, inversion, a recombination event, gene editing or gene silencing. In one embodiment, the locus is functionally silenced so that the mouse does not make a functional heavy chain. This can be as described in WO 2004/076618 (hereby incorporated by reference in its entirety).

By deletion in part is meant that the endogenous locus gene sequence has been deleted or disrupted, for example by an insertion, to the extent that no functional endogenous gene product is encoded by the locus, i.e., that no functional product is expressed from the locus. In another embodiment, the locus is functionally silenced.

In one embodiment, the mouse comprises a non-functional endogenous heavy chain locus, a non-functional endogenous lambda light chain locus and a non-functional endogenous kappa light chain locus. The mouse therefore does not produce any functional endogenous light or heavy chains. Thus, the mouse is a triple knockout (TKO) mouse. The transgenic mouse may comprise a vector, for example a Yeast Artificial Chromosome (YAC) for expressing a heterologous, preferably a human, heavy chain locus. For example, the YAC may comprise a plethora of unrearranaged human V_{H}, D and J genes in combination with mouse immunoglobulin constant region genes lacking C_{H}1 domains, mouse enhancer and regulatory regions. The human V_{H}, D and J genes are human V_{H}, D and J loci and they are unrearranged genes that are fully human.

Alternative methods known in the art may be used for deletion or inactivation of endogenous mouse or rat immunoglobulin genes and introduction of human V_{H}, D and J genes in combination with mouse immunoglobulin constant region genes lacking C_{H}1 domains, mouse enhancer and regulatory regions.

Transgenic mice can be created according to standard techniques as illustrated in the examples. Triple knock-out mice into which transgenes have been introduced to express immunoglobulin loci are referred to herein as TKO/Tg. In one embodiment, the mouse is as described in WO2016/062990.

Fusion proteins as described herein can be generated by linking a nucleic acid encoding a single variable heavy chain domain antibody that binds to CD137 to a nucleic acid encoding a single variable heavy chain domain antibody that binds to PD-1, for example using a nucleic acid sequence that encodes a peptide linker. Such fusion nucleic acid molecules are then expressed in suitable host cells.

### Exemplary Therapeutic Applications of the Binding Molecules

In another aspect, there is provided a pharmaceutical composition comprising a binding molecule as described herein and optionally a pharmaceutically acceptable carrier. A binding molecule as described herein or the pharmaceutical composition of the invention can be administered by any convenient route, including but not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, by inhalation, or topical, particularly to the ears, nose, eyes, or skin or by inhalation.

Parenteral administration includes, for example, intravenous, intramuscular, intraarterial, intraperitoneal, intranasal, rectal, intravesical, intradermal, topical or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the single domain antibody of the present invention or compositions and pharmaceutically acceptable carriers are sterile. Water is a preferred carrier when the binding molecule of the present invention is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition of the invention can be in the form of a liquid, e.g., a solution, emulsion or suspension. The liquid can be useful for delivery by injection, infusion (e.g., IV infusion) or sub-cutaneously.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

The composition can be in the form of a liquid, e. g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

In specific embodiments, it can be desirable to administer the composition locally to the area in need of treatment, or by intravenous injection or infusion.

The amount of the therapeutic that is effective/active in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of a single domain antibody of the present invention by weight of the composition. When intended for oral administration, this amount can be varied to range from about 0.1% to about 80% by weight of the composition. Preferred oral compositions can comprise from about 4% to about 50% of the single domain antibody of the present invention by weight of the composition.

Preferred compositions of the present invention are prepared so that a parenteral dosage unit contains from about 0.01% to about 2% by weight of the single domain antibody of the present invention. The invention also relates to a device, such as a pre-filled syringe which comprises a binding molecule of the invention.

For administration by injection, the composition can comprise from about typically about 0.1 mg/kg to about 250 mg/kg of the subject's body weight, preferably, between about 0.1 mg/kg and about 20 mg/kg of the subject's body weight, and more preferably about 1 mg/kg to about 10 mg/kg of the subject's body weight. In one embodiment, the composition is administered at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., human patients, being treated. Many medical treatments are effective for some, but not all, patients that undergo the treatment.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

As used herein, the term “effective amount” means an amount of the binding molecule as described herein, that when administered alone or in combination with an additional therapeutic agent to a cell, tissue, or subject, is effective to achieve the desired therapeutic or prophylactic effect under the conditions of administration.

The invention furthermore relates to a method for the prevention and/or treatment of cancer, comprising administering a binding molecule of the invention to a subject, said method comprising administering, to a subject in need thereof, a pharmaceutically active amount of a binding molecule and/or of a pharmaceutical composition of the invention. In particular, the invention relates to a method for the prevention and/or treatment of cancer, said method comprising administering, to a subject in need thereof, a pharmaceutically active amount of a binding molecule or a pharmaceutical composition of the invention.

The invention also relates to a binding molecule of the invention for use in the treatment of a disease, such as cancer, an immune disorder, neurological disease, inflammatory disorder, allergy, transplant rejection, viral infection, (e.g. chronic viral infection) immune deficiency, and other immune system-related disorder. In one embodiment, the disease is cancer. The cancer can be selected from a solid or non-solid tumor. For example, the cancer may be selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, urothelial carcinoma leukemia, prostate cancer, mesothelioma, adrenocortical carcinoma, lymphomas, such as such as Hodgkin's disease, non-Hodgkin's, gastric cancer, and multiple myelomas.

In one embodiment, the tumor is a solid tumor. Examples of solid tumors which may be accordingly treated include breast carcinoma, lung carcinoma, colorectal carcinoma, pancreatic carcinoma, glioma and lymphoma. Some examples of such tumors include epidermoid tumors, squamous tumors, such as head and neck tumors, colorectal tumors, prostate tumors, breast tumors, lung tumors, including small cell and non-small cell lung tumors, pancreatic tumors, thyroid tumors, ovarian tumors, and liver tumors. Other examples include Kaposi's sarcoma, CNS, neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma. Examples of vascularized skin cancers for which the antagonists of this invention are effective include squamous cell carcinoma, basal cell carcinoma and skin cancers that can be treated by suppressing the growth of malignant keratinocytes, such as human malignant keratinocytes.

In one embodiment, the tumor is a non-solid tumor. Examples of non-solid tumors include leukemia, multiple myeloma and lymphoma.

In one aspect, the cancer is identified as a PD-L1 positive cancer. In one aspect, the cancer is locally advanced unresectable, metastatic, or recurrent cancer.

Preferred cancers whose growth may be inhibited using the antibodies of the invention include cancers typically responsive to immunotherapy. Non-limiting examples of preferred cancers for treatment include melanoma (e.g., metastatic malignant melanoma), renal cancer (e.g. clear cell carcinoma), prostate cancer (e.g. hormone refractory prostate adenocarcinoma), breast cancer, colon cancer and lung cancer (e.g. non-small cell lung cancer).

In one embodiment, the cancer has progressed after another treatment, for example chemotherapy.

The multivalent molecules which include a PD-1 binding moeity that blocks PD-1 signaling and pharmaceutical compositions described herein are particularly useful for the treatment of cancers that are associated with cells (e.g., exhausted T cells, B cells, monocytes, etc.) that express abnormally high levels of PD-1. Other preferred cancers include those characterized by elevated expression of PD-1 and/or its ligands PD-L1 and/or PD-L2. In one embodiment, the cancer is selected from a cancer that has high levels of cancer-associated genetic mutations and/or high levels of expression of tumour antigens. In another embodiment, the cancer is selected from a cancer known to be immunogenic or that is able to become immunogenic upon treatment with other cancer therapies.

In another aspect, the invention relates to the use of a binding molecule of the invention in the treatment of disease. In another aspect, the invention relates to the use of a binding molecule of the invention in the manufacture of a medicament for the treatment of cancer.

In one aspect, the cancer is locally advanced unresectable, metastatic, or recurrent cancer.

The binding molecule of the invention may be administered as the sole active ingredient or in combination with one or more other therapeutic and/or cytotoxic moiety. In one embodiment, the binding molecule may be conjugated to a toxic moiety.

In one embodiment, the single domain antibody is used in combination with surgery.

Exemplary Combinations with Other Agents

The molecules or pharmaceutical composition of the invention, including monovalent or multivalent molecules, may be administered as the sole active ingredient or in combination with one or more other therapeutic agent. A therapeutic agent is a compound or molecule which is useful in the treatment of a disease such as cancer, an immune disorder, neurological disease, inflammatory disorder, allergy, transplant rejection, viral infection, (e.g. chronic viral infection) immune deficiency, and other immune system-related disorder.

In one embodiment, the disease is cancer. Thus, we also provide a combination therapy using a multispecific molecule as described herein together with another PD-1 therapy. The PD-1 therapy may be selected from a PD-1 inhibitor, such as an antibody molecule, such as Pembrolizumab (Keytruda), Nivolumab (Opdivo) or Cemiplimab (Libtayo) or a PD-L1 inhibitor. Alternatively, the PD-1 therapy may be a CAR-T therapy.

Examples of therapeutic agents include antibodies, antibody fragments, drugs, toxins, nucleases, hormones, immunomodulators, pro-apoptotic agents, anti-angiogenic agents, boron compounds, photoactive agents or dyes and radioisotopes. An antibody molecule includes a full antibody or fragment thereof (e.g., a Fab, F(ab′)2, Fv, a single chain Fv fragment (scFv) or a single domain antibody, for example a V_{H} domain, antibody mimetic protein or a protein that mimics the natural ligand of CD137.

The anti-cancer therapy may include a therapeutic agent or radiation therapy and includes gene therapy, viral therapy, RNA therapy bone marrow transplantation, nanotherapy, targeted anti-cancer therapies or oncolytic drugs. Examples of other therapeutic agents include other checkpoint inhibitors, antineoplastic agents, immunogenic agents, attenuated cancerous cells, tumor antigens, antigen presenting cells such as dendritic cells pulsed with tumor-derived antigen or nucleic acids, immune stimulating cytokines (e.g., IL-2, IFNa2, GM-CSF), targeted small molecules and biological molecules (such as components of signal transduction pathways, e.g. modulators of tyrosine kinases and inhibitors of receptor tyrosine kinases, and agents that bind to tumor-specific antigens, including EGFR antagonists), an anti-inflammatory agent, a cytotoxic agent, a radiotoxic agent, or an immunosuppressive agent and cells transfected with a gene encoding an immune stimulating cytokine (e.g., GM-CSF), chemotherapy. In one embodiment, administration is in combination with surgery. The binding molecule of the invention may be administered at the same time or at a different time as the other therapy, e.g., simultaneously, separately or sequentially.

### Exemplary Methods for Modulating Immune Response, Inhibiting Tumor Growth Etc

In yet another aspect, there is provided a method of modulating an immune response in a subject comprising administering to the subject the binding molecule or pharmaceutical composition described herein such that the immune response in the subject is modulated. Preferably, the binding molecule enhances, stimulates or increases the immune response in the subject.

In a further aspect, there is provided a method of inhibiting growth of tumor cells or promoting tumor regression in a subject, comprising administering to a subject a therapeutically effective amount of a binding molecule or a pharmaceutical composition described herein.

In a further aspect, there is provided a method for activating the downstream signalling pathway of CD137 comprising administering to a subject a binding molecule or a pharmaceutical composition described herein.

In a further aspect, there is provided a method for inducing T lymphocyte activation and/or proliferation comprising administering to a subject a binding molecule or a pharmaceutical composition described herein.

In a further aspect, there is provided a method for dual targeting of a cell expressing both CD137 and PD-1, or for localising the CD137 activation in the region of cells expressing PD-1 or localising the PD-1 pathway blocking in the region of cells expressed CD137 comprising administering to a subject a binding molecule comprising a single variable heavy chain domain antibody that binds to CD137 or a pharmaceutical composition described herein.

In a further aspect, there is provided a binding molecule comprising a single variable heavy chain domain antibody that binds to CD137 or a pharmaceutical composition described herein for dual targeting of a CD137 expressing cell and PD-1 expressing cell. For example, the binding molecule comprises a single variable heavy chain domain antibody that binds to CD137 described herein and a single variable heavy chain domain antibody that binds to PD-1 as described herein.

In a further aspect, there is provided a method for down-modulating PD-1 on the cell surface comprising administering to a subject a binding molecule described herein, such as a binding molecule comprising a single variable heavy chain domain antibody that binds to CD137 and comprising a single variable heavy chain domain antibody that binds to PD-1 or a pharmaceutical composition described herein. In one embodiment, the single variable heavy chain domain antibody that binds to PD-1 is a single variable heavy chain domain antibody that does not block PD-1 function when used on its own, that is when not used in a multispecific binding molecule with CD137. This can be selected from one of the molecules as set out in Tables 8, 9 or 10 or a molecule having at least 70%, 80,% 90% or 95% sequence homology thereto.

In a further aspect, there is provided a method for blocking PD-1 function comprising administering to a subject a binding molecule described herein, such as a binding molecule comprising a single variable heavy chain domain antibody that binds to CD137 and comprising a single variable heavy chain domain antibody that binds to PD-1 or a pharmaceutical composition described herein. In one embodiment, the single variable heavy chain domain antibody that binds to PD-1 is a single variable heavy chain domain antibody that does not block PD-1 function when used on its own, that is when not used in a multispecific binding molecule with CD137. This can be selected from one of the molecules as set out in Tables 8, 9 or 10 or a molecule having at least 70%, 80,% 90% or 95% sequence homology thereto.

In a further aspect, there is provided a method for simultaneously activating downstream signalling pathways of CD137 and PD-1 and independently leading to CD137 agonism or PD-1 downstream signalling inhibition comprising administering to a subject a binding molecule described herein, such as a binding molecule comprising a single variable heavy chain domain antibody that binds to CD137 and comprising a single variable heavy chain domain antibody that binds to PD-1 or a pharmaceutical composition described herein. In one embodiment, the single variable heavy chain domain antibody that binds to PD-1 is a single variable heavy chain domain antibody that does not block PD-1 function when used on its own, that is when not used in a multispecific binding molecule with CD137. This can be selected from one of the molecules as set out in Tables 8, 9 or 10 or a molecule having at least 70%, 80,% 90% or 95% sequence homology thereto.

In a further aspect, there is provided binding molecule comprising a single variable heavy chain domain antibody that binds to CD137 and comprising a single variable heavy chain domain antibody that binds to PD-1, for example as described herein for use in simultaneously activating downstream signalling pathways of CD137 and PD-1 and independently leading to CD137 agonism or PD-1 downstream signalling inhibition; dual targeting of a cell expressing both CD137 and PD-1, or for localising the CD137 activation in the region of cells expressing PD-1 or localising the PD-1 down-modulation in the region of cells expressed CD137; dual targeting of a CD137 expressing cell and PD-1 expressing cell or down-modulating PD-1 on the cell surface. In one embodiment, the single variable heavy chain domain antibody that binds to PD-1 is a single variable heavy chain domain antibody that does not block PD-1 function when used on its own, that is when not used in a multispecific binding molecule with CD137. This can be selected from one of the molecules as set out in Tables 8, 9 or 10 or a molecule having at least 70%, 80,% 90% or 95% sequence homology thereto.

### Exemplary Immunoconjugates

In another aspect, there is provided an immunoconjugate comprising a binding molecule described herein conjugated to at least one therapeutic and/or diagnostic agent i.e. an imagining agent.

### Exemplary Kits

In another aspect, the invention provides a kit for detecting cancer for diagnosis, treatment, prognosis or monitoring comprising a binding molecule of the invention. The kit may also comprise instructions for use. The kits may include a labeled binding molecule of the invention as described above and one or more compounds for detecting the label. Also provided is a binding molecule of the invention packaged in lyophilized form, or packaged in an aqueous medium. The kits may include reagent, (e.g. for reconstituting) and/or instructions for use and/or a device for administration.

Also provided is a binding molecule or pharmaceutical composition described herein with reference to the figures and examples. Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present disclosure, including methods, as well as the best mode thereof, of making and using this disclosure, the following examples are provided to further enable those skilled in the art to practice this disclosure. However, those skilled in the art will appreciate that the specifics of these examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is now further described in the non-limiting examples.

### EXAMPLES

### Example 1. Construction of Tg/TKO Mice

Triple knock-out mice carrying a human heavy-chain antibody transgenic locus in germline configuration within a background that is silenced for endogenous heavy and light chain antibody expression were created and immunised as previously described (WO2004/076618, WO2003/000737, Ren et al., Genomics, 84, 686, 2004; Zou et al., J. Immunol., 170, 1354, 2003 and WO2016/062990, WO2018/127709, WO2018/127710).

### Example 2. Immunisation Protocol

For CD137 immunisation, Tg/TKO mice aged 8-12 weeks were immunised with a human CD137-human Fc chimeric protein. PD-1 binding Humabody® V_{H} molecules were generated as described in WO2018/127709 and WO2018/127710.

### Example 3. Serum ELISA

Serum was collected from mice before and after immunisation and checked by ELISA for the presence of serum human CD137 reactive heavy chain antibodies in response to immunisation with CD137 antigen.

### Example 4. Generation of Libraries from Immunised Mice

Generation of libraries from immunised mice described above followed standard protocols of library generation and has been described elsewhere, e.g. WO2018/127709 and WO2018/127710.

### Example 5. Selection Strategies: CD137 Binding V_{H} Isolation and Optimisation

Preparation of library phage stocks and phage display selections were performed according to published methods (Antibody Engineering, edited by Benny Lo, chapter 8, p161-176, 2004; WO2018/127709).

### Example 6. Sequencing

Phage selection outputs were subcloned into a soluble expression vector and plates of representative clones from each output were sequenced. Following sequencing analysis, unique individual V_{H} clones were identified and assigned to sequence families based on CDR3 homology. All unique V_{H} were purified and characterised as described below. Further clones were generated by sequence optimisation of clone Humabody® 003BB1172-E01 respectively to revert sequence to germline.

Table 7 shows the sequences of 003BB1172-E01 and sequence optimised variants.

### Example 7. Preparation of Purified V_{H}

Humabody® V_{H} were purified from the supernatants of E. coli (with pJ401), Pichia pastoris (with pJ912, pD912 or pPZ-alpha) and Expi HEK293 (with pTT5 vector) by using the native protein properties or a C-terminal 6×HIS tag for affinity and other chromatographic purification according to standard procedures.

### Example 8: Screening of Purified Humabody® V_{H} for Binding to CHO Human CD137 Cells and Recombinant Full Length Human and Rhesus ECD and Human ECD Fragments

Following selections of the libraries, specific V_{H} that bound to CHO cells expressing human CD137, domains 1-2 of human CD137 ECD, domains 1-3 of human CD137 ECD and human and rhesus full length CD137 ECD were identified by screening of purified Humabody® V_{H}.

Binding of purified His-tagged V_{H} to CHO human CD137 cells and to CHO parent cells for determination of non CD137 specific binding was assessed using Fluorescence Microvolume Assay Technology (FMAT). Fluorescence emission was measured on the TTP Mirrorball plate reader in the FL2 (502 nm-537 nm) and FL5 (677-800 nm) channels following excitation at 488 nm and 640 nm. Data was gated on FL5 perimeter and peak intensity and the FL2 median mean fluorescence intensity of the gated data used for determination of V_{H} binding.

Binding of purified His-tagged V_{H} to full length human CD137 (Acro Bio 41B-H5258), domains 1-3 human CD137 (In-house), domains 1-2 human CD137 (In-house) and full-length rhesus CD137 (Sino Biologics 90847-K02H) was assessed by Homogeneous Time Resolved Fluorescence (HTRF) assay. For the HTRF assays all the recombinant CD137 proteins contained huFc tag and the samples and reagents were prepared in buffer containing PBS, 0.1% BSA and 0.4M Potassium Fluoride and Serial dilutions of the V_{H} were incubated with the individual recombinant CD137huFc proteins together with 1 nM anti-human Fc cryptate (Cisbio, 61HFCKLA), and 20 nM anti-HIS D2 (Cisbio, 61HisDLA) in black 384 shallow well plates (Costar 3676) and overnight at 4° C. The Time resolved fluorescent emission at 620 nm and 665 nm was measured following excitation at 337 nm on the BMG PHERAstar plate reader. The HTRF counts=(665 emission/620 nm emission)*10000 with the results expressed as Delta F % calculated according to manufacturers instruction.

### Example 9: Generation of Multivalent Constructs

DNA sequences encoding Humabody® V_{H} specific for CD137, a V_{H} specific for PD-1 and a V_{H} specific for Human Serum Albumin (HSA) were amplified by PCR. They were assembled into larger fragments, with the V_{H} sequences flanked by linkers encoding glycine/serine-rich sequences and ligated into an expression vector by a restriction enzyme-based method. Plasmids were transformed into microbial expression systems as per standard techniques. The presence of Humabody® V_{H} sequences was verified by a standard colony PCR technique. Insert sequences were then confirmed by Sanger sequencing using vector-specific and internal primers to ensure complete sequence coverage. Sequences for exemplary constructs for CD137 and PD-1 are shown herein.

TPP-1293 and TPP-1294 include PD-1 binders that block PD-1 binding to its ligand PD-L1 and PD-1 signalling. The other constructs include PD-1 binders that do not block PD-1 binding to PD-L1.

TPP-1293 and TPP-1294 have been prepared and are being expressed. Exemplary HSA binding single V_{H} domain antibodies for use in the constructs are shown below in table 13.

TABLE 13

| | | | | SEQ ID |
|---|---|---|---|---|
| | | | | Full |
| Name | CDR1 | CDR2 | CDR3 | V_{H} |
| HSA1 | SEQ ID | SEQ ID | SEQ ID | SEQ ID |
| | NO: 1820 | NO: 1821 | NO: 1822 | NO: 1823 |
| | HYAMH | GISWNGN | DSSLFIV | EVQLVES |
| | | KITYADS | GAPTFEH | GGGLVQP |
| | | VKG | | GRSLRLS |
| | | | | CAASGFT |
| | | | | FHHYAMH |
| | | | | WVRQAPG |
| | | | | KGLEWVS |
| | | | | GISWNGN |
| | | | | KITYADS |
| | | | | VKGRFTI |
| | | | | SRDNAKN |
| | | | | SLYLQMN |
| | | | | SLRAEDT |
| | | | | ALYYCVR |
| | | | | DSSLFIV |
| | | | | GAPTFEH |
| | | | | WGRGTLV |
| | | | | TVSS |
| HSA2 | SEQ ID | SEQ ID | SEQ ID | SEQ ID |
| | NO: 1824 | NO: 182 | NO: 1826 | NO: 1827 |
| | NYNMN | 5SISSAGT | DPHSTGW | EVQLVES |
| | | HIYYADS | YKDFDY | GGGLVKP |
| | | VKG | | GGSLRLS |
| | | | | CAASGFT |
| | | | | FSNYNMN |
| | | | | WVRQAPG |
| | | | | KGLEWVS |
| | | | | SISSAGT |
| | | | | HIYYADS |
| | | | | VKGRFTI |
| | | | | SRDNAKN |
| | | | | SLYLQMN |
| | | | | SLRAEDT |
| | | | | AVYYCAR |
| | | | | DPHSTGW |
| | | | | YKDFDYW |
| | | | | GQGTLVT |
| | | | | VSS |
| HSA13 | SEQ ID | SEQ ID | SEQ ID | SEQ ID |
| | NO: 1828 | NO: 1829 | NO: 1830 | NO: 1831 |
| | SYTMN | SISSSGR | DPRMVGN | EVQLLES |
| | | YIYYADS | PHEFDl | GGGLVKP |
| | | VKG | | GGSLRLS |
| | | | | CAASGFT |
| | | | | VSSYTMN |
| | | | | WVRQAPG |
| | | | | KGLEWVS |
| | | | | SISSSGR |
| | | | | YIYYADS |
| | | | | VKGRFTI |
| | | | | SRDNAKN |
| | | | | SLYLQMN |
| | | | | SLRAEDT |
| | | | | AVYYCAR |
| | | | | DPRMVGN |
| | | | | PHEFDIW |
| | | | | GQGTMVT |
| | | | | VSS |

Exemplary PSMA binding single V_{H} domain antibodies for use in the constructs with CD137 single V_{H} domain antibodies are shown below in table 14.

TABLE 14a

| | | | | SEQ ID |
|---|---|---|---|---|
| Name | CDR1 | CDR2 | CDR3 | full V_{H} |
| PSMA1 | SEQ ID | SEQ ID NO: | SEQ ID | SEQ ID |
| | NO: | 1833 | NO: | NO: 1835 |
| | 1832 | YISY | 1834 | EVQLVESG |
| | GYGMH | DGSN | DPAW | GGWQPGRS |
| | | KYYA | GLRL | LRLSCAAS |
| | | DSVK | GESS | GFSFSGYG |
| | | G | SYDF | MHWVRQAP |
| | | | DI | GKGLEWVA |
| | | | | YISYDGSN |
| | | | | KYYADSVK |
| | | | | GRFTISRD |
| | | | | NSKNTLYL |
| | | | | QMNSLRAE |
| | | | | DTAVYYCA |
| | | | | KDPAWGLR |
| | | | | LGESSSYD |
| | | | | FDIWGQGT |
| | | | | MVTVSS |
| PSMA2 | SEQ ID | SEQ ID | SEQ ID | SEQ ID |
| | NO: | NO: | NO: | NO: 1839 |
| | 1836 | 1837 | 1838 | EVQLLESG |
| | SYALS | SIGE | DGVH | GGLVQPGG |
| | | NDGT | | SLRLSCAA |
| | | TDYA | | SGFSFSSY |
| | | DAVK | | ALSWVRQA |
| | | G | | PGKGLEWV |
| | | | | SSIGENDG |
| | | | | TTDYADAV |
| | | | | KGRFTISR |
| | | | | DNSKNTLY |
| | | | | LQMNSLRV |
| | | | | EDTAVYYC |
| | | | | VKDGVHWG |
| | | | | QGTLVTVS |
| | | | | S |

TABLE 15
Exemplary multivalent constructs CD137 + PSMA

SEQ ID NO: 1840 TPP-799 PSMA-6GS-CD137-6GS-HSA Protein
SEQ ID NO: 1841 TPP-799 Nucleotide
SEQ ID NO: 1842 TPP-800 PSMA-6GS-CD137-6GS-HSA Protein
SEQ ID NO: 1843 TPP-800 Nucleotide
SEQ ID NO: 1844 TPP-801 PSMA-6GS- CD137-6GS-HSA Protein
SEQ ID NO: 1845 TPP-801 Nucleotide
SEQ ID NO: 1846 TPP-802 PSMA-6GS- CD137-6GS-HSA Protein
SEQ ID NO: 1847 TPP-802 Nucleotide
SEQ ID NO: 1848 TPP-805 PSMA-6GS- CD137-6-6GS-HSA Protein
SEQ ID NO: 1849 TPP-805 Nucleotide
SEQ ID NO: 1850 TPP-806 PSMA-6GS- CD137-6GS-HSA Protein
SEQ ID NO: 1851 TPP-806 Nucleotide

### Example 10. Testing of Purified V_{H}

The molecules tested in the experiments below are based on the TPP constructs and sequences as shown herein. Where appropriate, molecules comprised a C terminal sequence, such as a purification tag (e.g. His tag).

a) Binding Assays

Purified Humabody® V_{H} were tested for binding to human CD137 protein, tumour necrosis factor receptor family members OX40 and GITR (Glucocorticoid-induced TNFR-related), CD40 and HVEM, CHO human CD137 cells, CHO PD1 cells and CHO parent cells.

TABLE 16
Binding of V_{H} to CD137 and TNFR family members

| | ELISA | | | | |
|---|---|---|---|---|---|
| | Human | Human | Human | Human | Human |
| Humabody ® ID | CD137 | GITR | OX40 | CD40 | HVEM |
| 003BB172-E01 | + | − | − | − | − |
| 003BB172-E01-14 | + | − | − | − | − |

No binding (−),
Binding measured (+)

Specificity of binding for CD137 over the tumour necrosis factor receptor family members OX40, GITR (Glucocorticoid-induced TNFR-related), CD40 and HVEM was determined using an ELISA assay. Nunc Maxisorp plates were coated with 1 ug/ml human CD137-Fc recombinant protein (Acro Biosystems 41B-H5258), human GITR-Fc (R&D Systems cat no. 689-GR) human OX40-Fc (R&D Systems cat no. 3388-OX), human CD40 (R&D Systems cat no. CDO-H5253), human HVEM (R&D Systems cat no. HVM-H5258) in PBS overnight at 4° C. then washed twice with PBS. Non-specific protein interactions were blocked by incubation with 1% (w/v) skimmed milk powder (Marvel®) in PBS/0.1% Tween-20 for 1 hour at room temperature. Plates were washed twice with PBS then V_{H} or antibody control (1 ug/ml) added for 1 hour at room temperature. Following three washes with PBS/0.1% Tween-20 a 1:1000 dilution of anti His-HRP (V_{H} detection) or anti mouse-HRP (positive control mouse monoclonal antibody detection) was added in 1% Marvel/PBS/0.1% Tween-20. The detection antibodies were allowed to bind for 1 hour at room temperature then the plates were washed twice in PBS/0.1% Tween-20 and once in PBS. The ELISA was developed using TMB substrate and the reaction was stopped by the addition of 50 ul 0.5M H₂SO₄ solution. The absorbance at 450 nm was measured using the BMG Pherastar. Both V_{H} tested bound to CD137 but did not bind to GITR, OX40, CD40 or HVEM.

TABLE 17
Binding of Humabody ® V_{H} to CHO-CD137 and CHO-PD1 cells

| | FMAT/Mirrorball | | Flow Cytometry | | |
|---|---|---|---|---|---|
| | CHO huCD137 | CHO huPD1 | CHO huCD137 | CHO huPD1 | CHO |
| Humabody | EC50 (M) | EC50 (M) | EC50 (M) | EC50 (M) | parent |
| 003BB172-E01 | 1.73E−09 | ND | ND | ND | ND |
| 003BB172-E01-1 | 1.35E−09 | ND | ND | ND | ND |
| 003BB172-E01-2 | 1.74E−09 | ND | ND | ND | ND |
| 003BB172-E01-3 | 1.06E−09 | ND | ND | ND | ND |
| 003BB172-E01-4 | 1.33E−09 | ND | ND | ND | ND |
| 003BB172-E01-5 | 1.29E−09 | ND | ND | ND | ND |
| 003BB172-E01-6 | 1.08E−09 | ND | ND | ND | ND |
| 003BB172-E01-7 | 1.28E−09 | ND | ND | ND | ND |
| 003BB172-E01-8 | 1.08E−09 | ND | ND | ND | ND |
| 003BB172-E01-9 | 8.49E−10 | ND | ND | ND | ND |
| 003BB172-E01-10 | 1.22E−09 | ND | ND | ND | ND |
| 003BB172-E01-11 | 1.21E−09 | ND | ND | ND | ND |
| 003BB172-E01-12 | 1.21E−09 | ND | ND | ND | ND |
| 003BB172-E01-13 | 9.54E−10 | ND | ND | ND | ND |
| 003BB172-E01-14 | 1.29E−09 | ND | ND | ND | ND |
| TPP-942 | 1.57E−10 | 1.64E−10 | ND | ND | ND |
| TPP-943 | 2.07E−10 | 1.44E−10 | ND | ND | ND |
| TPP-950 | 7.35E−10 | 8.44E−10 | ND | ND | ND |
| TPP-951 | 4.19E−10 | 8.05E−10 | ND | ND | ND |
| TPP-957 | 1.17E−09 | 7.66E−10 | ND | ND | ND |
| TPP-958 | 4.99E−10 | 3.46E−10 | ND | ND | ND |
| TPP-969 | 1.71E−10 | 1.42E−10 | 1.16E−08 | ND | NB |
| TPP-971 | 2.23E−10 | 4.14E−10 | ND | ND | ND |
| TPP-972 | 5.33E−11 | 1.50E−10 | ND | 3.30E−08 | NB |
| TPP-973 | 7.81E−11 | 4.81E−10 | 5.95E−10 | 1.08E−07 | NB |
| TPP-975 | 3.90E−10 | 7.92E−10 | ND | ND | ND |
| TPP-982 | 8.96E−11 | 3.32E−10 | ND | ND | ND |
| TPP-983 | 4.43E−11 | 2.40E−10 | ND | ND | ND |
| TPP-984 | 3.14E−10 | 2.64E−10 | ND | ND | ND |
| TPP-985 | 3.82E−10 | 5.41E−10 | ND | ND | ND |
| TPP-1010 | 1.01E−10 | 1.62E−10 | 9.39E−09 | 5.31E−10 | NB |
| TPP-1023 | 1.45E−10 | 6.26E−10 | ND | ND | ND |
| TPP-1025 | ND | 1.23E−09 | ND | ND | ND |
| TPP-1027 | 6.72E−11 | 1.73E−10 | 1.47E−09 | 5.01E−09 | NB |
| TPP-1028 | 1.51E−10 | 5.83E−10 | 1.99E−09 | 2.27E−08 | NB |
| TPP-1245 | ND | ND | 4.404E−09 | 1.83E−07 | ND |
| TPP-1246 | ND | ND | 2.48E−09 | 2.05E−08 | ND |
| TPP-1247 | ND | ND | 3.39E−09 | 2.54E−08 | ND |
| TPP-1290 | ND | ND | 4.451E−09 | 2.27E−08 | ND |
| TPP-1293 | ND | ND | ND | 4.48E−08 | ND |

Not determined (ND),
No binding (NB)

Binding of His-tagged molecules to CHO human CD137, CHO parent and CHO human PD1 cells was assessed using Fluorescence Microvolume Assay Technology (FMAT). All reagents were prepared in FMAT assay buffer (pH 7.4) containing PBS, 0.1% Bovine Serum Albumin, 0.05% Sodium Azide. Serially diluted samples were transferred into 384 well black clear-bottomed assay plates (Costar cat. no. 3655) and incubated for a minimum of 2 hours at room temperature with 1.5 nM Anti-His (Millipore cat. no. 05-949), 3 nM Goat Anti-Mouse Alexa Fluor-488 (Jackson ImmunoResearch cat. no. 115-545-071) and 2000 cells/well pre-stained with DRAQ5 (Thermo Scientific cat. no. 62251). Fluorescence emission was then measured on the TTP Mirrorball plate reader in the FL2 (502 nm-537 nm) and FL5 (677-800 nm) channels following excitation at 488 nm and 640 nm. Data was gated on FL5 perimeter and peak intensity and the FL2 median mean fluorescence intensity of the gated data used for determination of V_{H} binding. Example EC₅₀ values for binding are shown in table 9. Monovalent CD137 specific Humabody® V_{H}, and trispecific molecules with a CD137 binding arm bound to CHO CD137 expressing cells. Trispecific Humabody® V_{H}, with a PD1 binding arm bound to PD1 expressing cells.

Binding of His-tagged V_{H} samples was tested in a washed FACS assay using CHO cells lines expressing human PD-1 and human CD137 that were previously generated using standard methods. Briefly, CHO PD-1, CHO CD137 and CHO parent cells were plated (5000/well) in FACS buffer (PBS+2% FBS+0.1% BSA+0.02% Azide) into a 96-well V-bottom plate. V_{H} samples were serially diluted in assay media (PBS+0.1% BSA), added to respective wells of the plate and cells were incubated on ice for 45 minutes, protected from light. Cells were washed twice with FACS buffer and a detection mix containing 20 nM biotinylated anti-His primary antibody and 40 nM Streptavidin Alexa Fluor 647 (AF647) secondary antibody was added. Cells were incubated on ice for 30 minutes, protected from light. Cells were washed twice with FACS buffer, fixed and analysed for AF647 median fluorescence using an Intellicyt iQue Screener Plus.

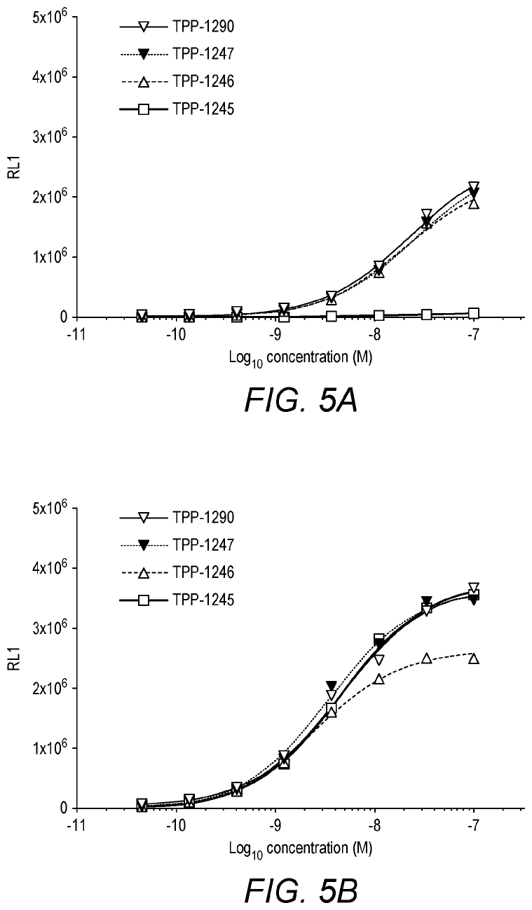

Binding of AlexaFluor AF488-labelled Humabody® V_{H} samples was tested in a washed FACS assay using CHO cell lines expressing human PD-1 and human CD137 that were previously generated using standard methods. Briefly, CHO PD-1 and CHO CD137 cells were thawed, counted, resuspended at 2e⁴/well in FACS buffer (PBS+2% FBS+0.1% BSA+0.02% Azide) and plated into a 96-well V-bottom plate. V_{H} samples were serially diluted in FACS buffer and added to respective wells of the plate. The plate was then incubated on ice for 30 minutes, protected from light. Cells were washed twice with FACS buffer and finally resuspended in FACS buffer containing Sytox RED as Live/Dead detector. Cells were then analysed for AF488 median fluorescence using an Intellicyt iQue Screener (FIG. 5).

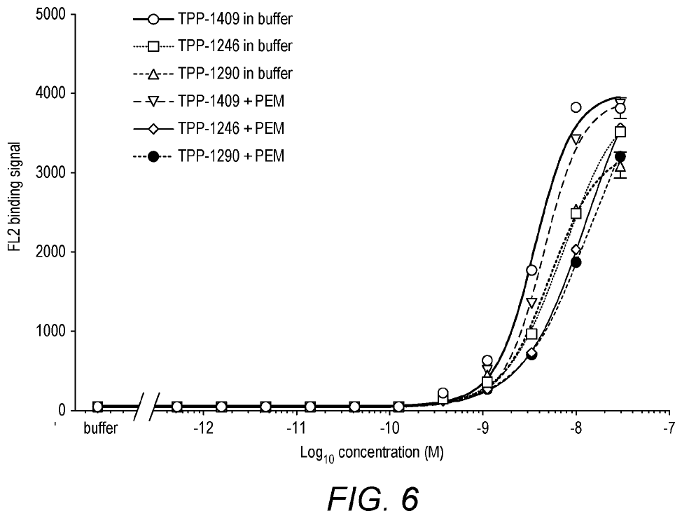

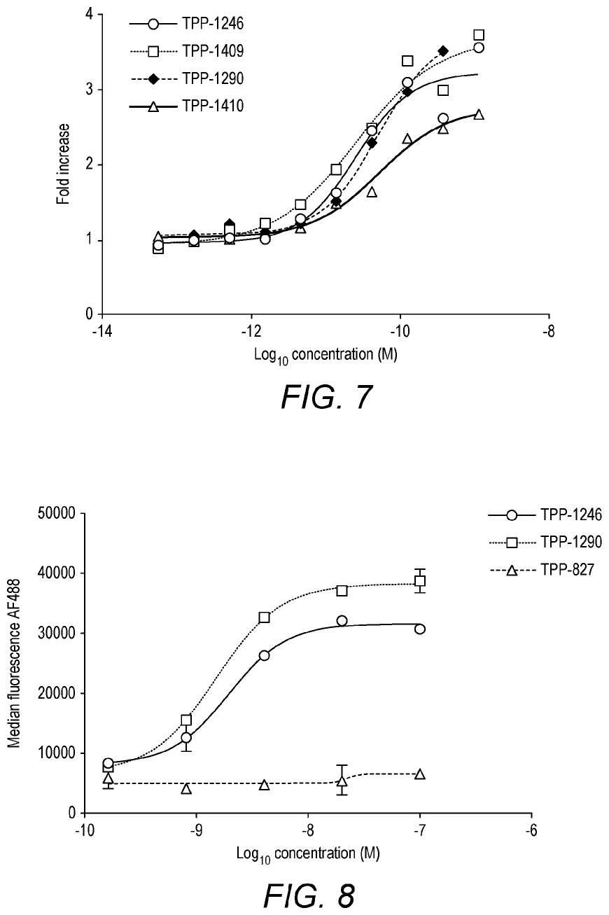

To confirm that our lead molecules target a different epitope on the PD1 receptor from the commercially available Pembrolizumab, a competition assay was setup using the FMAT (Fluorescence Microvolume Assay) Technology, which measures cell associated fluorescence within a defined volume at the bottom of the well of the assay plate. In this assay, binding of AF488-labelled Humabody® V_{H} was measured in presence of an excess (150 nM) of Pembrolizumab and compared to binding of Humabody® V_{H} in buffer. CHO cells stably expressing huPD1 were thawed at 37° C., counted, re-suspended in assay media (PBS+0.1% BSA+0.05% Sodium Azide) and plated at 1e5 cells/ml in a 384 well assay plate. Serial dilutions of test molecules were prepared in assay media in a 384 well plate and added to the assay plate, followed by addition of either buffer or excess of Pembrolizumab. After 2 hours incubation at room temperature, the plate was read with the TPP Mirrorball plate reader to measure FL2 emission (502-537 nm). Data was plotted with a 4 parameters non-linear regression equation: Y=Bottom+(X\hat{ }Hillslope)*(Top−Bottom)/(X\hat{ }HillSlope+EC50\hat{ }HillSlope) (FIG. 6).

TABLE 22
NanoBiT ® Cell based Protein Interaction Assay

| Humabody | EC50 (M) |
|---|---|
| TPP-1246 | 2.51E−11 |
| TPP-1290 | 5.145E−11 |
| TPP-1438 | 1.05E−10 |
| TPP-1439 | 1.481E−10 |
| TPP-1437 | 1.192E−09 |
| TPP-1409 | 2.588E−11 |
| TPP-1410 | 5.316E−11 |

The ability of the CD137-PD1 bispecific Humabody® V_{H} to bind in -cis to both targets simultaneously on the same cell was assessed in the NanoBiT® cell based protein interaction assay. The NanoBiT® technology provides a method for direct analysis of protein interactions in live cells. NanoBiT® assays rely on a two-subunit system based on NanoLuc® luciferase that can be used for intracellular detection of protein:protein interactions. The subunits are fused to proteins of interest, forming a functional enzyme that generates a bright, luminescent signal when the proteins interact. Two stable cell lines were generated in house with CD137 and PD1 individually fused to subunits of NanoLuc® luciferase. Serial dilutions of CD137-PD1 bispecific Humabody® V_{H} were prepared in assay media (HAM12+10% FBS) in a 96 well V bottom plate. 2.5×10⁵ cells were plated in a 96 flat bottom TC white plate and serially diluted samples added. After a 7-hour incubation at 37° C. in a CO₂ incubator the level of luciferase reporter expression was determined by addition of NanoGlo reagent (Promega N1120) and measurement of luminescent signal on the BMG Pherastar. Fold Increase over background values (buffer only) were calculated, and data was plotted with a 4 parameters nonlinear regression equation:

$$Y = Bottom + ( X \hat{} Hillslope ) * ( Top - Bottom ) / ( X \hat{} HillSlope + EC 50 \hat{} HillSlope ) .$$

TABLE 23
Binding to pre-stimulated human CD8+ cells
Average EC₅₀ values for binding of bispecific molecules with a CD137
and PD1 binding arm bound to pre-stimulated CD8+ cells are shown in
table below. Data shown is the average of binding from 2 separate donors.

| Humabody | EC50 (M) |
|---|---|
| TPP-1290 | 1.49E−09 |
| TPP-1246 | 2.22E−09 |

Binding of the labelled Humabody® V_{H} to PD1+/CD137+ cells was measured using flow cytometry. Peripheral blood mononuclear cells (PBMCs) were isolated from human blood by density gradient centrifugation then CD8+ T cells purified using a negative selection isolation kit according to the manufacturer's protocol (Stem cell technologies cat no 17953). T-cells were stimulated with PMA/lonomycin for 48 hours in RPMI media supplemented with 10% FBS, 2 mM Glutamine, 1× Pen/Strep. Cells were transferred into a 96 well v bottom plates, washed in staining buffer (PBS/1% BSA/0.05% Sodium Azide 10 μg/ml HSA) then incubated with serially diluted (starting from 100 nM) directly labelled Alexa Fluor-488 V_{H}. Cells were washed by centrifugation then antibody mix containing CD137 (Mouse anti human Biolegend catalogue no. 329910) and PD1 (Mouse anti human Biolegend catalogue no. 309820) were added followed by a 30-minute incubation on ice. A Live Dead near IR stain (Molecular Probes cat no. L10119) was used for discrimination of live cells. After further washing cells were fixed and fluorescence measured by flow cytometry (Table 23 and FIG. 8).

TABLE 24
Binding to stimulated cynomolgus T cells
Average EC50 values for binding of bispecific molecules with a CD137
and PD-1 binding arm bound to activated CD137+ T cells are shown
in the table below. Binding was measured by flow cytometry.

| | Cynomolgus (n = 3) | Human control (n = 1) |
|---|---|---|
| Humabody | EC50 (M) | EC50 (M) |
| TPP-1246 | 3.63E−09 | 3.75E−09 |
| TPP-1290 | 7.17E−09 | 6.87E−09 |

Binding of the labelled Humabody® V_{H} to CD137+ cells was measured using flow cytometry. Cryopreserved human and cynomolgus peripheral blood mononuclear cells were thawed in the presence of 25 U/ml Benzonase. Cells were then resuspended in RPMI media supplemented with 10% FBS, 2 mM Glutamine and 1× Pen/Strep and stimulated with 1 ug/ml platebound anti-CD3 plus 0.5 ug/ml anti-CD28. T cells were expanded over 8 days in media supplemented with IL-7/IL15 before a fraction were stimulated by PMA/ionomycin for 16 hrs to induce CD137 and PD-1 expression. PMA/ionomycin stimulated and untreated cells were mixed to give a heterogeneous population then transferred to a V-bottom plate (10⁵/well) and stained for viability (Molecular Probes cat.no. L10119). Cells were incubated on ice for in PBS/2 mM EDTA/1% cyno serum plus Alexa Fluor 488 labelled V_{H}, serially diluted from 100 nM. After 1 hr cells were washed (×2) and stained for expression of cell surface of CD137 (Biolegend cat.no. 309824) and PD1 (Biolegend cat. No. 329906) for 30 mins on ice. Cells were again washed (×2) before analysis of V_{H} binding to viable activated (CD137+) and non-activated (CD137-) T cells by flow cytometry (Table 24).

TABLE 18
Kinetics of binding of monovalent V_{H} to human CD137 and rhesus CD137

| | Human CD137 | | | Rhesus CD137 | | |
|---|---|---|---|---|---|---|
| CLONE NAME | KD (M) | kon(1/Ms) | kdis(1/s) | KD (M) | kon(1/Ms) | kdis(1/s) |
| 003BB172-E01 | 2.04E−08 | 6.31E+04 | 1.29E−03 | 2.75E−08 | 4.78E+04 | 1.31E−03 |
| 003BB172-E01-1 | 1.78E−08 | 1.20E+05 | 2.14E−03 | 2.19E−08 | 1.22E+05 | 2.67E−03 |
| 003BB172-E01-2 | 1.86E−08 | 7.32E+04 | 1.37E−03 | 2.59E−08 | 6.13E+04 | 1.59E−03 |
| 003BB172-E01-3 | 2.45E−08 | 6.60E+04 | 1.61E−03 | 2.48E−08 | 5.53E+04 | 1.57E−03 |
| 003BB172-E01-4 | 2.57E−08 | 7.16E+04 | 1.84E−03 | 3.22E−08 | 6.71E+04 | 2.16E−03 |
| 003BB172-E01-5 | 1.49E−08 | 9.06E+04 | 1.35E−03 | 1.15E−08 | 1.42E+05 | 1.64E−03 |
| 003BB172-E01-6 | 2.67E−08 | 8.66E+04 | 2.31E−03 | 3.90E−08 | 7.36E+04 | 2.87E−03 |
| 003BB172-E01-7 | 2.32E−08 | 8.59E+04 | 1.99E−03 | 4.07E−08 | 5.73E+04 | 2.33E−03 |
| 003BB172-E01-8 | 2.45E−08 | 6.60E+04 | 1.61E−03 | 4.14E−08 | 6.5E+04 | 2.69E−03 |
| 003BB172-E01-9 | 1.75E−08 | 1.16E+05 | 2.03E−03 | 2.30E−08 | 1.02E+05 | 2.35E−03 |
| 003BB172-E01-10 | 2.14E−08 | 9.11E+04 | 1.95E−03 | 3.69E−08 | 6.74E+04 | 2.49E−03 |
| 003BB172-E01-11 | 2.10E−08 | 1.13E+05 | 2.38E−03 | 3.31E−08 | 7.25E+04 | 2.40E−03 |
| 003BB172-E01-12 | 2.00E−08 | 9.44E+04 | 1.89E−03 | 3.86E−08 | 5.97E+04 | 2.30E−03 |
| 003BB172-E01-13 | 2.11E−08 | 9.87E+04 | 2.08E−03 | 2.28E−08 | 1.11E+05 | 2.53E−03 |
| 003BB172-E01-14 | 2.21E−08 | 9.21E+04 | 2.03E−03 | 2.59E−08 | 6.13E+04 | 1.59E−03 |

Binding kinetics of purified monovalent V_{H} molecules were measured on a ForteBio Octet RED 384 instrument. Human and Rhesus CD137-Fc tagged protein was diluted to 5 μg/ml in kinetics buffer (0.05% Tween, 1×PBS) and coupled to Protein G biosensors (ForteBio cat no. 18-5082) via the Fc tag. V_{H} were serially diluted (typically 1:2 dilution series starting with 200 nM, V_{H} at the highest concentration) and binding to the CD137-Fc-coupled Protein G biosensors measured. Binding kinetics were determined from the (blank subtracted) sensor gram trace using 1:1 binding models and ForteBio Octet Data Analysis 9.0 software. Example kinetic and binding affinity data obtained is shown Table 18 (monovalent V_{H}). In this assay format monomer V_{H} bound hCD137-Fc with affinities of between 14.9 nM and 26.7 nM and rhCD137-Fc with affinities between 11.5 nM and 41.4 nM.

TABLE 19
Kinetics of binding of CD137-PD1 Humabody ®
V_{H} to human CD137 and human PD1

| | Human CD137 | | | Human PD1 | | |
|---|---|---|---|---|---|---|
| Humabody | kon(1/Ms) | kdis(1/s) | KD (M) | kon(1/Ms) | kdis(1/s) | KD (M) |
| TPP-969 | 1.22E+05 | 2.55E−03 | 2.09E−08 | 6.85E+04 | 9.15E−04 | 1.34E−08 |
| TPP-972 | 1.54E+06 | 1.55E−05 | 1.00E−11 | 6.43E+04 | 7.61E−04 | 1.18E−08 |
| TPP-973 | 2.37E+05 | 2.57E−05 | 1.08E−10 | 1.95E+05 | 1.40E−02 | 7.16E−08 |
| TPP-1010 | 1.73E+05 | 3.66E−03 | 2.11E−08 | 4.98E+05 | 2.74E−03 | 5.50E−09 |
| TPP-1027 | 1.25E+06 | 2.87E−05 | 2.30E−11 | 4.13E+05 | 2.46E−03 | 5.95E−09 |
| TPP-1028 | 6.74E+05 | 1.90E−05 | 2.81E−11 | 4.03E+05 | 2.35E−03 | 5.83E−09 |
| TPP-1245 | 3.81E+05 | 1.79E−04 | 4.71E−10 | 8.69E+05 | 7.62E−02 | 8.76E−08 |
| TPP-1246 | 3.45E+05 | 1.87E−04 | 5.41E−10 | 2.41E+05 | 2.02E−03 | 8.38E−09 |
| TPP-1247 | 3.34E+05 | 9.85E−05 | 2.95E−10 | 2.76E+05 | 2.14E−03 | 7.77E−09 |
| TPP-1290 | 2.83E+05 | 1.85E−04 | 6.52E−10 | 3.45E+05 | 2.44E−03 | 7.07E−09 |
| TPP-1293 | 5.86E+04 | 1.51E−04 | 2.57E−09 | 9.15E+04 | 2.12E−04 | 2.31E−09 |
| TPP-1408 | 4.03E+04 | 3.83E−03 | 9.51E−08 | 2.55E+05 | 1.13E−03 | 4.45E−09 |
| TPP-1409 | 1.52E+05 | 3.07E−04 | 2.02E−09 | 5.80E+05 | 2.41E−03 | 4.16E−09 |
| TPP-1410 | 8.18E+04 | 1.21E−04 | 1.47E−09 | 2.96E+05 | 2.24E−03 | 7.57E−09 |
| TPP-1411 | 1.08E+05 | 2.08E−04 | 1.93E−09 | 5.37E+05 | 2.67E−03 | 4.98E−09 |
| TPP-1438 | 5.00E+05 | 1.10E−04 | 6.20E−10 | 5.27E+05 | 5.02E−05 | 9.51E−11 |
| TPP-1439 | 8.00E+05 | 1.07E−04 | 5.06E−10 | 7.50E+05 | 5.12E−05 | 6.83E−11 |

TABLE 25
Kinetics of binding of CD137-PD1 Humabody ® V_{H} to
cynomolgus macaque CD137 and cynomolgus macaque PD1

| | Cyno CD137 | | | Cyno PD1 | | |
|---|---|---|---|---|---|---|
| Humabody | kon(1/Ms) | kdis(1/s) | KD (M) | kon(1/Ms) | kdis(1/s) | KD (M) |
| TPP-1245 | 3.61E+05 | 2.63E−04 | 7.28E−10 | 4.93E+05 | 5.05E−02 | 1.02E−07 |
| TPP-1246 | 3.17E+05 | 2.67E−04 | 8.42E−10 | 2.27E+05 | 4.64E−03 | 2.05E−08 |
| TPP-1247 | 3.13E+05 | 1.70E−04 | 5.43E−10 | 2.93E+05 | 4.94E−03 | 1.69E−08 |
| TPP-1290 | 2.47E+05 | 2.55E−04 | 1.03E−09 | 3.10E+05 | 5.39E−03 | 1.74E−08 |
| TPP-1293 | 5.22E+04 | 2.26E−04 | 4.34E−09 | 1.19E+05 | 4.07E−04 | 3.41E−09 |

For Humabody® V_{H}, TPP-969, 972, 973, 1010, 1027 and 1028 the Biacore 8K instrument was used to study the interaction between Humabody® V_{H} with biotinylated human CD137-His and biotinylated human PD-1-His tagged protein by surface plasmon resonance (SPR). Multi cycle kinetics assays were then used to evaluate the kinetics and affinity of the interaction. Experiments were performed at 250° C. in HBS-EP+ assay buffer with a flow rate of 30 μl/minute. A streptavidin chip (Streptavidin (SA), Series S, GE Healthcare BR100531) was used to capture the biotinylated human CD137 or human PD-1 reaching a maximum of 75RU or 200RU respectively. A second flow cell without any captured antigen was used as the reference cell. A five point, 3-fold dilution series of Humabody® V_{H} was made based on the expected KD with a top concentration approximately 10-fold above the expected KD. The binding kinetics were followed by flowing these over the chip surface. The contact time for the binding steps was 360 or 180 seconds and the dissociation step was 3600 or 1200 seconds for human CD137 and PD1 respectively. After each run, the sensors were regenerated with glycine pH 1.5 to remove the captured Humabody® V_{H}. The data was fitted to a 1:1 binding model after reference subtraction using the Biacore Evaluation software. Example kinetic and binding affinity data obtained is shown Table 19.

For Humabody® V_{H}, TPP-1245, 1246, 1247, 1290, 1293, 1408, 1409, 1410, 1411, 1438 and 1439, recombinant Fc-tagged ectodomains of human CD137 (Acro Biosystems 41B-H5258) and PD1 (Acro Biosystems PD1-H5257) or cynomolgus CD137 (R&D S9324-4b) and PD1 (Acro Biosystems PD1-C5254) were captured on a Protein G sensor chip (GE Healthcare 29179315). Binding of Humabody® V_{H} constructs was measured using a single-cycle kinetics method using the BIAcore 8K instrument with 90 second sample injections with a sample concentration range of low nanomolar to high nanomolar. Dissociation was observed for at least 600 seconds and the samples were run at a flow rate of at least 50 μl/min. The Protein G sensor surface was regenerated with a 20 second injection of 10 mM Glycine buffer, pH 1.5 (GE Healthcare BR100354). Data were analysed using a 1:1 binding model in Biacore Insight Evaluation software, including Humabody® V_{H} constructs with two PD-1 arms. Example kinetic and binding affinity data obtained is shown Tables 19 and 25.

b) Functional Activity

### Jurkat Reporter Assay

The ability of monovalent V_{H}, to act as CD137 agonists was assessed in a reporter gene assay using Jurkat cells expressing CD137 and an NF-kB luciferase reporter gene. Their activity was compared to bivalent and trivalent molecules which have increased potential for avid interactions and to bispecific molecules consisting of CD137 V_{H} linked to a V_{H} that bound to the antigen PSMA or PD-1. In the bispecific molecule, CD137 agonism resulted from co-engagement of both CD137 and either the cell expressed PSMA or PD1.

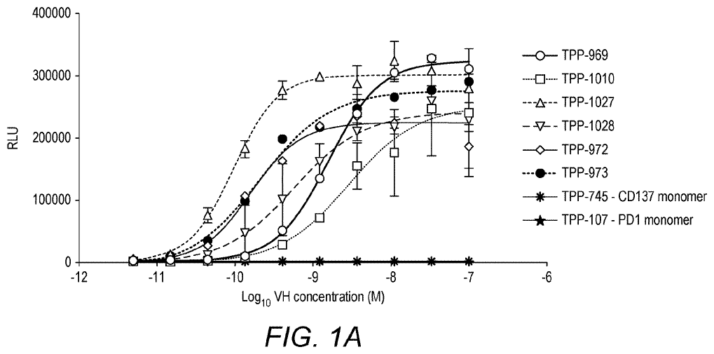

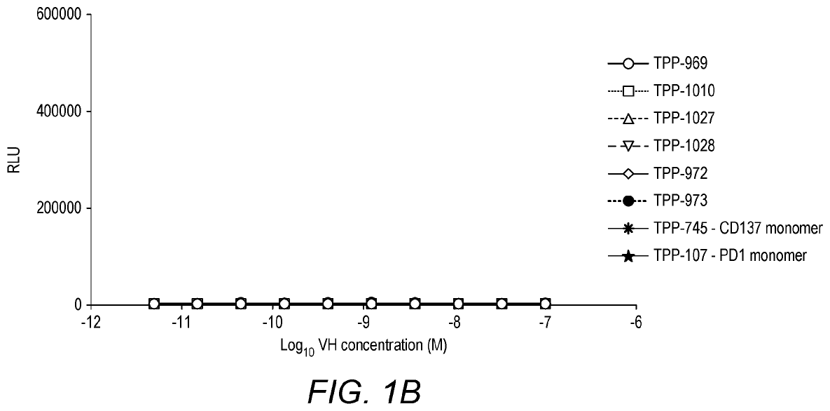

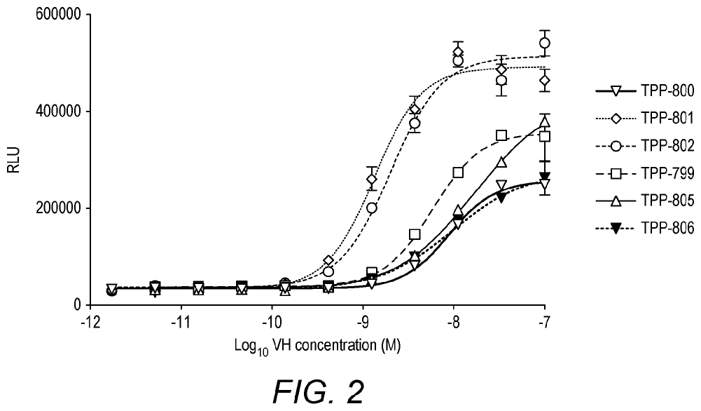

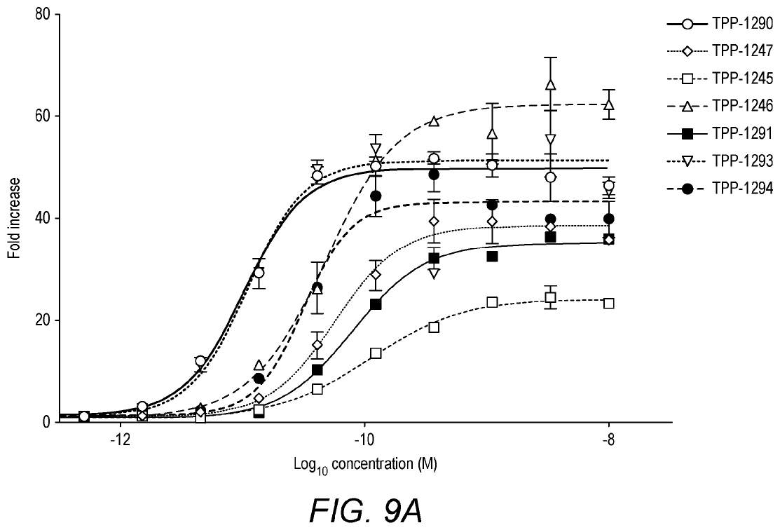

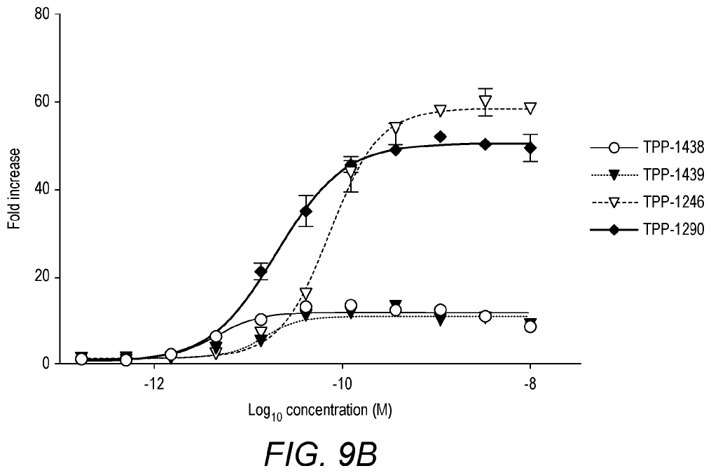

PSMA or PD1 expressing cells or parental (non PSMA/PD1) expressing (5000/well) were plated overnight in media (RPMI 1640 supplemented with 10% FBS, 2 mM L-Glutamine, 1× Pen/Strep) into 384 well, white flat bottomed tissue culture treated plates. Serially diluted monovalent V_{H}, multivalent V_{H} and PSMA or PD-1/CD137 targeting bispecific molecules were prepared in media and added to the wells followed by Jurkat human CD137 NF-kB luciferase reporter gene cells (Promega). After a 5-6 hour incubation at 37° C. in a CO₂ incubator the level of luciferase reporter expression was determined by addition of BioGlo reagent (Promega G7940) and measurement of luminescent signal on the BMG Pherastar. FIG. 1 shows that monovalent V_{H}, did not increase reporter gene activity and thus do not have agonistic CD137 activity in this assay. Bispecific molecules activated the Jurkat reporter cells in the presence of PD-1 expressing cells (FIG. 1A plus FIGS. 9A and 9B) or PSMA expressing cells (FIG. 2) but not when co-cultured with parental (non PD-1) expressing cells (FIG. 1B). This demonstrates that dual target engagement of PSMA or PD-1 and CD137 with a monovalent CD137 binding arm results in agonistic CD137 activity.

TABLE 20
Activity in Jurkat-CD137/CHO-PD-1 Reporter Assay

| | Jurkat-CD137/CHO-PD-1 |
|---|---|
| Humabody | Reporter Assay EC50 (M) |
| TPP-942 | 5.74E−08 |
| TPP-943 | 2.57E−08 |
| TPP-950 | 4.30E−08 |
| TPP-951 | 1.41E−08 |
| TPP-957 | 1.10E−08 |
| TPP-958 | 1.09E−08 |
| TPP-969 | 1.11E−08 |
| TPP-971 | 7.18E−09 |
| TPP-972 | 3.21E−10 |
| TPP-973 | 2.54E−10 |
| TPP-975 | 1.15E−08 |
| TPP-982 | 5.70E−10 |
| TPP-983 | 5.56E−10 |
| TPP-984 | 1.18E−08 |
| TPP-985 | 2.17E−08 |
| TPP-1008 | 3.61E−09 |
| TPP-1009 | 3.47E−08 |
| TPP-1010 | 2.29E−09 |
| TPP-1011 | 4.11E−09 |
| TPP-1012 | 5.29E−09 |
| TPP-1014 | 5.48E−10 |
| TPP-1015 | 5.07E−10 |
| TPP-1016 | 6.98E−10 |
| TPP-1017 | 2.10E−10 |
| TPP-1018 | 2.73E−10 |
| TPP-1023 | 4.37E−10 |
| TPP-1025 | 5.09E−10 |
| TPP-1027 | 2.38E−10 |
| TPP-1028 | 3.12E−10 |
| TPP-1052 | 3.26E−09 |
| TPP-1245 | 1.129E−10 |
| TPP-1246 | 7.288E−11 |
| TPP-1247 | 5.829E−11 |
| TPP-1286 | 5.302E−11 |
| TPP-1290 | 2.000E−11 |
| TPP-1291 | 8.089E−11 |
| TPP-1293 | 1.129E−11 |
| TPP-1294 | 3.227E−11 |
| TPP-1295 | 8.751E−12 |
| TPP-1409 | 1.343E−10 |
| TPP-1438 | 4.658E−12 |
| TPP-1439 | 1.454E−11 |

TABLE 21
Activity in Jurkat-CD137/CHO-PSMA Reporter Assay

| | Jurkat-CD137/CHO-PSMA |
|---|---|
| Humabody | Reporter Assay EC50 (M) |
| TPP-799 | 5.60E−09 |
| TPP-800 | 8.50E−09 |
| TPP-801 | 1.30E−09 |
| TPP-802 | 2.00E−09 |
| TPP-805 | 1.60E−08 |
| TPP-806 | 9.30E−09 |

A Jurkat reporter cell line expressing human PD-1 and a luciferase reporter gene under the control of a promoter with an NFAT response element and a CHO cell line expressing a T-Cell Receptor activator and human PDL-1 under the control of a tetracycline inducible promotor were generated by standard methods.

Cells were prepared in bulk, then frozen and stored in liquid nitrogen. CHO human PDL-1/TCR activator cells were plated overnight (2000 cells/well) in Hams F12 containing 10% FBS, and 1 μg/ml tetracycline into 384 well, white flat-bottomed tissue culture treated plates (Corning 3570). Humabody® V_{H} samples were serially diluted in assay media (RPMI+2% FBS) and added to the plates followed by the Jurkat PD-1 reporter cells (5,000 cells/well). After a 6-hour incubation at 37° C. in a CO₂ incubator the level of luciferase reporter expression was determined by addition of NanoGlo reagent (Promega N1120) and measurement of luminescent signal on the BMG Pherastar. The data is expressed as fold over background where background is the cells with buffer only wells

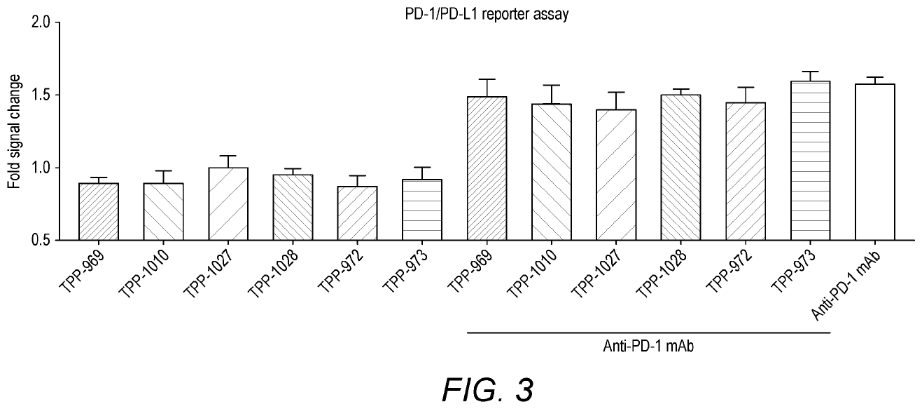

FIG. 3 shows that antagonistic activity of a PD1 mAb in a PD1/PDL1 reporter assay was not impacted by the presence of the bispecific CD137-PD1 Humabody® V_{H}.

### Example 11: Stimulation of Primary Cells

TABLE 26
CD8+ T-cell activation assay - IL-2 release
EC50 values from a representative experiment for IL-2 release
with CD137-PD1 bispecific molecules are shown in table below.

| Humabody/Antibody | EC50 (M) |
|---|---|
| TPP-1246 | 1.76E−10 |
| TPP-1290 | 1.99E−10 |
| TPP-1293 | 1.46E−10 |
| Urelumab analog | 3.97E−10 |

### CD8+ T-Cell Activation Assay—IL-2 Release

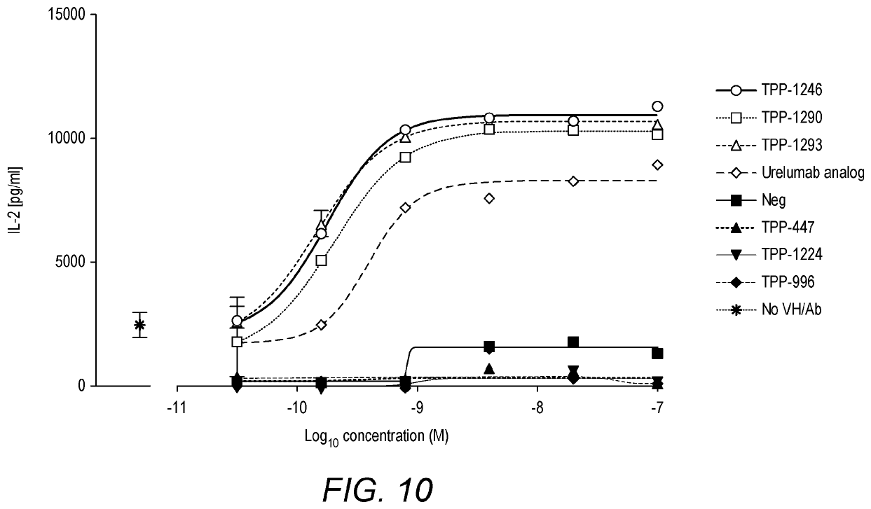

Different bispecific molecules were tested for their ability to induce IL-2 release from CD8+ T cells in a co-culture assay. CHO PD-1 expressing cells were resuspended in media (RPMI 1640 supplemented with 10% FBS, 2 mM L-Glutamine, 1× Pen/Strep, 1 ug/ml tetracycline) and seeded at a density of 20000 per well onto 96 well flat bottom plates that had been pre-coated with 5 ug/ml anti CD3 antibody (Life Tech cat no. 16-0037-85). Cells were allowed to adhere overnight at 37° C., 5% CO₂. Peripheral blood mononuclear cells (PBMCs) were isolated from human blood by density gradient centrifugation and CD8+ T cells purified using a negative selection isolation kit according to the manufacturer's protocol (Stem Cell, cat no 17953). Humabody® V_{H}, molecules and Urelumab analog were prepared in media and added together with the T cells (100000 cells/well) to the assay plates. Supernatants were harvested after a 72-hour incubation at 37° C., 5% CO₂ and IL-2 levels quantified using a human IL-2 assay kit according to the manufacturer's instructions (Cisbio Cat no. 64IL2PEB) (Table 26 and FIG. 10).

### Stimulation of Superantigen-Activated Cells

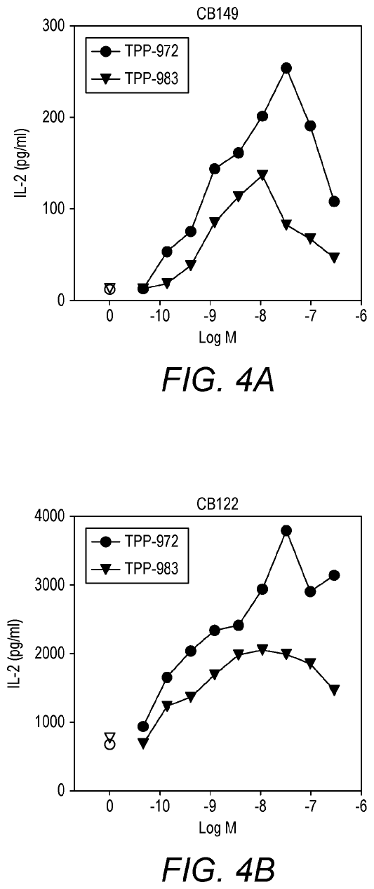

PBMCs from healthy donors were stimulated with 1 ng/ml SEB (Staphylococcal enterotoxin B) for 16 hours prior to treatment. PBMCs were washed and plated at 100,000 cells per well. Humabodies were diluted serially into complete RPMI 1640 media before being added to each well. Additional SEB diluted in RPMI was also added to give a final concentration of 0.1 ng/ml SEB. Cells were cultured for 3 days at 37° C. and 5% CO₂. Following incubation, plates were centrifuged and cell-free supernatant was harvested and IL-2 was measured using DuoSet ELISA reagents on the MSD platform. FIG. 4 demonstrates the ability of the CD137-PD1 bispecific molecules to stimulate IL-2 production from SEB activated PBMCs.

### PBMC Activation Assay—Cytokine Secretion

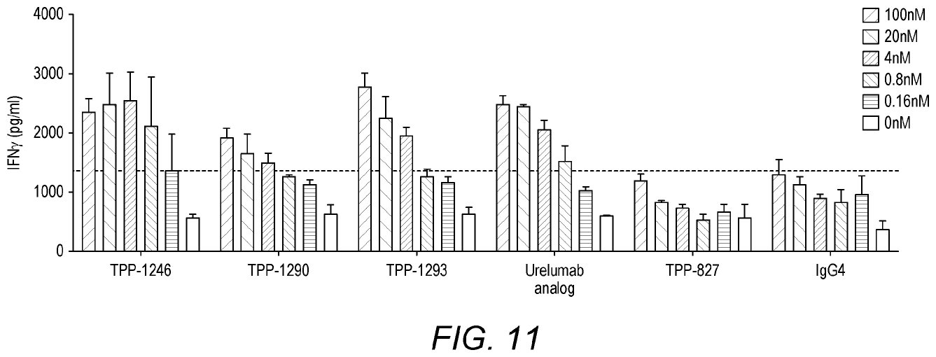

PBMCs were isolated from healthy donor blood (NHS-BT) by density gradient centrifugation using Lymphoprep (StemCell Technologies), according to standard protocols. Cells were washed using sterile PBS, counted, and resuspended at a concentration of 1×10⁶ cells/ml in RPMI-1640 medium with L-gluatmine supplemented with 10% fetal bovine serum and 1% penicillin/streptomycin (assay medium). 50 μl (50,000 cells/well) of cells were plated into 96-well flat bottom plates previously coated overnight with 1 μg/ml anti-CD3 (OKT3 clone). Humabody® V_{H} samples were serially diluted in assay medium at 2× the final concentration and 50 μl was added to respective wells. Plates were incubated for 4 days at 37° C. in a 5% CO₂ incubator. On day 4, supernatants were harvested and IFN-γ levels quantified by HTRF using a human IFN-γ assay kit (Cisbio) according to the manufacturer's protocol. Delta ratio values of standard curves and samples were graphed and interpolated using GraphPad Prism (FIG. 11).

### Repetitive Stimulation Assay—Cytokine Secretion

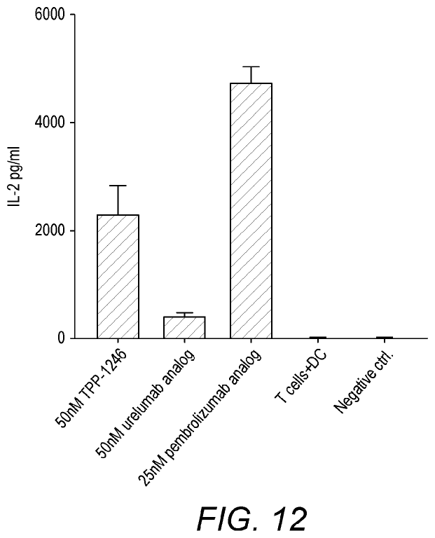

PBMCs from healthy donors were repetitively stimulated with plate bound anti-CD3 and soluble anti-CD28 antibodies at final concentration of 0.5 ug/ml for 7 days. On day 7 repetitively stimulated T cells were washed and co-cultured with autologous DC in presence of 1 ng/ml SEB (Staphylococcal enterotoxin B) and test compounds. Humabody® V_{H} and controls (Pembrolizumab analog, Urelumab analog and negative control) were tested at one concentration, in quadruplicates. The compounds were diluted into X-Vivo media+5% FBS before being added to each well. Cells were cultured for 2 days at 37° C. and 5% CO₂. Following incubation, plates were centrifuged, and cell-free supernatant was harvested, and IL-2 was measured using DuoSet ELISA reagents on the MSD platform. To investigate an impact of TPP-1246 on CD137 and PD-1 expression the cells were collected and stained post assay with fluorescence labelled commercially available antibodies: anti-PD-1 (EH12.2H7 clone), anti-CD137 (4B4-1clone), anti-CD8 (RPA-T8) and LDiRN. The cells treated with Pembrolizumab analog were used for gating PD-1+ cells, as it competes with EH12.2H7 clone for the same epitope. The cells treated with Urelumab analog were used for gating CD137+ cells, as it competes with 4B4-1 clone for the same epitope (FIG. 12).

### Repetitive Stimulation Assay—Receptor Expression

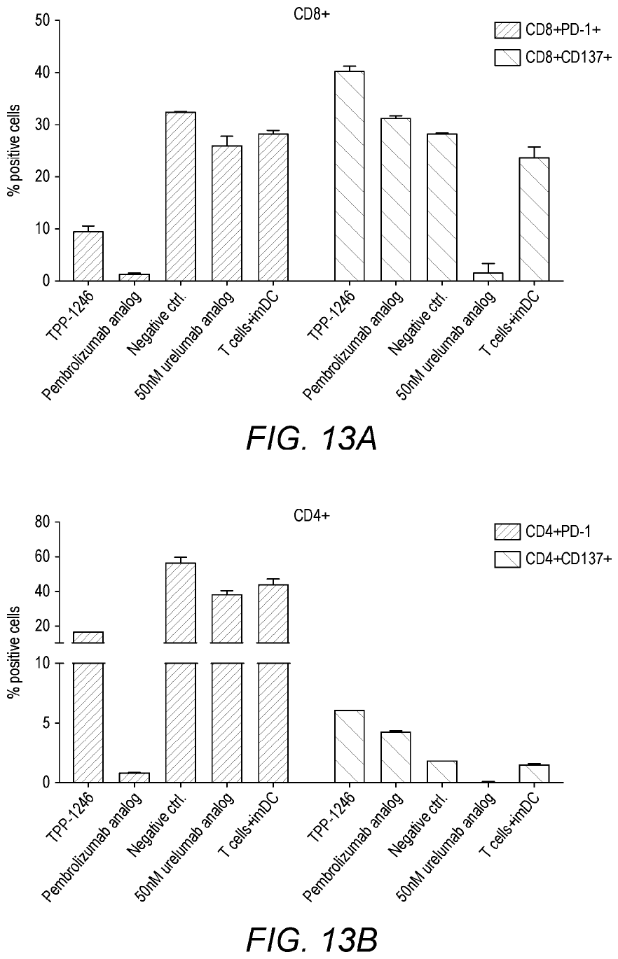

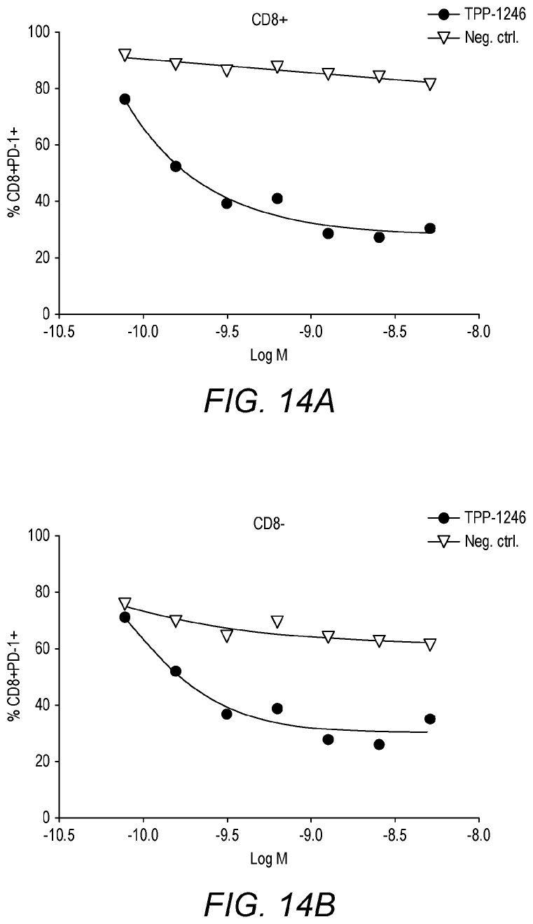

PBMCs from healthy donors were co-cultured with allogeneic DC (10:1 ratio) in the presence of 1 ng/ml SEB (Staphylococcal enterotoxin B) and CD137×PD-1 Humabody. The PBMCs prior to functional assay were stained with CellTrace™ Violet Cell Proliferation Kit (Thermo Fisher cat. no. C34557) according to the manufacturer's instructions to trace multiple generations using dye dilution by flow cytometry. PBMC were plated at 100000 cells per well. Humabody® V_{H} and controls were diluted serially into complete AIM-V media before being added to each well. Cells were cultured for 4 days at 37° C. and 5% CO₂. After 4 days of co-culture, the cells were collected and stained with fluorescence labelled commercially available antibodies: anti PD-1 (EH12.2H7 clone), anti-CD137 (4B4-1clone), anti-CD8 (RPA-T8) and LDiRN. All antibodies were from Biolegend. The divided cells were gated on CellTrace™ Violet. Data acquisition was performed using an iQue flow cytometer, and the analysis was performed using FlowJo software (FIGS. 13A & 13B and 14).

### Exhaustion Assay

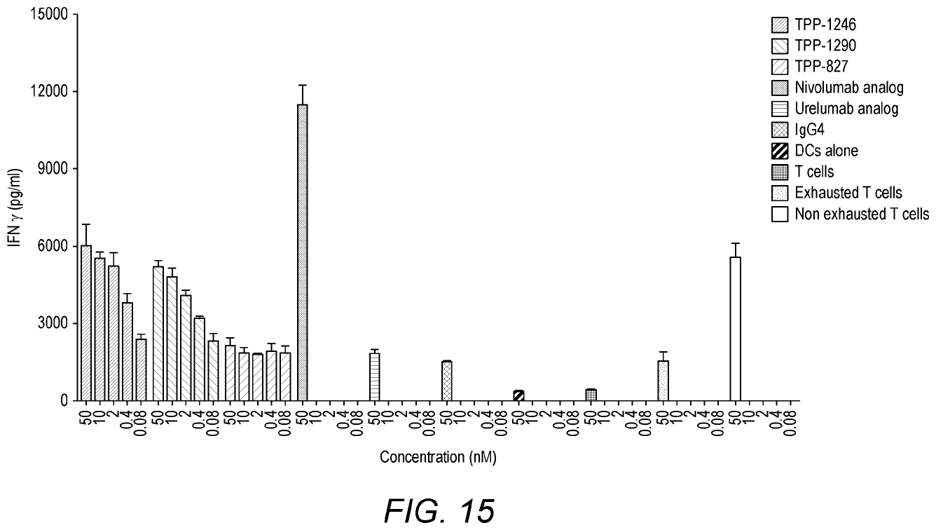

Monocytes were isolated from healthy PBMC donors using a human monocyte kit (Miltenyi Biotec) and cultured in serum free medium supplemented with interleukin 4 (IL-4) and granulocyte-macrophage colony-stimulating factor (GM-CSF) for 5 days after which monocytes were differentiated into immature DCs (iDCs). In addition, Pan T cells were also isolated from healthy PBMC donors using a human Pan T cell Kit (Miltenyi Biotec) then seeded at 1×10⁶ cells/ml in 24-well plates in the presence of CD3/CD28 tetramers (Immunocult; Stemcell Technologies; 10991). Every two days, cells were washed, resuspended at 1×10⁶ cells/ml in 24-well plates with CD3/CD28 tetramers (Immunocult; Stemcell Technologies; 10991). After 7 days, cells were washed and rested for 24 hs; after which cells were co-cultured with autologous iDC in presence or absence of the V_{H} at concentrations in a 96 well culture plate. Lastly, a solution of SEB at 0.0001 μg/ml in culture media was added to the culture plate followed by a 72-hour incubation at 37° C. and 5% CO₂. The cell culture supernatant was then harvested and cryogenically stored (−180° C.) until further use for IFNγ quantification which was measured via HTRF (Homogenous time resolved Fluorescence assay) technology. The HTRF assay was performed using a human IFN γ kit (Cisbio Catalogue no. 62H1 FNGPEH). Thawed supernatant samples were diluted in culture media 10 times. For the standard, a 7-point standard curve of two-fold serial dilutions (4000-46.4 μg/ml) in culture media and then 16 μl of either supernatant or standard was added to a 384 well plate. This was followed by the addition of 4 μl of IFN γ antibodies mixture, consisting of a donor europium cryptate labelled antibody and an acceptor XL labelled antibody. The plate was incubated overnight at room temperature protected from light. Following the incubation, the plates were read using the Pherastar microplate plate reader (BMG lab tech) and the IFN γ levels determined according the manufacturer's instructions (FIG. 15).

### Example 12: In Vivo Efficacy Study

TABLE 27
MC38 tumour model in C57BL/6-Pdcd1^{tm1(PDCD1)}Tnfrsf9^{tm1(TNFRSF9)}
mice (Crown Bioscience Inc.). Experimental mice (n = 8
per group) were treated with PBS, 3 mg/kg Q2D TPP-1315 or 5
mg/kg Q3D Urelumab analog. Tumour growth was measured twice
weekly, and the average tumour volume ± SEM as shown.

| | Day | | | | | | | | | |
|---|---|---|---|---|---|---|---|---|---|---|
| | 0 | | 3 | | 6 | | 10 | | 13 | |
| Group | Mean | SEM | Mean | SEM | Mean | SEM | Mean | SEM | Mean | SEM |
| PBS | 79.0 | 5.4 | 165.5 | 23.3 | 384.6 | 52.1 | 920.6 | 187.2 | 1782.8 | 337.2 |
| TPP-1315 | 79.0 | 5.2 | 152.5 | 11.0 | 270.8 | 31.5 | 539.4 | 81.2 | 971.3 | 129.6 |
| 3 mg/kg | | | | | | | | | | |
| Urelumab | 79.0 | 5.2 | 178.6 | 18.0 | 301.7 | 36.5 | 549.2 | 111.5 | 835.9 | 176.3 |
| 5 mg/kg | | | | | | | | | | |

TPP-1315 is the same as TPP-1246 except that the HSA binding V_{H} (191-E02-2) has been replaced with another half life extending moiety. The in vivo efficacy study utilised double knock-in HuGEMM immunocompetent mice from Crown Bioscience, whose extracellular regions of mouse PD-1 and CD137 are humanised to human PD-1 (hPD-1) and human CD137 (hCD137). The mice were inoculated subcutaneously in the right rear flank with 1×10⁶ MC38 cells to generate tumours. Following inoculation, mouse health was monitored daily including behaviours such as mobility, food and water consumption, body weight, eye, and hair matting. Study group randomisation was performed using “Matched distribution” method (StudyDirector™ software, version 3.1.399.19) when the mean tumour volume was approximately 79 mm³. At randomisation, each mouse was allocated into a study group, and treatment was initiated immediately.

Mice were treated with vehicle only (PBS), 3 mg/kg of a surrogate TPP-1246 molecule that has an alternative albumin-binding domain (TPP-1315) or 5 mg/kg of an Urelumab analogue. Mice were dosed every second day (Q2D) with PBS or TPP-1315 or every third day (Q3D) with the Urelumab analog for a total of 9 doses. Following randomisation/treatment initiation, caliper measurements of each tumour were taken twice per week and tumour volumes determined using the formula V=(L×W×W)/2, where L is the longest tumour dimension, and W is the longest tumour dimension perpendicular to the L. The study was terminated once the PBS group's mean tumour volume exceeded 2000 mm³.

Before the above described study was undertaken the protocol was reviewed and approved by Crown Biosciences' Institutional Animal Care and Use Committee (IACUC) and the care and use of the animals was conducted in accordance with the regulations of the Association for Assessment and Accreditation of Laboratory Animal Care (AAALAC).

### Example 13 Pharmacokinetics Analysis of Single Intravenous Dose of Half-Life Extended Constructs in the Double Transgenic Humanised FcRn/HSA Mouse

Briefly, male or female GenOway Human HSA/FcRn Tg mice were dosed with a single intravenous injection of compounds, listed in the table no 4 (n=3) at either 1 or 2 mg/kg via tail vein. Some of the constructs contained purification/detection tag such as: polyhistidine or FLAG tag. Blood samples were collected at pre-dose and at 0.083 h, 1 h, 8 h, 24 h, 48 h, 72 h and 96 h post drug administration via the saphenous vein. At 168 h post dose all animals were euthanised and blood was collected. Plasma was separated and stored at −80° C. until an assay was carried out. Plasma samples were analysed on the Gyrolab immunoassay platform, using as capture human CD137 and either human CD137Dylight650, human PD-1 Dylight650 or anti-Flag-AF647 rabbit mAb (NEB, cat#15009S) as detection. Data was analysed using Gyros to obtain compound concentrations in plasma. Pharmacokinetic analysis of data was done using PK Solver 2.0, an Excel add on. Results of the study show that compounds have a half-life in the range of 20 to 52 hours when dosed at 2 mg/kg intravenously in human HSA/FcRn Tg mice.

TABLE 28
Summary table for pK parameters.

| | Dose | Cmax | Half-life |
|---|---|---|---|
| Number | (mg/kg) | [ug/ml] | [h] |
| TPP-1010 | 2 | 53.7 | 20.3 |
| TPP-1027 | 2 | 54.5 | 19.9 |
| TPP-1028 | 2 | 61.3 | 51.6 |

### Example 14 PK Study in Cynomolgus Monkey

Prior to initiating the cynomolgus monkey PK study a review of replacement, reduction, and refinement considerations, as well as ethical and scientific justification (e.g. target expression/homology versus human, dose levels, etc), and risks, was conducted both at Crescendo Biologics and the contract research organisation. The animal work in this cynomolgus monkey PK study was conducted under a UK Home Office Project License at a contract research organisation based in the UK. The Home Office license governing this study strictly specifies the limits of severity of effects on the animals. The procedures in the protocol did not cause any effects which exceeded the severity limit of the procedure.

Briefly, three male cynomolgus macaque were dosed with 4 mg/kg of Humabody construct listed in the table (TPP-1246), the study was carried out in Charles River Study. Serum samples were taken at Predose (−24 hrs), 1 hr, 2 hrs, 4 hrs, 8 hrs, 24 hrs, 48 hrs, 72 hrs, 120 hrs, 168 hrs, 216 hrs, 264 hrs, 312 hrs, 360 hrs, 408 hrs and 504 hrs post dose from all test subjects and frozen prior to testing. PK analysis was performed on the serum samples using assays developed at Crescendo.

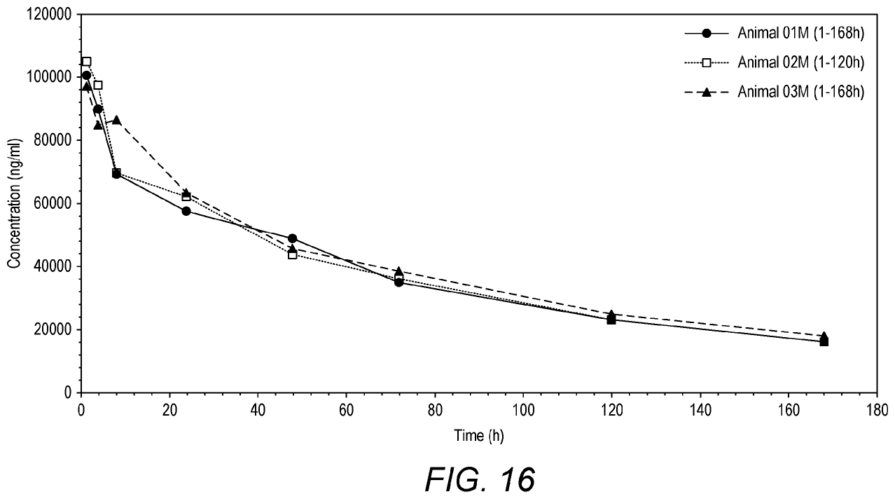

The PK assay utilises the Gyrolab Xplore immunoassay platform using a sandwich immunoassay format; the analyte (listed in the table) is immobilized by biotinylated CD137 antigen and is detected by dyLight650 labelled PD-1. The assay was optimised and established to confirm range and reproducibility and the sample analysis was completed in accordance with the established assays. The PK analysis was performed on the PK data from the following timepoints: Animal 01: 1 to 168 hrs, Animal 02: 1 to 120 hrs and Animal 03: 1 to 168 hrs. The reported PK parameters are the mean from the 3 individuals for each result. The T1/2 of TPP-1246 in Cyno Serum has been demonstrated to be 84.5 hrs±7.58 hrs. Data is shown in FIG. 16.

TABLE 29
PK parameter estimates following a single intravenous
dose of TPP-1246 in cynomolgus macaque

| | Compound | Dose | Cmax | Half-life |
|---|---|---|---|---|
| TPP | description | (mg/kg) | [ug/ml] | [h] |
| TPP-1246 | CD137A-6GS-HSA2- | 4 | 100.6939 | 84.51278 |
| | 6GS-PD138G7(G109D) | | | |

1. An isolated binding molecule comprising
a) a single variable heavy chain domain antibody that binds to CD137 and
b) a single variable heavy chain domain antibody that binds PD-1
wherein the single variable heavy chain domain antibody that binds to CD137 does not cause CD137 signalling when bound to CD137 as a monospecific entity.

2. The isolated binding molecule according to claim 1 wherein the single variable heavy chain domain antibody that binds to PD-1 does not block PD-1 function when used as a monospecific entity.

3. The isolated binding molecule according to claim 1 wherein the single variable heavy chain domain antibody that binds to PD-blocks PD-1 binding to one of its ligands.

4. The isolated binding molecule according to a preceding claim wherein the single variable heavy chain domain antibody that binds to CD137 comprises a set of CDR1, 2 and 3 sequences selected from CDR1, 2, 3 sequences shown in tables 2, 3 or 4 or a sequence with at least 75%, 80%, 90% or 95% homology thereto.

5. The isolated binding molecule according to a preceding claim wherein the single variable heavy chain domain antibody that binds to CD137 comprises a V_{H} sequence as shown in table 2 or a sequence with at least 75%, 80%, 90% or 95% homology thereto.

6. The isolated binding molecule according to a preceding claim wherein the single variable heavy chain domain antibody that binds to CD137 comprises a V_{H} sequence as shown in table 3, 4 or 7 or a sequence with at least 75%, 80%, 90% or 95% homology thereto.

7. The isolated binding molecule according to a preceding claim wherein the moiety that binds to PD-1 is selected from an antibody, an antibody fragment, an antibody mimetic or other polypeptide.

8. The isolated binding molecule according to claim 7 wherein said antibody fragment is selected from a Fab, F(ab′)2, Fv, a single chain Fv fragment (scFv), a single domain antibody or fragment thereof.

9. The isolated binding molecule according to claim 8 wherein said single domain antibody is a V_{H} single domain antibody.

10. The isolated binding molecule according to claim 7 wherein said V_{H} single domain antibody that binds PD-1 and does not block PD-1 function and comprises a comprises a set of CDR1, 2 and 3 sequences selected from CDR1, 2, 3 sequences shown in tables 8, 9 or 10 or a sequence with at least 75%, 80%, 90% or 95% homology thereto.

11. The isolated binding molecule according to any of claims 1, 2, 4 to 10 wherein the single variable heavy chain domain antibody that binds to PD-1 does not block PD-1 function and comprises a full length sequence as listed in table 8, 9 or 10 or a sequence with at least 75%, 80%, 90% or 95% homology thereto.

12. The isolated binding molecule according to any of claims 1, 3, 4 to 9 wherein the single variable heavy chain domain antibody that binds to PD-1 and blocks PD-1 binding to one of its ligands and comprises a CDR or a set of CDRs 1, 2 and 3 selected from table 11 and/or comprises a full length sequence as listed in table 11 or a sequence with at least 75%, 80%, 90% or 95% homology thereto.

13. The isolated binding molecule according to any preceding claim wherein a single domain antibody that has a sequence as shown in table 2, or a sequence with at least 75%, 80%, 90% or 95% sequence homology thereto is linked to a single domain antibody that has a sequence as shown in table 8 or a sequence with at least 75%, 80%, 90% or 95% sequence homology thereto, optionally using a peptide linker.

14. The isolated binding molecule according to any preceding claim capable of binding CD137 with an affinity with a Kd of at least about 10-6 M to about 10-12M.

15. The isolated binding molecule according to any preceding claim capable of binding PD-1 with an affinity with a Kd of about 10-6 M to about 10-12M.

16. The isolated binding molecule according to any preceding claim wherein the single variable heavy chain domain antibody that binds to CD137 is linked to the moiety that binds PD-1 optionally via by a peptide linker or other moiety.

17. The binding molecule according to claim 16 wherein said linker is selected from a (G4S)n linker wherein n is an integer selected from 1 to 50.

18. The isolated binding molecule according to any preceding claim conjugated to one or more moiety selected from a toxin, enzyme, radioisotope, half-life extending moiety, label, therapeutic molecule and other chemical moiety.

19. The isolated binding molecule according to claim 18 wherein said half-life extending moiety is selected from the group consisting of an albumin binding moiety, a transferrin binding moiety, a polyethylene glycol molecule, a recombinant polyethylene glycol molecule, human serum albumin, a fragment of human serum albumin, and an albumin binding peptide and single domain antibody that binds to human serum albumin.

20. The isolated binding molecule according to any preceding claim wherein the single variable heavy chain domain antibody which binds to human CD137 is obtained or obtainable from a transgenic rodent that expresses a transgene comprising human V, D and J regions.

21. The single variable heavy chain domain antibody according to claim 20 wherein said rodent does not produce functional endogenous light and heavy chains.

22. A pharmaceutical composition comprising a binding molecule according to preceding claim and a pharmaceutical carrier.

23. A binding molecule according to any of claims 1 to 21 or a pharmaceutical composition according to claim 19 for use in the treatment of disease.

24. A binding molecule according to any of claims 1 to 21 or a pharmaceutical composition according to claim 22 wherein said disease is cancer, an immune disorder, neurological disease, inflammatory disorder, allergy, transplant rejection, viral infection, (e.g. chronic viral infection) immune deficiency, or other immune system-related disorder.

25. A method for treating cancer comprising administering a therapeutically effective amount of a binding molecule according to any of claims 1 to 21 or a pharmaceutical composition according to claim 22.

26. A nucleic acid molecule comprising a nucleic acid sequence encoding the binding molecule according to any of claims 1 to 21.

27. A vector comprising nucleic acid molecule according to claim 26.

28. A host cell comprising a nucleic acid molecule according to claim 26 or a vector according to claim 27.

29. The host cell according to claim 25 wherein said host cell is a bacterial, yeast, viral or mammalian cell.

30. A method for producing a binding molecule according to any of claims 1 to 21 comprising expressing a nucleic acid encoding said binding molecule in a host cell and isolating the binding molecule from the host cell.

31. A kit comprising a binding molecule according to any one of claims 1 to 21 or a pharmaceutical composition according to claim 22.

32. A use of a binding molecule according to any one of claims 1 to 21 or a pharmaceutical composition according to claim 22 for simultaneously activating downstream signalling pathways of CD137 and PD-1.

33. A method for co-stimulating downstream signalling pathways of CD137 and PD-1 comprising administering a binding molecule according to any of claims 1 to 21 or a pharmaceutical composition according to claim 22.

34. A use of a binding molecule according to any of claims 1 to 21 or a pharmaceutical composition according to claim 22 for inducing a local T cell response in or in the vicinity of a PD-1 positive cell or tissue.

35. An isolated binding molecule comprising
a) an antibody or fragment thereof that binds to CD137 and
b) an antibody or fragment thereof that binds to PD-1.

36. A V_{H} single domain antibody that binds CD137 having a set of CDRs 1, 2 and 3 selected from table 7 and/or which comprises a full length sequence as listed in table 7.

37. A multispecific protein comprising a V_{H} single domain antibody according to claim 36.

38. The multispecific protein according to claim 37 wherein said protein binds to PSMA or PD-1.

39. A binding molecule selected from table 12.
